(19) United States
(12) Patent Application Publication
(10) Pub. No.: US 2024/0002871 A1
(43) Pub. Date: Jan. 4, 2024

(54) RNA Vectors with Hairpin-like Inserts

(71) Applicants: University of Maryland, College Park, College Park, MD (US); Silvec Biologics Inc., Gaithersburg, MD (US)

(72) Inventors: Anne Elizabeth Simon, Upper Marlboro, MD (US); Changyong Jang, Greenbelt, MD (US); Elizabeth Carino, Alexandria, VA (US); Feng Gao, College Park, MD (US)

(73) Assignees: University of Maryland, College Park, College Park, MD (US); Silvec Biologics Inc., Gaithersburg, MD (US)

(21) Appl. No.: 18/311,495

(22) Filed: May 3, 2023

Related U.S. Application Data
(60) Provisional application No. 63/338,290, filed on May 4, 2022.

(51) Int. Cl. C12N 15/82

(52) U.S. Cl. CPC C12N 15/8249; C12N 2796/00

(57) ABSTRACT

The present disclosure relates to a viral vector suitable for introducing a therapeutic agent, such as a peptide, protein or small RNA, into a host or otherwise treating a host. The vector may include an exogenous RNA segment with a hairpin-like structure or having two or more base-paired regions separated by one or more non-base-paired regions. The exogenous RNA segment may have a secondary structure, minimum free energy, average positional entropy or other attributes within specified ranges, or with values similar to one or more hairpin-like structures of a reference wild type virus. Optionally, the RNA vector may be derived from the reference wild type virus or a relative of the reference wild type virus. In some examples, the vector is capable of capable of systemic and phloem-limited movement and replication within a host plant. In some examples, the reference wild type virus is an umbravirus-like associated RNA.

### CROSS-REFERENCE TO RELATED APPLICATIONS

This application claims priority to U.S. Provisional Patent Appln. Ser. No. 63/338,290 (filed on May 4, 2022; pending), which application is hereby incorporated by reference herein in its entirety.

### STATEMENT REGARDING FEDERALLY SPONSORED RESEARCH OR DEVELOPMENT

This invention was made with government support under 20207002933198 awarded by the United States Department of Agriculture (USDA) and under MCB2034359 awarded by the National Science Foundation (NSF). The United States government has certain rights in this invention.

### REFERENCE TO SEQUENCE LISTING

This application includes one or more Sequence Listings pursuant to 37 C.F.R. 1.821 et seq., which are disclosed in computer-readable media (file name: 30059-P72448US02_SequenceListing.xml, created on Sep. 19, 2023, and having a size of 215,368_bytes), which file is herein incorporated by reference in its entirety. The sequences in the listing are RNA sequences, but use “t” to denote uracil pursuant to WIPO st.26 standards.

### FIELD OF THE INVENTION

The present disclosure relates to a vector having an exogenous RNA segment. The vector may be suitable for introducing a therapeutic agent, such as a peptide, a protein or a small RNA, into a host. In some examples the host is a plant, wherein movement of the vector (but not necessarily the agent) is optionally limited to the phloem and the agent may be targeted to control or manage a plant disease or condition.

### BACKGROUND OF THE INVENTION

Both general and highly targeted anti-microbial agents have been developed for animals (e.g., humans) whose circulatory systems provide a delivery system for widespread application throughout the animal. In contrast, much less research has been conducted to develop general or targeted therapeutic agents for non-genetically modified plants since lack of a simplified circulatory system complicates delivery throughout the host plant. This is especially problematic in large, long-lived trees (e.g., citrus), where injection of anti-microbial agents may be rapidly diluted. As a result, few solutions exist for treating systemic plant infections or conditions beyond external application of pesticides, e.g., to control the pathogen's vector during the growing season, foliar applications to strengthen a plant's health in general, or expensive, short-duration injection of agents targeting the pathogen or vector.

Plant industries are at substantial risk from various pathogens. Particularly concerning are diseases and conditions affecting the citrus industry. Huanglongbing (HLB), also known as Citrus Greening, is the most serious citrus disease globally. HLB is associated with three species of the bacterium Candidatus Liberibacter spp. (asiaticus, africanus, and americanus) and is transmitted by two psyllid species, Asian citrus psyllid (ACP) (Diaphorina citri, Kuwayama) and African citrus psyllid (Trioza erytreae, Del Guercio). HLB is graft-transmissible and spreads naturally when a bacteria-containing psyllid feeds on a citrus tree and deposits the pathogenic bacteria into the phloem where the bacteria reproduce. The infected tree reacts by producing excessive callose in its phloem in order to isolate the bacteria, which restricts the flow of photoassimilates and can ultimately kill the tree. Once a tree is infected, there is no cure. While the diseased fruit pose no health threat to humans, HLB has devastated millions of acres of citrus groves throughout the world. In the United States alone, ACP and CL asiaticus (CLas) have decimated the Florida citrus industry, causing billions of dollars of crop losses within a very short time span. Moreover, HLB has spread into every citrus producing region in the United States. Most infected trees die within a few years after infection, and fruit develops misshapen and off flavored and thus is unsuitable for consumption. According to the United States Department of Agriculture (USDA), the entire citrus industry is at substantial risk.

Consideration of plant physiology aids in the development and implementation of strategies for managing plant diseases and conditions. The vascular system of plants is the key conduit for the movement of sugars and amino acids, as well as signaling molecules such as small ribonucleic acids (RNAs), messenger RNAs (mRNAs), proteins, peptides and hormones, which are required for a large number of developmental processes and responses to biotic and abiotic stress (FIG. 1) (Lee, J. Y. and Frank, M. (2018), Plasmodesmata in phloem: different gateways for different cargoes, Curr Opin Plant Biol 43:119-124; Tugeon, R. and Wolf, S. (2009), Phloem Transport: Cellular Pathways and Molecular Trafficking, Ann Rev Plant Biol 60:207-221). Many RNAs comprise a portion of these transiting molecules, and thousands of companion cell mRNAs can be isolated from neighboring enucleated sieve elements, where they are transported bidirectionally by osmotically generated hydrostatic pressure from source (sugar generating) tissue to sink (sugar utilizing) tissue such as roots and shoot tips (Folimonova, S. Y. and Tilsner, J. (2018), Hitchhikers, highway tools and roadworks: the interactions of plant viruses with the phloem, Curr Opin Plant Biol 43:82-88; Ham, B. K. and Lucas, W. J. (2017), Phloem Mobile RNAs as Systemic Signaling Agents, Annual Rev Plant Biol 68:173-195). As much as 50% of the companion cell transcriptome is believed to be engaged in facilitating the movement of such molecules (Kim, G. et al. (2014), Genomic-scale exchange of mRNA between a parasitic plant and its hosts, Science 345:808-811; Thieme, C. J. et al. (2015), Endogenous Arabidopsis messenger RNAs transported to distant tissues, Nature Plants 1(4):15025; Yang, Y. et al. (2015), Messenger RNA exchange between scions and rootstocks in grafted grapevines, BMC Plant Biol 15, 251), which raises various questions with regard to how and why such a substantial subset of mRNAs are moving long-distances. For example, how selective is the process of RNA movement? If there is selection, how is it facilitated? Are transiting RNAs modified (e.g., methylated)? Are transiting RNAs found in any particular subcellular location before exiting into the SE? Are there “address codes” for transiting RNAs to particular locations? Are transiting RNAs bound by specific proteins and are there specific interacting sequences? How much of the flow of mRNAs is biologically meaningful and how much is non-selective, since sink cells are presumably capable of transcribing the same mRNAs?

Confusion in the mRNA movement literature is pervasive. Some studies have indicated that the major determinant of RNA mobility is their abundance in companion cells (Kim, G. et al. (2014), Genomic-scale exchange of mRNA between a parasitic plant and its hosts, Science 345:808-811; Thieme, C. J. et al. (2015), Endogenous Arabidopsis messenger RNAs transported to distant tissues, Nature Plants 1(4):15025; Yang, Y. et al. (2015), Messenger RNA exchange between scions and rootstocks in grafted grapevines, BMC Plant Biol 15, 251). Mathematical modeling has been used to propose a non-selective, Brownian diffusion model for mRNA movement based mainly on their abundance, with half-life and transcript length also playing roles (Calderwood, A. et al. (2016), Transcript Abundance Explains mRNA Mobility Data in Arabidopsis thaliana, Plant Cell 28:610-615). However, other studies have reached opposing conclusions, finding that mRNA abundance in companion cells does not correlate with movement (Xia, C. et al. (2018), Elucidation of the Mechanisms of Long-Distance mRNA Movement in a Nicotiana benthamiana/Tomato Heterograft System, Plant Physiol 177:745-758). In addition, while it is generally assumed that the phloem does not contain RNases that target the transiting RNAs (Morris, R. J. (2018), On the selectivity, specificity and signaling potential of the long-distance movement of messenger RNA, Curr Opin Plant Biol 43:1-7), Xia et al. also found that most mobile mRNAs are degraded and never reach the root or upper stem. Other studies found that the presence of a predicted tRNA-like structure is associated with over 11% of mobile mRNAs (Zhang, W. N. et al. (2016), tRNA-Related Sequences Trigger Systemic mRNA Transport in Plants, Plant Cell 28:1237-1249), suggesting that mobile mRNAs might harbor specific “zip-codes”. However, other abundant mRNAs containing similar tRNA-like motifs were not mobile (Xia, C. et al. (2018), Elucidation of the Mechanisms of Long-Distance mRNA Movement in a Nicotiana benthamiana/Tomato Heterograft System, Plant Physiol 177:745-758). Thus, prior studies have failed to identify and develop a model system consisting of a highly abundant, mobile RNA whose movement is traceable in living tissue under different cellular conditions.

Plant viruses, many of which move through the plant as a ribonucleoprotein complex (vRNP), have evolved to use the same pathway as used by mobile endogenous RNAs. Plant viruses can accumulate in substantial amounts, and most initiate infection in epidermal or mesophyll cells and then move cell-to-cell through highly selective intercellular connectors called plasmodesmata, which allow for continuity between the cytoplasm of neighboring cells (FIG. 1; see also Lee, J. Y. and Frank, M. (2018), Plasmodesmata in phloem: different gateways for different cargoes, Curr Opin Plant Biol 43:119-124; Schoelz, J. E. et al. (2011), Intracellular transport of plant viruses: finding the door out of the cell, Mol Plant 4:813-831). Long-distance systemic movement (leaf-to-leaf) requires that the virus enters companion cells, where replication takes place, followed by progeny exit into sieve elements by transiting through the specialized, branched plasmodesmata that connect companion cells and sieve elements. Once tubular sieve elements are reached, viruses move passively with the phloem photoassimilate stream and establish systemic infections upon exiting (Folimonova, S. Y. and Tilsner, J. (2018), Hitchhikers, highway tolls and roadworks: the interactions of plant viruses with the phloem, Curr Opin Plant Biol 43:82-88).

For viruses that transit through the phloem as viral nucleoproteins (vRNPs), movement is similar to that of host mRNAs. All plant viruses encode at least one movement protein necessary for movement, which binds to viral RNA and also dilates plasmodesmata. Thus, host mRNA movement also likely requires similar host-encoded movement proteins. Viral movement proteins are non-specific RNA binding proteins. However, questions remain with regard to how vRNPs load into the phloem and unload in distal tissues, although reprograming companion cell gene expression may be required (Collum, T. D. et al. (2016), Tobacco mosaic virus-directed reprogramming of auxin/indole acetic acid protein transcriptional responses enhances virus phloem loading, Proc Natl Acad Sci USA 113:E2740-E2749). If mRNA trafficking is so widespread and non-specific, it has remained unclear why RNA viruses require their own encoded movement proteins. Some researchers have suggested that RNA viruses require movement proteins if they move as preformed replication complexes that include a large RNA-dependent RNA polymerase (Heinlein, M. (2015), Plant virus replication and movement, Virology 479:657-671), which is beyond the size-exclusion limit (˜70 kDa) of companion cell plasmodesmata. It has also remained unclear why and how some viruses are phloem-limited. For example, phloem-limited closteroviruses have at least 3 movement proteins, and phloem-limitation can be relieved by over-expressing the silencing suppressor and downregulating host defenses (Folimonova, S. Y. and Tilsner, J. (2018), Hitchhikers, highway tolls and roadworks: the interactions of plant viruses with the phloem, Curr Opin Plant Biol 43:82-88), suggesting that phloem-limitation is a complex process for some viruses. Phloem-limitation can also be an active process (as opposed to lack of a cell-to-cell movement protein). For example, altering a domain of the Potato leaf role virus movement protein conferred the ability to exit the phloem (Bendix, C., and Lewis, J. D. (2018), The enemy within: phloem-limited pathogens, Mol Plant Path 19:238-254).

A direct connection between host movement of mRNAs and vRNP movement was established when the origin of plant virus movement proteins was solved. A pumpkin protein (RPB50) related to the Cucumber mosaic virus movement protein was discovered that was capable of transporting its own mRNA, as well as other mRNAs, into the phloem (Xoconostle-Cazares, B. et al. (1999), Plant paralog to viral movement protein that potentiates transport of mRNA into the phloem, Science (New York, NY) 283:94-98; Ham, B. K. et al. (2009), A polypyrimidine tract binding protein, pumpkin RBP50, forms the basis of a phloem-mobile ribonucleoprotein complex, Plant Cell 21:197-215). A complex population of these endogenous movement proteins, known as non-cell-autonomous proteins (NCAPs), have been proposed as being responsible for the long-distance phloem trafficking of mRNAs (Gaupels, F. et al. (2008), Nitric oxide generation in Vicia faba phloem cells reveals them to be sensitive detectors as well as possible systemic transducers of stress signals, New Phytol 178:634-646; Gomez, G. et al. (2005), Identification of translocatable RNA-binding phloem proteins from melon, potential components of the long-distance RNA transport system, Plant J 41:107-116; Kim, M. et al. (2001), Developmental changes due to long-distance movement of a homeobox fusion transcript in tomato, Science (New York, NY) 293:287-289; Pallas, V. and Gomez, G. (2013), Phloem RNA-binding proteins as potential components of the long-distance RNA transport system, Front Plant Sci 4:130; Yoo, B. C. et al. (2004), A systemic small RNA signaling system in plants, Plant Cell 16:1979-2000).

Since their discovery (Deom, C. M. et al. (1987), The 30-kilodalton gene product of tobacco mosaic virus potentiates virus movement, Science (New York, NY) 237:389-394), a number of viral movement proteins have been identified that are responsible for intracellular trafficking of vRNPs to the plasmodesmata, as well as for cell-to-cell and long-distance movement (Tilsner, J. (2014), Techniques for RNA in vivo imaging in plants, J Microscopy 258(1):1-5). For some viruses (e.g., umbraviruses), cell-to-cell and long-distance movement is associated with multiple movement proteins (Ryabov, E. V. et al. (2001), Umbravirus-encoded proteins both stabilize heterologous viral RNA and mediate its systemic movement in some plant species, Virology 288:391-400). For example, closteroviruses such as Citrus tristeza virus contain three movement proteins. However, for many viruses, all movement activities are thought to be associated with a single movement protein.

Delivering engineered therapeutic agents into plants for combating diseases, insects or other adverse conditions (e.g., HLB and/or the carrier insects) using virus vectors is an established means of introducing traits such as resistance to pathogens or other desired properties into plants for research purposes. Various methods of providing vectors to plants are known in the art. This is often achieved by delivery of the virus vector into a plant cell's nucleus by Agrobacteria tumefactions-mediated “agroinfiltration,” which may result in a modification of that cell's genome, or by delivering the virus vector directly into a cell's cytoplasm, which results in infection without a requirement for genomic modification. In the case of agroinfiltration of RNA viruses, the cDNA of the viral genome is incorporated into the T-DNA, which Agrobacteria delivers into the plants. Such T-DNA includes further regulatory DNA components (e.g., promoter for RNA polymerase), which allow for transcription of the viral genome within plant cells. The incorporated virus, containing therapeutic DNA inserts, is transcribed into RNA within the plant cells, after which the virus behaves like a normal RNA virus (amplification and movement).

Thus, to act as an effective vector, a virus should be engineered to accept inserts without disabling its functionality and to ensure that the engineered virus is able to accumulate systemically in the host to a level sufficient to deliver and in some cases express the insert(s). These inserts, whether having open reading frames (ORFs) that will be translated into proteins or non-coding RNAs that will be used for a beneficial function, should be delivered into the targeted tissue in a manner that is effective and sufficiently non-toxic to the host or to any downstream consumption of the host or the environment. However, only a limited number of viral vectors exist that meet the above criteria and these are available for only certain plants (e.g., citrus tristeza virus for citrus). Unfortunately, there is either no known suitable viral vector, or only suboptimal viral vectors, for most plants, particularly for long lived trees and vines. Moreover, maintaining stability of short sequences inserted into a vector has raised numerous challenges. As apparent from prior research on virus vectors, the ability to stabilize inserted sequences utilizing conventional methodologies has not been successful, particularly for long periods (e.g., months or years). Conventional vectors quickly evolve or mutate, discarding all or part of the inserted sequences since the native virus (without inserts) is generally more fit than viral vectors containing such inserts.

Thus, the ability to implement RNA or DNA therapies on a broad basis is substantially limited with existing technologies. Over 1,000 plant viruses have been identified with many plants subject to infection by multiple viruses. For example, citrus trees are subject to citrus leaf blotch virus, citrus leaf rugose virus, citrus leprosis virus C, citrus psorosis virus, citrus sudden death-associated virus, citrus tristeza virus (CTV), citrus variegation virus, citrus vein enation virus and citrus yellow mosaic virus, among others. However, CTV, the causal agent of catastrophic citrus diseases such as quick decline and stem pitting, is currently the only virus that has been developed as a vector for delivering agents into citrus phloem.

CTV is a member of the genus Closterovirus. It has a flexuous rod-shaped virion composed of two capsid proteins with dimensions of 2000 nm long and 12 nm in diameter. With a genome of over 19 kb, CTV (and other Closteroviruses) are the largest known RNA viruses that infect plants. It is a virulent pathogen that is responsible for killing, or rendering useless, millions of citrus trees worldwide, although the engineered vector form is derived from a less virulent strain, at least for Florida citrus trees (still highly virulent in California trees). Prior studies have purportedly demonstrated that CTV-based vectors can express engineered inserts in plant cells (U.S. Pat. No. 8,389,804; US 20100017911 A1). However, it has not been commercialized due to its inconsistent ability to accumulate in plants and achieve its targeted beneficial outcome. It is thought that CTV's inability to replicate to sufficiently high levels and heat sensitivity limits its ability to generate a sufficient quantity of RNA for treatment.

Thus, CTV-based vectors have a very limited ability to deliver an effective beneficial payload where needed. Moreover, CTV is difficult to work with due to its large size. CTV is also subject to superinfection exclusion, wherein a CTV-based vector is unable to infect a tree already infected with CTV. In addition, strains suitable for one region (e.g., Florida) are unsuitable for varieties of trees in another region (e.g., California). CTV also encodes three RNA silencing suppressors making its ability to generate large amounts of siRNAs problematic. Despite such problems, CTV is the only viral vector platform available for citrus trees.

Accordingly, there is a need for an alternative infectious agent, optionally that solves some or all of the above-noted problems, and which is capable of introducing a desirable property and/or delivering a therapeutic agent(s) into a plant, optionally for an extended period of time, particularly a long-lived plant such as a tree or vine.

### SUMMARY OF THE INVENTION

Viral vectors may be derived from a wild-type virus and modified with an exogenous insert. The presence of some or all of the wild-type viral genome allows for replication of the vector, either in the host being treated or in manufacture outside of a host. The exogenous insert provides an active agent to achieve some form of activity from the vector. In some examples, the exogenous insert includes RNA that will be converted by the plant into a small interfering RNA (siRNA), which is typically 21-24 nucleotides (nt) in length. In one conventional method, the siRNA sequence is included in a base-paired, double-stranded (“hairpin”) structure. The siRNA sequence extends along one side of the hairpin, and a complementary base-paired sequence extends along the other side of the hairpin. The two sides of the hairpin are separated by an apical loop. Small hairpins are easier to work with, and so the size of the siRNA haipin is typically less than 60 nt.

Hairpins can be highly stable in a structural sense. For example, each base may have minimal or no positional entropy (PE), and each base pair may have a high probability of forming. However, fully base-paired hairpins, particularly hairpins with many G-C base pairs, can result in vectors with low stability for replication, i.e. the vectors are unable to maintain the inserts within their genome as they replicate. As the vector replicates, the hairpins may be deleted from some progeny of the vector. Progeny without the hairpin may be closer to the wild type vector. Since the wild type virus has evolved to be optimally fit, vector progeny without the hairpin may out compete vector progeny with the hairpin until eventually the hairpin in lost. Accordingly, while a viral vector with a hairpin can remain effective for a period of time, the stability of viral vectors may be further improved. Further, it is desirable to increase the size of the targeting sequence. Even when producing a VIGS vector, although the inserts are cut into segments of 21-24 nt in the plant it is beneficial to provide a longer targeting sequence that may be cut into more than one 21-24 nt sequence, or a targeting sequence complementary to multiple targets.

This specification describes RNA inserts for a vector that are hairpin-like, or include a hairpin-like region, but not are not conventional fully base-paired hairpins (i.e., the hairpin-like inserts are not fully base-paired outside of their apical loop). In at least some examples, these inserts carry larger targeting sequences and/or provide increased stability for replication than previously described inserts. Increased stability may involve a lower incidence of replicates that have deleted the insert at a given time point, or by replicates with the insert being detectable in the host for a longer period of time. The words stable and unstable may be used herein as relative terms. Inserts described as unstable may have some stability and could be useful for some applications. Hairpins that are described as stable are stable relative to other hairpins, but less stable than stable hairpin-like structures.

This specification also describes vectors that include an exogenous segment. In some examples, the exogenous segment is a hairpin-like structure having two or more base-paired regions, one or more non-base-paired regions separating the base-paired regions, and an apical loop at the end of one of the based paired regions. In some examples, the exogenous segment further complies with one or more design guidelines or parameters. These guidelines or parameters for the exogenous segment may include: the average positional entropy (APE) is in the range of 0.01 to 0.75; the length of the exogenous segment is 300 nt or less; the maximum length of a base-paired region is 19 base pairs; the maximum APE of a base-paired region is 0.8; the maximum number of consecutive G:C pairs in a base-paired region is 4; the maximum number of bases in a non-base-paired region is 20; the ΔG of the exogenous segment is within a range of −5 to +15 kcal/mol or within 10 kcal/mol (+ or −) of the ΔG of a naturally occurring hairpin of similar length; the standard deviation of PE is less than 0.5, less than 15% of bases have a PE greater than 1; the largest PE of any base is not greater than 2.0; and the insert, not considering the apical loop, is 65-90% base-paired. Although inserts are preferably hairpin-like structures, optionally a hairpin may be designed according to one or more of the design guidelines or parameters described herein.

In some examples, a vector includes an exogenous segment that has been designed to mimic a model hairpin-like structure in a wild type virus. In some examples, the exogenous segment mimics the secondary structure of the wild type hairpin-like structure by having a similar arrangement of base-paired regions and non-base-paired region. In some examples, the exogenous segment mimics the secondary structure of the wild-type hairpin-like structure by having a similar ΔG relative to length. Optionally, the vector is derived from the wild type virus having the model hairpin-like structure, or one or more relatives of that wild type virus. A mimicked exogenous segment may also follow one or more of the guidelines or parameters described above. Alternatively, a mimicked exogenous segment may follow one or more analogous guidelines or parameters derived from a study of hairpin-like structures in a wild type virus that the vector is derived from, or one or more relatives of that wild type virus.

In the detailed description, we describe the complete secondary structure of citrus yellow vein associated virus (CYVaV) and some or all of the secondary structure of some of its related umbravirus-like associated RNA (ulaRNA). CYVaV and other ulaRNA are able to replicate and move systemically in plants, in many cases with minimal or no symptoms of infection. CYVaV and other ulaRNA can be developed into vectors to control vascular diseases in trees and vines and/or to target plant pathogens such as insects, fungi and other viruses. In some examples, a vector is derived from a ulaRNA, a Class 2 ulaRNA or CYVaV. These vectors may have exogenous inserts that comply with one or more design guidelines or parameters described above, or mimic a naturally occurring hairpin-like structure in a ulaRNA, a Class 2 ulaRNA or CYVaV. Although the detailed description is focused on ulaRNA, the invention is not limited to ulaRNA.

The present disclosure is also related to a modified vector, for example r a virus induced gene silencing (VIGS) vector. The vector is modified by the addition of a heterologous element that comprises one or more nucleotide sequences not found in the wild-type virus (exogenous segments). The exogenous segment(s) may substantially mimic the secondary structure of a portion, for example a hairpin-like portion, of the wild type vector (e.g. virus), or of a relative of the wild type vector. A hairpin-like portion and its corresponding exogenous segment may each comprise two or more base-paired regions and one or more non-base-paired regions. In some examples, a hairpin-like portion and exogenous segment each comprise one or more non-base-paired regions each located between two base-paired regions. In some examples, the non-base-paired region may be single sided or double sided and form a “loop” or “bulge” between two base-paired regions in the secondary structure of the vector. In some examples, the exogenous segment may have a sequence with many, for example 60 or more, 70 or more, 80 or more or 100 or more, bases, or changes in bases relative to the wild type vector. The exogenous segment may thereby contain an active (i.e. targeted) sequence that is 28 nt or more, 30 nt or more, 35 nt or more, 40 nt or more, or 50 nt or more, in length. In some examples, the exogenous segment contains at least one non-base-paired region. In some examples, an exogenous segment has a minimum free energy similar to a double stranded or hairpin-like portion of the wild type vector. In some examples, an exogenous segment has an active portion, for example an siRNA targeted to a pathogen or a gene silencing RNA targeted at a host plant.

In some examples, an exogenous segment is inserted into a vector a) in the location of, and as a replacement for, a hairpin-like structure that the exogenous segment mimics, b) in the location of, and as a replacement for, a hairpin-like structure that the exogenous segment does not mimic (wherein the exogenous segment mimics a different hairpin-like structure optionally of the vector or a relative of the vector), or c) at a location that previously did not have any hairpin-like structure (wherein the exogenous segment mimics a hairpin-like structure, optionally of the vector or a relative of the vector).

The present disclosure also relates to a novel infectious agent(s) capable of delivering and stably maintaining an exogenous insert(s) into a plant, compositions comprising a plant infected by the disclosed agent(s), and methods and uses relating thereto. The disclosed agents are sometimes referred to herein as “independently mobile RNAs” or “iRNAs.” Despite being infectious single-stranded RNAs, some iRNAs do not encode for any movement protein(s). They also do not encode RNA silencing suppressors, which are a key characteristic of plant viruses. In addition, unlike virtually all plant RNA viruses, with the exception of umbraviruses, and contrary to some definitions of a virus, iRNAs also do not encode a coat protein for encapsidating the RNA into virions, which is a requirement for vectored movement of viruses from plant to plant. Despite the lack of movement protein expression in some examples, iRNAs are able to move systemically within the phloem in a host plant. As compared to viruses, iRNAs have additional advantageous properties, such as: the ability to accumulate to levels exceeding those of most known plant viruses; possessing a relatively small size, e.g., being only about two-thirds the size of the smallest plant RNA virus and thus much easier to work with compared to such conventional plant RNA viruses; and exhibiting the inability to spread on their own to other plants (given their inability to encode for any coat protein). The lack of a silencing suppressor allows the immune system of the host plant to slice or break up the iRNA, thereby releasing siRNAs from the exogenous inserts of the iRNA into the plant. iRNA include umbravirus-like associated RNA (ulaRNA).

In accordance with disclosed embodiments, an infectious agent comprises an RNA, e.g. an iRNA, which may contain one or more engineered insert(s), sometimes referred to herein as a heterologous segment(s) or alternatively as exogenous segment(s), which, for example, triggers in a plant expression of a targeted peptide, protein(s) and/or produces targeted siRNA or other non-coding RNA that are cleaved from the vector for beneficial application, and/or delivers a therapeutic agent into the plant, and/or otherwise effectuates or promotes via such targeting or delivery a beneficial or desired result. Aspects of the present disclosure include: an iRNA-based vector for delivery of targeted anti-pathogenic agents; an anti-bacterial enzybiotic targeted at bacteria infecting a plant or bacteria required by the insect vector; an enzybiotic that is generated from an internal ribosome entry site (IRES) of the tobacco etch virus (TEV) (TEV IRES); incorporation of siRNAs into the iRNA genome; incorporation of inserts into a lock and dock structure to stabilize the base of a scaffold that supports the inserts; incorporation of sequences that may be cut into siRNAs into an iRNA genome that has been modified to enhance the structural stability of the local region to counter the destabilizing effects of the inserts; incorporation of an siRNA that disrupts or kills a targeted insect vector; incorporation of an siRNA that mitigates the negative impacts of a tree's callose production; incorporation of an siRNA that mitigates the plant's recognition of the pathogen; incorporation of an siRNA or other agent that targets bacterial, viral or fungal pathogens; and incorporation of an insert that triggers a particular plant trait (e.g., dwarfism). Thus, the infectious agents and compositions disclosed herein possess superior and advantageous properties as compared to conventional technologies.

The iRNA-based vectors of the present disclosure are suitable for use as a general platform for expression of various proteins and/or delivery of small RNAs into the phloem of citrus and other host plants. In some implementations, a citrus yellow vein associated virus (CYVaV)-based vector is provided, which accumulates to massive levels in companion cells and phloem parenchyma cells. The vectors of the present disclosure may be utilized to examine the effects of silencing specific gene expression, e.g., in the phloem (and beyond) of trees. In addition, CYVaV may be developed into a model system for examining long-distance movement of mRNAs through sieve elements. Since CYVaV is capable of infecting virtually all varieties of citrus, with few if any symptoms generated in the infected plants, movement of RNAs within woody plants may be readily examined.

In accordance with disclosed embodiments, the present disclosure is directed to a plus-sense single stranded ribonucleic acid (RNA) vector comprising a replication element(s) and a heterologous segment(s), wherein the RNA vector lacks a functional coat protein(s) open reading frame(s) (ORFs) and optionally lacks a functional movement protein ORF. The RNA vector is capable of movement in a host plant, for example systemic movement, movement through the phloem, long-distance movement and/or movement from one leaf to another leaf. In some implementations, the RNA vector also lacks any silencing suppressor ORF(s). In some implementations, the RNA vector comprises a 3′ Cap Independent Translation Enhancer (3′ CITE) comprising the nucleic acid sequence(s) of SEQ ID NO:4 and/or SEQ ID NO:5. In some embodiments, the 3′ CITE comprises the nucleic acid sequence of SEQ ID NO:3.

In some embodiments, the replication element(s) of the RNA vector comprises one or more conserved polynucleotide sequence(s) having the nucleic acid sequence of: SEQ ID NO:10, SEQ ID NO:11, SEQ ID NO:12, SEQ ID NO:13, and/or SEQ ID NO:14. In some implementations, the replication element(s) additionally or alternatively comprises one of more conserved polynucleotide sequence(s) having the nucleic acid sequence of: SEQ ID NO:15 and/or SEQ ID NO:16.

In some embodiments, the RNA vector is derived from citrus yellow vein associated virus (CYVaV) (SEQ ID NO:1) or an iRNA or ulaRNA relative thereof, for example a Class 2 ulaRNA. The RNA vectors of the present disclosure are capable of systemic and phloem-limited movement and replication within a host plant. The RNA vectors of the present disclosure are functionally stable for replication, movement and/or translation within the host plant for at least one month after infection thereof, more preferably for at least 3 months, at least 6 months, at least 12 months, or at least 2 years, after infection thereof. In preferred embodiments, the RNA vectors and inserts thereof are functionally stable for the life of the host plant (e.g. 5-10 years or more).

In some embodiments, the heterologous segment(s) of the RNA vector of the present disclosure comprises a polynucleotide that encodes at least one polypeptide selected from the group consisting of a reporter molecule, a peptide, and a protein or is an interfering RNA. In some implementations, the polypeptide is an insecticide or an insect control agent, an antibacterial, an antiviral, or an antifungal. In some implementations, the antibacterial is an enzybiotic. In some implementations, the antibacterial targets a bacterium Candidatus Liberibacter species, e.g. Candidatus Liberibacter asiaticus (CLas).

In some embodiments, the heterologous segment(s) of the RNA vector of the present disclosure comprises a small non-coding RNA molecule and/or an RNA interfering molecule. In some implementations, the small non-coding RNA molecule and/or the RNA interfering molecule targets an insect, a bacterium, a virus, or a fungus. In some implementations, the small non-coding RNA molecule and/or the RNA interfering molecule targets a nucleic acid of the insect, the bacterium, the virus, or the fungus. In some implementations, the small non-coding RNA molecule and/or the RNA interfering molecule targets a virus, for example a virus selected from the group consisting of Citrus vein enation virus (CVEV) and Citrus tristeza virus (CTV). In some implementations, a targeted bacteria is Candidus Liberibacter asiaticus (CLas). In some implementations, the iRNA comprises an siRNA hairpin or hairpin-like structure that targets and renders the targeted bacteria non-pathogenic.

It should be understood that the RNA vector may include multiple heterologous segments, each providing for the same or different functionality. In some embodiments, the heterologous segment(s) is a first heterologous segment, wherein the RNA vector further comprising a second heterologous segment(s), wherein the replication element(s) is intermediate the first and second heterologous segments.

In some embodiments, the heterologous segment(s) of the RNA vector of the present disclosure comprises a polynucleotide that encodes for a protein or peptide that alters a phenotypic trait. In some implementations, the phenotypic trait is selected from the group consisting of pesticide tolerance, herbicide tolerance, insect resistance, reduced callose production, increased growth rate, and dwarfism.

The present disclosure is also directed to a host plant comprising the RNA vector of the present disclosure. The host plant may be a whole plant, a plant organ, a plant tissue, or a plant cell. In some implementations, the host plant is in a genus selected from the group consisting of citrus, vitis, ficus and olea. In some implementations, the host plant is a citrus tree or a citrus tree graft.

The present disclosure also relates to a composition comprising a plant, a plant organ, a plant tissue, or a plant cell infected with the RNA vector of the present disclosure. In some implementations, the plant is in a genus selected from the group consisting of citrus, vitis, ficus, malus, and olea. In some implementations, the plant is a citrus tree or a citrus tree graft.

The present disclosure also relates to a method for introducing a heterologous segment(s) into a host plant comprising introducing into the host plant the RNA vector of the present disclosure. In some embodiments, the step of introducing the heterologous segment(s) into the host plant comprises grafting a plant organ or plant tissue of a plant that comprises the RNA vector of the present disclosure to a plant organ or plant tissue of another plant that does not comprise the RNA vector prior to said introduction. The RNA vectors of the present disclosure are capable of systemically infecting the host plant.

The present disclosure is also directed to a process of producing in a plant, a plant organ, a plant tissue, or a plant cell a heterologous segment(s), comprising introducing into said plant, said plant organ, said plant tissue or said plant cell the RNA vector of the present disclosure. In some embodiments, the plant is in a genus selected from the group consisting of citrus, vitis, ficus and olea.

The present disclosure also relates to a kit comprising the RNA vector of the present disclosure.

The present disclosure is also directed to use of the RNA vector(s) of the present disclosure for introducing the heterologous segment(s) into a plant, a plant organ, a plant tissue, or a plant cell. The present disclosure is also directed to use of the host plant(s) of the present disclosure, or use of the composition(s) of the present disclosure, for introducing the RNA vector(s) into a plant organ or plant tissue that does not, prior to said introducing, comprise the RNA vector. In some implementations, the step of introducing the RNA vector comprises grafting a plant organ or plant tissue of a plant that comprises the RNA vector to a plant organ or plant tissue of another plant that does not comprise the RNA vector.

The present disclosure is also directed to a method of making a vector for use with a plant comprising the steps of inserting one or more heterologous segment(s) into an RNA, wherein the RNA is selected from the group consisting of: CYVaV; a relative of CYVaV; other RNA vectors having least 50% or at least 70% RdRp identity with CYVaV; and another iRNA or ulaRNA. The present disclosure also relates to a vector produced by the disclosed method(s).

The present disclosure also relates to the use of an RNA molecule as a vector, wherein the RNA is selected from the group consisting of: CYVaV; a relative of CYVaV; other RNA vectors having at least 50% or at least 70% RdRp identity with CYVaV; and, another iRNA or ulaRNA. In some implementations, the RNA is used in the treatment of a plant, for example the treatment of a viral or bacterial infection of a plant, for example the treatment of CTV infection or Citrus Greening in a citrus plant, or in the control of insects that are vectors and/or feed on the plant. The RNA is modified with one or more inserted heterologous segment(s), for example an enzybiotic or an siRNA.

The present disclosure is also directed to the use of an RNA molecule characterized by being in the manufacture of a medicament to treat a disease or condition of a plant, wherein the RNA is selected from the group consisting of: CYVaV; a relative of CYVaV; other RNA vectors having at least 50%, or at least 70%, identity with the RdRp of CYVaV; and, another iRNA or ulaRNA. In some implementations, the disease or condition is a viral or bacterial infection of a plant, for example CTV or Citrus Greening in a Citrus plant.

The present disclosure is also directed to an RNA molecule for use as a medicament or in the treatment of a disease or condition of a plant, wherein the RNA is selected from the group consisting of: CYVaV; a relative of CYVaV; other RNA vectors having at least 50% or at least 70% RdRp identity with CYVaV; and, another iRNA or ulaRNA.

The present disclosure is also related to a ribonucleic acid (RNA) vector, for example a plus-sense single stranded ribonucleic acid (RNA) vector, comprising one or more heterologous segment(s), wherein said heterologous element(s) is attached to the main structure of the RNA vector through a lock and dock structure, optionally a branched structure comprising an insert site for the heterologous element and a relatively stable and/or locking structure that does not participate in folding of the heterologous element or the main structure of the RNA vector. In some implementations, the RNA vector is an iRNA or ulaRNA-based vector or a virus-based vector. In some implementations, a lock portion of the lock and dock structure comprises a scaffold normally used for crystallography. In some implementations, the lock and dock structure comprises a branched element, wherein a stem and a branch of the branched element are located within a relatively stable structure forming the lock, such as a tetraloop-tetraloop dock, e.g., a GNRA tetraloop docked into its docking sequence, and another branch of the branched element comprises an insert site for the heterologous element. In some implementations, the heterologous element is a hairpin or an unstructured sequence.

The present disclosure is also related to an iRNA or ulaRNA-based vector having one or more heterologous segment(s) having a sequence that targets a particular pathogen, e.g., such as a virus, a fungus, or a bacteria. In some implementations, the siRNA is effective against a plant pathogenic bacterium. In some implementations, the siRNA targets a Candidatus Liberibacter species such as Candidatus Liberibacter asiaticus (CLas).

The present disclosure is also related to a vector having a heterologous element comprising a hairpin or hairpin-like structure having a sequence on one side complementary to a sequence within citrus tristeza virus (CTV) or an unstructured sequence complementary to the plus or minus strand of CTV. In some implementations, the sequence within CTV is conserved in multiple CTV strains. In some implementations, the sequence one on side of the hairpin or hairpin-like structure is complementary with a sequence in multiple CTV strains, or all known CTV strains, despite differences in CTV sequences. The present disclosure is also related to a plant having a sour orange rootstock and an iRNA or ulaRNA-based vector having a heterologous element that targets Citrus tristeza virus.

The present disclosure is also related to a method for introducing a heterologous segment(s) into a host plant comprising introducing into said host plant an iRNA or ulaRNA-based vector after a) encapsidating the iRNA or ulaRNA vector in a capsid protein other than the capsid protein of CVEV, or b) by coating the iRNA or ulaRNA with phloem protein 2 (PP2) from sap extracted from cucumber, citrus or other plant, c) by using dodder to take up sap from infected laboratory host and transmit to a secondary host, e) by encapsidating the iRNA or ulaRNA in virions of CVEV and infecting plants by stem slashing or stem peeling, or f) by feeding CYVaV-containing virions to a CVEV-specific aphid vector and then allowing the aphids to feed on trees.

The present disclosure is also related to an iRNA or ulaRNA-based vector comprising one or more inserts at one or more of positions 2250, 2301, 2304, 2317, 2319, 2330, 2331, 2336, 2375 and 2083 of a CYVaV-based RNA. All of these locations are in the 3′ UTR. Inserts up to 200 nt, or possibly more, can be inserted. Multiple inserts may be used in a single vector. In some implementations, the iRNA or ulaRNA-based vector is stabilized, for example by converting G:U pairs to G:C pairs in the 3′UTR structure. In some implementations, the insert is appended to a truncated hairpin at the 5′ end of the 3′ UTR.

The present disclosure is also related to a method of making a ribonucleic acid (RNA) vector comprising stabilizing the 3′ UTR structure of a parental construct and inserting one or more destabilizing heterologous segment(s) into the stabilized parental construct.

The present disclosure describes many CYVaV-based vectors, but in some implementations analogous vectors and/or inserts are produced using another iRNA or ulaRNA or an unrelated RNA or virus as the starting material or sequence. In these implementations, descriptions relating to CYVaV may be modified accordingly. For example, positions described for CYVaV may be substituted with a corresponding position in another type of iRNA or ulaRNA or RNA or virus.

In some implementations, an iRNA or ulaRNA-based vector or a virus-based vector is constructed using starting material (i.e., an iRNA or ulaRNA or virus) obtained from the wild, or multiplied cloned or otherwise reproduced from starting material obtained from the wild. The starting material is modified, for example to change, delete and/or replace, one or more elements of the wild-type structure and/or to add one or more inserts. In other implementations an iRNA or ulaRNA-based vector or virus-based vector is synthetic. For example, an iRNA or ulaRAN-based vector or virus-based vector may be made by creating a synthetic replica of the wild-type RNA and then modifying the synthetic replica, or directly creating a synthetic replica of a modified RNA.

The present disclosure is also related to a method of making a ribonucleic acid (RNA) vector comprising truncating a hairpin or hairpin-like structure in a parental construct and inserting one or more heterologous segment(s) into the truncated parental construct.

The present disclosure is also related to compositions and methods comprised of combinations or sub-combinations of one or more other compositions or methods described herein, to compositions produced by methods described herein, to methods of making compositions described herein, and to methods of treating plants using compositions described herein.

The present disclosure relates to a single stranded RNA vector suitable for introducing a therapeutic agent such as a small RNA into a host plant, or otherwise treating a host plant. The vector, such as iRNA or ulaRNA as described herein, optionally does not encode for any movement protein and does not encode for a coat protein, but is capable of capable of systemic and phloem-limited movement and replication within the host plant. The vector may be modified to include an siRNA effective against a bacterial plant pathogen. The plant pathogen may be, for example, Pseudomonas syringae, Envinia amylovora and Liberibacter asiaticus. The siRNA may be, for example, a complement of the adenylate kinase (ADK) or gyrase subunit A (GyrA) gene of the bacteria. Alternatively, the wild type vector may be introduced into the plant to inhibit or control a bacterial infection in the plant by way of non-specific siRNA created by the RNA silencing or transitive silencing mechanism of the plant. Alternatively, the vector may be modified to include an insert that increases a silencing mechanism of the plant, for example an insert that is a complement to a plant virus. For example, CYVaV or another iRNA or ulaRNA with an insert that complements a portion of citrus tristeza virus (CTV) may be introduced into a citrus tree to treat citrus greening.

### BRIEF DESCRIPTION OF THE DRAWINGS

The priority application of the present application contains at least one drawing/photograph executed in color. Copies of such color drawing(s) will be provided by the United States Patent & Trademark Office upon request and payment of the required fee.

FIG. 1 illustrates schematically the movement pathways through the vascular system of plants (Lee, J. Y. and Frank, M. (2018), Plasmodesmata in phloem: different gateways for different cargoes, Curr Opin Plant Biol 43:119-124).

FIG. 2 is a phylogenic tree based on the amino acid (Panel A) and nucleotide (Panel B) sequence of RdRp from umbravirus-like associated RNAs (ulaRNAs), 6 tombusvirus-like associated RNAs (tlaRNAs) and 24 viruses from the umbravirus, tombusvirus and betacarmovirus genera. Branch numbers indicate bootstrap support in percentage out of 1000 replicates. The scale bar denotes nucleotide/protein substitutions per site. Both trees were mid-point rooted. BabVQ: babaco virus Q (MN648673); CMoV: virus (FJ188473); CMoMV: carrot mottle mimic virus (U57305); CYVaV: citrus yellow vein associated virus (JX101610); EMaV-1 and EMaV-2: Ethiopia maize-associated virus (MN715238, MF415880); ETBTV: Ethiopian tobacco bushy top virus (KJ918748); GRV: groundnut rosette virus (MG646923); IxYaV2: ixeridium yellow mottle associated virus 2 (KT946712); OPMV: opium poppy mosaic virus (EU151723); OULV: opuntia umbra-like virus (MI-1579715); PMeV2: papaya meleira virus 2 (KT921785); PUV: papaya umbra virus (KP165407); PMMoV: patrinia mild mottle virus (MI-1922775); PEMV2: pea enation mosaic virus 2 (U03563); RCUV: red clover umbravirus (MG596237, MG596235); SULV: sugarcane umbra-like virus (MN868593); SbaVA: strawberry associated virus A (MK211274); TBTV: tobacco bushy top virus (KX216407).

FIG. 3 illustrates schematically the genome organization of CYVaV and similar RNA molecules (Panel A). ORFs encoding for proteins involved in replication are identified in darker grey (p33 and p94 for PEMV2; p21 and p81 for CYVaV; p35 and p86 for PMeV2-ES; p31 and p85 for PUV; p29 and p89 for TBTVa). Umbravirus PEMV2 also possesses ORFs encoding for proteins p26 and p27 involved in movement (identified in light grey boxes). Frameshifting ribosome recording site (FS) and readthrough ribosome recoding site (RT) are also identified. Levels of CYVaV plus (+) strands in infiltrated N. benthamiana leaves (Panel B, top) and systemic leaves (Panel B, bottom) are shown. Levels of the RNA-dependent RNA polymerase (RdRp) synthesized by frameshifting in vitro in wheat germ extracts of full-length CYVaV and PEMV2 are shown (Panel C). The difference in levels of p94 from PEMV2 as compared to p81 polymerase produced by CYVaV is significant. The frameshifting site of CYVaV is one of the strongest known in virology and believed to be responsible for its exceptionally high accumulation.

FIG. 4 illustrates schematically in Panel A gene organization of ulaRNAs and umbraviruses. Genomes of the smaller ulaRNAs and umbravirus PEMV2 are shown in Panel B. ORF1 and the −1PRF product (the RdRp) are found in all umbraviruses and ulaRNAs. The umbravirus ORF3 product is the long-distance movement protein and suppressor of NMD whereas the ORF4 product is required for cell-to-cell movement. ORF5, found in OULV, EMaV and SULV, and ORF5-1, found only in SbaVA, code for proteins with movement protein motifs and are possibly translated from an sgRNA. CYVaV differs from OULV, EMaV and SULV by the absence of two fragments, which for OULV are 145 and 138 nt. The current length of SULV and EMaV are unknown, due to issues at the 50 and 30 ends (see text), which is represented by black boxes at the 30 end of their genomes. Percentages shown denote sequence identify with CYVaV. FS, −1PRF site. Genomes of corn, papaya and babaco ulaRNAs are also shown. Genome organization of tomato bushy stunt virus (TBSV; tombusvirus) and two tlaRNAs, carrot red leaf virus associated RNA (CRLVaRNA) and beet western yellows virus ST9 strain (ST9aRNA) are shown in Panel C. The ulaRNA relatives all have inserts in the 3′UTR and other nucleotide changes that result in the generation of an ORF that encodes for a protein (p21.2) of unknown function.

FIG. 5 shows RNA levels from agro-infiltrated leaves of Nicotiana benthamiana. CVEV (lanes 1-2), CVEV+CYVaV (lanes 3-5) and CYVaV (lanes 5-8) in leaves of Nicotiana benthamiana. Accumulation of CYVaV increased substantially in the presence of putative helper virus CVEV. Plus-strands are shown above. rRNA loading controls are shown below. p14 silencing suppressor was co-infiltrated in all leaves.

FIG. 6 shows RNA levels from another experiment with agroinfiltrated leaves of Nicotiana benthamiana. CYVaV or CVEV or CYVaV+CVEV agroinfiltrated into leaves of N. benthamiana. CYVaV was encapsidated in virions of CVEV, and virions were isolated one week later and the encapsidated RNAs subjected to PCR analysis.

FIG. 7 shows yellowing symptoms of CYVaV (Panel A) and CYVaV+CVEV (Panel B), which are limited to citron (pictured), lemon, and lime.

FIG. 8 shows the systemic and phloem-limited movement of CYVaV in N. benthamiana, wherein CYVaV is confined to the vascular system of the plant. Fluorescence in situ hybridization (FISH) imaging detecting plus strands of CYVaV were stained pink (with areas generally shown herein with dashed white lines and circles) are shown in Panels A-G, including longitudinal and cross-sectional views of petioles (Panels A-D) and root tissue (Panels E-G). Tissue was stained with DAPI. Companion cells (CC), phloem parenchyma cells (PPC) and sieve elements (SE), and xylem (XL) are identified. Note that the iRNA is completely restricted to the SE, CC and PPC. Blue (shown herein as dark grey or black areas) is from DAPI staining of endogenous DNA. CYVaV is symptomless in virtually all tested citrus.

FIG. 9A illustrates schematically the RNA structure for full-length CYVaV (SEQ ID NO:1). CYVaV transcripts were synthesized using T7 RNA polymerase, denatured, snap cooled and then treated with NMIA or DMSO as described in the Materials and Methods. Ten primers labeled with 6FAM were used for reverse transcription of the SHAPE modified samples and PET was used for sequencing ladder samples. Data that was obtained from 2 to 3 repeats of the primer sets were analyzed using QuSHAPE software. Colors denote SHAPE reactivity. The structure was divided into three domains (D1, D2 and D3) for ease of presentation. Structures referred to in the text are numbered in gray. Gray lines denote key base-paired helices that were highly conserved in both sequence and structure among the Class 2 ulaRNAs, and that were important in conceptualizing the final structure. The location of the initiation codon for p21, p21 termination codon (UGA) and p81 termination codon UGA are shown. Two putative tertiary interactions (see text) are denoted by curved lines. Note that subsequent figures show enlargement of key regions of the structure and SHAPE data. The recoding frameshift site (see FIG. 10) is identified by boxed single solid line region, and the ISS-like (I-shaped structure) 3′CITE (see FIG. 11) is identified by boxed dashed line region. For example, a region for accommodating inserted hairpin(s) is shown by boxed double line region.

FIG. 9B illustrates schematically a comparison of the CYVaV RNA structure with structures for other Class 2 ulaRNAs. Designations of CYVaV structures (Pr, H5, H4a and H4b) are highly conserved and denoted for each genome structure. Inserted segments not found in CYVaV are shown in dark grey. Open circle, closed circle and star denote ORF1 initiation site, ORF1 termination site and ORF2 termination site, respectively. Open triangle and closed triangle denote start site and termination site for ORF5, respectively.

FIG. 9C illustrates schematically a comparison of the CYVaV and CYVaV-Delta RNA structures.

FIG. 10 illustrates schematically the structure of the recoding frameshift sites in CYVaV and PEMV2 (Panel A). CYVaV has multiple conformations of the structures in this region (see FIG. 9A) with only one shown. Slippery site is identified by boxed dashed line, and stop codon bases are in black circles. Bases identified by boxed solid line engage in long-distance interaction with the 3′ end.

FIG. 11 illustrates schematically the ISS-like 3′ Cap Independent Translation Enhancer (3′CITE) of CYVaV. The structure of the 3′ end of CYVaV is shown. The 3′CITE is illustrated at the left-most portion shown and with bases circled. Sequence identified by boxed solid line engages in the long-distance RNA:RNA interaction with the recoding site.

FIG. 12 illustrates results from a trans-inhibition assay. Full-length CYVaV was translated in vitro in the presence of 10-fold molar excess of a truncated version of the ISS (ISS_{S}) or full-sized ISS (ISS_{L}).

FIG. 13 demonstrates that CYVaV does not encode a silencing suppressor. Referring to Panels A and B, N. benthamiana 16C plants were agroinfiltrated with a construct expressing green fluorescent protein (GFP) (which is silenced in these plants) and either constructs expressing CYVaV p21 or p81, or constructs expressing known silencing suppressors p19 (from TBSV) or p38 (from TCV). Only p19 and p38 suppress the silencing of GFP, allowing the green fluorescence to be expressed (infiltrated regions identified by circled dashed line in Panel B). Referring to Panel C, northern blot probed with GFP oligonucleotide showed that GFP RNA is still silenced in the presence of p21 or p81.

FIG. 14 demonstrates replication of CYVaV in Arabidopsis protoplasts. An infectious clone of CYVaV was generated. Wild-type RNA transcripts (CYVaV) or transcripts containing a mutation in the recoding slippery site that eliminates the synthesis of the RdRp (CYVaV-fsm), and thus does not replicate, were inoculated onto Arabidopsis protoplasts. RNA was extracted and a Northern blot performed 30 hours later. Note that inoculated transcripts of CYVaV-fsm were still present in the protoplasts at 30 hours (whereas in a traditional virus they would be undetectable after 4 hours). Plus strands are shown in Panel A, and minus strand replication intermediate is shown in Panel B.

FIG. 15 demonstrates replication of CYVaV in N. benthaminana. Referring to Panel A, the level of CYVaV accumulating in the infiltrated leaves of N. benthamiana as determined by Northern blot is shown. Referring to Panel B, plants infiltrated with CYVaV sporadically showed systemic symptoms (see FIG. 16). These plants accumulated high levels of CYVaV. Referring to Panel C, the level of CYVaV in individual leaves of a systemically infected plant is shown. Leaves 4 and 5 were agroinfiltrated with CYVaV. Note the substantial accumulation of CYVaV in the youngest leaves.

FIG. 16 shows symptoms of N. benthamiana systemically infected with CYVaV. Leaves 4 and 5 were agroinfiltrated with CYVaV. The first sign of a systemically infected plant is a “cupped” leaf (Panel A), which was nearly always leaf 9. In the following few weeks, leaf galls emerged at the apical meristem and each node of the plant (Panel B). An uninfected plant (Panel C, left) and an infected plant (Panel C, right) of the same age are shown. Systemically infected plants also had root galls (Panel D), containing a substantial amount of CYVaV as evidenced by Northern plant blot (Panel E).

FIG. 17 is an image of a tomato plant at 53 days post-infection (left) with a plant of the same age (right), and demonstrating the exceptional host range of CYVaV. Sap from a systemically-infected N. benthamiana plant was injected into the petiole of a tomato plant. One of four plants showed very strong symptoms and was positive for CYVaV by PCR analysis.

FIG. 18 demonstrates that CYVaV binds to a highly abundant protein extracted from the phloem of cucumber. Referring to Panel A, labeled full-length CYVaV bound to a prominent protein in this northwestern blot. Proteins were renatured after SDS gel electrophoresis. This protein is believed to be a known, highly conserved RNA binding protein containing an RRM motif that is known to chaperone RNAs from companion cells into sieve elements in the phloem of cucumber. Referring to Panel B, no binding was seen when the proteins remained denatured after electrophoresis.

FIG. 19 demonstrates that CYVaV is capable of expressing an extra protein from its 3′UTR using a TEV IRES. The location of three separate inserts (in three separate constructs) of nanoluciferase downstream of the Tobacco etch virus (TEV) internal ribosome entry site (IRES) are shown (Panel A). In vitro translation was measured in wheat germ extracts for the three constructs (Panel B). Note the location of the nanoluciferase protein (Nluc) is near the bottom of the gel. Expression of nanoluciferase was measured in protoplasts in vivo (Panel C). Full-length RNA transcripts of the constructs (Panel A) were transformed into protoplasts; 18 hours later, total protein was extracted and nanoluciferase activity measured in a luminometer.

FIG. 20 illustrates a an insert at position 2250. A schematic representation of CY2250sfPDS60 is shown in Panel A. The location of the insert in the secondary structure of CYVaV is shown in Panel B, which location corresponds to a region for accommodating inserted hairpins, such as shown by double line box in FIG. 9A. Data from wheat germ extract in-vitro translation assay of T7 transcripts from CYVaV-wt, and CYVaV virus-induced gene silencing (VIGS) vectors containing different amounts of sequence at position 2250 are shown in Panel C. For example, construct sfPDS60 demonstrated excellent systemic movement in plants. Northern blot analysis of total RNA isolated from A. thaliana protoplasts infected by CYVaV wt and CYVaV VIGS vectors. CYVaV-GDD negative control is shown in Panel D. (+) represents plus-strands and (−) are minus strand replication intermediates. An image of N. benthamiana infected by CY2250sfPDS60 is shown in Panel E. RT-PCR products from local leaf and systemic leaf are shown in Panel F. The primer set amplify positions 1963-2654 in the 3′ region of CYVaV. The sequence of the insertion region (underlined) of the vector collected from systemic leaf is shown in Panel G, with dashed line boxed sequences on either side of the insert forming the stem of the hairpin.

FIG. 21 illustrates an insert at position 2301. A schematic representation of CY2301sfPDS60 is shown in Panel A. The location of the insert in the secondary structure of CYVaV is shown in Panel B, and corresponds to a region for accommodating inserted hairpins, such as shown by double line box in FIG. 9A. Data from wheat germ extract in-vitro translation assay of T7 transcripts from CYVaV-wt, and CYVaV VIGS vectors containing different amounts of sequence at positions 2301 and 2319 are shown in Panel C. For example, construct PDS60 demonstrated excellent systemic movement in plants. Northern blot analysis of total RNA isolated from A. thaliana protoplasts infected by CYVaV wt and CYVaV VIGS vectors. CYVaV-GDD and negative control. is shown in Panel D. (+) represents plus-strands and (−) are minus strand replication intermediates. An image of N. benthamiana infected by CY2301sfPDS60 is show in Panel E. RT-PCR products from local leaf and systemic leaf are shown in Panel F. The primer set amplify positions 1963-2654 in the 3′ region of CYVaV. The sequence of the insertion region of the virus vector collected from systemic leaf is shown in Panel G, with dashed line boxed sequences forming the stem of the hairpin.

FIG. 22 illustrates an insert at position 2319. A schematic representation of CY2319sfPDS60 is shown in Panel A. The location of the insert in the secondary structure of CYVaV is shown in Panel B, and corresponds to the region for accommodating inserted hairpins shown by double line box in FIG. 9A. Data from wheat germ extract in-vitro translation assay of T7 transcripts from CYVaV-wt, and CYVaV VIGS vectors containing different amounts of sequence at position 2301 and 2319 are shown in FIG. 21, Panel C. Northern blot analysis of total RNA isolated from A. thaliana protoplasts infected by CYVaV wt and CYVaV VIGS vectors. CYVaV-GDD and negative control is also shown in FIG. 21, Panel D. An image of N. benthamiana infected by CY2319sfPDS60 is shown in Panel C. RT-PCR products from local leaf and systemic leaf is shown in Panel D. The primer set amplify positions 1963-2654 in the 3′ region of CYVaV.

FIG. 23 illustrates the location of a 60 nt insertion (non-hairpin) onto the ORF of the RdRp of CYVaV (Panel A). The location of the insert is indicated by the black arrow. A stop codon, indicated by the black hexagon, was engineered just upstream of the insert to truncate the RdRp. Northern blot of plus-strand RNA levels in Arabidopsis protoplasts is shown in Panel B. CYVaV-GDD is a non-replicating control.

FIG. 24 illustrates a lock and dock sequence for stabilizing the base of inserts. Referring to Panel A, tetraloop GNRA (GAAA) docking with its docking sequence generates an extremely stable structure, and represents a basic lock and dock sequence. Referring to Panel B, use of a scaffold consisting of a docked tetraloop (analogous to the similar structure sometimes used as a crystallography scaffold) is shown. Referring to Panel C, a unique lock and dock structure is shown. Inserts (hairpins or non-hairpin sequences) may be added to the restriction site (as identified by dashed line box). Circled bases in the sequences are the docking sequences for the GAAA tetraloop.

FIG. 25 illustrates that stabilizing the local 3′UTR structure is highly detrimental, but insertion of a destabilizing insert nearby restores viability. Referring to Panel A, a schematic representation of CYVaV-wt. CYVaV-wt 3′ stb is the parental stabilized construct containing 6 nt changes converting G:U pairs to G:C pairs. Two insertions of 60 nucleotides were added to the stabilized parental construct at positions 2319 and 2330 forming CY2319PDS60_3′ stb and CY2330PDS60_3′ stb. Nucleotide changes made to stabilize the structure and generate CYVaV-wt 3′ stb are circled in Panel B. Insertion sites are indicated by the arrows for each constructs: left arrow in Panel A indicating insertion site for construct CY2319PDS60_3′ stb; right arrow in Panel A indicating insertion site for construct CY2330PDS60_3′stb. Referring to Panel C, data is shown from wheat germ extract in-vitro translation assay of T7 transcripts from the constructs shown in Panel A. Note that p81 levels (the frame-shift product) is strongly affected by stabilizing this region. Referring to Panel D, northern blot analysis of total RNA isolated from A. thaliana protoplast infected by CYVaV-wt, CYVaV-wt 3′ stb, CY2319PDS60_3′ stb, CY2330PDS60_3′ stb, and CYVaV-GDD (non-replicating control) is shown. (+) represents plus-strands and (−) are minus strand replication intermediates.

FIG. 26 demonstrates targeting of host gene expression by a CYVaV VIGS construct. A normal, non-infected leaf without an gene for GFP is shown in Panel A, wherein chloroplasts fluoresced bright red when observed under ultraviolet light (shown as dark grey in Panel A). A leaf expressing GFP is shown in Panel B, and appeared dull orange with green stems in coloration under UV light (shown as lighter grey in Panel B). A leaf expressing GFP and infected with an exemplary VIGS construct is shown in Panel C, wherein infected leaves demonstrated effective gene silencing with siRNAs targeting and silencing GFP mRNA via the phloem in leaf vasculature. As shown in Panel D, after 14 days the VIGS construct migrated throughout the host plant (including the leaf shown in Panel C, identified by arrows), wherein siRNAs responsible for GFP gene silencing were distributed throughout the leaves and plant.

FIG. 27 illustrates a CYVaV VIGS vector that targets CTV. N. benthamiana infected with CTV-GFP (CTV expressing GFP) was used as root stock grafted to wild-type CYVaV (CYVaVwt) or CYVaV-GFPhp₂₃₀₁ scions (Panel A). A hairpin targeting GFP (Panel B) is inserted in construct CYVaV-GFPhp₂₃₀₁. The CYVaVwt scion had no effect on CTV-GFP infecting newly emerging rootstock leaves (Panel A, center image). However, green flecks were absent in stipules when CYVaV-GFPhp₂₃₀₁ was present in the scion (Panel A, right image), demonstrating that movement of CYVaV-GFPhp₂₃₀₁ down into the root stock inhibited progression of the CTV infection. When CYVaVwt was present in the root stock, new leaves from the CTV-GFP scion fluoresced green under UV light, demonstrating that widespread CTV infection was continuing unabated (Panel C, middle image). When CYVaV-GFPhp₂₃₀₁ was in the root stock, the upper leaves in all CTV-GFP-infected scions were either partially or nearly fully absent of GFP flecks (Panel C, right image). RT-PCR of the gray region (Panel C, right image, circled A) and green region (circled B) in the leaves absent of GFP flecks indicated that high levels of CYVaV-GFPhp₂₃₀₁ correlated with red fluorescence (region A), with such tissue having between 3,000-fold and 440,000-fold less CTV compared to green region (region B), as shown graphically in Panel D. Fully infected N. benthamiana were agroinfiltrated with CYVaV carrying a hairpin that targeted a conserved sequence in the CTV genome (Panel F). After four days, CTV levels were about 10-fold lower in the infiltrated tissue as compared with tissue infiltrated with CYVaV wild-type (Panel E). Leaves co-infiltrated with CTV-GFP and CYVaV wild-type or CYVaV with a different CTV genome-targeting hairpin (Panel H) showed significant reductions in CTV-GFP at 6 days post-infiltration (Panel G).

FIG. 28 illustrates the infection of cucumber (Panel A) and tomato (Panel B) plants with CYVaV. Panel A, left most image, shows an uninfected cucumber cotyledon (mock) and a cucumber cotyledon agroinfiltrated with CYVaV; the image was taken about 2 months after infection, with both plants grown under similar conditions. Panel A, upper and lower images on the right, shows enlarged views of the boxed areas in the left image. Panel B shows an uninfected tomato plant (mock) and a tomato plant infected with CYVaV; the image was taken about 50 days after infection, with both plants grown under similar conditions.

FIG. 29 illustrates structure and sequences of lock and dock structures 1 and 2 (L&D1 and L&D2, respectively) in accordance with the present disclosure (Panel A). A gel image of RT-PCR result is shown in Panel B: First/left lane: RT-PCR from systemically infected plant containing CYVaV and Lock and Dock1; Second/right lane: PCR using the plasmid construct as a template. Sequencing the band showed high stability of the L&D1 and L&D2 scaffold structures. Sequencing confirmed no evidence of any change in the RNA after one month in plants.

FIG. 30 illustrates CYVaV binding to phloem protein 2 (PP2) in cucumber and N. benthamiana phloem. Referring to Panel A, phloem exudates from uninfected (mock) and two CYVaV-infected cucumber (CYVaV 1 and 2) were collected, crosslinked with formaldehyde (Input) and then used for pull down assays using streptavidin beads with and without attached 5′-biotinylated CYVaV probes (Probe and No Probe, respectively). SDS PAGE gel was stained with Coomassie Blue. Referring to Panel B, samples from A were subjected to electrophoresis and then transferred to nitrocellulose membranes and analyzed by Western Blot using polyclonal antibody to cucumber PP2 (CsPP2) (upper panel). Panel B, lower panel, is the Ponceau S-stained membrane. Referring to Panel C, total RNA recovered from pulldown assay before RNase treatment was subjected to RT-PCR to verify the presence of CYVaV. (+), RNA from CYVaV-infected N. benthamiana; (−), RNA from an uninfected cucumber plant. Similar assays were conducted utilizing N. benthamiana infected with CYVaV or PEMV2 (Panels D, E and F). For PEMV2 pull down, PEMV2-specific probes were attached to beads.

FIG. 31 illustrates the structure and sequence of CYVaV (SEQ ID NO:1) from position numbers 1889-2341. Potential insert positions at 2250, 2301, 2319, 2330 and 2336 are shown, each with an adjacent pair of bases in a light gray circle. The structures and sequences of lock and dock 1 and lock and dock 2 (FIG. 29), and/or another lock and dock structure in accordance with the present disclosure, may be inserted, e.g., at any of the five positions 2250, 2301, 2319, 2330 and/or 2336 (identified by arrows) or other insert locations described herein.

FIG. 32 illustrates N. benthamiana 16C plant infected with CYVaV with GFP 30 nt insert at position 2301, and N benthamiana 16C plant infected with CYVaV with L&D1+GFP 30 nt insert at position 2301. N benthamiana 16C plant infected by only CY2301GFP30s (without lock and dock structure) is shown in Panel A. VIGS effect was not detected. Sequencing alignment between input CYVaV (CY2301GFP30) and the CYVaV accumulating in systemic tissue is shown in Panel B. The later CYVaV contains a 19 nt deletion acquired during infection showing the construct was not stable. N. benthamiana 16C plant infected with CY2301 LD1GPF30s where the 30 nt sequence was inserted into L&D1 at position 2301 is shown in Panel C. Obvious GFP silencing (plant fluorescing red; shown as darker gray in Panel C) by the VIGS vector was observed. Sequence alignment between CY2301LD1GFP30s infected plant and the original construct (Panel D) showed that L&D1 enhanced the stability of the 30 nt insertion. The 30 nt hairpin GFP sequence (plus-sense orientation) is shown in Panel E.

FIG. 33 illustrates the lock and dock 1 (L&D1) (CYm2250LD1) and L&D1+a 30 nt unstructured sequence targeting Callose Synthase (CYm2250LD1Ca1_30as) inserted into CYVaV with a truncated hairpin at a position designated as position 2250 before the truncation. N. benthamiana plant infected by CYm2250LD1 is shown in FIG. 33, Panel A, which contains L&D1 at the end of a truncated hairpin. The addition of this insert at the end of the complete hairpin present in the wild-type molecule was not found to be stable. Sequencing alignment (FIG. 33, Panel B) between CYm2250LD1 in infected tissue (RT-PCR) and the original construct shows complete stability. N. benthamiana 16C plant infected by CYm2250LD1asCa17_30as (CYVaV containing L&D1 with the 30 nt siRNA insert targeting Callose Synthase 7 mRNA expression) is shown in FIG. 33, Panel C. Sequence alignment (FIG. 33, Panel D) between CYm2250LD1Ca1730as accumulating in the infected plant (RT-PCR) and the original construct showing that the 30 nt insert was stable within L&D1. The 30 nt Callose synthase 7 siRNA sequence (antisense orientation) that targets the Callose Synthase that is active in phloem is shown in FIG. 33, Panel E.

FIG. 34 illustrates the secondary structure of a construct including two insertions (CY2301LD2/2330CTV6sh). One insert is a hairpin targeting CTV6 and the other is an empty L&D2 in 2301 (Panel A). N. benthamiana infected with CY2301LD2/2330CTV6sh is shown in Panel B. RT-PCR result from CY 2301LD2/2330CTV6sh-infected plant is shown in Panel C. The top band had both inserts and was the same as the original infiltrated construct. The lower band has a deletion in L&D2. The data showed that the two inserts were tolerated.

FIG. 35 illustrates another lock and dock structure and plant infected therewith. Extending base-pairing at the base of the disclosed lock and dock structures improved stability of larger unstructured inserts. Base-pairing was extended in L&D1 (Panel C) thereby resulting in a third lock and dock structure (L&D3). N. benthamiana plant infected with L&D3 at position 2301 (CY2301LD3) is shown in Panel A. RT-PCR from the symptomatic leaf of infected plant showing a single band (no obvious deletions) is shown in Panel B. Sequence alignment of CYVaV with L&D1 in position 2301 with RT-PCR sequencing of CY2301LD3 from infected plant is shown in Panel C. No instability was detected.

FIG. 36 illustrates an insert in CYVaV at position 2375. N. benthamiana plant infected by CY2375LD1 (CYVaV with the L&D1 inserted at position 2375) is shown in Panel A. RT-PCR from the symptomatic leaf of the infected plant is shown in Panel B. The sequencing result of the larger band was identical to the original sequence. However, the sequence of the short band revealed the partial deletion of L&D1. The secondary structure of the insertion site is shown in Panel C. The polynucleotide sequences shown in FIG. 36, Panel C are cagaccuuuguuacuuccaacac (SEQ ID NO:64) and cuggauuuccuguguuuuggaaguggaag (SEQ ID NO:65).

FIG. 37 demonstrates the ability of CYVaV-derived siRNAs to silence gene expression of Pseudomonas syringae expressing GFP (GFP-Pst) in co-infiltrated leaves of N. benthamiana plants. Growth of GFP-Pst was monitored by examining GFP fluorescence using confocal imaging at 4 days post-infiltration (FIG. 37, Panel A). Quantitative analysis of the intensity of GFP expressed by GFP-Pst. Mean±SE (n=8 [8 leaves from 4 plants]) was conducted (FIG. 37, Panel B). Different letters indicate significant differences determined by ANOVA post hoc (P<0.05). Images show infiltrated area from representative leaves photographed 9 days after infection with GFP-Pst (FIG. 37, Panel C).

FIG. 38 demonstrates the ability of tobacco rattle virus vector (TRV)-derived siRNAs to silence gene expression of Erwinia amylovora in co-infiltrated leaves of N. benthamiana plants. Half of the systemic leaves were challenged with Erwinia amyloyora strain 273 (EA273, diluted to OD₆₀₀=0.0005 in 10 mM MgCl₂) by infiltration 7 days later (FIG. 38, Panel A). Graphs were taken and the bacterial titers were quantified 3 days after infection (FIG. 38, Panel B). WT, EA273 on wild-type N. benthamiana; TRV, EA273 on TRV-infected N. benthamiana; TRV.MurA1, EA273 on TRV-MurA1 (MurA gene fragment 1) infected N. benthamiana; TRV.MurA2, EA273 on TRV-MurA2 (MurA gene fragment 2 and different from MurA1) infected N. benthamiana; TRV.GyrA1, EA273 on TRV-GyrA1 (EA GyrA gene fragment 1) infected N. benthamiana; TRV.GyrA2, EA273 on TRV-GyrA2 (GyrA gene fragment 2 and different from GyrA1) infected N. benthamiana. siRNAs targeting GyrA reduced Erwinia levels 1000-fold.

FIG. 39 demonstrates the ability of specific siRNAs to inhibit the growth of Erwinia but not E. coli. in vitro.

FIG. 40 demonstrates the ability of non-specific siRNAs to significantly inhibit growth of Liberibacter crescens (Lcr) proliferation in vitro. siRNAs were derived from −500 bp long dsRNA via in vitro transcription of GFPuv, Lcr-Gy and Lcr-ADK genes. Water or siRNAs at 20 ng/μl and 100 ng/μl were added to fresh medium (blank) or Lcr cultures with a density of OD₆₀₀=0.001. bacterial growth was photographed at 16 days after treatment. (FIG. 40, Panel A). Bacterial growth measured by a UV spectrophotometer (FIG. 40, Panel C).

FIG. 41 demonstrates further the ability of non-specific siRNAs to significantly inhibit Pseudomonas syringae growth in vitro. siRNA targeting GFPuv, Pst-Gy, Pst-ADK and Lcr-ADK did not kill the bacteria directly, but still showed significant growth inhibition. Green bacteria (shown in white or light grey): living bacteria; Red bacteria (shown in open circles): dead bacteria.

FIG. 42 illustrates stabilization of siRNA targeting GFP in CYVaV genome. Panels A, B and C show the complete hairpin structures targeting GFP mRNA in Nicotiana benthamiana isolate 16C, which expresses GFP. Panel A: 21 nt GFPsh (sh: small hairpin) with the stabilizing loop sequence (ucaagag) from the pSuper vector (Brummelkamp T R, et al, 2002 DOT: 10.1126/science.1068999). The minimum free energy (ΔG) of the structure is −39.30 kcal/mol. The polynucleotide sequence shown in Panel A is tgaagcggcacgact tcttcaatcaagagatgaagaagtcgtgccgcttca (SEQ ID NO:68). Panel B: 26 nt GFPsh with the same loop sequence as in A (ΔG is −51.20 kcal/mole. The polynucleotide sequence shown in Panel B is tgaagcggcacgacttcttcaagagcatcaagagagctctt gaagaagtcgtgccgcttca (SEQ ID NO:69). Panel C: 30 nt GFPsh. The loop sequence is gaauuc and ΔG is −63.80 kcal/mol. Finding PCR bands smaller than expected indicates that these hairpin structures are not stable after systemic infection. Furthermore, the relative band intensity of the small band (vector with insert deleted) and the bigger band (vector with intact insert) in the PCR results suggests that structural stability of the hairpin is reversely correlated with its maintenance in the vector after systemic infection. The polynucleotide sequences shown in Panel C are tgaagcggcacgacttcttca agagcgccaga (SEQ ID NO:70) and tctggcgctcttgaagaagtcgtgccgcttca (SEQ ID NO:71). Panel D: The secondary structure of CYVaV established by SHAPE data (left) and the reference structure to be mimicked, CY2220-2280 (in dashed gray box, right). The ΔG of this structure is −31.50 kcal/mol. The polynucleotide sequence shown in Panel D is ggttagggtaactcacataccttcttccataactggaaaaggtcgtgtgagcaacctaacc (SEQ ID NO:72). Panel E: GFPmmck59. Total 59 nt mimicked structure includes 28 nt siRNA of sense orientation (circled in gray). GFPmmck59 (tgaagcggcacgacttctt caagagcgataactcgccttgacagaagtccaacgcttca (SEQ ID NO:73)) was inserted at position 2304 in CYVaV (arrow), was stable after systemic infection, and was functional in silencing GFP (gray in picture indicates tissue where GFP is silenced). The RT-PCR result and the sequencing chromatogram proved the exceptional stability of the insert after systemic infection. *The minimum free energy was calculated using RNAfold Webserver: http://rna.tbi.univie.ac.at//cgi-bin/RNAWebSuite/RNAfold.cgi*. The polynucleotide sequence shown in Panel E is:

(SEQ ID NO: 74)
aaaaggtcgtgtgagcaacctaaccagttaatgtaggtgt
ctttccgtatgaagcggcacgacttcttcaagagcgataa
ctcgccttgacagaagtccaacgcttcatctagtcaggat
ggtaagcaacccgtttatctgtacggcgctcacccgtggg
taggaag.

FIG. 43 demonstrates the stability of two exemplary mimic inserts in the CYVaV genome. Panel A: Locations of the inserts in one of our two-insert constructs. The natural hairpin at 2219 is deleted (i.e. bases 2220-2280 are deleted) and one of the two mimic inserts (targeting Callose Synthase 7) is inserted at that location (i.e between wild type 2219 and 2281). The second mimic insert (targeting CTV) is at 2304 (wild type numbering). The sequence of Mmck6.1 is SEQ ID NO:75. The sequence of CS7-V2.2 is SEQ ID NO:76. Panel B: In vitro translation of this construct (gray asterisks) in wheat germ extracts compared with WT CYVaV. Panel C: Sequencing the population of the two-hairpin construct at 4 weeks postinfiltration.

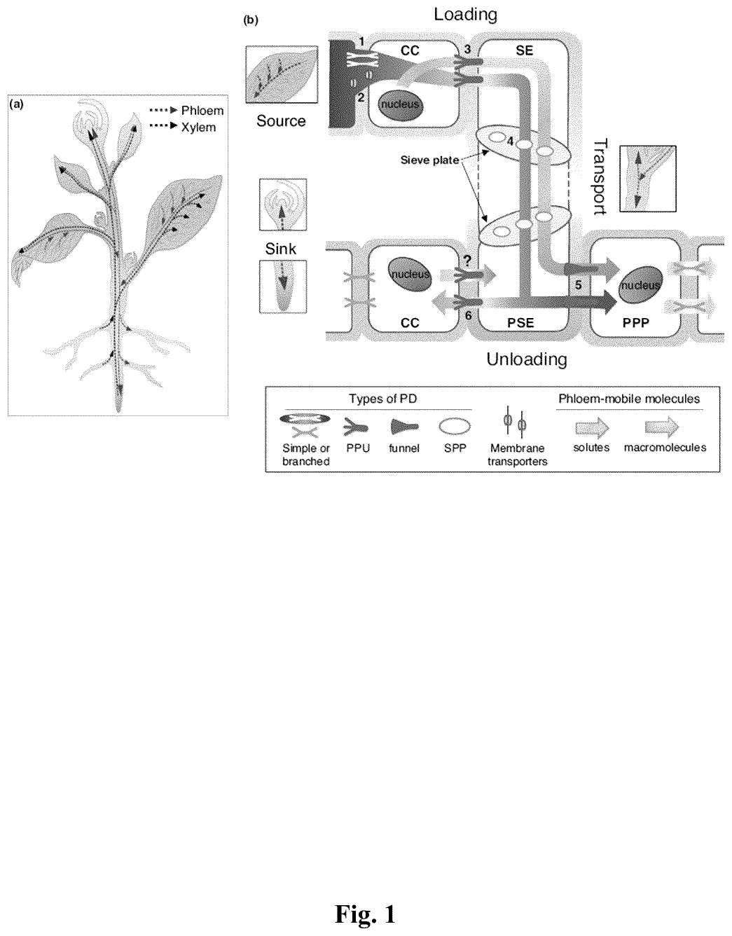

The polynucleotide sequence of F3-C3 #3 is:

(SEQ ID NO: 77)
actagaggaagtgttgacgaaatgtaatgttccagttatg
gaatcattaatttcgtccatcacttcctacggaaagacac
ctacattaactggtatcatttgcagacggaagattggcag
ttatgccaaatcttcgtctgcccattgataccaac.

The polynucleotide sequence shown in Panel D is:

(SEQ ID NO: 78)
ttggttagggtaactcacataccttcttccatatgaagcg
gcacgactatctagtcannntggtaagcannncgtttatc
tgt.

No variants are detectable. Many more combinations of two-insert hairpins were made and all are stable. As shown in Panel C, RT-PCR should indicate a single amplified product, with batch sequencing showing little to no heterogeneity. For comparison, sequencing of an unstable construct is shown in Panel D.

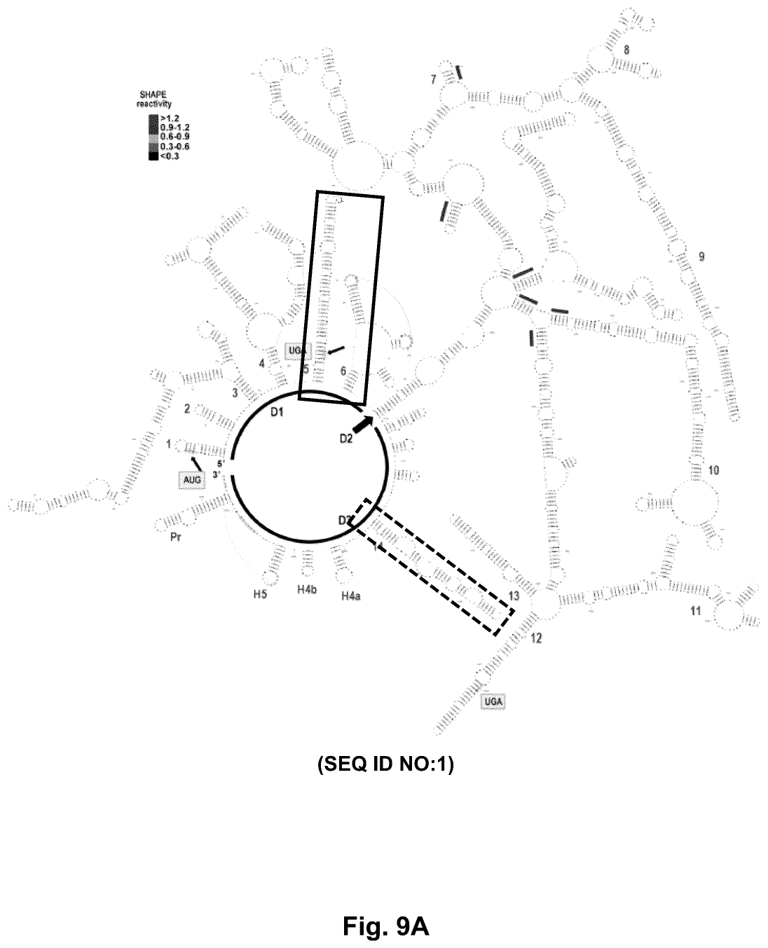

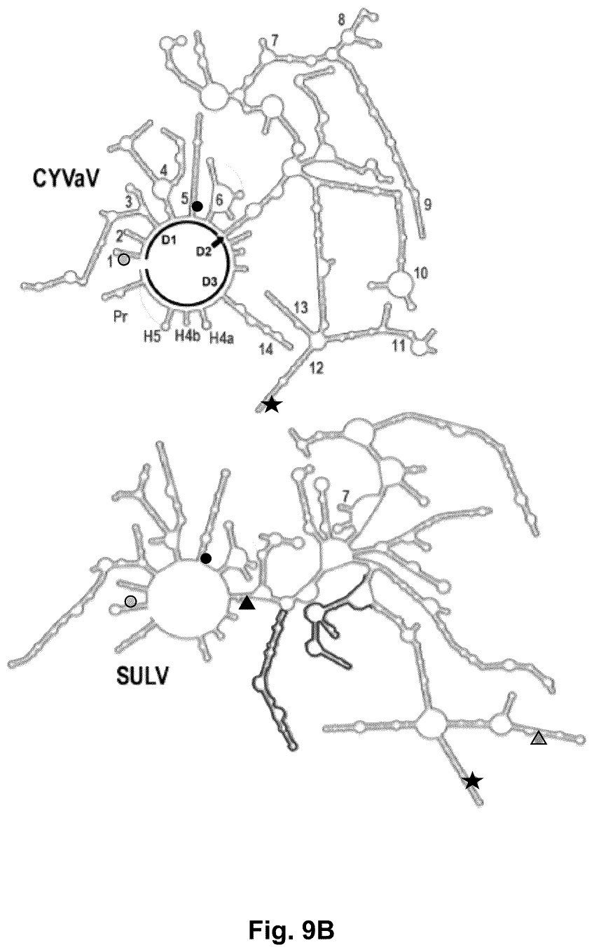

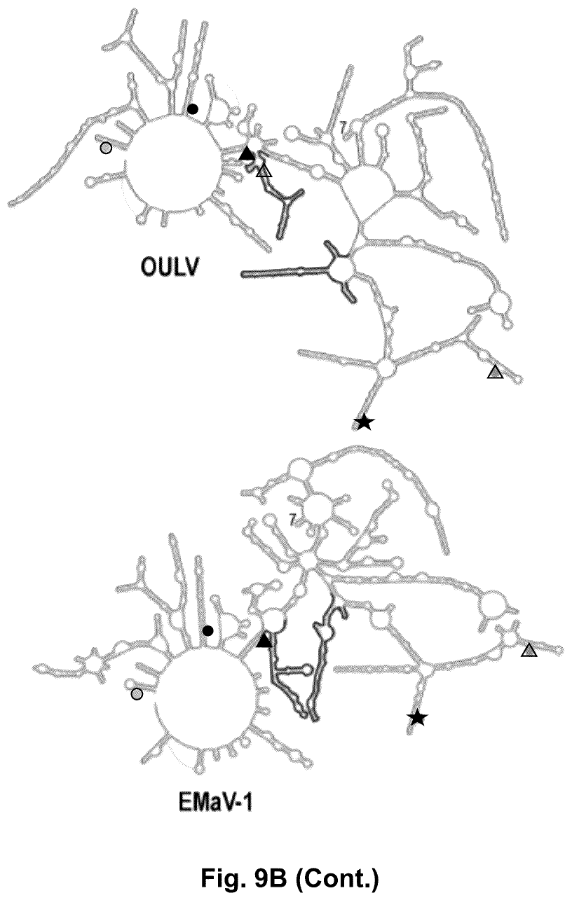

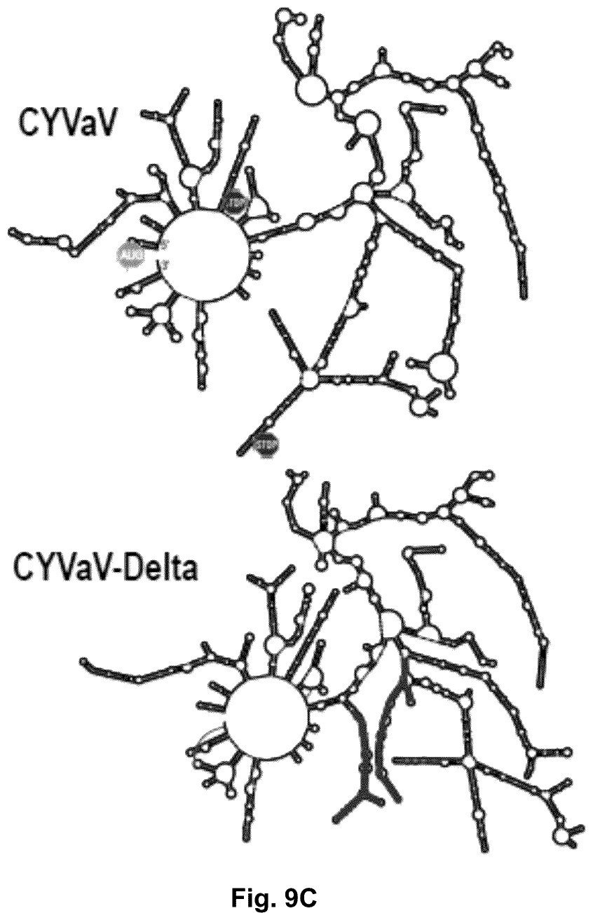

FIG. 44 illustrates wild-type (WT) CYVaV and its nearest relative, CYVaV-Delta, from cannabis. CYVaV-Delta has two inserts (see FIG. 9C) and other changes that allows it to encode an additional ORF

FIG. 45 illustrates schematically active and inactive structures of the recoding structure element (RSE) regions in WT CYVaV, CYVaV-Delta, and engineered CYVaV_{RF}. Active structures are shown in Panels A, C and E, and inactive structures in Panels B, D and F. The inactive recoding structure is the predominant structure of CYVaV, with CYVaV frameshifting at a rate of 30%. The inserted residue at position 2631, which is not present in CYVaV WT but present in CYVaV-Delta, is circled in gray. Other residues in gray form the critical long-distance interaction required for the active structure. Some residues circled in gray pair in the active structure. CYVaV_{RF} is WT CYVaV engineered with four alterations that singly are detrimental for frameshifting but together restore the balance between inactive and active structures, which restores frameshifting to normal levels (see Table 1). i2631A and U664C stabilize the inactive structure. A652G is needed to keep U664C from stabilizing the inactive structure and A604U stabilizes the active structure. Hairpin inserts are slightly destabilizing to the inactive structure of CYVaV. This knowledge was the key to designing alterations to our vector that allow the vector+inserts to be more stable than CYVaV with no (or fewer) inserts, but with the same alterations (i.e, loss of inserts produces an inactive structure that is too stable). The polynucleotide sequences presented in Panels C-F are, respectively, SEQ ID NO:120-123) shown below. The portions of the sequences not shown in Panels A-F are the 5′ end of CYVaV (SEQ ID NO: 1).

SEQ ID NO: 120:
aggtcgcggcgaccttgtggctaactaagaccttgtctga
caaggccttggcccgccaccagggttttcgcgatttgcag
tgattggggtcgacgggctagaggctaaagcagtgcctct
agctgcaggactccgactgcttccggttccgcggcccgga
caaagccgacggctgtctcagaccttgttactaccaaccc
ctcgtgcacaatttgtgaatcacgcgaactcggctaataa
ccttgggcgcgtgatgaccacacgagtgatgcagtacaaa
ggccgagacccgatccaaccctctcaagaagccctaacca
aacttaaccttcggatagccgagctatataagtcgagacc
atccaccgtctatccgttgagttatgaagggtttctcaac
tgctacgaaggccgacagcgtactcgttacgctcaagccg
tcgagcagctgttgaggtccactctagaacctaaagatgc
gagagttgaaacgttcattaagaacgagaagtttgattgg
gcgttgaaaggggaggaggctgatcctcgtgcaatccaac
caaggaagccgaaatatctggctgaggttggacggtggtt
taaacctttggagcgaatcatctacaaggatcttagtaag
cggttgtatggcgagggtggtgaaccttgtattgccaaag
gcttaaatgcactagaatctggggcgactttgaggcgcaa
atgggagaagttttcttctcctgtttgcgtctctcttgac
gcttccaggttcgacctgcatgtaagtgttggcatgctta
agtttacacacaagctgtacgactactactgcaagtctcc
caccctccaacgttatctcaaatggacactccgcaatcac
ggcactgcctcctgtaaagaattgtcatatggttatgagg
tagaaggccgaagaatgagtggtgacatggacaccgcatt
gggcaactgcgtcatcatgtcgatactaacatggtttatg
cttagtgagcttggcattaagcatgaattattcgacaatg
gtgacgactgcttgttcatttgcgagtcaaaagacgtccc
cagccccgaggtgatcacgaactggttttcggactttggg
tttgtggtcaggttggaaggcgtcacgtccgtgtttgagc
gtatagagttttgccaaacttccccagtatggactgagag
gggttggctgatgtgtaggaacattaagtcgttgagtaag
gaccttacgaatgtcaattcgtgcacgggctccacaattg
aatatactcactggttaaaagccgtggggaagtgcgggtc
aatactcaatgctggtgtgcctatatttcagtcctttcac
aacatgctggaaaggcttggtactaactctcgtatagatc
gcggggtgttctttaaatctgggctagtcaatctcattcg
tgggatggacagacaacctgacgttaagatcaccacttcc
gctcgtctttctttcgaagtggcattcgggattacacctg
gaatgcaattggctattgaacggtactatgactcagtcat
gggcccgctgggtaaaatagaaacaactaaatggccaata
gaactaagaaaggaatacgagtacggaagcgagtggtacg
aagacttaggcgtcctaggatgagcagggacatcggttta
ccgatggcccccgttcagaataggatggctagggcttcgt
tggttagggaaacctatataccttcttccaccacaactgg
taaggaggctgtgtgggcaacctggccaattaatgtaggt
gtctttccgcaactagccacgatagtaagcaacccgttat
actggcgtgtgaccagtgtgcaagtggcaatggagccagc
aacgtcaacctctacccaactctgtggtgtaggtctcagt
acatctggggcttacaactcaggggaagggttcgggaaca
cgtttaagctcttgcgctcttgtaactatacgagacgctc
acccgtgggagggaacgtgatggtcaagtggcccatcagc
atgccctacattcttaatgatgatgcgcacaagaccacag
gtctaacagcttgtgtggtcattgctgtcaccaatccagg
tgccatcactggccaatcttgggcggagattcaattgaat
gtagagtacgtagtgggtacttagacagtctgcgggcttg
ggaacgacgccccgctagcaacgtactgctctcctaccgg
actggtagccgagctgttatcttggagcgatagcactgtg
agcctcaccctgcgcgcgatggacgtgttgcgtgaccctc
acagatttgtgaaactctatagagtagttccgcgagccag
aagggaggatggcgacctggcgaaatccaggagctctggg
gggcttgtactcagagtagcaatactgctttagactgtta
gtttgatgaaccacgcgtgtcacgtggggagaactaacag
cgccc
SEQ ID NO: 121:
ccacatcgcgcaggtcgcggcgaccttgtggctaactaag
accttgtctgacaaggccttggcccgccaccagggttttc
gcgatttgcagtgattggggtcgacgggctagaggctaaa
gcagtgcctctagctgcaggactccgactgcttccggttc
cgcggcccggacaaagccgacggctgtctcagaccttgtt
actaccaacccctcgtgcacaatttgtgaatcacgcgaac
tcggctaataaccttgggcgcgtgatgaccacacgagtga
tgcagtacaaaggccgagacccgatccaaccctctcaaga
agccctaaccaaacttaaccttcggatagccgagctatat
aagtcgagaccatccaccgtctatccgttgagttatgaag
ggtttctcaactgctacgaaggccgacagcgtactcgtta
cgctcaagccgtcgagcagctgttgaggtccactctagaa
cctaaagatgcgagagttgaaacgttcattaagaacgaga
agtttgattgggcgttgaaaggggaggaggctgatcctcg
tgcaatccaaccaaggaagccgaaatatctggctgaggtt
ggacggtggtttaaacctttggagcgaatcatctacaagg
atcttagtaagcggttgtatggcgagggtggtgaaccttg
tattgccaaaggcttaaatgcactagaatctggggcgact
ttgaggcgcaaatgggagaagttttcttctcctgtttgcg
tctctcttgacgcttccaggttcgacctgcatgtaagtgt
tggcatgcttaagtttacacacaagctgtacgactactac
tgcaagtctcccaccctccaacgttatctcaaatggacac
tccgcaatcacggcactgcctcctgtaaagaattgtcata
tggttatgaggtagaaggccgaagaatgagtggtgacatg
gacaccgcattgggcaactgcgtcatcatgtcgatactaa
catggtttatgcttagtgagcttggcattaagcatgaatt
attcgacaatggtgacgactgcttgttcatttgcgagtca
aaagacgtccccagccccgaggtgatcacgaactggtttt
cggactttgggtttgtggtcaggttggaaggcgtcacgtc
cgtgtttgagcgtatagagttttgccaaacttccccagta
tggactgagaggggttggctgatgtgtaggaacattaagt
cgttgagtaaggaccttacgaatgtcaattcgtgcacggg
ctccacaattgaatatactcactggttaaaagccgtgggg
aagtgcgggtcaatactcaatgctggtgtgcctatatttc
agtcctttcacaacatgctggaaaggcttggtactaactc
tcgtatagatcgcggggtgttctttaaatctgggctagtc
aatctcattcgtgggatggacagacaacctgacgttaaga
tcaccacttccgctcgtctttctttcgaagtggcattcgg
gattacacctggaatgcaattggctattgaacggtactat
gactcagtcatgggcccgctgggtaaaatagaaacaacta
aatggccaatagaactaagaaaggaatacgagtacggaag
cgagtggtacgaagacttaggcgtcctaggatgagcaggg
acatcggtttaccgatggcccccgttcagaataggatggc
tagggcttcgttggttagggaaacctatataccttcttcc
accacaactggtaaggaggctgtgtgggcaacctggccaa
ttaatgtaggtgtctttccgcaactagccacgatagtaag
caacccgttatactggcgtgtgaccagtgtgcaagtggca
atggagccagcaacgtcaacctctacccaactctgtggtg
taggtctcagtacatctggggcttacaactcaggggaagg
gttcgggaacacgtttaagctcttgcgctcttgtaactat
acgagacgctcacccgtgggagggaacgtgatggtcaagt
ggcccatcagcatgccctacattcttaatgatgatgcgca
caagaccacaggtctaacagcttgtgtggtcattgctgtc
accaatccaggtgccatcactggccaatcttgggcggaga
ttcaattgaatgtagagtacgtagtgggtacttagacagt
ctgcgggcttgggaacgacgccccgctagcaacgtactgc
tctcctaccggactggtagccgagctgttatcttggagcg
atagcactgtgagcctcaccctgcgcgcgatggacgtgtt
gcgtgaccctcacagatttgtgaaactctatagagtagtt
ccgcgagccagaagggaggatggcgacctggcgaaatcca
ggagctctggggggcttgtactcagagtagcaatactgct
ttagactgttagtttgatgaaccacgcgtgtcacgtgggg
agaactaacagcgccc
SEQ ID NO: 122:
aggtcgcggcgaccttgtggttaactaggaccttgcatga
caaggccttggctcgccaccagggttttcgcgatttgcag
tgattggggtcgacgggctagaggctaaagcagtgcctct
agcttctggactccgactgcttccggttccgcggcccgga
caaagccgacgactgtctcagaccttgttacttccaacac
ctcgtgctcaattcgtgaatcacgcgtgctcggctaacaa
ccttggacgtgtgatgaccacacgtgtgttgcagtacaag
ggccgagatccgatccttccctcttctgaagcccttcacc
gacttaaccttcggatagctgagctatataggtctagacc
ttctaccgtctatccattaagttatgaagggtttctcaat
tgctatgaaggccgacagcgtactcgttacgcccaagccg
tcgagcagttgatgcggtccactcttgagccgaaagatgc
gcgagttgaaacgttcattaagaacgagaaatttgactgg
gcgttgaaaggggaggaggctgatcctcgagcaatccaac
caaggaagccgaaatatttggctgaggttggacggtggtt
caaacctttggagcgaatcatctacaaggatctcagtaaa
aggttgtatggtgagggtgctgagccgtgtatcgccaaag
gcctaaatgcattagaatctggagcgactttgaggcgcaa
atgggagaagttttcttctccagtttgcgtttctctcgac
gcttccaggttcgacctgcatgtaagcgttggcatgctaa
agttcacacacaagctatatgactattactgtaagtctcc
cactctccagcgctatctcaaatggacactccgcaaccat
ggcgtcgcctcctgcaaagaattgtcatatgagtatgagg
ttgttggccggagaatgagtggtgacatggacactgcatt
gggcaactgcgtcattatgtcgatacttacatggtttatg
cttagtgaacttggcattaagcatgaattattcgataatg
gtgacgattgtttgttcatttgcgagtctcacgacgtccc
cagccccgaggtaattacaaactggttttcggactttggg
tttgtggttaggttggaaggcgtcacgtccgtgtttgagc
gtattgagttttgccaaacttccccagtatggactgagag
gggttggctgatgtgtaggaatattaagtcattgagtaaa
gaccttacgaatgttaattcgtgcacgggctccacgattg
aatatacccactggttgaaagcagtgggaaagtgcgggtc
aatactcaatgctggtgtacctatatttcagtcctttcac
aacatgctggaaaggcttggcactaactctcgtattgatc
gaggggttttcttcaaatcagggctagttaatctcattcg
tgggatggacaggcagcctgacgttgacatcactacttcc
gctcggctttctttcgaagtggcattcgggataacacccg
ggatgcaattggctattgaacggtactatgactctgtcat
gggctcgctgagtaaaatagaaacaactaagtggccaatt
gaactaagaaaggaatacgaacacggaagtgagtggtacg
aggacttaggcgtcctaggatgaatagggtcattggttta
ccgatgatacctgttcagaataggattgctcgagcttcgt
tggttagggtaactcacataccttcttccataactggaaa
aggtcgtgtgagcaacctaaccagttaatgtaggtgtctt
tccgtatctagtcacgatggtaagcaacccgtttatctgt
acggcgctcacccgtgggtaggaaggtgaaggttttgtgt
cctttaggtcttggacagtctgcgggcttgggaacgacgc
cccgctagcaacgtactgctctcctaccggactggtagct
taattgtcatcttggagcgatagcactgtgggcctcaccc
ttcgcgcgttggacgtgttgcgtgccccccacagatttgt
gaaactctatggagcagttccgcgagccagaagggaggat
ggccgcctggcgtaatccaggagctctggggggcttgtac
tcagagtagcatactgctttagactgttaactttatgaac
cacgcgtgtcacgtggggagagttaacagcgccc
SEQ ID NO: 123:
ccatatcgctcaggtcgcggcgaccttgtggttaactagg
accttgcatgacaaggccttggctcgccaccagggttttc
gcgatttgcagtgattggggtcgacgggctagaggctaaa
gcagtgcctctagcttctggactccgactgcttccggttc
cgcggcccggacaaagccgacgactgtctcagaccttgtt
acttccaacacctcgtgctcaattcgtgaatcacgcgtgc
tcggctaacaaccttggacgtgtgatgaccacacgtgtgt
tgcagtacaagggccgagatccgatccttccctcttctga
agcccttcaccgacttaaccttcggatagctgagctatat
aggtctagaccttctaccgtctatccattaagttatgaag
ggtttctcaattgctatgaaggccgacagcgtactcgtta
cgcccaagccgtcgagcagttgatgcggtccactcttgag
ccgaaagatgcgcgagttgaaacgttcattaagaacgaga
aatttgactgggcgttgaaaggggaggaggctgatcctcg
agcaatccaaccaaggaagccgaaatatttggctgaggtt
ggacggtggttcaaacctttggagcgaatcatctacaagg
atctcagtaaaaggttgtatggtgagggtgctgagccgtg
tatcgccaaaggcctaaatgcattagaatctggagcgact
ttgaggcgcaaatgggagaagttttcttctccagtttgcg
tttctctcgacgcttccaggttcgacctgcatgtaagcgt
tggcatgctaaagttcacacacaagctatatgactattac
tgtaagtctcccactctccagcgctatctcaaatggacac
tccgcaaccatggcgtcgcctcctgcaaagaattgtcata
tgagtatgaggttgttggccggagaatgagtggtgacatg
gacactgcattgggcaactgcgtcattatgtcgatactta
catggtttatgcttagtgaacttggcattaagcatgaatt
attcgataatggtgacgattgtttgttcatttgcgagtct
cacgacgtccccagccccgaggtaattacaaactggtttt
cggactttgggtttgtggttaggttggaaggcgtcacgtc
cgtgtttgagcgtattgagttttgccaaacttccccagta
tggactgagaggggttggctgatgtgtaggaatattaagt
cattgagtaaagaccttacgaatgttaattcgtgcacggg
ctccacgattgaatatacccactggttgaaagcagtggga
aagtgcgggtcaatactcaatgctggtgtacctatatttc
agtcctttcacaacatgctggaaaggcttggcactaactc
tcgtattgatcgaggggttttcttcaaatcagggctagtt
aatctcattcgtgggatggacaggcagcctgacgttgaca
tcactacttccgctcggctttctttcgaagtggcattcgg
gataacacccgggatgcaattggctattgaacggtactat
gactctgtcatgggctcgctgagtaaaatagaaacaacta
agtggccaattgaactaagaaaggaatacgaacacggaag
tgagtggtacgaggacttaggcgtcctaggatgaataggg
tcattggtttaccgatgatacctgttcagaataggattgc
tcgagcttcgttggttagggtaactcacataccttcttcc
ataactggaaaaggtcgtgtgagcaacctaaccagttaat
gtaggtgtctttccgtatctagtcacgatggtaagcaacc
cgtttatctgtacggcgctcacccgtgggtaggaaggtga
aggttttgtgtcctttaggtcttggacagtctgcgggctt
gggaacgacgccccgctagcaacgtactgctctcctaccg
gactggtagcttaattgtcatcttggagcgatagcactgt
gggcctcacccttcgcgcgttggacgtgttgcgtgccccc
cacagatttgtgaaactctatggagcagttccgcgagcca
gaagggaggatggccgcctggcgtaatccaggagctctgg
ggggcttgtactcagagtagcatactgctttagactgtta
actttatgaaccacgcgtgtcacgtggggagagttaacag
cgccc

FIG. 46 illustrates the stability of a sequence copied from a region of OULV (OULV 2349-2491, 143nt, ΔG is −55.60 kcal/mol) and inserted into the CYVaV genome. Referring to Panel A, left image, the secondary structure of opuntia umbra-like virus (OULV) RNA genome is shown; the enlarged chimeric sequence (marked with light-brown circle) and its secondary structure is in the dashed box below. Referring to Panel A, middle image, the secondary structure of CYVaV is shown; the enlarged structure of the insertion site in CYVaV genome is in the dashed box below and the insertion point is marked with a gray arrow. Referring to Panel A, right image, the secondary structure of CY23300ULV (CYVaV with OULV 2349-291 inserted between 2330/2331 of CYVaV) is shown; the enlarged secondary structure of CYVaV 2330 location with chimeric OULV insert is in the dashed box is shown. RT-PCR results using the cDNA of the three infected plants by CY23300ULV are shown in Panel B. Samples are labeled as CY23300ULV-1, CY2330OULV-2, and CY2330OULV-3, respectively, with sequencing identification presented in parentheses. PCR was performed using a primer set targeting the region from 1996 to 2452 of CYVaV genome. pET17CY23300ULV and pCB301CY23300ULV were used for positive control. Sequence alignment results of the three samples are shown in Panel C. The presented sequence of CYVaV wt is SEQ ID NO:1 (2276-2372). The presented sequences of CBC460, CBC461 and CBC462 are identical (SEQ ID NO:79):

taaccagttaatgtaggtgtctttccgtatctagtcacga
tggtaagcaacccgtgcgtgttacgtcagtgacaatcgcc
atggaaccggcaatgtctacctctacccaacactgtgggg
taggactcgctactagcggggcctactcttcaggtgaggg
ttttggtaacttgttcaagtacaagcgcgcttgtaacttt
atctgtacggcgctcacccgtgggtaggaaggtgaaggtt

The presented sequence of the OULV-insert is (SEQ ID NO:80):

tgcgtgttacgtcagtgacaatcgccatggaaccggcaat
gtctacctctacccaacactgtggggtaggactcgctact
agcggggcctactcttcaggtgagggttttggtaacttgt
tcaagtacaagcgcgcttgtaact

Chromatogram images of the three samples above are shown in Panel D. The overall sequencing quality was very stable, except the thymine (T) at position 117 of the insert. The chromatogram signal showed weak noise of adenine (A), which may indicate that a small population of CY23300ULV may have A instead of T at position 117.

FIG. 47 illustrates the stability of duplicated internal structure (1132-1329) of CYVaV at 2220/2280 location in CYVaV genome at 8 weeks after systemic infection. Referring to Panel A, left image, the schematic image for the construction strategy is shown; the original hairpin-like structure between the location 2220 and 2280 was removed and the long-hairpin-like structure spanning from nucleotide positions 1132 to 1329 was cloned at the deleted region. Referring to Panel A, right image, the overall structure of CY2220_(1132-1329)_2280 is shown, which is the outcome of the left image. Referring to Panel B, RT-PCR using systemically infected tissues by CY2220_(1132-1329)_2280 is shown. In the gel image, the size of PCR bands from infected tissues were identical to the positive control (plasmid construct). PCR was performed using a primer set targeting the region from positions 1996 to 2452. Referring to Panel C, sequencing results are shown of PCR products and their alignment to the original sequence, CYVaV 1132-1329.

The presented sequence of the duplicated-insert (1132-1329) corresponds to SEQ ID NO:1 (1132-1329). The presented sequence of CY2220 (1132-1329) 2280-1 and CY2220 (1132-1329) 2280-2 are identical (SEQ ID NO:81):

attgctcgagcttcgttgcaguaaaagguuguauggugagggugcugag
ccguguaucgccaaaggccuaaaugcauuagaaucuggagcgacuuuga
ggcgcaaaugggagaaguuuucuucuccaguuugcguuucucucgacgc
uuccagguucgaccugcauguaagcguuggcaugcuaaaguucacacac
aagcuauaugacuauuacugcagttaatgtaggtgtctttccgt

Referring to Panel D, for more detailed information about the stability of the internally duplicated CYVaV construct, the PCR products from infected tissue were cloned into pMiniT2.0 cloning vector and a total of 44 clones were sequenced to get higher resolution for virus population after long-term infection. The sequencing information revealed that most of the population of the virus was very stable. The internally copied-insert of 29 clones out of 44 samples was identical to the original sequence. 13 out of 44 clones only had single nucleotide changes. The single base change written in black parentheses indicates that the base change happened in a single clone. Among the 13 clones, 2 clones had the same base change at nucleotide location 119 [(T119C)X2]. Multiple base changes in individual clones were marked as the same color. 1 out of 44 clones had two base changes and was marked by orange color, C49T, T111C. 1 out of 44 clones had three base changes and was marked by green color, T111C, T121C and T120C. Except for T113C and T187C, most of the base changes happened near the boundary between loop and stem. There was no major deletion or substitution from all of the samples. The polynucleotide sequence presented in Panel D (SEQ ID NO:124):

cagtaaaaggttgtatggtgagggtgctgagccgtgtatcgccaaaggc
ctaaatgcattagaatctggagcgactttgaggcgcaaatgggagaagt
tttcttctccagtttgcgtttctctcgacgcttccaggttcgacctgca
tgtaagcgttggcatgctaaagttcacacacaagctatatgactattac
tg

FIG. 48 shows sequence alignment results of samples of FIG. 47 shown in Panels A, B, C, D and E thereof. The presented sequence of the Duplicated-insert (1132-1329) corresponds to SEQ ID NO:1 (1132-1329). The presented sequences of CBC135, CBC135, CBC137, CBC138, CBC142, CBC143, CBC144, CDE966, CDE968, CDE969, CDE970, CDE972, CDE975, CDE979, CDF018, CDF019, CDF022, CDF023, CDF024, CDF025, CDF026, CDF030, CDF031, CDF032, CDF038, CDF039, CDF040, CDF043, CDF044, and CDF045 are identical (SEQ ID NO:82):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CBC139 (G22T) is (SEQ ID NO:83):

ggtcattggtttaccgatgatacctgttcagaataggattgctcgagct
tcgttgcagtaaaaggttgtatggtgatggtgctgagccgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CBC140 (C33T) is (SEQ ID NO:84):

ggtcattggtttaccgatgatacctgttcagaataggattgctcgagct
tcgttgcagtaaaaggttgtatggtgagggtgctgagctgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE967 (T119C) is (SEQ ID NO:85):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgttcctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE974 (G62C) is (SEQ ID NO:86):

ggtcattggtttaccgatgatacctgttcagaataggattgctcgagct
tcgttgcagtaaaaggttgtatggtgagggtgctgagccgtgtatcgcc
aaaggcctaaatgcattacaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE977 (T111C, T121C, T170C) is (SEQ ID NO:87):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagcttgcgtttccctcgacgcttccaggttcga
cctgcatgtaagcgttggcatgctaaagctcacacacaagctatatgac
tattactgcagttaatgtaggtgtctttccgtatctagtcacgatggta
agcaac

The presented sequence of CDE978 (T35C) is (SEQ ID NO:88):

ggtcattggtttaccgatgatacctgttcagaataggattgctcgagct
tcgttgcagtaaaaggttgtatggtgagggtgctgagccgcgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE980 (C49T, T111C) is (SEQ ID NO:89):

ggtcattggtttaccgatgatacctgttcagaataggattgctcgagct
tcgttgcagtaaaaggttgtatggtgagggtgctgagccgtgtatcgcc
aaaggtctaaatgcattagaatctggagcgactttgaggcgcaaatggg
agaagttttcttctccagcttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDF015 (C174T) is (SEQ ID NO:90):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcatacacaagctatatgac
tattactgcagttaatgtaggtgtctttccgtatctagtcacgatggta
agcaac

The presented sequence of CDF016 (G125A) is (SEQ ID NO:91):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgtttctctcaacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDF017 (C57T) is (SEQ ID NO:92):

ggtcattggtttaccgatgatacctgttcagaataggattgctcgagct
tcgttgcagtaaaaggttgtatggtgagggtgctgagccgtgtatcgcc
aaaggcctaaatgtattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDF020 (T148C) is (SEQ ID NO:93):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgtttctctcgacgcttccaggttcga
cctgcacgtaagcgttggcatgctaaagttcacacacaagctatatgac
tattactgcagttaatgtaggtgtctttccgtatctagtcacgatggta
agcaac

The presented sequence of CDF037 (T119C) is (SEQ ID NO:94):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgttcctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDF041 (T187C) is (SEQ ID NO:95):

ggtcattggtttaccgatgatacctgttcagaataggattgctcgagctt
cgttgcagtaaaaggttgtatggtgagggtgctgagccgtgtatcgccaa
aggcctaaatgcattagaatctggagcgactttgaggcgcaaatgggaga
agttttcttctccagtttgcgtttctctcgacgcttccaggttcgacctg
catgtaagcgttggcatgctaaagttcacacacaagctatacgactatta
ctgcagttaatgtaggtgtctttccgtatctagtcacgatggtaagcaac

The presented sequence of CDF047 (T113C) is (SEQ ID NO:96):

ggtcattggtttaccgatgatacctgttcagaataggattgctcgagctt
cgttgcagtaaaaggttgtatggtgagggtgctgagccgtgtatcgccaa
aggcctaaatgcattagaatctggagcgactttgaggcgcaaatgggaga
agttttcttctccagttcgcgtttctctcgacgcttccaggttcgacctg
catgtaagcgttggcatgctaaagttcacacacaagctatatgactatta
ctgcagttaatgtaggtgtctttccgtatctagtcacgatggtaagcaac

The presented sequence of CDF049 (T101C) is (SEQ ID NO:97):

ggtcattggtttaccgatgatacctgttcagaataggattgctcgagctt
cgttgcagtaaaaggttgtatggtgagggtgctgagccgtgtatcgccaa
aggcctaaatgcattagaatctggagcgactttgaggcgcaaatgggaga
agtttccttctccagtttgcgtttctctcgacgcttccaggttcgacctg
catgtaagcgttggcatgctaaagttcacacacaagctatatgactatta
ctgcagttaatgtaggtgtctttccgtatctagtcacgatggtaagcaac

FIG. 49 shows: A. Several members of different classes of ulaRNAs are shown. ORFs p20, p21, p22, p31, p33, p35 to the left of the RdRp and p78, p80, p81, p83, p85, p86 and p94 (RdRp) are replication proteins and ORFs p22, p26, p27, p43 and p23 to the right of the RdRp are (putative) movement proteins. The additional embedded ORF p12 and p13 in monocot-infecting Class 2 ulaRNAs is of unknown function. CYVaV1 (also called CYVaV, SEQ ID NO:1) differs from other Class 2 ulaRNAs by not encoding a movement protein due to several large deletions (and other changes). B. Levels of 1-ribosomal frameshifting for CYVaV1 compared with umbravirus PEMV2. C. Secondary structure of full-length CYVaV1. Each “dot” represents a nucleotide. The furthest left arrow points to the initiation codon for p21. The other two arrows are the two termination codons. Numbers denote the natural CYVaV1 hairpin-like structures that serve as templates for the mimic hairpin-like structures. When duplicated and inserted into different locations in the CYVaV1 3′UTR, these hairpin-like structures were stably maintained at all time points assayed until senescence of the host plant.

FIG. 50 shows: Multiple short and long-distance tertiary interactions connect distal sequences with the 1-frameshifting recoding region of CYVaV1. The “slippery” sequence that causes ribosomes to shift back one nucleotide and continue translation is boxed. Another box denotes the p21 stop codon. One of four highly conserved structures in the recoding region is shown, and only a few of the tertiary interactions are present in this particular conformation. Nucleotides are shaded according to SHAPE reagent reactivity. The presented polynucleotide sequence comprises residues 64-2407 of SEQ ID NO:1.

FIG. 51A shows the sequence and secondary structure of a hairpin-like insert mmck6.2 (SEQ ID NO:129).

FIG. 51B shows RT-PCT analysis and Sanger sequencing chromatographs from Mexican lime leaves infected with recombinant CYVaV1 having mmck6.2 after 12 months.

FIG. 51C shows: A. Northern blot of RNA extracted from two infected citrus plants after about one year of infection with recombinant CYVaV1 including mmck6.2. B. Reverse-transcriptase (RT)-PCR is used to amplify a segment of the infecting VIGS vector. For this plant, 10 leaves were assayed. Only a single product of the correct size was found after 15 months of infection. C. Total RNA from young leaves of the infected plant is subjected to batch sequencing of the insert region. Top box is from 15-month citrus showing no indication of single “double peaks” that would denote base changes or multiple double peaks that would indicate a portion of the population contain deletions. In addition, at least 10 clones are generated for individual sequencing and no changes from the initial VIGS vector must be found. D. Photo of infected Mexican lime plant. E. Nucleotide sequence of insert:

(SEQ ID NO: 98)
gtttcagtatttcgtcatcaccttcctaacggaagtgatggacgaaatta
atgaaacc.

FIG. 52 shows: A. Flow chart for how the data in B was generated. Note that color coding shown on the inserts (from RNAFold) are relative entropy values for the specific insert and go from low entropy to high entropy. Positional entropy values for each nucleotide, obtained from the folding program, go from 0 (white) to 2.6 (dark gray) and are absolute values that allow for comparisons between hairpins. Positional entropy values for individual residues are then rearranged for better visualization of the entire insert (far right). B. APE values for various hairpin-like structures (and some additional non-hairpin-like structures) inserted into CYVaV. Sorted positional entropy is shown for each insert (dark gray to lightest gray). The first four hairpin-like structures (boxed) were natural duplicates of hairpin-like structures 1 through 4 (see FIG. 49 Panel C). Other hairpin-like structures and non-hairpin-like inserts are arranged in order of descending APE (from 1.30 to 0.07). Mimic hairpin-like structures begin with a number (1 or 4) that identifies the hairpin-like structure being mimicked. Thick lines beneath each lane denote the length of each hairpin (number of nucleotides shown at right). Asterisks denote inserts that were not stable in the VIGS vector. Double asterisk denotes a hairpin-like structure that had three consecutive G:C pairs at the base and five consecutive G:C pairs in the apical region contributing to the very low APE. Eliminating two nucleotides on the 3′ side of the hairpin in the apical region generated a stable hairpin-like structure with no consecutive apical G:C pairs and the APE value changed to 0.13. Thick black arrow denotes an unstable hairpin-like insert with a relatively low APE value of 0.39 that contained a large number of higher entropy nucleotides.

FIG. 53 shows some elements of RNA secondary structure.

FIG. 54 shows sequences and secondary structure of A—wild type CYVaV hairpin-like structure 4 of FIG. 49 (SEQ ID NO:99) and exogenous hairpin-like structures B—mmck15 (SEQ ID NO:100); C—mmck8 (SEQ ID NO:101); D—mmckpsvD (SEQ ID NO:102); and E—mmckpsvE (SEQ ID NO:103).

FIG. 55 shows sequences and secondary structure of A—wild type CYVaV hairpin-like structure 4 of FIG. 49 (SEQ ID NO:99) and exogenous hairpin-like structures B—PRSVmmck1 (SEQ ID NO:104) and C—PSRVmmck2 (SEQ ID NO:105).

FIG. 56 shows sequences and secondary structure of A—wild type CYVaV hairpin-like structure 1 of FIG. 49 (SEQ ID NO:106) and exogenous hairpin-like structures B—PDS-mmck-1 (SEQ ID NO:107) and C—PDS-mmck-2 (SEQ ID NO:108).

FIG. 57 shows sequences and secondary structure of A—wild type CYVaV hairpin-like structure 1 of FIG. 49 (SEQ ID NO:106) and exogenous hairpin-like structures B—LcrGyr-mmck5 (SEQ ID NO:109) and C—LcrGyr-mmck3 (SEQ ID NO:110).

FIG. 58 shows sequences and secondary structure of A—wild type CYVaV hairpin-like structure 1 of FIG. 49 (SEQ ID NO:106) and exogenous hairpin-like structures B—Clas-GyrAmmck-5′ (SEQ ID NO:111) and C-Clas-GyrAmmck-3′ (SEQ ID NO:112).

FIG. 59 shows sequences and secondary structure of A—wild type CYVaV hairpin-like structure 1 of FIG. 49 (SEQ ID NO:106) and exogenous hairpin-like structures B—CS 7 (SEQ ID NO:113).

FIG. 60 shows sequences and secondary structure of A—wild type CYVaV hairpin-like structure 1 of FIG. 49 (SEQ ID NO:106) and exogenous hairpin-like structures B—Erwinia GyrA (SEQ ID NO:114).

FIG. 61 shows SHAPE changes resulting from insertion of CY2301GFP30sh into CYVaV. The presented polynucleotide sequence is SEQ ID NO:37.

FIG. 62 shows the sequence and secondary structure of MF250GFP30ext, an unstable insert targeting GFP. The presented polynucleotide sequence is (SEQ ID NO:115):

gguuaggguaacucacugaagcggcacgacuucuucaagagcgccauuca
gugagcaaccuaacc.

FIG. 63 shows the sequence and secondary structure of GFPmmck59, a stable insert targeting GFP. The presented polynucleotide sequence is (SEQ ID NO:116):

tgaagcggcacgacttcttcaagagcgataactcgccttgacagaagtcc
aacgcttca.

FIG. 64 shows the sequence and secondary structure of GFPmmck63, an unstable insert targeting GFP. The presented polynucleotide sequence is (SEQ ID NO:117):

tgaagcggcacgacttcttcaagagcgccataatcggcgccttgacagaa
gtccaacgcttca.

FIG. 65 shows the sequence and secondary structure of CTV-insert-natural-V2.2, a naturally occurring hairpin-like structure in CTV. The presented polynucleotide sequence is (SEQ ID NO:118):

ggggguuuauguuuggcaaagaaaguguuggaacuguuagucaagcgggu
gguugaaucguuuucucguuugaagcggaaaaccgcucguuuaacguccu
ucgcuaauuuguugcuugcgaggcucuc.

FIG. 66 shows RT-PCR and Sanger data for recombinant CYVaV1 with insert-natural-V2.2 infecting N. benthamiana after 3 weeks. The presented polynucleotide sequence is (SEQ ID NO:119):

tcgttgggggtttatgtttgcaaagaaagtgttggaactgttagtcaagc
gggtggttgaatcgtttctcgtttgaagcggaaaaccgctcgtttaacgt
ccttcgctaatttgttgcttgcgaggctctcagttaat

FIG. 67 shows an unstable insert BBLv2-1 (SEQ ID NO. 132 and SEQ ID NO:133).

### DETAILED DESCRIPTION OF EMBODIMENTS

The present disclosure relates to one or more vectors having novel exogenous segments, compositions comprising a plant infected by the vector(s), and uses and methods relating thereto. In some examples, the exogenous segments are provided in combination with“independently mobile RNAs” or “iRNAs” or other viruses or derivatives thereof. In accordance with disclosed embodiments, the iRNAs, for example ulaRNAs, are capable of infecting plants and encoding for an RNA polymerase to sustain their own replication, but lacking the ability to encode for a coat protein. In addition, iRNAs do not code for any RNA silencing suppressors and some do not code for any movement protein.

As used herein, a “host” refers to a cell, tissue or organism capable of being infected by and capable of replicating a nucleic acid. A host may include a whole plant, a plant organ, plant tissue, a plant protoplast, and a plant cell. A plant organ refers to a distinct and visibly differentiated part of a plant, such as root, stem, leaf, seed, graft or scion. Plant tissue refers to any tissue of a plant in whole or in part. Protoplast refers to an isolated cell without cell walls, having the potency for regeneration into cell culture, tissue or whole plant. Plant cell refers to the structural and physiological unit of plants, consisting of a protoplast and the cell wall.

As used herein, “nucleic acid sequence,” “polynucleotide,” “nucleotide” and “oligonucleotide” are used interchangeably and refer to a polymeric form of nucleotides of any length. Polynucleotides may have any three-dimensional structure, and may perform any function. A “gene” refers to a polynucleotide containing at least one open reading frame that is capable of encoding a particular polypeptide sequence. “Expression” refers to the process by which a polynucleotide is transcribed into mRNA and/or the process by which the transcribed mRNA is translated into peptides, polypeptides, or proteins.

A vector “derived from” a particular molecule means that the vector contains genetic elements or sequence portions from such molecule. In some embodiments, the vector comprises a replicase open reading frame (ORF) from such molecule (e.g., iRNA). One or more heterologous segment(s) may be added as an additional sequence to the vectors of the present disclosure. In some implementations, said heterologous segment(s) is added such that high level expression (e.g., of a particular protein or small RNA) is achieved. The resulting vector is capable of replicating in plant cells by forming further RNA vector molecules by RNA-dependent RNA polymerization using the RNA vector as a template. An iRNA vector may be constructed from the RNA molecule from which it is derived (e.g., CYVaV).

As used herein, the terms “infection” or “capable of infecting,” with respect to a vector of the present invention, include the ability of such vector to transfer or introduce its nucleic acid into a host, such that the nucleic acid or portion(s) thereof is replicated and/or proteins or other agents are synthesized or delivered in the host. Infection also includes the ability of a selected nucleic acid sequence to integrate into a genome of a target host.

As used herein, the term a “phenotypic trait” refers to an observable, measurable or detectable characteristic or property resulting from the expression or suppression of a gene or genes. Phenotype includes observable traits as well as biochemical processes.

As used herein, the term “endogenous” refers to a polypeptide, nucleic acid or gene that is expressed by a host. “Heterologous” refers to a polypeptide, nucleic acid or gene that is not naturally expressed by a host. A “functional heterologous ORF” refers to an open reading frame (ORF) that is not present in the respective unmodified or native molecule and which can be expressed to yield a particular agent such as a peptide, protein or small RNA. For being expressible from the vector in a plant, plant tissue or plant cell, the vector comprising a functional heterologous ORF comprises one or more subgenomic promoters or other sequence(s) required for expression.

As used herein, the term “insert” refers to an exogenous RNA segment, typically of material length (e.g. 40 nucleotides or more, or 60 nucleotides or more), located between two bases in the genetic sequence of a reference RNA molecule. The reference RNA molecule may be a wild type molecule (e.g. the genome of a virus or sub-viral RNA) or a molecule derived from a wild type molecule. No act of insertion is necessarily required, for example a molecule may be synthesized according to a sequence including the sequence of the insert with no intermediary synthesis or collection of the wild type molecule.

As used herein, the term “hairpin” (FIG. 53) refers to a primarily base paired segment of RNA that comprises a fully base-paired region (alternatively called a “stack”) and an apical loop between the bases on opposite sides of the stack.

As used herein, the term “hairpin-like structure” refers to a primarily base paired segment of RNA having multiple stacks separated by non-base-paired regions, alternatively called “bulges” or “internal loops” (sometimes called “loops” for brevity when the distinction from an apical loop is apparent from the context), and an apical loop between the bases on opposite sides of the stacks. A loop may be symmetric, with an equal number of bases on opposite sides of the loop, or asymmetric, with a different number of bases on opposite sides of the loop. The term “bulge” may be used as an alternative name for a loop, or may be used in some contexts to refer specifically to an asymmetric loop having one or more non-paired bases on only one side. A bulge or loop thereby two stacks. Referring to FIG. 53, one example of a hairpin-like structure includes the structure having 4 stacks, 3 internal loops (one of which is also a bulge) and an apical loop extending upwards and to the right from the junction. Examples of hairpin-like structures are also shown in FIGS. 54-60.

As used herein, the term “junction” refers to a non-base-paired region that separates three or more stacks. A simple junction separating three stacks may create, for example, a Y-shaped or T-shaped secondary structure. A stack connected to a junction may be part of a hairpin, a hairpin-like structure, or a stem.

As used herein, the term “stem” refers to a structure that extends from a junction that does not terminate in an apical loop. A stem may connect a junction to another junction, or may connect a junction to the single stranded regions between stems (i.e., single stranded regions that are not apical loops, internal loops or junctions). Referring to FIG. 53, the junction separates a stem below the junction from two hairpin-like structures above the junction.

As used herein, the term “RNA vector” refers to a vector including RNA. The RNA vector may be, for example, a viral vector or a sub-viral vector. In some examples, the RNA is a plus-sense single stranded RNA, wherein the term single stranded RNA may include folded RNA with double stranded or base-paired regions. The RNA vector may be derived from a virus or from sub-viral RNA. For example, an RNA vector may be derived from CYVaV, CYVaV-delta, OULV or another ulaRNA; CTV; or, TRV. In some examples, the RNA vector may include proteins expressed by the RNA vector.

Hairpins, hairpin-like structures, and junctions are elements of the secondary structure of RNA molecules. The secondary structure describes the folding of single stranded RNA into base-paired and non-base-paired regions. An RNA molecule may also be defined by its tertiary structure. The tertiary structure includes interactions, i.e. base pairing, of segments of the RNA separated in a manner other than by way of junctions or apical loops. In some cases the tertiary structure includes long-distance interactions between RNA segments separated by many bases and/or intermediate hairpins or hairpin-like structures.

Various assays are known in the art for determining expression of a particular product, including but not limited to: hybridization assays (e.g. Northern blot analysis), amplification procedures (e.g. RT-PCR), and array-based technologies. Expression may also be determined using techniques known in the art for examining the protein product, including but not limited to: radioimmunoassay, ELISA (enzyme linked immunoradiometric assays), sandwich immunoassays, immunoradiometric assays, in situ immunoassays, western blot analysis, immunoprecipitation assays, immunofluorescent assays, GC-Mass Spec, and SDS-PAGE.

An “exogenous RNA segment”, alternatively called an “exogenous segment” or “heterologous segment”, refers to a segment of RNA inserted into a native molecule, whereby the source of the exogenous RNA segment is different from the native molecule. The source may be another virus, a living organism such as a plant, animal, bacteria, virus or fungus, a chemically synthesized material, or a combination thereof. The exogenous RNA segment may provide any function appropriate for a particular application, including but not limited to: a non-coding function RNA, a coding function in which the RNA acts as a messenger RNA encoding a sequence which, translated by the host cell, results in synthesis of a peptide (e.g., a molecule comprising between about 2 and 50 amino acids) or a protein (e.g. a molecule comprising 50 or more amino acid) having useful or desired properties.

As used herein, the term “movement protein” refers to a protein(s) required for cell-to-cell and/or long distance movement. “Coat protein” refers to protein(s) comprising or building the virus coat.

As used herein, “virus” includes traditional or conventional viruses and related infectious agents that might or might not satisfy all elements of some definitions of a virus, for example because they do not encode a coat protein. For example, viruses include umbraviruses and umbravirus-like associated RNA (also called “ulaRNA”), for example CYVaV. Virus also includes derivatives, variants or parts of a virus, for example sub-viral RNA, defective RNA, subgenomic RNA and RNA multimers (e.g. dimers), that may be infectious in a host alone or in the presence of the full virus or a helper virus.

Optionally, an RNA vector may be used for virus-induced gene silencing (VIGS) or in a manner analogous to VIGS. In the treatment of plants, virus-induced gene silencing makes use of a virus, for example a plus-strand RNA plant virus, as a vector to exploit natural antiviral defenses (e.g. RNA silencing) that target foreign RNA to provide protection against plant pathogens and/or alter plant gene expression. Briefly, double-stranded (ds) RNA regions of viral genomes are substrates for cytoplasmic dsRNA-specific, dicer-like RNases (DCLs) that generate small 21-24 nt (per side) dsRNAs. The dsRNA is known as a small interfering (si)RNA. One of the two viral siRNA strands, which alone may also be referred to as an siRNA, is incorporated into a protein complex called “RISC”, and complementary base-pairing between the siRNA strand and an intact viral genome leads to hybridization-site cleavage by RISC complex Argonaut enzymes. Additionally, the RISC/siRNA complex can amplify the number of siRNAs available for targeting by priming complementary strand synthesis on the viral RNA genome, a process that uses an endogenous cytoplasmic RNA-dependent RNA polymerase (RDR6) and host protein SGS3 to generates fully dsRNA products. The dsRNA is then cleaved into secondary siRNAs by DCLs for further RISC-mediated target cleavage. The intercellular mobility of siRNAs in plants allows siRNA to enter the vascular system for migration to distal parts of the plant, leading to the spread of the silencing signal.

Viral RNA genomes that are engineered to contain exogenous inserts such as hairpins generate additional siRNAs from the inserted sequences. These additional siRNAs can be designed to target viruses and other plant pathogens such as nematodes, fungi, and feeding insects due to efficient cross-kingdom transfer of siRNAs into organisms that contain similar RNA silencing systems. Although bacteria do not have the enzymatic components of RNA silencing, they are still able to take up siRNAs and genes are silenced by an unknown mechanism. VIGS-generated siRNAs can therefore be designed to protect the plant against a wide variety of harmful pathogens and pests without modifying the plant genome. In addition, VIGS is used by many laboratory researchers to reduce expression of specific plant genes at the post-transcriptional level to study the ensuing biological consequences. Since plant siRNAs must contain a high degree of complementarity with their target RNA to induce silencing, VIGS produces few, if any, off-target effects. Importantly, the safety of siRNAs for human/animal consumption has been verified by many studies. Ideally, a VIGS vector is mild or asymptomatic in the intended host and retains the insert for a time sufficient to allow treatment of the host.

Over 50 different viruses have been developed as VIGS vectors with these goals in mind, underlying the importance of VIGS technology for both basic and applied agricultural science. However, a critical, unresolved problem is that plant virus vectors engineered to contain any foreign inserted sequence are unstable (e.g. unstable in replication), even when inserts are small hairpins. Over time (usually days to a few weeks), viral progeny emerge in the population with most if not all of the inserted sequence deleted, frequently together with surrounding viral genomic sequences. The enzyme responsible for these deletions is thought to be the virus-encoded RdRp, an enzyme capable of similar recombination-type events leading to the generation of defective truncated viral RNAs that are commonly associated with both plant and animal RNA viruses. Virus fitness may be reduced significantly by the addition of structured sequences, even when these sequences are inserted into regions of the genome known to be devoid of any critical viral functions. Some hairpin inserts are more stable than others, even when they are similarly sized and inserted into the same location in the viral vector geome. Instability of VIGS inserts is regarded in the art as a complex, unsolvable conundrum that is negating the use of this valuable biotechnology for most agricultural purposes. To our knowledge no insert-containing VIGS vector has been made sufficiently stable for usage in long-lived trees and vines, many of which are under considerable threat by bacteria, fungal, viral and other pathogens. For example, in the next decade, all openly grown citrus trees will be infected by Candidus Liberibactor asiaticus, the presumptive causal agent of lethal Citrus Greening disease.

This specification describes, among other things, a plant (e.g. citrus) VIGS vector based on a subviral, umbravirus-like RNA (ulaRNA) originally discovered infecting four limequat trees in the 1950's. This ulaRNA vector, known as citrus yellow vein associated virus (CYVaV or CYVaV1: 2.7 kb), is a plus-sense subviral RNA that contains only the first two open reading frames (ORFs) found in all members of the Tombusviridae; a replication-required protein, and a ribosome-recoding (−1 frameshifting) extension product identified as the viral RNA-dependent RNA polymerase (RdRp) (FIG. 49, Panel A). Surprisingly, CYVaV1 is missing ORFs that encode proteins associated with nearly all other plant viruses, including capsid proteins (for virion formation), movement proteins (for traversing the plasmodesmatal connections between plant cells), and RNA silencing suppressors (for evading RNA silencing defenses). CYVaV1 is one of a collection of recently discovered ulaRNAs, most of which (unlike CYVaV1) also encode one of several different putative movement proteins (FIG. 49, Panel A). Despite only encoding proteins necessary for replication, CYVaV1 systemically invades the vascular system of at least citrus, grapevine, tobacco, hemp, cucumber and watermelon, likely by using a host RNA movement protein. Many endogenous RNA movement proteins are involved in the transport of a substantial number of plant mRNAs through the vascular system and CYVaV1 has been isolated from sap associated with one such protein, PHLOEM PROTEIN 2, known to transport non-coding, circular RNA viroids. Interestingly, ulaRNAs with up to 90% similarity with CYVaV1 (e.g. OULV and CYVaV-Delta) are also capable of independent movement in the absence of their encoded movement protein, although the rate of infection may be altered.

This specification describes viral vectors, for example vectors derived from CYVaV1 or other ulaRNAs. These vectors have stable inserts, for example inserts that may persist for a month or more, three months or more, or a year or more, in the host plant.

To generate a CYVaV1 VIGS vector, we used selective 2′-hydroxyl acylation analyzed by primer extension (SHAPE) RNA structure probing and phylogenetic structure comparisons to solve the secondary structure of full-length CYVaV1 (2.7 kb) (FIG. 49, Panel C). Once the structure was known, the 3′UTR was deletion mapped to find sites that did not disrupt important functions. Using a model plant that is a host for most viruses (Nicotiana benthamiana) and infecting plants via Agrobacteria tumefaciens-mediated vacuum infiltration, the stability of CYVaV1 with hairpin inserts in one of five compatible locations was determined using RT-PCR and direct sequencing. Inserts varied from unstable (i.e., the majority of CYVaV1 accumulating in plants 3-weeks post-infiltration were missing all or part of the insert) to mostly stable (nearly all CYVaV1 retained the entire insert but deleted versions were discernable).

Before we could understand factors involved in insert instability, more information was needed on the basic biology of CYVaV1 and other ulaRNAs. Like many viruses, all ulaRNAs use −1 ribosomal frameshifting for synthesis of their RdRp. In vitro translation using wheat germ extracts revealed an astonishing 30% frameshifting rate for CYVaV1, compared with 2 to 5% for other members of the Tombusviridae (FIG. 49, Panel B). We hypothesize that the high frameshifting rate for CYVaV1 is required to synthesize a large number of progeny given the absence of a protecting RNA silencing suppressor. Moreover, all CYVaV1 containing unstable inserts had reduced frameshifting levels, even though the frameshift site is located 1.7 kb upstream of the nearest insertion site. In addition, a correlation was found between mostly stable inserts and more modest reductions in frameshifting rates. When mapping sequences important for the high rate of frameshifting, we found that critical sequences spanned over 80% (2.2 kb) of the CYVaV1 genome, with four long-distance tertiary interactions and two proximal tertiary interactions connecting local and distal sequences with the recoding site (FIG. 50). These interactions contributed to multiple, highly conserved alternative RNA conformations in the recoding region. Initial efforts to increase stability of constructs with hairpin inserts centered on enhancing the stability of the active conformation of the recoding site, and while stability of inserts was significantly enhanced, low levels of deleted variants were still detectable by RT-PCR. To understand why inserts were affecting frameshifting, we compared high-throughput SHAPE RNA structure probing of full-length WT CYVaV1 and CYVaV1 with inserts of varying stabilities. We found that even small, fully base-paired hairpins caused substantial flexibility changes in residues throughout the entire genome, with unstable hairpins causing significantly more flexibility changes than ones that were mostly stable. Many flexibility changes were located in unpaired internal and terminal loops and in key RNA structures, including those at the recoding site, the cap-independent translation enhancer located in the 3′UTR and a highly conserved Y-shaped structure upstream of the recoding site that participates in one of the tertiary interactions. These results led to one of two paradigm-shifting hypotheses to explain why foreign hairpins in RNA virus vectors are not entirely stable: The conformation of the entire CYVaV1 genome has evolved to be metastable and highly interactive through canonical and non-canonical tertiary interactions, and hairpin-like structures throughout the dynamic genome structure are perfectly tuned thermodynamically to maintain the genome-wide conformation required for critical virus functions. In other words, natural hairpin-like structures emanating from the genome “backbone” are evolved to maintain a critical genome-wide metastable conformation. Insertion of a foreign (and thus non-optimized) hairpin stresses this integrative structure, leading to cascading destabilization that impacts critical tertiary interactions necessary to perform required functions such as frameshifting. The natural ability of error prone, non-processive RdRp to delete all or part of the interfering structure re-establishes viral genome homeostasis and thus enhances fitness.

To begin testing this hypothesis, we inserted exact duplicates of four natural CYVaV1 hairpin-like structures from coding and non-coding regions into 3′UTR locations that had “kicked out” some foreign hairpins. These hairpin-like structures ranged in size from 33 to 198 nt (labeled 1 to 4 in FIG. 49 Panel C). Stability of the vectors after 5 to 8 weeks in plants was measured by: (1) RT-PCR using primers outside of the insert; (2) batch sequencing the vector in newly emerged systemic leaves; and (3) direct sequencing of up to 10 cloned CYVaV1 vectors (FIG. 51). In every case, vectors containing any of these four duplicated hairpin-like structures were completely stable and no insert loss was detected. We then designed hairpin-like structures that would “mimic” structures 1 and 4 for insertion in the vector. This involved generating hairpin-like structures with different sequences while maintaining elements of the secondary structure or conformation (i.e., the positions of paired bases and loops, apical loop size) and approximate minimum free energy (delta G or ΔG) (as measured by RNA folding programs) of the hairpin being mimicked. Designed hairpin-like structures typically had an experimental targeting sequence (the one desired in the RISC complex) on the 5′ side of the hairpin-like structure, and 3′ side nucleotides that would fulfil the desired shape and ΔG parameters. In one exception a targeting sequence extended from a region of the 5′ side, through the apical loop, to a region of the 3′ side. Of 52 vectors containing different mimic hairpin-like structures that were tested for stability in N. benthamiana, 50 were completely stable at the assayed time point (5 to 8 weeks after infiltration). Indeed, one such insert was found to still be stable after over a year in citrus (FIG. 51).

We next endeavored to define properties associated with insert stability. We examined the four natural inserts that were stable (which we used as models for mimicked inserts); four non-mimic inserts that were unstable; one non-mimic sequence of 198 nt that was unstable; 50 mimic hairpin-like structures that were stable; and two mimic structures that was unstable. We found that all stable hairpin-like structures were composed of nucleotides that together gave a low, but non-zero, average positional entropy (APE) value for the hairpin (FIG. 52). The formula for calculating the positional entropy of an individual nucleotide (i) is S(i)=−Σpᵢⱼ log (pᵢⱼ). The positional entropy of each nucleotide in a naturally occurring or exogenous sequence can be determined, for example, using the perl script “replot.pl” from the RNAfold program in the Vienna RNA package (https://www.tbi.univie.ca.at/RNA/). The APE is calculated by adding the positional entropy of the individual nucleotides (e.g. as extracted from the EPS file produced by the RNAfold program) and dividing by the number of nucleotides. For naturally occurring hairpin-like structures in CYVaV1, individual nucleotide entropy values vary from 0 to 2.6, where nucleotides that are always predicted to be either unpaired or paired with the same partner have a positional entropy value of 0 and nucleotides with more than one possible pairing partner, or that may be paired or un-paired, have higher entropy values. Positional entropy values were extracted for all nucleotides present in each CYVaV1 segment of interest using the RNAFold web server. Individual nucleotides were color coded from white (entropy value=0) to darkest gray (entropy value=2.6) (FIG. 52, Panel A). Positional information was then sorted and the average positional entropy (APE) for each hairpin-like structure was determined (FIG. 52, Panel B) with the following results.

The four natural model hairpin-like structures that were all stable had APE values of: 0.19 (Hairpin-like structure 1); 0.10 (Hairpin-like structure 2); 0.12 (Hairpin-like structure 3); and 0.15 (Hairpin-like structure 4). All 50 stable mimic hairpin-like structures had APE values ranging from 0.07 to 0.32, which was substantially lower than nearly all of the unstable inserts. Most of the stable hairpin-like exogenous segments also had minimum free energy (DG) similar to (i.e within 10 of) a naturally occurring hairpin-like structure in CYVaV1 of similar (e.g. within 10%) length.

Four unstable hairpin-like inserts had APE values of 1.3, 1.16, 0.83, and 0.39. The unstable insert with APE=0.39 (referred to as M2250gfp30ext herein) was noted to have an 9 high positional entropy (PE greater than 1.0) and 4 very high positional entropy (PE greater than 1.5) nucleotides, with 8 (out of 66 nucleotides total) clustered near the apical loop, which may have contributed to the lack of stability. A deleted (stable) variant found accumulating in plants had a deletion of the apical loop, allowing the high positional entropy residues to form a new apical loop. Another unstable insert (198 nt) was not designed to assume any particular structure and had an APE value of 0.90. An unstable insert with low APE value (0.07; two asterisks in FIG. 52, Panel B) had three consecutive G:C pairs at the stem base and five consecutive G:C pairs in the apical region. Deleting 2 nt from the 3′ side of this hairpin (i.e., deletions did not affect the target sequence on the 5′ side) converted the hairpin from unstable to stable (APE increased slightly to 0.15).

These results strongly suggest that insert APE, optionally together with insert ΔG relative to the insert size, represent important parameters for designing stable inserts. Furthermore, results may be optionally improved by considering one or more additional stability parameters, for example the number of consecutive G:C pairs and number or clustering of high entropy nucleotides. Without intending to be limited by theory, these findings led to our second hypothesis to explain insert instability of VIGS vectors: High hairpin APE values, which imply that the hairpin is metastable and likely assuming multiple conformations with similar high ΔG values negatively impacts the processivity of the RdRp leading to polymerase pausing, which enhances the probability of a recombination event that excises some or all of the insert, thereby leading to unstable inserts. Without intending to be limited by theory, our overarching hypothesis then can be summarized as follows: Hairpin-like structures in viral genomes have evolved to have low APE values, possibly along with specific ΔG relative to the size of the hairpin-like structure, to maintain the tertiary structure of the metastable full-length genome and processivity of the replicating polymerase. Exogenous segments inserted into a viral vector must conform to these properties, which may or may not be virus-specific, to maintain viral vector fitness.

While vector stability for replication is also important in many applications, the ability to design stable inserts for virus vectors is important for allowing VIGS to be used in long-lived plants (e.g. trees and vines) for extended times against pathogens.

We expect that the parameters for stable inserts described herein extend to inserts used in other RNA vectors derived from virus. Very little is known about the secondary structure of most viruses. For all but three viruses, the secondary structure is known for only portions of the virus. However, we note that, to the extent that secondary structures are known, naturally occurring viruses generally do not contain long fully based-paired regions. Considering the experiments and observations described herein, it seems likely that stable inserts for other virus-derived vectors will follow the principles described herein. In the event that one or more numerical ranges described herein to not apply to vectors derived from another virus, we expect that the general technique of mimicking a natural-occuring hairpin-like structure will be useful. For example, the ΔG relative to length and/or APE and/or secondary structure of a wild-type hairpin-like structure may be used as a guide to designing a stable insert for a vector derived from the same wild-type virus. Given the similarities that we have observed between different ulaRNA, and the stability of a hairpin-like structure from one ulaRNA (OULV) when inserted into another ulaRNA (CYVaV1), we expect both the general technique of mimicking and the numerical parameters described herein to apply throughout ulaRNA. For example, we expect that an insert described herein that is stable in CYVaV1 will also be stable when inserted at least into any other ulaRNA. Further, given the observations and examples described herein regarding TRV and CTV, we expect that an insert described herein that is stable in CYVaV1 will also be stable when inserted at least ino CTV and TRV.

The secondary structure of RNA hairpin-like structures includes one or more of base-paired stacks, internal loops (symmetric or asymmetric), apical loops, and junctions (FIG. 53). We hypothesize that thermodynamic features of hairpin-like and other structures contribute to the stability of the entire CYVaV genome, and structures of natural hairpin-like and other regions reflect the end point of long-term thermodynamic evolution. By understanding parameters that have influenced virus secondary structure evolution, foreign hairpin-like structures can be designed with similar features for insertion into virus vectors without loss of fitness or RdRp processivity that otherwise would lead to insert loss.

Solving the secondary structure of CYVaV1 revealed: (1) the longest fully base-paired region was found in Structure 2 (FIG. 49 Panel C) and has 13 continuous base pairs. Structure 2 also has the largest (7 base) apical loop and smallest DG (−13.1 kcal/mol) (FIG. 52), (2) the length of the longest hairpin-like structure is 198 nt (Structure 1 in FIG. 49 Panel C); (3) the length of the longest sequence without local Watson-Crick (canonical) or Watson-Crick (wobble) base-pairing is 21 nt (this sequence is not part of a hairpin-like structure); and (4) the highest number of consecutive G:C pairs in a stack in the CYVaV1 genome is three. Moreover, as described above, the APE of four natural hairpin-like sequences that were studied ranged from 0.10 to 0.19, and the APE of stable hairpin-like mimics ranged from 0.07 to 0.32, values that were lower than those of nearly all unstable inserts. As used herein, the term “base-paired” includes Watson-Crick (canonical) base pairs (i.e. C:G and A:U pairs) and Watson-Crick (wobble) base pairs (i.e. G:U base pairs) when G:U pairing is predicted by the minimum free energy structure.

One hairpin-like insert with a comparatively low APE (GFPmmck63, APE of 0.25) was unstable. However, this insert had five continuous G-C pairs. Another hairpin-like structure with a comparatively low APE (M2250gfp30ext, APE of 0.39) was also unstable. However, this insert had 9 bases (13.8%) with high entropy (PE greater than 1.0), 4 bases with PE over 1.5, and a standard deviation of entropy of 0.52. The instability of these examples may not indicate the maximum APE that may results in a stable hairpin-like insert. A hairpin-like insert with a similar or higher APE, but without the continuous G-C pairs, large percentage of high entropy bases and/or high standard deviation of PE, might be stable. Stable haripin-like inserts may have a maximum APE of 0.75, 0.65. 0.5, 0.4, 0.39, 0.36 or 0.32. Minimum APE of a stable hairpin-like structure may be 0.01, 0.02, 0.03, 0.04, 0.05, 0.06 or 0.07. A range of APE may optionally be provided by any combination of these minimum and maximum APE values, or any APE values described in the examples.

Based on the observation of wild type hairpin-like structures and experiments involving exogenous hairpin-like structures, various additional parameters may be useful for insert stability: (1.1) the length limit of a fully base-paired region; (1.2) the length limit of the entire hairpin-like structures; (1.3) the length limit of consecutive G:C pairs; (1.4) the maximum number of non-paired bases in a symmetrical or asymmetrical internal loop; and, (1.5) the largest average positional entropy of a cluster (i.e. 10 per side) of base-paired nucleotides or an entire base-paired segment. For an RNA vector derived from a wild type virus, an insert may be used that does not exceed one or more of these parameters, or other parameters described herein, as determined by the wild type virus. In some examples, parameters derived from a wild type virus may be modified, for example by 50%. For an insert in a ulaRNA-derived vector, for example a CYVaV 1-derived vector, a hairpin-like insert may have one or more of: nor more than 19, nor more than 13, or no more than 10, consecutive fully paired bases; no more than 300 nt, nor more than 200 nt, or no more than 198 nt bases; no more than 4, or no more than 3, continuous G:C pairs; no more than 20 or no more than 15 bases in a loop (i.e. counting bases on both sides of a loop); an APE of no more than 0.8, or no more than 0.6, in any base-paired region. Optionally, a hairpin-like structure as described immediately above for insertion into a ulaRNA may be inserted into a vector derived from another wild type virus.

Regarding the maximum number of consecutive fully paired bases, the presence of A-U base pairs may allow for larger values. In CYVaV, the largest base-paired region (13 base pairs) had an A-U rich region. Stacks without A-U rich regions in naturally occurring hairpin-like structures in CYVaV had no more than 10 consecutive paired bases.

The limit on the APE of the entire insert tends to result in inserts having at most a small number of bases with high positional entropy. Alternatively or additionally, one or more other guidelines may be used, including: a) the insert does not have any bases with positional entropy greater than 2.0 or greater than 1.5, optionally with an exception that a large insert (e.g. an insert with more than 100 nucleotides) may have a small number (e.g. 1 or 2 or 3) of nucleotides with PE greater than 1.5, b) the insert does not have more than 15%, or does not have more than 10%, of bases with positional entropy greater than 1.0, and c) the standard deviation of the positional entropy of the bases in an insert is 0.5 or less or 0.4 or less, for example in a range of 0.1 to 0.4.

An insert is typically designed by designing a targeting sequence, which provides most or all of a first side of the insert (typically not including the apical loop as part of either side). A second side of the insert, on the opposite side of the apical loop, is designed to be mostly, but not entirely, complementary to the first side. For example, the second side may be 65-90% or 70-85% complementary with the first side. The lack of complete complementarity provides alternating base-paired regions and non-base-paired regions. Each of the base paired regions may have 19 or less, 13 or less or 10 or less base pairs. Each of the base paired regions should also not have more than 4 consecutive G-C base pairs, and optionally not more than 4 consecutive A-U base pairs. Direction of the base pairings is not considered, for example a G-C pairing followed by a C-G pairing is considered to be two consecutive G-C base pairings. Although other parameters described herein may be considered, in many examples hairpin-like structures are stable when designed considering only 65-90% or 70-85% complementary between the first side and the second side of the insert; each of the base paired regions having 19 or less, 13 or less or 10 or less base pairs; and each of the base paired regions have no more than 4 consecutive G-C pairs or no more than 3 consecutive G-C base pairs, and optionally no more than 4 consecutive A-U base pairs. If a hairpin-like structure designed according to these principles is unstable, one or more other parameters or guidelines described herein may be considered to produce a more stable insert.

In some examples, a targeting sequence may extend into the apical loop or across the apical loop from one side of the insert to the other. However, this type of structure may not be possible for all targeting sequences, and might not be beneficial (despite the potentially increased length of targeting sequence) with all targeting sequences, i.e. siRNA targeting sequences.

The limit on the APE of a base-paired region helps to avoid clustering of high positional entropy bases, e.g. bases with positional entropy of more than 1.0. Although the presence of individual high positional entropy bases may not result in a high APE, inserts are less stable when the high positional entropy bases are numerous or clustered near each other. Other factors that may be derived, for example from an analysis of wild type hairpin-like structures and/or examples of stable exogenous hairpin-like structures, to indicate clustering include, for example, the percentage of nucleotides with a high positional entropy (e.g. a positional entropy greater than 1.0); the standard deviation of positional entropies, and the number of bases with very high positional entropy.

In some examples, an insert may exceed one or more of the parameters described above or derived from a wild type virus. For example, we stacked one hairpin-like mimic onto the majority of Structure 3 (in its natural location), resulting in a stable hairpin-like structure of 247 nt (FIG. 49 Panel B). The entirety of this hairpin-like structure was not exogenous, however it suggests that the maximum length of a natural hairpin-like structure might be exceeded. We also introduced a stable insert with 4 consecutive G:C pairs into CYVaV1, whereas a maximum of 3 are found in natural CYVaV hairpin-like structures. An insert with 5 consecutive G:C pairs was not stable. Further, we have used exogenous inserts with APE greater than the APE of natural CYVaV hairpin-like structures. The examples above suggest that in some examples a natural parameter may be exceeded, for example by up to one half, or by up to one third.

Folding the first 2400 nt of tobacco rattle virus (TRV) RNA2 revealed a low entropy hairpin from positions 1727 to 1925. The APE value for this hairpin is 0.32 and the ΔG is −61.5 kcal/mol. This can be compared to similarly sized Structure 1 of CYVaV (APE=0.19; ΔG=−89.2 kcals/mol). In another example, a naturally occurring hairpin-like structure was located in CTV and inserted into CYVaV1 and infected into a benthamina plant (see example further below). The resulting vector was stable when assayed after three-weeks (experiment is ongoing). These observations suggest that inserts designed with reference to other wild type virus may be used in CYVaV or other ulaRNA, and that inserts designed with reference to CYVaV or other ulaRNA, or otherwise as described herein, may be useful in other viral vectors, for example other vector derived from plus-sense RNA plant viruses. Even if the resulting vectors are not as stable as CYVaV1-derived vectors, they may be more stable for replication than comparable vectors with conventional (i.e., fully base-paired) hairpins.

Designing hairpin-like structures that have the appropriate attributes, such as average positional entropy, may start with design of the targeting sequence (the one desired in the RISC complex) on one side, for example the 5′ side, of the hairpin. This is matched by a partially complementary sequence on the other side, for example the 3′ side, that will produce the correct values, e.g. average positional entropy values and or ΔG relative to length, for the hairpin-like structure. The partially complementary sequence may have, for example 65-90% or 70-85% complementarity with the targeting sequence. Optionally, the partially complementary sequence may be designed to result in a hairpin-like structure similar in shape to a natural hairpin-like structure being mimicked and/or incorporate other parameters described herein.

The specification also describes optional modifications to make the vector less fit when the insert is deleted such that replicates with the insert out-compete replicates that have deleted the insert. This method is called “reverse fitness”. Normally, the wild-type vector (with no inserts) is most stable. Altering the vector by over stabilizing its structure (in the sense of structural stability rather than replication stability), for example at or near an insertion site, creates a vector that reverts to a metastable (preferred) structure when containing hairpin inserts. In this way, loss of an insert generates an overly stable RNA (in the structural sense), which is less fit and would be lost from the population. However, these optional “reverse fitness” modifications have been found unnecessary in various examples with stable inserts described herein since the inserts are not deleted with replication, or the inserts are deleted at a very low rate.

As discussed above, In addition to testing the effects of mutations that stabilized the backbone (vector) structure in the vicinity of the inserts and stabilizing the active structure of the ribosome recoding (−1 frameshifting site), the inventors decided to test if inserting duplicates of natural hairpin-like structures into the insert sites would be stable. Despite the size of some of these structures, all four of the duplicate hairpin-like structures used were stable in the insert sites. Next, the inventors discovered that mimicking the secondary structures and approximate DG of the natural hairpin-like structures with exogenous sequences also resulted in vectors that did not lose the inserted sequences. The inventors then investigated the properties of the natural hairpin-like structures, finding that various parameters such as the positional entropy of individual nucleotides, average positional entropy of the overall hairpin, and DG were relevant to stability. Unstable inserts typically had either high positional entropy, higher ΔG, multiple consecutive G:C pairs, or high positional entropy nucleotides that were clustered together. Using these principles, exogenous inserts with targeting sequences were designed that could be inserted into vectors and that were stable (i.e. retained in progeny) even without any reverse fitness modifications described herein. However, one or more reverse fitness modifications may be optionally used in combination with otherwise stable inserts.

In another optional aspect of reverse fitness, a hairpin-like or other structure of the wild type virus is removed in combination with adding one or more inserts. The insert or inserts may be added all in other locations, or an insert may be added in the former location of the structure that was removed. For example, a hairpin-like structure of the wild type virus may be removed and replaced with an exogenous hairpin-like structure. Optionally, the exogenous hairpin-like structure is similar in one or more aspects to the hairpin-like structure that was removed, for example in length, minimum free energy (ΔG), average positional entropy, or secondary structure. Alternatively, the exogenous hairpin-like structure is unlike the hairpin-like structure that was removed, but is similar in one or more ways (e.g. length, minimum free energy (ΔG), average positional entropy, or secondary structure) to another hairpin-like structure in the wild type virus, or a relative of the wild type virus, or the exogenous hairpin-like structure is otherwise a hairpin-like structure as described herein.

At least some of the principles described herein relating to positional entropy may also apply to the lock and dock structures described herein, and in particular to hairpin-like structures within or attached to a lock and dock structure. However, the lock and dock structures are a distinct type of insert since they provide a tertiary structural feature. For example, the GAAA tetraloop of the lock and dock provides a binding motif that induces tertiary folding that is not present in the hairpin-like structures described herein. A lock and dock when analyzed as a whole (i.e. considering the lock and dock and a hairpin-like structure attached to the lock and dock together) may operate with one or more exceptions to the principles described herein. For example, it seems that while lock and dock structures benefit from having an APE as described herein, more clustering of high entropy bases may be tolerated, particularly clustering in the terminal loop region. Individual high entropy bases also seem to be tolerated in the terminal loop region. Lock and dock structures may also be stable with a standard deviation of positional entropies of over 0.4. Optionally, when considering wild type hairpin-like or other structures to determine design principles for exogenous inserts, wild type structures having tertiary interactions or special functions may be excluded from consideration. Alternatively, the principles described herein may be applied separately to a hairpin-like structure attached to a lock and dock. The factors described herein may also be applied separately to a hairpin-like structure included as part of a Y-shaped, V-shape, or T-shaped insert or other insert with a junction.

Similar to umbraviruses, iRNAs do not possess a functional coat protein(s) ORF and/or otherwise encode for any coat protein. In addition, the RNA polymerase of iRNAs is similar to that of umbraviruses. However, unlike umbraviruses, iRNAs do not possess a functional movement protein(s) ORF and/or otherwise encode for any cell-to-cell movement protein(s) or any long-distance movement protein(s) that serves as a stabilization protein for countering nonsense mediated decay.

Conventional viruses lacking coat proteins are generally less stable inside a plant cell given their genomes are vulnerable to the host RNA silencing defense system. However, iRNAs are surprisingly stable in the intracellular environment, which is an important characteristic for an effective vector. iRNAs are also restricted to the inoculated host plant in the absence of a specific helper virus, since without associated virions they are not transmissible by an insect vector. It is believed that iRNAs are encapsidated into virions only when in the presence of a specific helper virus, e.g., such as an enamovirus, including Citrus vein enation virus (CVEV), which is a rarely seen virus in the United States.

In disclosed embodiments, a recombinant plus-sense single stranded RNA vector is provided that comprises a replication element(s) (e.g., a portion(s) of the vector molecule responsible for replication) and a heterologous segment(s). The RNA vectors of the present disclosure are capable of accumulating to high levels in phloem, and are capable of delivering a therapeutic agent(s) such as a protein, a peptide, an antibacterial and/or an insecticide (e.g., siRNAs) directly into the plant tissue. In certain implementations, the RNA vector is derived from an iRNA molecule, which lacks the ability to encode for any coat protein(s) or movement protein(s). For example, the vector is derived from and/or includes structural elements of the iRNA molecule known as Citrus yellow vein associated virus (CYVaV), an unclassified molecule associated with yellow-vein disease of citrus. CYVaV and CYVaV-like RNA molecules are widespread in numerous plants, e.g., including but not limited to limequat citrus, strawberry, hops, switchgrass, corn, hemp, fig trees, prickly pear cactus, and sugarcane. CYVaV and CYVaV-like RNA molecules are generally asymptomatic and without a helper virus in such plants.

Thus, disclosed embodiments provide for an iRNA-based vector built on or derived from a plus-sense single-stranded RNA molecule using genetic components, optionally with inserts that mimic genetic components, from an iRNA molecule, e.g., CYVaV or a relative thereof, for example a Class 2 ulaRNA. In addition, the present disclosure is directed to kits and/or mixtures comprising an iRNA-based (e.g. a CYVaV-based) vector(s). Such mixtures may be in a solid form, such as a dried or freeze-dried solid, or in a liquid, e.g. as aqueous solution, suspension or dispersion, or as gels. Such mixtures can be used to infect a plant, plant tissue or plant cell. Such kits and mixtures may be used for successfully infecting a plant(s) or plant cell(s) with the iRNA-based vectors of the present disclosure and/or for expression of heterologous proteins or delivery of other therapeutic agents to such plant or plant cell(s).

The present disclosure also relates to a plant, plant tissue, or plant cell comprising said iRNA-based vector as disclosed herein, and/or a plant, plant tissue, or plant cell comprising a therapeutic agent or heterologous polypeptide encoded or delivered by said vector. The present disclosure also provides for methods of isolating such heterologous polypeptide from the plant, plant tissue, or plant cell. Methods for isolating proteins from a plant, plant tissue or plant cell are well known to those of ordinary skill in the art.

CYVaV was found in four limequat trees in the 1950s independent of any helper virus (Weathers, L. (1957), A vein yellowing disease of citrus caused by a graft-transmissible virus, Plant Disease Reporter 41:741-742; Weathers, L. G. (1960), Yellow-vein disease of citrus and studies of interactions between yellow-vein and other viruses of citrus, Virology 11:753-764; Weathers, L. G. (1963), Use of synergy in identification of strain of Citrus yellow vein virus, Nature 200:812-813). Further analysis and sequencing of CYVaV was conducted years later by Georgios Vidalakis (University of California, Davis, CA; GenBank: JX101610). Dr. Vidalakis's lab conducted analysis on samples collected from previously established tree sources (Weathers, L. G. (1963), Use of synergy in identification of strain of Citrus yellow vein virus, Nature 200:812-813) and maintained in the disease bank of the Citrus Clonal Protection Program (CCPP). Studies by the Vidalakis lab to characterize CYVaV were inconclusive. However, many of the infected samples containing CYVaV also contained the enamovirus citrus vein enation virus (CVEV); it was relatively common in the 1950s through 1980s for CCPP personnel to mix infect plants with yellow-vein and vein enation for symptom enhancement.

CYVaV is a small (˜2.7 kb) iRNA molecule composed of a single, positive sense strand of RNA. It replicates to extremely high levels, is very stable, is limited to the phloem, and has no known mechanism of natural spread. As such, CYVaV is ideal as a vector platform for introducing an agent(s) into a plant host, e.g., such as a small RNA (e.g., non-coding RNA molecule of about 50 to about 250 nt in length) and/or proteins for disease and/or pest management. The production of proteins that bolster (or silence) defenses, antimicrobial peptides that target bacterium, and/or small RNAs that target plant gene expression or the insect vectors of disease agents provide an effective management strategy. To be efficacious, the proteins and small RNAs should be produced in sufficient quantities and accumulate to sufficient levels in the phloem, particularly small RNAs designed to be taken up by targeted insects or fungal pathogens.

CYVaV is only transmissible in nature with a helper virus but may be moved from tree to tree by grafting, and has been shown to infect nearly all varieties of citrus with the exception of hearty orange, including but not limited to infecting citron, rough lemon, calamondin, sweet orange, sour orange, grapefruit, Rangpur and West Indian lime, lemon, varieties of mandarin, varieties of tangelo, and kumquat. It produces a yellowing of leaf veins in the indicator citron tree and has no or very mild yellow vein symptoms in sweet orange and other citrus with no reported impact on fruit quality, or otherwise causing harm to trees.

The polynucleotide sequence (bases 1 to 2692) of CYVaV is presented below (SEQ ID NO:1):

| | |
|---|---|
| ggguaaauau ggauccuuca ucuuugcccc gugccuguug gcaucaugcc | 50 |
| agacaggugu uucgagcauc aacuagcuuc ucaagagagg ugguucgcgc | 100 |
| ugcucguaga uggguuacca ugcccaccag ucgccaugca uaugacuuuu | 150 |
| caacgagucu aggcauugug auugcugagc cugcagcucg uuuacgacgc | 200 |
| cgucugcccu cuguacgaaa gugcgcagag aaguuaguag uccacaagca | 250 |
| agucgacacu uugguggacg aauggugcuc uggaauuccc aacccugaua | 300 |
| ucguagaagu ugguugggca cuccgucuga gggaccguuu cggucuuccu | 350 |
| cccgcuucug agccuacccg gcucaguggu gagagauggg ugcucaaaca | 400 |
| acucaauggg guagauccug agucauggaa ugcugaucuu gguaggucag | 450 |
| uucauaucca aggagacuac gccccaggga ggaaugccca uaucgcucag | 500 |
| gucgcggcga ccuugugguu aacuaggacc uugcaugaca aggccuuggc | 550 |
| ucgccaccag gguuuucgcg auuugcagug auuggggucg acgggcuaga | 600 |
| ggcaaaagca gugccucuag cuucuggacu ccgacugcuu ccgguuccgc | 650 |
| gacccggaca aagucgacga cugucucaga ccuuguuacu uccaacaccu | 700 |
| cgugcucaau ucgugaauca cgcgugcucg gcuaacaacc uuggacgugu | 750 |
| gaugaccaca cguguguugc aguacaaggg ccgagauccg auccuucccu | 800 |
| cuucugaagc ccuucaccga cuuaaccuuc ggauagcuga gcuauauagg | 850 |
| ucuagaccuu cuaccgucua uccauuaagu uaugaagggu uucucaauug | 900 |
| cuaugaaggc cgacagcgua cucguuacgc ccaagccguc gagcaguuga | 950 |
| ugcgguccac ucuugagccg aaagaugcgc gaguugaaac guucauuaag | 1000 |
| aacgagaaau uugacugggc guugaaaggg gaggaggcug auccucgagc | 1050 |
| aauccaacca aggaagccga aauauuuggc ugagguugga cggugguuca | 1100 |
| aaccuuugga gcgaaucauc uacaaggauc ucaguaaaag guuguauggu | 1150 |
| gagggugcug agccguguau cgccaaaggc cuaaaugcau uagaaucugg | 1200 |
| agcgacuuug aggcgcaaau gggagaaguu uucuucucca guuugcguuu | 1250 |
| cucucgacgc uuccagguuc gaccugcaug uaagcguugg caugcuaaag | 1300 |
| uucacacaca agcuauauga cuauuacugu aagucuccca cucuccagcg | 1350 |
| cuaucucaaa uggacacucc gcaaccaugg cgucgccucc ugcaaagaau | 1400 |
| ugucauauga guaugagguu guuggccgga gaaugagugg ugacauggac | 1450 |
| acugcauugg gcaacugcgu cauuaugucg auacuuacau gguuuaugcu | 1500 |
| uagugaacuu ggcauuaagc augaauuauu cgauaauggu gacgauuguu | 1550 |
| uguucauuug cgagucucac gacgucccca gccccgaggu aauuacaaac | 1600 |
| ugguuuucgg acuuuggguu ugugguuagg uuggaaggcg ucacguccgu | 1650 |
| guuugagcgu auugaguuuu gccaaacuuc cccaguaugg acugagaggg | 1700 |
| guuggcugau guguaggaau auuaagucau ugaguaaaga ccuuacgaau | 1750 |
| guuaauucgu gcacgggcuc cacgauugaa uauacccacu gguugaaagc | 1800 |
| agugggaaag ugcgggucaa uacucaaugc ugguguaccu auauuucagu | 1850 |
| ccuuucacaa caugcuggaa aggcuuggca cuaacucucg uauugaucga | 1900 |
| gggguuuucu ucaaaucagg gcuaguuaau cucauucgug ggauggacag | 1950 |
| gcagccugac guugacauca cuacuuccgc ucggcuuucu uucgaagugg | 2000 |
| cauucgggau aacacccggg augcaauugg cuauugaacg guacuaugac | 2050 |
| ucugucaugg gcucgcugag uaaaauagaa acaacuaagu ggccaauuga | 2100 |
| acuaagaaag gaauacgaac acggaaguga gugguacgag gacuuaggcg | 2150 |
| uccuaggaug aauaggguca uugguuuacc gaugauaccu guucagaaua | 2200 |
| ggauugcucg agcuucguug guuaggguaa cucacauacc uucuuccaua | 2250 |
| acuggaaaag gucgugugag caaccuaacc aguuaaugua ggugucuuuc | 2300 |
| cguaucuagu cacgauggua agcaacccgu uuaucuguac ggcgcucacc | 2350 |
| cguggguagg aaggugaagg uuuugugucc uuuaggucuu ggacagucug | 2400 |
| cgggcuuggg aacgacgccc cgcuagcaac guacugcucu ccuaccggac | 2450 |
| ugguagcuua auugucaucu uggagcgaua gcacuguggg ccucacccuu | 2500 |
| cgcgcguugg acguguugcg ugccccccac agauuuguga aacucuaugg | 2550 |
| agcaguuccg cgagccagaa gggaggaugg ccgccuggcg uaauccagga | 2600 |
| gcucuggggg gcuuguacuc agaguagcau ucugcuuuag acuguuaacu | 2650 |
| uuaugaacca cgcgugucac guggggagag uuaacagcgc cc | 2692 |

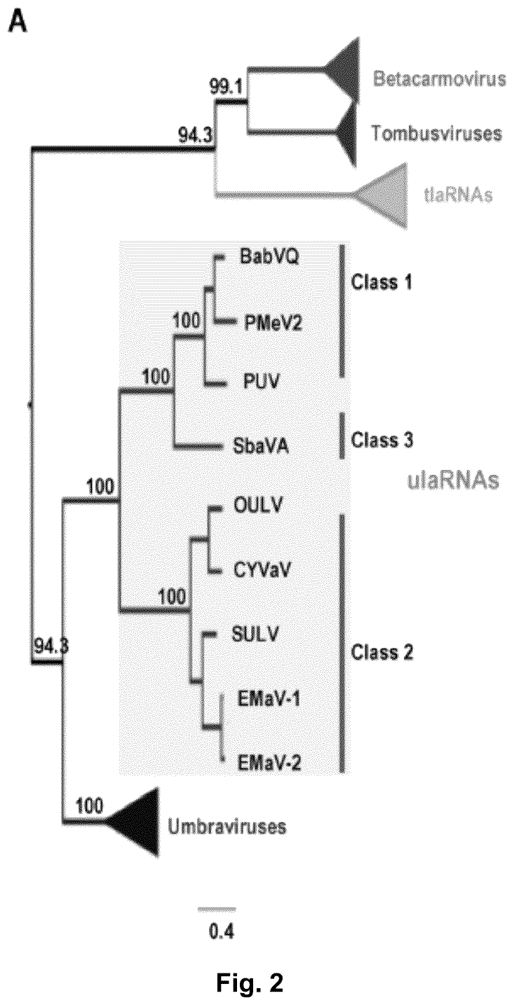

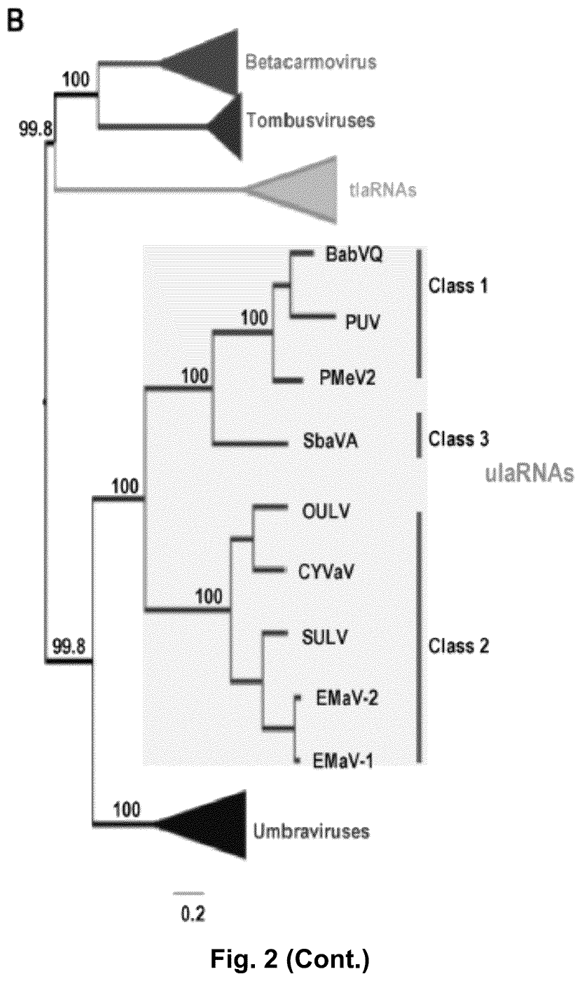

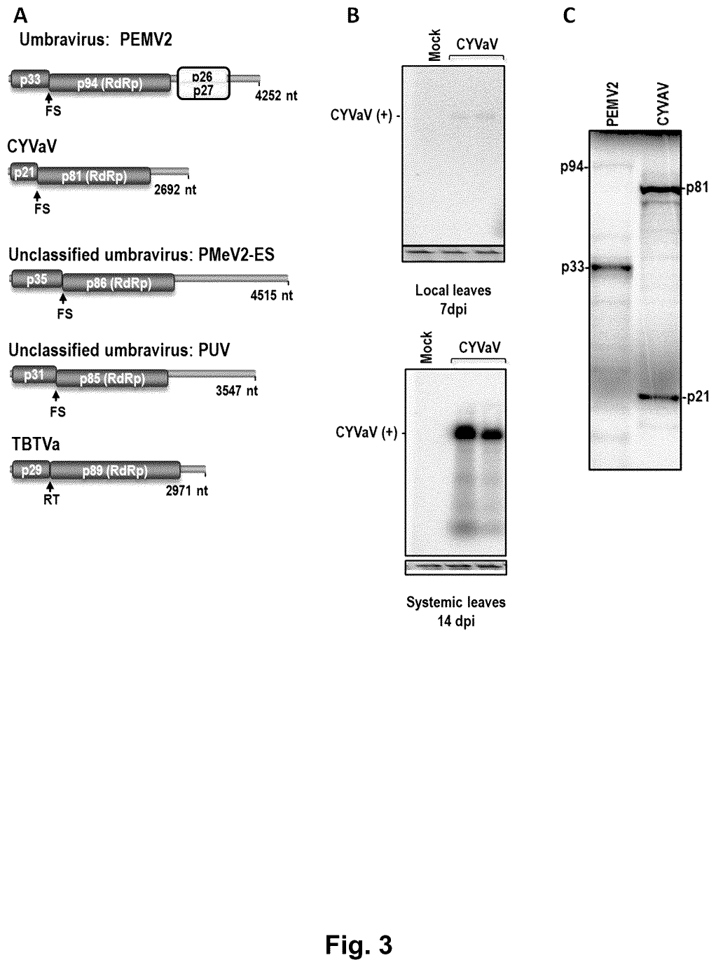

Relatedness of CYVaV with other viruses including Tombusviridae viruses and umbraviruses is shown in FIG. 2. Given the close relationship of CYVaV to umbraviruses, CYVaV and its relatives have been called umbravirus-like RNA (“ulaRNA”) in the art. The ulaRNA may be separated into 3 classes. Genome organization of CYVaV and similar RNA molecules is illustrated in FIG. 3, Panel A, including PEMV2, PMeV2-ES (GenBank: KT921785), PUV (GenBank: KP165407.1), and TBTVa (GenBank: EF529625.1). The RdRp of CYVaV is most closely related to the umbravirus Pea enation mosaic virus RNA2 (PEMV2). Examination of 5′ and 3′ sequences of CYVaV revealed considerable similarity to those of umbraviruses, confirming that CYVaV is indeed a complete infectious agent. CYVaV has a plus-sense single stranded RNA genome that only encodes two proteins involved in replication: p21, a replicase-associated protein in related molecules; and p81, the RNA-dependent RNA polymerase (RdRp) that is synthesized by a ribosome recoding (frameshift) event (FIG. 3, Panel A). Levels of the RNA-dependent RNA polymerase (RdRp) synthesized by frameshifting in vitro are shown for PEMV2 and CYVaV. The difference in levels of p94 (RdRp) from PEMV2 as compared to p81 from CYVaV is significant (FIG. 3, Panel C). The frameshifting site of CYVaV is one of the strongest known in virology and believed to be responsible for its exceptionally high accumulation.

The polynucleotide sequence of the 3′ end of CYVaV (bases 2468 to 2692) is presented below (SEQ ID NO:2):

ucu uggagcgaua gcacuguggg ccucacccuu
cgcgcguugg acguguugcg ugccccccac agauuuguga
aacucuaugg agcaguuccg cgagccagaa gggaggaugg
ccgccuggcg uaauccagga gcucuggggg gcuuguacuc
agaguagcau ucugcuuuag acuguuaacu uuaugaacca
cgcgugucac guggggagag uuaacagcgc cc

The polynucleotide sequence of the 3′ Cap Independent Translation Enhancer (3′ CITE) of CYVaV (bases 2468 to 2551) is presented below (SEQ ID NO:3):

ucu uggagcgaua gcacuguggg ccucacccuuc
gcgcguugg acguguugcg ugccccccac agauuuguga
aacucuaugg a

The 3′ end (and 3′ CITE) of CYVaV comprises the following conserved polynucleotide sequence(s) (bolded and underlined above):

| (SEQ ID NO: 4) | |
|---|---|
| | auagcacug; |
| | and/or |
| (SEQ ID NO: 5) | |
| | gauuuguga. |

The polynucleotide sequence of CYVaV that encodes for protein p21 (bases 9 to 578) is presented below (SEQ ID NO:6):

au ggauccuuca ucuuugcccc gugccuguug
gcaucaugcc agacaggugu uucgagcauc aacuagcuuc
ucaagagagg ugguucgcgc ugcucguaga uggguuacca
ugcccaccag ucgccaugca uaugacuuuu caacgagucu
aggcauugug auugcugagc cugcagcucg uuuacgacgc
cgucugcccu cuguacgaaa gugcgcagag aaguuaguag
uccacaagca agucgacacu uugguggacg aauggugcuc
uggaauuccc aacccugaua ucguagaagu ugguugggca
cuccgucuga gggaccguuu cggucuuccu cccgcuucug
agccuacccg gcucaguggu gagagauggg ugcucaaaca
acucaauggg guagauccug agucauggaa ugcugaucuu
gguaggucag uucauaucca aggagacuac gccccaggga
ggaaugccca uaucgcucag gucgcggcga ccuugugguu
aacuaggacc uugcaugaca aggccuuggc ucgccaccag
gguuuucgcg auuugcag

The amino acid sequence of protein p21 is presented below (SEQ ID NO:7):

MDPSSLPRACWHHARQVFRASTSESREVVRAARRWVTMPTSRHAYDESTS
LGIVIAEPAARLRRRLPSVRKCAEKLVVHKQVDTLVDEWCSGIPNPDIVE
VGWALRLRDRFGLPPASEPTRLSGERWVLKQLNGVDPESWNADLGRSVHI
QGDYAPGRNAHIAQVAATLWLTRTLHDKALARHQGERDLQ

The polynucleotide sequence of CYVaV that encodes for protein p81 (bases 752 to 2158) is presented below (SEQ ID NO:8):

augaccaca cguguguugc aguacaaggg
ccgagauccg auccuucccu cuucugaagc ccuucaccga cuuaaccuuc ggauagcuga
gcuauauagg ucuagaccuu cuaccgucua uccauuaagu uaugaagggu uucucaauug
cuaugaaggc cgacagcgua cucguuacgc ccaagccguc gagcaguuga ugcgguccac
ucuugagccg aaagaugcgc gaguugaaac guucauuaag aacgagaaau uugacugggc
guugaaaggg gaggaggcug auccucgagc aauccaacca aggaagccga aauauuuggc
ugagguugga cggugguuca aaccuuugga gcgaaucauc uacaaggauc ucaguaaaag
guuguauggu gagggugcug agccguguau cgccaaaggc cuaaaugcau uagaaucugg
agcgacuuug aggcgcaaau gggagaaguu uucuucucca guuugcguuu cucucgacgc
uuccagguuc gaccugcaug uaagcguugg caugcuaaag uucacacaca agcuauauga
cuauuacugu aagucuccca cucuccagcg cuaucucaaa uggacacucc gcaaccaugg
cgucgccucc ugcaaagaau ugucauauga guaugagguu guuggccgga gaaugagugg
ugacauggac acugcauugg gcaacugcgu cauuaugucg auacuuacau gguuuaugcu
uagugaacuu ggcauuaagc augaauuauu cgauaauggu gacgauuguu uguucauuug
cgagucucac gacgucccca gccccgaggu aauuacaaac ugguuuucgg acuuuggguu
ugugguuagg uuggaaggcg ucacguccgu guuugagcgu auugaguuuu gccaaacuuc
cccaguaugg acugagaggg guuggcugau guguaggaau auuaagucau ugaguaaaga
ccuuacgaau guuaauucgu gcacgggcuc cacgauugaa uauacccacu gguugaaagc
agugggaaag ugcgggucaa uacucaaugc ugguguaccu auauuucagu ccuuucacaa
caugcuggaa aggcuuggca cuaacucucg uauugaucga gggguuuucu ucaaaucagg
gcuaguuaau cucauucgug ggauggacag gcagccugac guugacauca cuacuuccgc
ucggcuuucu uucgaagugg cauucgggau aacacccggg augcaauugg cuauugaacg
guacuaugac ucugucaugg gcucgcugag uaaaauagaa acaacuaagu ggccaauuga
acuaagaaag gaauacgaac acggaaguga gugguacgag gacuuaggcg uccuagga

The amino acid sequence of protein p81 is presented below (SEQ ID NO:9):

MTTRVLQYKGRDPILPSSEALHRLNLRIAELYRSRPSTVYPLSYEGFLNC
YEGRQRTRYAQAVEQLMRSTLEPKDARVETFIKNEKFDWALKGEEADPRA
IQPRKPKYLAEVGRWFKPLERIIYKDLSKRLYGEGAEPCIAKGLNALESG
ATLRRKWEKFSSPVCVSLDASRFDLHVSVGMLKFTHKLYDYYCKSPTLQR
YLKWTLRNHGVASCKELSYEYEVVGRRMSGDMDTALGNCVIMSILTWFML
SELGIKHELFDNGDDCLFICESHDVPSPEVITNWFSDFGFVVRLEGVTSV
FERIEFCQTSPVWTERGWLMCRNIKSLSKDLTNVNSCTGSTIEYTHWLKA
VGKCGSILNAGVPIFQSFHNMLERLGTNSRIDRGVFFKSGLVNLIRGMDR
QPDVDITTSARLSFEVAFGITPGMQLAIERYYDSVMGSLSKIETTKWPIE
LRKEYEHGSEWYEDLGVLG

The replication element of CYVaV (e.g., that encodes for protein p81) comprises the following conserved polynucleotide sequence(s) (highlighted and underlined above):

(SEQ ID NO: 10)
cguuc;
(SEQ ID NO: 11)
gaacg;
(SEQ ID NO:12)
gguuca;
(SEQ ID NO:13)
ggag;
and/or
(SEQ ID NO: 14)
aaauggga.

In addition, CYVaV may additionally comprise the following conserved polynucleotide sequence(s) (highlighted and underlined above):

(SEQ ID NO: 15)
ucgacg;
and/or
(SEQ ID NO: 16)
cuccga.

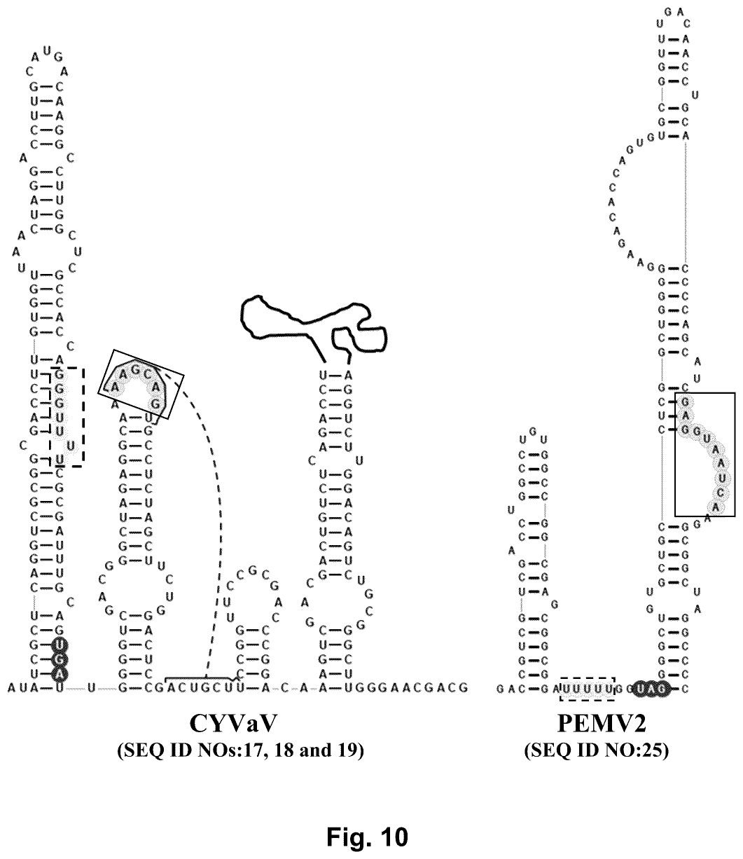

The polynucleotide sequences of recoding frameshift sites of CYVaV (see also FIG. 10) is presented below:

(SEQ ID NO: 17)
ucgcucaggucgcggcgaccuugugguuaacuaggaccuugcaugacaag
gccuuggcucgccaccaggguuuucgcgauuugcagugauuggggucgac
gggcuagaggcaaaagcagugccucuagcuucuggacuccgacugcuucc
gguuccgcgacccgga
(SEQ ID NO: 18)
caaagucgacgacugucucagaccu
(SEQ ID NO: 19)
aggucuuggacagucugcgggcuugggaacgacg

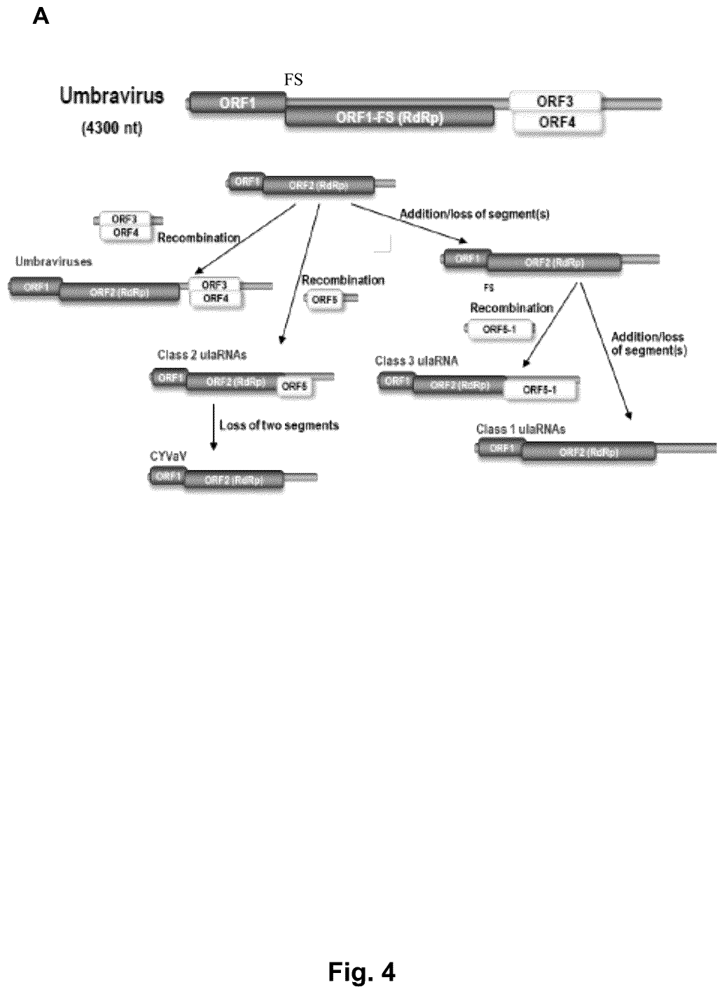

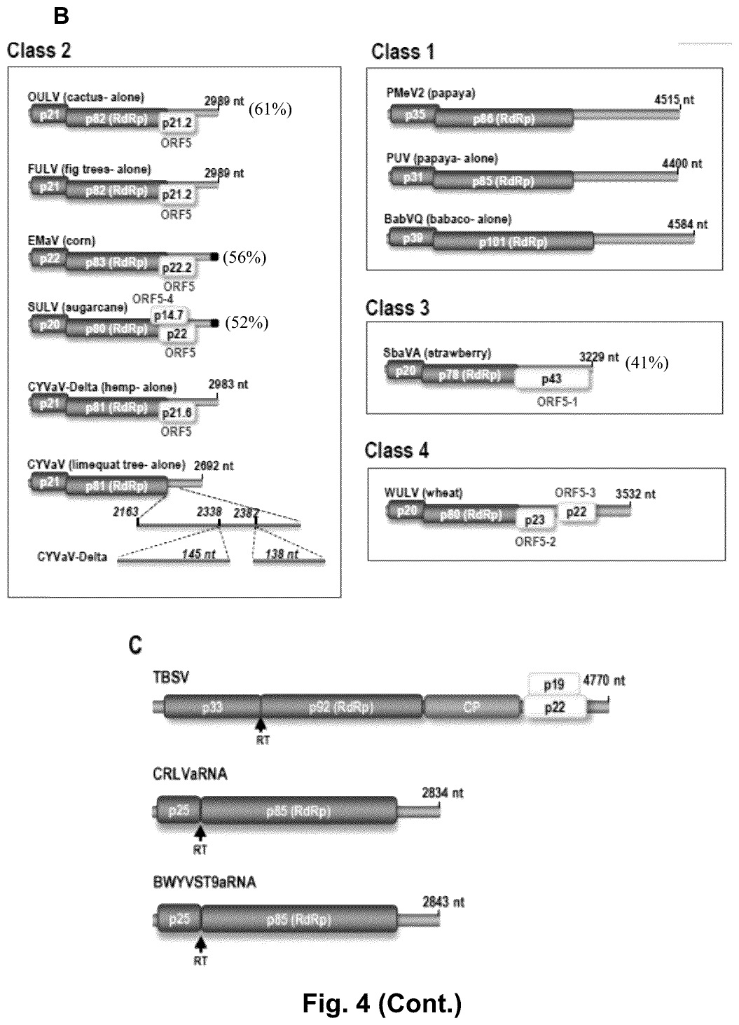

Highly similar iRNAs have also been found in Opuntia (GenBank: MH579715), fig trees, and Ethiopian corn (FIG. 4), suggesting an unusually large or possibly even unlimited host range for the RNA vectors disclosed herein.

The polynucleotide sequence of a similar iRNA identified in a fig tree (sometimes referred to herein as “iRNA relative 1” or “iRNA r1”) is presented below (SEQ ID NO:20):

aaauauggauucgauaucaaugcccgucgccugcuggucaaaagccaggcaggucuugcguacaccag
cuaacuuuuccaaagggguagugaaggcugcguaccggugggucaacaugcccagagccaaauauguc
agagaugucuccacgagucuuggcauaguugucgcugagccuguugcugccgugcgccguuagaugcc
uucgauaagcagccuugcggaggaguugguaacacgccagagcgucgacacucugguggacgauuggu
gucucggacuuuccaacccugacaacaacguggagguugguugggcacuucgucugagggaccgcuuu
ggucuuccucccgccucugagcccacaaggcucaguggugagagaugggugcuuaaacaacucaaugg
gguagacccggagucguggaauguugaucugcaaagcguuuucgaagacgcucaggaugacuuccauc
gggacuacgccccaaggaggaaugcccaaaucgcucaaauugcggcaacccuauggcuuacaaagacc
uuagucgauaaggcuuuagcacgccaucaggauuuucgcaguuugcagugauuggggucgacgggcua
gaggcuaaagcagugccucuggcugcuggacuccgacugcuuccgguuccgcggcccggacaaagccg
acggcugucucaaaccuugcuacucccuacuccccgugcucaauuugucaaucacgcuaacucaggua
auaauuuggggcguguuuugaccacacgggugaugcaauacaaaggccgagacccgauacuacccucc
caggaagcccugcgcaaacuuaaccuucggauaggacaguuguauaagucuagaccauccacugucua
uccccugaguuaugauggguuucuuaauuguuaugauggccgacagcguacucgcuacgcucaugccg
ucgagcaauugaugggugccgcucugaccccaaaagaugcgcgaguugagacguucauuaagaacgag
aaguuugauugguuguugaagggagacgaggcugauccucgugcaauccaaccuaggaagccgaaaua
uuuggccgagguuggucgaugguucaaaccguuggagcgaaucaucuacaaggaucucaguuugcguu
uguacggugauaacgcugaaccuugcauugccaaaggcuuaaaugcauuggaaucaggggcuacguug
agacguaaaugggaaaaguucgcuaauccuguuuguguuucauuggaugcuucucguuucgaccugca
cguaaguguuggcuuguuaaaguucacgcauaaauuguacaacuauuacugcaagucucccacucuuc
aacgauaucucaaauggacacuccgcaacuccgguaucgccuccuguaaggaaaaaucauaugcguau
gagguugaaggccguagaaugaguggcgacauggacaccgcauuaggcaacuguaucaucaugagauu
auuaacuugguuuaugcuuagcgaacuuggcgugcggcaugagcuuuucgauaauggugaugacuguu
uguuuauuugugaaaaagaagacguuccuagugcugagguaaucacgaacugguuuacggauuuuggg
uuugugguuaagcuagaaggcgucacguccguguuugagcgcauugaguucugucagaccucaccagu
auggacugcgaggggauggcugauguguagaaacaucaagucauugaguaaagauuuaacgaauguua
auucgugcacugguucugccguugaauacacucauugguugaaggcggugggcaaguguggaucuaua
cucaaugcuggugugcccauauuucaguccuuucacaacauguuggucagguugggcacgaauucgcg
uauagaucgcgggguauucuuuagguguggacuuguuaaucucauucugggauggacagacaaccuga
aaguugagaucacuacuuccgcucgucuuucuuuugaaguggcauucgggaucacucccggcaugcaa
uuggcuauugagcaauuuuaugacucagucgugggcccucuggguaaaauaaaaucuguaaaauggcc
aauagaucuaagaaaggaauacgauuacggaagcgcgugguucgaagaccaaggcguccuagggugaa
caaggaacucggauuaccgaugacaccuguucaaacuagaaugguucggucaacguugaccaaggaga
ccaacauaccuucuacugcaaauagcggucgggaggcuguuugggcuuguuggccaaucaacuuuagu
gucuuuccgcaacuagccucacucgugaauaaaccguuauacuggcguguguccagugugcaaguugc
aauggagccggcgaugucuacuuccacccaacauuguggaguuggucucaguucuucuggggccuuca
cuaacggugauggguucgguaacgucuuuaagcucuugcguucuuguaacuauacgcggcgcucuccc
gugggaggaaacgugauggucaaauggcccaucugcaugcccuucauucuuaacgaugaugcgcacaa
gaacacaggauuaaccgccugugugaucauugcagucaccaauacuggugugcuaacuggucaaucuu
ggacggagauucuuuugaauguggaguauguagugggugcauagacagucugcgggcuugggaacgac
gccccgcuagcaacguacugcucuccuaccggacugguagccguuuaguuaucuuggagcgauagcac
ugugagccucacucaacgcgcgauggacguggcgagugccccucagagauuugugaaacucuauagag
cuauuucgcgagccagaagggaggauggccaccugguguaagccagguauccccggggggcuuguacu
cggggucgcauuacugcuuagaccacaagguaggguucgcaucuuggaacugacccuaugaccuugug
ggugcccuaaccggacugguagccguuuaauaucuuggagcgauuagcacgugugagcccucacucaa
cggcgcgauuggacguggcgagugccccucagaguaaucugcagagcuccggcagucgugggaggcaa
ggca

The polynucleotide sequence of an iRNA identified in another fig tree (sometimes referred to herein as “iRNA relative 2” or “iRNA r2”) is presented below (SEQ ID NO:21):

cucccacgacugccggagcucugcagaauuccaccggggguaccuggcuuacaccagguggccauccu
cccuucuggcucgcggaauagcucuauagaguuucacaaaucucugaggggcacucgccacguccauc
gcgcguugagugaggcucacagugcuaucgcucccagaauucgggauaaauauggaagaaacuucuuu
gcccaaagccugcuggaucaaaagccaggcaggucuugcguacaccagcuaacuuuuccaaaggggua
gugaaggcugcguaccggugggucaacaugcccagagccaaauaugucagagaugucuccacgagucu
uggcauaguugucgcugagccuguugcugccgugcgccgucagaugccuucgauaagcagccuugcgg
aggaguugguaacacgccagagcgucgacacucugguggacgauuggugucucggacuuuccaacccu
gacaacaacguggagguugguugggcacuucgucugagggaccgcuuuggucucccucccgccucuga
gcccacaaggcucaguggugagagaugggugcuuaaacaacucaauggaguagacccggaaucuugga
augacgacuaugcguucgaagacgcucaggaggauuuucaacgggaauacgucccgggaaggaaugcc
cauauugcugcaacugcggcaacucuauggcugacaaagaccuuguaugacaaggcuuuaguucgcca
ucaggguuuucgcaguuugcagugauuggggucgacgggcuggaggcuaaagcagugccuccagcugc
uggacuccgacugcuuccgguuccgcggcccggacaaagccgacggcugucucagaccuuacuacuuc
cuacuccccgugcuacuuuugucaaucaugcaaauucaggcaauaaucuugagcguguuuugaccaca
cgggugaugcaauacaaaggccgagacccgauacuacccucccaggaagcccugcgcaaacuuaaccu
ucggauaggacaguuguauaagucuagaccauccacugucuauccccugaguuaugauggguuucuua
auuguuaugauggccgacagcguacucgcuacgcucaugccgucgagcaauugaugggugccgcucug
accccaaaagaugcgcgaguugagacguucauuaagaacgagaaguuugauugguuguugaagggaga
cgaggcugauccucgugcaauccaaccuaggaagccgaaauauuuggccgagguuggucgaugguuca
aaccguuggagcgaaucaucuacaaggaucucaguuugcguuuguacggugauaacgcugaaccuugc
auugccaaaggcuuaaaugcauuggaaucaggggcuacguugagacguaaaugggaaaaguucgcuaa
uccuguuuguguuucauuggaugcuucucguuucgaccugcacguaaguguuggcuuguuaaaguuca
cgcauaaauuguacgacuauuacugcaagucucccacucuucaacgauaucucaaauggacacuccgc
aacuccgguaucgccuccuguaaggaaaaaucauaugcguaugagguugaaggccguagaaugagugg
cgacauggacaccgcauuaggcaacuguaucaucaugacgauauuaacuugguuuaugcuuagcgaac
uuggcgugcggcaugagcuuuucgauaauggugaugauuguuuguucauuugcgaagaaaaagacgua
ccuagccccgagacgaucaugaacugguuugcggauuuuggguuugugguuagguuagaaggcgucgu
guccguguuugagcgcauugaguucugccaaacaucgccuauauggacugaucgagguuggcugaugu
guagaaacaucaagucuuugaguaaggaucuuacgaacguuaauucgugcacuggcuccacuguugaa
uacacccauugguugaaagcaguuggaaaguguggaucggugcucaaugcgggugugccuauauuuca
gucauuucacaacauguugaugcgauuggguacgaauucgcguauagaucgcgggguauucuuuaggu
guggacuuguuaaucucauucgugggauggacagacaaccugaaguugagaucacuacuuccgcucgu
cuuucuuuugaaguggcauucgggaucacucccggcaugcaauuggcuauugagcaauuuuaugacuc
agucgugggcccucuggguaaaauaaaaucuguaaaauggccaauagaucuaagaaaggaauacgauu
acggaagcgcgugguucgaagaccaaggcguccuagggugaacaaggaacucggauuaccgaugacac
cuguucaaacuagaaugguucggucaacguugaccaaggagaccaacauaccuucuacugcaaauagc
ggucgggaggcuguuugggcuuguuggccaaucaacuuuagugucuuuccgcaacuagccucacucgu
gaauaaaccguuauacuggcguguguccagugugcaaguugcaauggagccugcaaugucuucuucca
cccaacauugugguguuggucucaguucuucuggggccuucacauaacggugauggguucgguaacgu
cuuuaagcucuugcguucuuguaacuauacgcggcgcucucccgugggaggaaacgugauggucaaau
ggccuaucugcaugcccuucauucuuaacgaugaugcgcacaagaacacaggauuaaccgccugugug
aucauugcagucaccaauacuggugugcuaacuggucaaucuuggacggagauucuguugaaugugga
guauacgccccgcuagcaucguacugcucuccuaccggacugguagccguuuaguuaucuuggaguga
uagcacuguggggccacauuugacgcgcauuggacgcagacaaugucccuccacagauuugugaaucu
cuauggagcuguaaccucggucucucuauagcuuguccgaacaggaaauggacauaaaauaauugcug
uuccaacacguuguguugguaaagaaguuauagauguggugcgccagacaaguggauggcaaccugga
guaauccaggcgcucuggggggcuuauacucggagugcauuacugcuuuagaccguuaaucucaagaa
ccaugugugucgcauggggaggauuaacggcgcccaauucccuuguuaguuuagguacgccuuggucu
ucgaaccacgc

The polynucleotide sequence of an iRNA identified in maize (sometimes referred to herein as “iRNA relative 3” or “iRNA r3”) is presented below (SEQ ID NO:22):

gggguaaauauggagaaccagcacacccauguuugcccacggucguuccugcgaaccugcagggcgau
ccucgcggcuccagccaacuacggucgugauguggucaaaaucgccuacaaaugggcaucacgaaacc
ccgccaccgccccccgaaguguccgagaauccaucggggucguugucggaagcgcuguggacuucuug
agcgcuccucgcaagcguuuagaagaccgcgcagagcaguuggugcaagacgaccgggucgaccggau
cguccgcgagugggagcuaggaaccgcugacucccgaauuccggaaguugagugggcauaccgucugc
gcgaccgcuucggcgucguguccgccagcgagccugcuaggcaaacuggugagaggugggugcucaag
caacuagagggauuggaggggggggaguuccgcugcauacccauugagccauucuuuggugaugcacc
ggcccccguccauagcccugggagcaacagcgugauugcugcuauugcggcgacccuuuggaugacgc
cuacccgccuugaccgggcguugagacgucaccaggguuuucgcaacuagcggugaucggagucgacg
gagugucugcuuuagcggugcaggcaucuucugaacuccgaccgcuacggguugggcgaccccgucaa
agucgacgucguucguggucucugacuaugccagcacccaaguccuguuucgugaaccacgcuaacuc
ugaccacaaucucaaaacggucauggaaaacagggugcucaaguacaaaggccaagaacccgcaaagc
cccggguagaagccuauaagcagcucuaugaaaggauacgaccgcgauaucguucucuaccugacacg
gucuauccucuaucauaugauggcuuccucaagugcuacuccggacguaggcgaacacgauacgaaca
ggccguccaggaguugagaaacgcgccacucacacccgaagaugcugucguuuccacguucaucaaga
acgagaaauucgauuggcuccaaaagaaagaacuugcggaucccagagcuauccaaccucggaaaccg
aaauaccuggccgaaguugggaggugguucaagccucuggagcacauaauguauaaagacuuggcaaa
acgguuguacggucaggaugcguugccuugcauagcgaaagggcugaacgcuagagaaacggcugaag
ugcuccgagccaaaugggacaaguucgcuucucccguuugcgucucgcuggaugccagucgguucgau
cugcauguaaguccugacgcauugcgguuuacgcaccgccuguaccacaaguauugccaaagucggca
acuccgcaaguaccuagaauggacgcugagaaacgcuggcgucgccucauguccugaaagcgcuuauc
aguaugagguugaggggagacgcaugaguggcgacauggacaccgcacucggcaacugcguacuuaug
cucugcuugacauggaacuuccucgaucaacauaacaucaagcaugagauaauggacaacggagauga
cugcuuguucaucugugaagcugccgaugugccaaccgacaagcaaaucauggacuacuaccucgacu
uuggguucgugguucgguuggaaggaaaggugucuguguucgagcgaauagaguucugucaaaccagu
ccgguguugacugcuaauggauggcguaugguuagaaauuugaaguccauugcgaaggaccucugcaa
ugugaacauggcgacugggucacucagugaauacacugcguggcuuaaagccgugggaaucuguggua
gaauccugaacgaugggguuccaaucuucuccgccuuccacaacaugcuggugcgacauggaacgaac
ucacgaauagauagagcgguguucugggaauguggacugacaaacuugaucaaaggcaugaguuucga
gcaacuggaaaucacugucgcugcgcgcgaguccuuuuaucuggcauacgguaucacaccggcgagac
aacucgcgauugaagaguauuacgacucacuccagggcccgguggguaaaauacaacuucaugaaugg
ccacuacaacucaaagaggaauacgcgugcggcgccgagugguucgaaggagacggcgagcgggcuug
aggcccgcuggcuugcccuucgugcccggcagcucucgcacgguucggacugcgcucguccucgagaa
ccacuugccgauguccucggcacaguugggucaagaggccguugcguauucuaucccgugcaauguuc
gaaacaugccuacgauccugacucucgccaccacuccgcucuauuggcguaucaccgccaucacuguc
gcgauggagccugcaaaguccacaucgacccaaauugccgguguggggaaugcugauucauuucaguc
ugccaccuacaacgguuuugggaacguguuuaagaaaaugcgcgcuuugaauuucgugagacgcucgg
cgcccggaggcaaucuucagguacgcuggccuaucaauauggacuggaucuccgcauccgacacggac
aaggauagcacaaaagugcccucgcuauucuuugccgugaccaacccaggugugaucgaaaccaaaca
aggggacagugaggccugguuggaaugggaguuggagcuggaguacauaguuggaggcuaggaacgac
ugcccgcuugagaucgacucucccguggugagguaccacccacucagcugugucagccgguuggagaa
acucuggugcgauagcacuguuggccccugccuagcgugugcugugggaaagccccaacagauuugug
aaacacuggaguugucgacccgcgagacgugcggcucgaguugucgcuuccccgugaggggggcugcc
gggggguagagaaauauucccgguauuuauccgcuaagaccuacgcgcgacgaaacuggcg

Note that iRNA relatives (e.g., iRNA r1, iRNA r2, and iRNA r3) may comprise conserved polynucleotide sequence(s) (bolded and underlined above): auagcacug (SEQ ID NO:4); and/or gauuuguga (SEQ ID NO:5). For example, the iRNA molecule comprises both of conserved polynucleotide sequence(s): auagcacug (SEQ ID NO:4); and gauuuguga (SEQ ID NO:5).

In addition, iRNA relatives (e.g., iRNA r1, iRNA r2, and iRNA r3) may comprise conserved polynucleotide sequence(s) (bolded and underlined above): cguuc (SEQ ID NO:10); gaacg (SEQ ID NO:11); gguuca (SEQ ID NO:12); ggag (SEQ ID NO:13); and/or aaauggga (SEQ ID NO:14). For example, the iRNA molecule comprises all of conserved polynucleotide sequence(s): cguuc (SEQ ID NO:10); gaacg (SEQ ID NO:11); gguuca (SEQ ID NO:12); ggag (SEQ ID NO:13); and aaauggga (SEQ ID NO:14).

Further, iRNA relatives (e.g., iRNA r1, iRNA r2, and iRNA r3) may comprise conserved polynucleotide sequence(s) (bolded and underlined above): ucgacg (SEQ ID NO:15); and/or cuccga (SEQ ID NO:16). The iRNA molecule may comprise both conserved polynucleotide sequence(s): ucgacg (SEQ ID NO:15); and cuccga (SEQ ID NO:16). In some embodiments, the iRNA molecule are highly related to CYVaV (or to iRNA r1, iRNA r2, or iRNA r3), and comprise a polynucleotide sequence having 50%, 60%, 70% or more identity for the recoding site for synthesis of RdRp thereof, e.g., 75% or 85% or 90% or 95% or 98% identify of the RdRp of CYVaV (or of iRNA r1, iRNA r2, or iRNA r3).

Thus, in accordance with disclosed embodiments, an RNA vector (e.g., derived from an iRNA molecule) comprises a frameshift ribosome recoding site for synthesis of the RNA-dependent RNA polymerase (RdRp). In addition, the RNA vector may include a 3′ end comprising a polynucleotide sequence that terminates with three cytidylates ( . . . CCC). The penultimate 3′ end hairpin may also contain three guanylates in the terminal loop ( . . . GGG . . . ). Further, the 3′ CITE includes an extended hairpin or portion thereof that binds to Eukaryotic translation initiation factor 4 G (eIF4G) and/or Eukaryotic initiation factor 4F (eIF4F).

In certain embodiments, an RNA vector comprises a 3′CITE comprising conserved sequences auagcacug (SEQ ID NO:4) and gauuuguga (SEQ ID NO:5). The RNA vector may also comprise one or more of the following polynucleotide sequences (conserved sequences of identified iRNA molecules): cguuc (SEQ ID NO:10) and gaacg (SEQ ID NO:11); and/or gguuca (SEQ ID NO:12) and ggag (SEQ ID NO:13); and/or aaauggga (SEQ ID NO:14). Alternatively, or in addition, the RNA vector may comprise one or both of the following polynucleotide sequences (conserved sequences of identified iRNA molecules): ucgacg (SEQ ID NO:15) and cuccga (SEQ ID NO:16).

Identified iRNA relatives all have inserts in the 3′UTR and other nucleotide changes that result in the generation of an ORF that encodes a protein (p21.2) of unknown function. One differentiating characteristic of iRNAs such as CYVaV from any plant virus (FIG. 2) is that iRNAs do not encode any movement protein(s), which is characteristic of all known plant viruses including umbraviruses. Nor do iRNAs such as CYVaV require any helper virus for systemic movement through plants, including tested citrus and Nicotiana benthamiana (a laboratory model plant).

In contrast, PEMV2, as with all umbraviruses, encodes for two movement proteins: p26 (long-distance movement) and p27 (cell-to-cell movement) (FIG. 3, Panel A). p26 is also a stabilization protein that protects the genome from nonsense mediated decay (NMD), and is required for accumulation at detectable levels of PEMV2 in single cell protoplasts (Gao, F. and Simon, A. E. (2017), Differential use of 3 ′ CITEs by the subgenomic RNA of Pea enation mosaic virus 2, Virology 510:194-204). Umbraviruses are unusual viruses as they do not encode a coat protein or RNA silencing suppressor, but rather rely on a helper virus for these functions. For PEMV2, the helper virus is the enamovirus PEMV1.

The polynucleotide sequence of PEMV2 is presented below (SEQ ID NO:23):

ggguauuuau agagaucagu augaacugug ucgcuaggau caagcggugg uucacaccug
acuucacccc uggcgagggc gugaagucua gagcucaacu ggaaagagag cuggauccca
ccugggcgcu ucucgugugc caagaacgag cgcgucguga ugcugacagu auugcuaaug
agugguacga gggcagcaug gagugcaacc uccuuauccc ucggcccaca accgaggaug
uauuuggccc cuccaucgcc ccugagccug uggcucuagu ggaggaaacu acccguuccc
gcgcgccgug cguggauguc ccugccgagg aguccuguaa gucagcggag auugauccug
uugaucucgc caaguucgac ucccuccauc gucgccuguu ggcugaagcc aacccuugca
gggaaauggu ucugugggug ccuccuggcc uaccagcaga gcgcgacguc cugcccaggg
cacguggggu gauaaugauc cccgaagucc cugccucugc acauaccuug uccgugaagg
uuauggaggc ugugcgguug gcacaggaag ucuuggcauc ccuugccaag agggccuuag
agaaaagguc uacaccaacc cuuaccgccc aggcccagcc agaggcuacc cugucggggu
gcgacuaccc guaucaggag acuggagcag cagccgcgug gauaacgccu ggcugcauug
ccauggagcu cagagccaaa uuuggcgucu gcaaacgcac ccccgcaaac uuagagaugg
ggagucgcgu cgcccgcgag cuccugcggg auaacugugu cacuugcagg gagaccacgu
gguacaccag ugccauugcu guggaccugu gguugacccc gaccgucguc gaccuggccu
guggccggcg agcggcggau uuuugguagg ggcugugcug ccucggcugg gggaagacac
cagugugcgg uuugacaacc ugcaccccag caucgaggua aucaaggcgg cuaggccccg
cccaacccag aggaugucgu uccaaaucga cguugugcgu ccucuuggag auuuuggugu
gcacaacaac ucccuuguua accuagccag gggaauuaau gaaagggugu ucuacacgga
caaugcuagg acagaacccc uccagccuaa gguucccuuc cccucaucac gggagcuaaa
aaccuucaga gucaccccuu ggaccaugga uaggguugug gagaguuaca caggguccca
gcgcacucgc uaugcuaacg cgcgggacag cauauuaucc aacccucuga gucccaaaga
ugcgcggguc aagacguuug ucaaagcuga aaagauaaau uucacagcca aaccugaccc
cgccccucgu gugauacagc cuagggaucc acgauucaac auuguccugg cuaaauacau
caagccuuug gagccaaugu uguacaaagc acuggggaaa cuuuacaagu accccgcagu
ugcuaagggg uuuaacgcgg uugagacggg ggagaucauc gccggcaagu ggcggugcuu
caaagauccu gucgucgugg gauuagacgc uucccgauuu gaucagcaug uaucugucga
ggcguugcag uucacccacg cgguguacag aggguucauc aagucacggg aguuuaacaa
ccuccuacag augauguaca ccaaccgugg ccuagggucc gcuaaggacg gauucguccg
uuacaagguu aaagguagac gcaugagcgg ugacauggac accuccuugg gcaacugugu
gcucauggug uugcucacca ggaaccuuug caagguucua ggcaucccgc acgagcucuu
caacaauggu gaugauugca ucgucuuuuu cgaucguugc cacuuggaga aguucaacaa
ugcugucaag acuuauuuug cggaccuagg guuuaagaug aagguggaac cgccgguuga
cguguuggag aaaauagagu ucugccaaac gcagccuauc uaugacgggg agaaguggcg
caccgugcgu ugcaucucga guaucggaaa agauugcuca uccguuauua guugggacca
auuggagggg ugguggaaug ccaucgccca gaguggucug gcugugugug gcggaaugcc
gauauacacg ucguucuacc gguggcuagc acgggccggu aagaguggga ccaaguguca
gucacacccc uuguggaaaa acgagggguu gaauugguac aggaugggga uggaccuuuc
ucaugagguu aauguuaccc cucaggcgcg ccugucuuuc uucgcggguu uugguauuuc
ccccccgaug caggucgcca uugaggcgcu guaugacaag cugccuccac cgucccccca
ccaugguccu ccgguuaagg cuguaacaca gcgaguguuc accaauuauu ucacgccgga
aagcgccugu guuagcauga gcacgaauga agacaacaaa ucugacuuug cuguuuacgg
cccugugccu acagugaugu cucuuugugc ucaguguuag gcucuuaaau uuuagcgaug
gcgugacacg guuacacccu gaauugacag gguacagauc aagggaagcc ggggagucac
caacccaccc ugaaucgaca gggcaaaaag ggaagccggg caccgcccac guggaaucga
ccacgucacc uuuucgcguc gacuaugccg ucaacacccu uucggcccgc cagccuagga
caauggcggu agggaaauau augacgauaa ucauuaaugu caauaacgac gagcgcaagc
aaccagaagg agcuacuggc agcucuguac ggcgagguga caauaaaaga acucgaggaa
acaaaccucg gagucaucac cccgguucgc gcgaacgaaa agguuacaau caccccucuc
cuacccccaa aaacucaaag cagggucagc uccguacuga agcgguucag gagcacccga
aacacggggg gacugcuuuc cguagagaaa guggugguag uguucacccc ucacaucccc
gacgacgugc uaggagaggu ggagauaugg cuccacgaca gcauccuccc ccaccucggg
agcgucggac caagacugaa acucaagcug agcgaagggc ccaagcucuu agcguucuac
ccacccuacu cgauugcauu gggggacucg aucucgggcc agccgagguc cuucuccauu
gucaccgagc uguucgaagg caacuucgca ccggggugca gcccauucag ccuguuccuc
auguggaguc cacgcaucga agcagugacc cacaacuacu ugagucgucc accacgugcu
cugccaauuu gcagaacgau ggugcgggac gcguuaucgg agguggcauc ccaacagcaa
uaccugaagg gagcgauguc gaacagguau gccaugccuc ucacuacggg ugauggccag
cauagagcca ugaagggggc ucccagugcc cuuccaccaa cgggggugug uacccaggcu
ucuaagugag gcuucgcuuc ccgccggaag accgcggcgg uucuguuccu cccacaggag
uacggcaaca acccaccuug ggaaaguggg gaccccagca cuaacuccuu uaacuaggcg
ggcguguugg uuacaguagg aggggacagu gcgcaucgaa acugagcccc accacaacuc
ucauccacgg ggugguuggg acgcaggugu cggagggauc gccagcccuc aggauaguga
gcucccgcag agggauaagc uaucucccug cgacguagug guagaacacg ugggauaggg
gaugaccuug ucgaccgguu aucggucccc ugcuccuucg agcuggcaag gcgcucacag
guucuacacu gcuacuaaag uugguggugg augucucgcc caaaaagauc acaaacgcgc
gggacaaggu cccuuccacc uucgccgggu aaggcuagag ucagcgcugc augacuauaa
cuugcggccg auccaguugc acgacuggug gucccccuca gugucucggu ugucugccga
gugggcggug gucggauucc accacacccu gccacgaggu gcguggagac uuggccaguc
uaggcucguc guaauuaguu gcagcgacgu uaaucaaccc guccgggcau auaauaggac
cgguugugcu ucuuccuccc uucuuagcca ggugguuacc ucccuggcgc cc

The polynucleotide sequence of the intergenic plus region of PEMV2 (bolded and underlined above) is presented below (SEQ ID NO:24):

guuagcauga gcacgaauga agacaacaaa ucugacuuug
cuguuuacgg cccugugccu acagugaugu cucuuugugc
ucaguguuag gcucuuaaau uuuagcgaug gcgugacacg
guuacacccu gaauugacag gguacagauc aagggaagcc
ggggagucac caacccaccc ugaaucgaca gggcaaaaag
ggaagccggg caccgcccac guggaaucga ccacgucacc
uuuucgcguc gacuaugccg ucaacacccu uucggcccgc
cagccuagga caauggcggu agggaaauau aug

The polynucleotide sequences of recoding frameshift sites of PEMV2 (bases 881 to 1019; see also FIG. 10) is presented below (SEQ ID NO:25):

gaccgucgucgaccuggccuguggccggcgagcggcggauuuuugguagg
ggcugugcugccuggcugggggaagacaccagugugcgguuugacaaccu
gcaccccagcaucgagguaaucaaggcggcuaggcccc

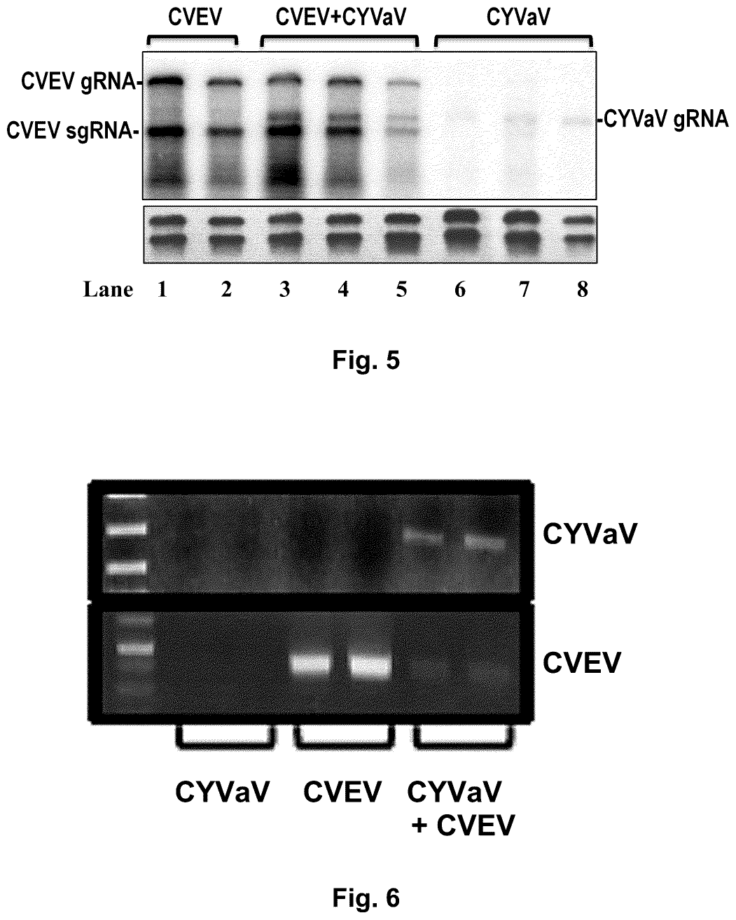

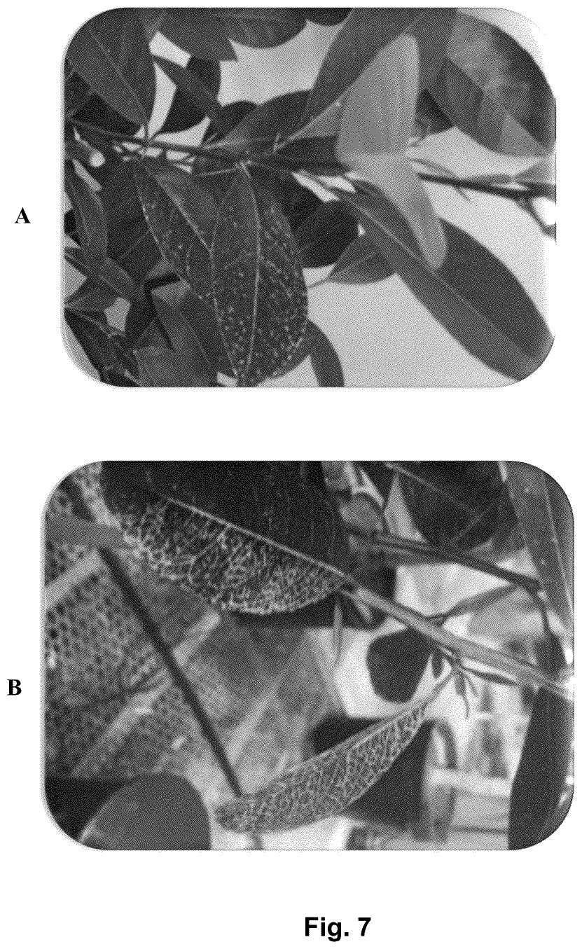

CYVaV unexpectedly replicates very efficiently in Arabidopsis thaliana protoplasts despite not encoding p26 (or any other movement protein), which is required for accumulation of PEMV2 because of its ability to also counter NMD (see, e.g., May et al. (2020) “The Multifunctional Long-Distance Movement Protein of Pea Enation Mosaic Virus 2 Protects Viral and Host Transcripts from Nonsense Mediated Decay,” mBio 11:300204-20; https://doi.org/10.1128/mBio.00204-20). Indeed, CYVaV was unusually stable, much more stable than most traditional viruses. CYVaV also produced an astonishingly high level of p81 in wheat germ extracts, at least 50-fold more than the p94 orthologue from PEMV2 (FIG. 3, Panel C). When CYVaV was agro-infiltrated into leaves of Nicotiana benthamiana, it replicated in the infiltrated tissue but accumulation was relatively weak (FIG. 3, Panel B, top; FIG. 5, lanes 6-8). No replication was achieved with manual inoculation. However, when CYVaV was co-infiltrated with the enamovirus Citrus vein enation virus (CVEV), accumulation improved substantially in these cells (FIG. 5, lanes 3-5; see also FIG. 6). In citrus, yellowing symptoms of CYVaV+CVEV (FIG. 7, Panel B) were more vibrant as compared to symptoms exhibited by CYVaV alone (FIG. 7, Panel A).

CYVaV had no synergistic effect with any other combination of citrus virus tested. Additional studies showed that CVEV may be utilized as a helper virus for CYVaV in order to allow for transmission from tree to tree. CVEV was likely responsible for the presence of CYVaV in the original limequat trees; however, CVEV is known to be very heat sensitive and thus was likely lost from the limequat trees during a hot summer.

CYVaV moved sporadically into upper, uninoculated leaves and accumulated at extremely high levels, sometimes visible by ethidium staining on gels. Symptoms that began in the ninth leaf of the major bolt comprised stunting, leaf curling, and deformation of floral tissue. Leaves in axillary stems also began showing similar symptoms around the same time. This astonishing result demonstrated that CYVaV moves systemically in the absence of any encoded movement protein(s), which is not possible by traditional plant viruses. Experiments showed that CYVaV moves systemically in N. benthamiana and is strictly confined to the phloem, replicating only in companion cells and phloem parenchyma cells. In citrus, CYVaV is 100% graft-transmissible, but difficult to transmit in other forms.

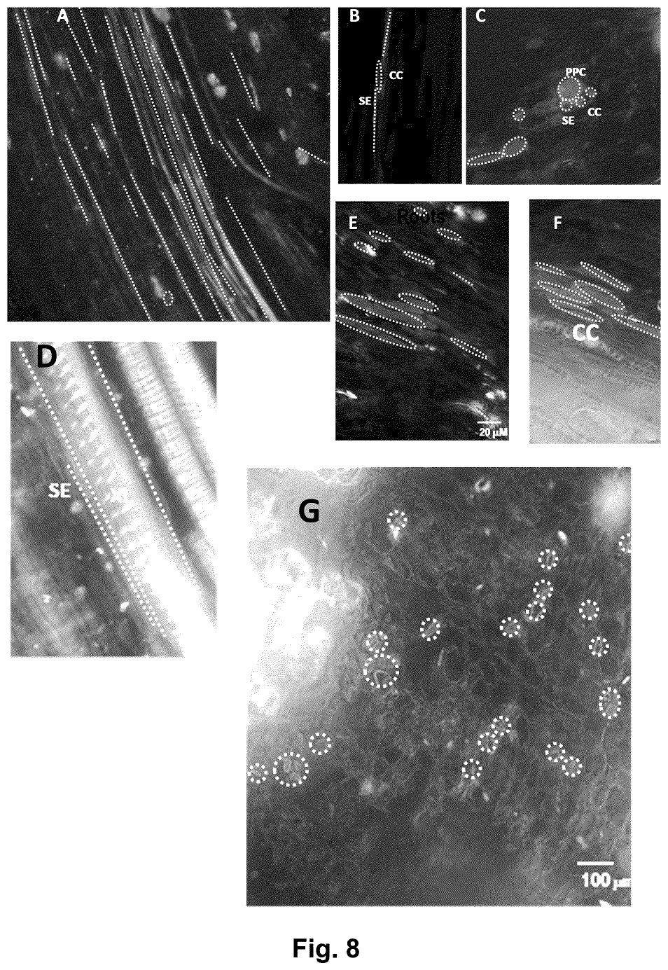

Fluorescence in situ hybridization (FISH) of symptomatic leaf tissue and roots confirmed that CYVaV is confined to phloem parenchyma cells, companion cells and sieve elements (FIG. 8, Panels A-G), which is characteristic of a phloem-limited virus. CYVaV levels were extremely high in the petioles of symptomatic tissue and sometimes visible in ethidium-stained gels of total RNA. Although symptoms are more severe in N. benthamiana, CYVaV has been found to be virtually symptomless in all varieties of citrus tested. Indeed, the most severe symptom was found on citron, the indicator tree for citrus viruses, and consisted of very minor gold flecking on leaves scattered throughout the tree.

Phloem-limited movement of CYVaV explains why it is readily graft-transmissible, but not easily transmissible by any means. CYVaV lacks any encoded movement protein(s) as noted above. Instead, CYVaV utilizes host plant endogenous movement protein phloem protein 2 (PP2), and the pathway for transiting between companion cells, phloem parenchyma cells, and sieve elements.

In addition, since host range is believed to involve compatible interactions between viral movement proteins and host plasmodesmata-associated proteins, it is believed that CYVaV is capable of transiting through the phloem of numerous other woody and non-woody host plants using PP2 as it is a very conserved host endogenous movement protein(s). As such, CYVaV provides an exceptional model system for examining RNA movement (e.g., in N. benthamiana and/or citrus) and for use as a vector for numerous applications. Experiments confirmed that CYVaV moves systemically in a host plant and is limited to the phloem, and is readily graft-transmissible but not readily transmissible between plants in other forms.

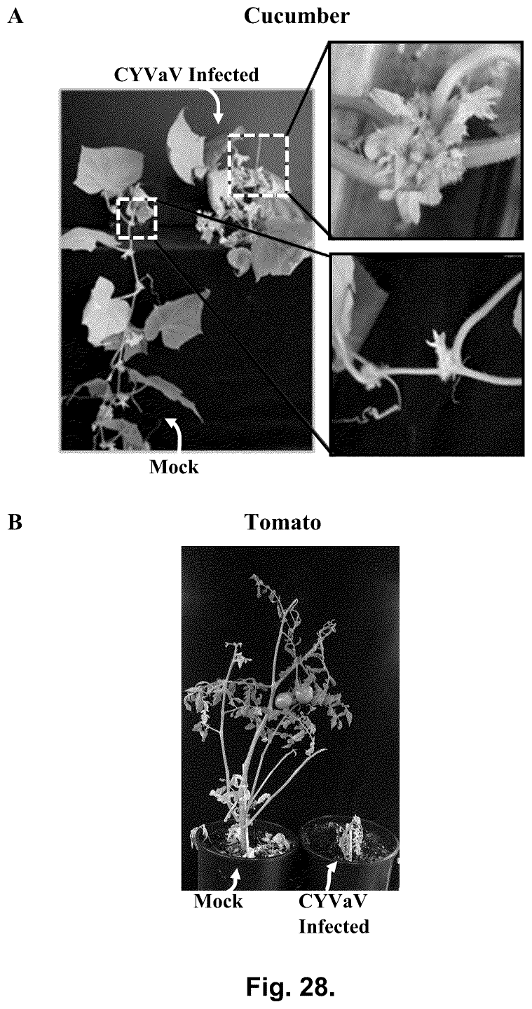

Systemic infection by CYVaV was also observed in tomato, cucumber and melon. Referring to FIG. 28, Panel A shows an uninfected cucumber plant (mock) and a plant infected by CYVaV by way of agroinfiltration about two months earlier, both grown under the same conditions. The infected plant shows effects of CYVaV infection indicating systemic movement of CYVaV and systemic infection of the cucumber plant. In the infected plant, the stem distance between nodes is drastically reduced such that multiple flowers are located in a cluster. This sign of infection is also observed in N. benthamiana and appears to be characteristic of CYVaV infection of some rapidly growing plants. FIG. 28, Panel B, shows an uninfected tomato plant and a tomato plant infected with CYVaV about 53 days earlier, both plants grown under the same conditions. The tomato plant was infected by injecting sap from a CYVaV-infected N. benthamiana plant into the vasculature of the tomato plant. The infected plant shows a lack of growth indicating systemic movement of the CYVaV and systemic infection of the tomato plant. The infection of N. benthamiana, cucumber, tomato and other plant species mentioned herein, and the natural occurrence of CYVaV and iRNA relatives, indicates that iRNA appear have a wide host range. The ability of CYVaV to bind to phloem protein 2 (PP2), as described herein, also suggests a wide host range since PP2 is found in an extremely large number of plant species and thus provides a mechanism for systemic movement of CYVaV and other iRNAs through many plant types.

Citrus trees have a complex reproductive biology due to apomixis and sexual incompatibility between varieties. Coupled with a long juvenile period that can exceed six years, genetic improvement by traditional breeding methods is complex and time consuming. The present disclosure overcomes such problems by providing an iRNA-based (e.g., CYVaV-based) vector engineered to include therapeutic siRNA inserts. iRNAs such as CYVaV are unique among infectious agents given they encode a polymerase yet move like a viroid (small circular non-coding RNA that also uses PP2 as a movement protein), and thus are capable of transiting through plants other than citrus. Thus, in addition to citrus, the iRNA-based vectors of the present disclosure may be developed for other woody plants (e.g., trees and legumes), and in particular olive trees and grapevines.

In accordance with disclosed embodiments, CYVaV is utilized in the development of a vector for delivery of small RNAs and proteins into citrus seedlings and N. benthamiana. The procedure utilized for CYVaV vector development was similar to that utilized by the present inventors for engineering betacarmovirus TCV to produce small RNAs (see Aguado, L. C. et al. (2017), RNase III nucleases from diverse kingdoms serve as antiviral effectors, Nature 547:114-117). Exemplary and advantageous sites for adding one, two, three, or more small RNA inserts designed to be excised by RNase III-type exonucleases were identified. Exemplary sites in the CYVaV molecule for inserts include positions 2250, 2301, 2319, 2330, 2336, 2083 and 2375. A small hairpin was expressed directly from the genome that targets GFP expressed in N. benthamiana plant 16C, which silenced GFP.

In accordance with disclosed embodiments, iRNA vectors disclosed herein may contain small RNA inserts with various functionality including: small RNAs that target an essential fungal mRNA; small RNAs that target an insect for death, sterility, or other incapacitating function; small RNAs that target gene expression in the host plant; small RNAs that target plant pathogenic bacteria; small RNAs that target CTV; and small RNAs that target CVEV (as this virus together with CYVaV causes enhanced yellow-vein symptoms) or other virus pathogen(s). In addition, the disclosed vectors may include other small RNAs and/or therapeutic agents known in the art. Thus, a phloem-restricted iRNA-based vector may be engineered to produce small RNAs that have anti-bacterial and/or anti-fungal and/or anti-insect and/or anti-viral properties, which provides for a superior treatment and management strategy compared to current methodologies.

CYVaV vectors may be applied manually to infected or uninfected trees by cutting into the phloem and depositing the vector either as RNA, or by agroinfiltration, or after encapsidation in the coat protein of CVEV or another virus, following citrus inoculation procedures well known to those of skill in the art, e.g. such as procedures developed and used routinely under the Citrus Clonal Protection Program (CCPP). Such procedures are routine for inoculation of CTV and other graft-transmissible pathogens of citrus. Since CYVaV does not encode a capsid protein, no virions are made and thus no natural tree-to-tree transmission of CYVaV is possible. When CYVaV is encapsidated in CVEV or other viral coat protein, no other component of CVEV or other virus is present.

A plant may be infected with an iRNA-based vector by way of agroinfiltration without cutting onto the phloem, for example by agroinfiltration into the leaves of the plant. An iRNA-based vector is not a mere replicon that, once injected into a plant cell, is not expected to leave the plant cell. The goal of agroinfiltration of an iRNA-based vector into, for example, the leaf of a plant is not to install the iRNA-based vector in plants cells near the agroinfiltration site, but rather to have at least some of the iRNA-based vector reach the plant's vasculature and thereafter move systemically through the plant. Typically, when agroinfiltrated into the leaf of a plant, only a portion of the agroinfiltrated iRNA-based vector will reach the plant vasculature and be effective for infecting the plant. In the case of plants recalcitrant to agroinfiltration, the agroinfiltration may be performed first in a related species more susceptible to agroinfiltration followed by grafting from the more susceptible species to the target species. For example, Citrus limon may be more susceptible to agroinfiltration than various species of orange trees. Alternatively or additionally, a species recalcitrant to agroinfiltration may be pretreated to make them more susceptible to agrofiltration. For example, agroinfiltration into Citrus plants may be facilitated by first inoculating the intended agroinfiltration site with an actively growing culture of Xanthomonas citri subsp. citri (Xcc) suspended in water, as described for example in Jia and Wang (2014). Xcc-facilitated agroinfiltration of citrus leaves: a tool for rapid functional analysis of transgenes in citrus leaves. Plant Cell Rep. 33:1993-2001.

When infecting the vasculature of a plant directly, for example by way of contact with a cut in the phloem, the iRNA-based vector may be stabilized with a capsid protein of another type of virus. In some examples, the iRNA-based vector is encapsidated with the coat protein of CVEV, which is believed to be a helper virus able to encapsidate CYVaV in nature. In some examples, one or more iRNA-based vector molecules are encapsidated in a self-assembling capsid protein not naturally associated with CYVaV. For example, methods of assembling capsid protein from cowpea chlorotic mottle virus with RNA molecules of various sizes are described in Cadena-Nava, R. D. et al. (2012), Self-assembly of viral capsid protein and RNA molecules of different sizes: requirement for a specific high protein/RNA mass ratio, J. Virol. 86:3318-3326.

Once a first plant has been infected with an iRNA-based vector, another plant may be infected by grafting a part of the first plant to the other plant, or by injecting sap from the first plant into the other plant, or by linking the phloem of two plants through a parasitic dodder plant. Grafting in particular allows for transferring the iRNA-based vector over long distances and with long periods of time (e.g., one day or more) between cutting the graft from the first plant and adding the graft to the second plant. In some examples, an iRNA-based vector is transferred between strains or species by way of sap taken from a plant of one strain or species and injected into the vasculature of another plant of a different strain or species. In some examples, an iRNA-based vector is transferred between strains or species by way of a graft taken from a plant of one strain or species and grafted to another plant of a different strain or species.

A first plant (optionally called in some cases a mother tree) infected with an engineered iRNA-based vector can be used to produce grafts for transmitting the iRNA-based vector to other plants either as a preventative or to treat an infection already present in the other plant. The first plant can also be used to produce seedlings (for example by grafting from the first tree to seedlings of the first plant or another plant) which are used to propagate plants having the iRNA-based vector. Once in a seedling, the iRNA-based vector replicates and moves through the plant as it grows.

As noted above, CYVaV has only two ORFs: a 5′ proximal ORF that encodes replication-required protein p21; and a frame-shifting extension of p21, whereby a ribosome recoding element allows ribosomes to continue translation, extending p21 to produce p81, the RNA-dependent RNA polymerase. The organization of these two ORFs is similar to the organization of similar ORFs in viruses in the Tombusviridae and Luteoviridae. However, all viruses in these families, and indeed in all known plant RNA viruses, encode movement proteins or are associated with a secondary virus that encodes a movement protein(s). The ability to encode movement proteins, or associate with a second virus that encodes a movement protein(s), had long been considered a requirement for movement from cell-to-cell and also for transiting through the phloem to establish a systemic infection. As such, the use of iRNAs as vectors had not been proposed, and indeed iRNA molecules were previously considered unsuitable for use as an independent vector due to the lack of any encoded movement protein and belief that they were not independently mobile.

As such, the capacity for independent systemic movement of iRNAs throughout a plant's phloem despite not coding for or depending on any exogenous movement protein(s) is quite surprising. The CYVaV-based vectors of the present disclosure unambiguously and repeatedly demonstrated (via fluorescence in situ hybridization and other techniques) systemic movement without the aid of any helper virus. Young, un-infiltrated (systemic) tissue displayed highly visible symptoms on N. benthamiana, including leaf galls and root galls. The disclosed vectors utilize endogenous host movement protein(s) for mobility. In this regard, host phloem protein(s) (25 kDa phloem protein 2 (PP2) and/or 26 kDa Cucumis sativus phloem protein 2-like) known to traffic host RNAs into sieve elements (see Balachandran, S. et al. (1997), Phloem sap proteins from Cucurbita maxima and Ricinus communis have the capacity to traffic cell to cell through plasmodesmata, PNAS 94(25):14150-14155; Gomez, G. and Pallas, V. (2004), A long-distance translocatable phloem protein from cucumber forms a ribonucleoprotein complex in vivo with Hop stunt viroid RNA, J Virol 78(18):10104-10110) were likely shown to interact with CYVaV using Northwestern blots in vitro and RNA pull-downs from infected phloem sap in vivo. Thus, since known plant viruses encode (or are dependent on) a movement protein, iRNAs are quite different structurally and functionally from traditional plant viruses.

In addition to CYVaV, other RNAs of similar size and that encode a polymerase may be utilized in the development of similarly structured iRNA-based vectors (see, e.g., Chin, L. S. et al. (1993), The beet western yellows virus ST9-associated RNA shares structural and nucleotide sequence homology with Tombusviruses. Virology 192(2):473-482; Passmore, B. K. et al. (1993), Beet western yellows virus-associated RNA: an independently replicating RNA that stimulates virus accumulation. PNAS 90(31):10168-10172). As noted above, other iRNA relatives (e.g., iRNA r1, iRNA r2, and iRNA r3, identified in Opuntia, Fig trees, and Ethiopian corn, respectively) and that encode proteins p21 and p81 (FIG. 4) may be utilized for vector development.

Although CYVaV is present in the GenBank database (GenBank: JX101610), iRNAs do not belong to any known classification of virus given they lack cistrons that encode movement proteins. Nor are iRNAs dependent on a helper virus for systemic movement within a host. Moreover, iRNAs lack cistrons that encode coat proteins. iRNAs are also dissimilar to viroids, although both are capable of systemic movement in the absence of encoded movement proteins. Viroids are circular single stranded RNAs that have no coding capacity and replicate in the nucleus or chloroplast using a host DNA-dependent RNA polymerase. The vast majority of the tiny viroid genome, typically including about 300 to 400 nucleotides (nt), is needed for the viroid's unusual existence. In addition, viroids do not code for any proteins, which makes them unsuitable for use as vectors. In contrast, iRNAs code for their own RNA-dependent RNA polymerase (RdRp).

iRNAs may be categorized in two classes: a first class is characterized by a frameshift requirement to generate the RdRp and RNA structures proximal to the 3′ end that resemble those of umbraviruses. A second class is characterized by a readthrough requirement to generate the RdRp and 3′ RNA structures that resemble those of Tombusviruses. CYVaV is a member of the first class with properties similar to umbraviruses including a frameshifting recoding site and similar structures at the 3′ end, and similar sequences at the 5′ end. iRNA members of the second class have always been discovered in association with a helper virus.

A recent publication by the inventor(s) herein, Liu et al., Structural Analysis and Whole Genome Mapping of a New Type of Plant Virus Subviral RNA: Umbravirus-Like Associated RNAs, 2021, 13:646, provides another description of iRNAs and/or similar or related RNAs. This entire publication is incorporated herein by reference. This publication refers to such RNA molecules as umbravirus-like associated RNAs (ulaRNAs). The ulaRNAs are divided in this publication into three classes. CYVaV is part of the second class of the ulaRNA taxonomy. iRNA as described herein may include other ulaRNAs, for example other Class 2 ulaRNAs.

Additional ulaRNAs have been located since the publication of the paper described above. Class 1 ulaRNA exhibit frameshifting, do not encode a movement protein, and typically have lengths in the range of 4.0-4.6 kb. Known Class 1 ulaRNA currently include BabVQ (babaco). Class 3 ulaRNA exhibit frameshifting, encode a movement protein and typically have lengths in the range of 3.2-3.5 kb. Known Class 3 ulaRNA currently include SbaVA (strawberry) and WULV (wheat). Class 2 ulaRNA exhibit frameshifting, might or might not encode a movement protein, and typically have lengths in the range of 2.7-3.1 kb. Class 2 ulaRNA may be further divided into monocot and dicot based on the plants that they are found in. Monocot Class 2 ulaRNA encode two proteins after the RdRp. Dicot Class 2 ulaRNA encode 0 or 1 protein after the RdRp. Known monocot class 2 ulaRNA include TULV (teosinte), MULV (corn), JgULV (Johnsongrass), SULV (sugarcane), EMaV-2 (corn) and EMaV-1 (corn). Known dicot ulaRNA include OULV (opuntia), FULV (fig), CYVaV (citrus) (also called CYVaV1 herein), CYVaV-delta (hemp) (alternatively called CYVaV2) and CYVaV-RB. PMeV2 (papaya) and PUV (papaya) were previously classified as umbraviruses prior to the introduction of the term ulaRNA, although they are unlike other umbravirus in that no movement protein ORF has been located in them. PMeV2 and PUV are currently considered to be Class 1 ulaRNA. FIG. 3 shows the prior classification of PMeV2 and PUV as umbraviruses. Open reading frames of WULV are still under investigation, and WULV may be part of a putative fourth class of ulaRNA as indicated in FIG. 4. Alternatively, WULV may be considered a Class 3 ulaRNA.

ulaRNA have two ORFs related to replication. Some ulaRNA also have an ORF related to putative movement proteins. An additional embedded ORF in monocot-infecting Class 2 ulaRNAs is of unknown function. The ORF of some ulaRNA have motifs that are similar to movement proteins in other viruses. ORF5 present in Class 2 ulaRNA have consensus motifs of one class of viral movement proteins. ORF 5-1 in Class 3 ulaRNA has a consensus motif of a different movement protein class. However, both CYVaV-Delta and Opuntia are able to infect N. benthamiana plants with ORF 5 expression suppressed. Infection of CYVaV-Delta decreases without ORF5 expression, but OULV infection increases without ORF5 expression. The function of ORF5 is thus not entirely certain.

It is expected that more ulaRNA will be identified in the future and can also be used as described herein. ulaRNAs are the only viruses other than umbraviruses that do not encode a coat protein. Accordingly, ulaRNAs may be identified by the absence of an open reading frame (ORF) that encodes a coat protein and that they are not an umbravirus. Further, if ulaRNAs have an ORF that encodes a movement protein, this ORF is adjacent to or overlapping with the RdRp. In comparison, movement proteins of umbraviruses are separated from their RdRp. Accordingly, ulaRNAs may be identified by their absence of an ORF encoding a coat protein and that their ORF encoding a movement protein, if any, is adjacent to or overlapping with the ORP encoding the RdRp. All ulaRNA located to date also do note encode a silencing suppressor, which may be used as a further characteristic to identify a ulaRNA. Future ulaRNA may also be identified by their similarity with known ulaRNA using taxonomic methods known in the art.

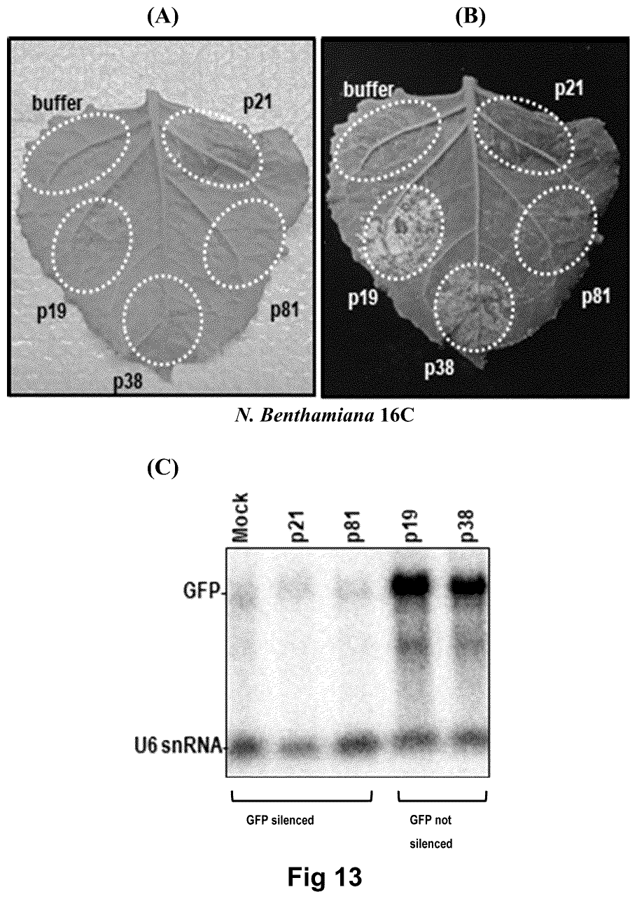

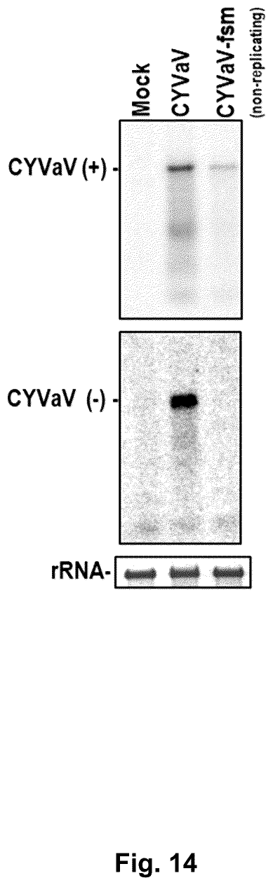

iRNAs and ulaRNAs provide a number of benefits as compared to conventional viral vectors. For example, iRNAs and ulaRNAs are relatively small, making them easier to structurally and functionally map and genetically manipulate. In contrast, viruses such as CTV are 8-fold larger, making them more cumbersome to use as a vector. iRNAs can replicate and accumulate to unexpectedly high levels (e.g., visible by ethidium staining on gels and 4% of reads by RNAseq), which is critical for the vector's ability to deliver a sufficient amount of therapeutic agent(s) into the target plant. In addition, iRNAs and ulaRNAs are much more stable than many viruses despite not encoding a coat protein or silencing suppressor (FIG. 13), which allows for a long lifespan in the host plant and thus provides benefit over an extended period.

iRNAs and ulaRNAs are also limited to the host's phloem, which is especially useful for targeting pathogens that either reside in, or whose carriers feed from, or whose symptoms accumulate in, the phloem since the payload will be targeted to where it is most needed. By moving independent of movement proteins in at least some examples (whose interactions with specific host proteins is the primary factor for determining host range), iRNAs and ulaRNAs are able to transit within a broader range of hosts, thereby increasing the applicability of a single vector platform. Given the lack of coat protein expression and the dispensability of a helper virus for systemic plant infection, iRNAs and ulaRNAs cannot be vectored from plant-to-plant and instead are introduced directly into the phloem via grafting. The lack of a coat protein prevents formation of infectious particles and thus unintended reversion to wild type infectious agents into the environment. This is particularly beneficial for streamlining regulatory approval as regulators are often concerned with the possible uncontrolled transmission of introduced biological agents.

iRNAs and ulaRNAs are also virtually benign in citrus, unlike viruses like CTV whose isolates can be highly pathogenic. Using a common virus as a vector, such as CTV, runs the risk of superinfection exclusion, where trees previously infected and/or exposed to that virus are not able to be additionally infected by the same virus acting as the vector (e.g., most citrus trees in the USA are infected with CTV). Thus, avoiding superinfection exclusion, at a minimum, requires additional steps to the process that makes it more expensive and cumbersome.

The present disclosure also provides for novel therapeutic, prophylactic, or trait enhancing inserts that are engineered into the iRNA or ulaRNA vector. A variety of inserts are provided, including inserts that target a particular pathogen, an insect, or a manifestation of the disease(s). Alternatively, or in addition, inserts are provided that strengthen or improve plant health and/or enhance desired characteristics of the plant.

The disclosed infectious agents are capable of accumulation and systemic movement throughout the host plant, and can thus deliver therapies throughout a host over a substantial time period. Characteristics of the disclosed agents are therefore highly beneficial for treating numerous specific diseases. Using an infectious agent composed of either RNA or DNA has an additional advantage of being able to code for therapeutic proteins or peptides that would be expressed within infected cells and/or by engineering the infectious agent to contain a specific sequence or cleavable portion of its genetic material to serve as an RNA-based therapeutic agent.

Products with antimicrobial properties against plant pathogens can take a number of formats and are produced through ribosomal (defensins and small bacteriocins) or non-ribosomal synthesis (peptaibols, cyclopeptides and pseudopeptides). The best known are over 900 cationic antimicrobial peptides (CAPs), such as lactoferrin or defensin, which are generally less than 50 amino acids and whose antimicrobial properties are well known in the art. CAPs are non-specific agents that target cell walls generally, with reported effects against bacteria and fungi. CTV engineered with an insert designed to express defensin has received approval for release by the USDA in Florida, but its widespread efficacy is unknown. Moreover, the isolate of CTV used for the vector makes it unsuitable for trees growing in some regions (e.g., California).

RNA therapies that target virus pathogens are also in widespread development in plants. These therapies use non-coding small interfering RNAs (siRNAs), which are generated from the genome of the plant, and thus include genetic modification of the host. In addition to negative viewpoints of some growers and consumers to genetic modification of citrus trees, the length of time to generate genetically modified trees is measured in decades and may ultimately not have the same attributes (texture/color/taste) as varieties developed over decades, and thus is not a solution to current, time sensitive agricultural diseases, in addition to being very expensive to develop and potentially impacting the quality of the fruit.

siRNAs can be used to target bacteria in plants, for example the Candidus Liberibacter asiaticus (CLas) bacteria. Plant pathogenic bacteria can be targeted using siRNAs that are produced in plants, taken up by the bacteria, and directly reprogram gene expression in the bacteria as described for example by Singla-Rastogi et al. (2019) Plant small RNA species direct gene silencing in pathogenic bacteria as well as disease protection, bioRxiv preprint post, Dec. 3, 2019, doi: https://www.biorxiv. org/content/10.1101/863902v1. In some implementations, CYVaV or another iRNA based vector is provided that contains siRNA hairpins that target a bacteria such as Candidus liberibacter asiaticus and render the bacteria non-pathogenic. For example, an siRNA hairpin provided to a plant by an iRNA based vector may be taken up the CLas or another bacteria in the plant and control gene expression in the bacteria, thereby killing the bacteria and/or inhibiting an increase of the bacterial population. Compared to an enzybiotic which might have, for example, about 500 bases, an siRNA in the form of a hairpin is considerably smaller (<60 bases) and is more likely to be stable in an iRNA based vector.

It is commonly believed that bacteria do not take up siRNA. However, Singla-Rastogi et al. (2019) describes examples in which small interfering RNA targeted against some specific genes were taken up by Pseudomonas syringae and cause a 50% reduction in the population of Pseudomonas syringae. The inventors have confirmed that the conventional belief is at least partially correct. For example, in experiments conducted by the inventors, E. coli did not take up siRNA. However, as described in the examples herein, small RNA are taken up by some bacteria. In particular, bacteria are taken up by Pseudomonas syringae, Erwinia amylovora and Liberibacter crescens. These three bacteria are all gram negative bacteria that infect plants. However, since these three bacteria are otherwise unrelated to each other, they indicate that small RNA can be taken up by bacteria that infect plants generally, or at least by gram negative bacteria that infect plants. P. syringae is a plant pathogen that causes, for example, bacterial canker in almond trees. Erwinia amylovora is a plant pathogen that causes, for example, fire blight in apple trees, pear trees and some other trees in the Rosaceae family. Liberibacter crescens is a relative of Liberibacter asiaticus and, based on their experiments with Liberibacter crescens, the inventors believe that Liberibacter asiaticus will also take up small RNA.

The small RNA used to control bacteria (i.e. to make bacteria non-pathogenic or kill bacteria) may be less 60 nt, less than 50 nt, less than 40 nt, less than 30 nt, less than 25 nt. The small RNA used to control bacteria may be more than 10 nt or more than 20 nt. The small RNA used to control bacteria may be in the range of 21-24 nt. Longer RNA, for example 100 nt, were not taken up by bacteria in the experiments described herein.

An siRNA is typically designed to be a complement to a part of RNA or DNA associated with the target organism intended to be treated or controlled by the siRNA (“specific siRNA”). For example, as shown in the examples herein, Pseudomonas syringae, Envinia amylovora and Liberibacter crescens can all be controlled by small RNA that are complements of genes (including complements of messenger RNA) of the bacteria. In particular, these bacteria were controlled, for example by 1000 fold reductions in their population in infected plants, by specific siRNA that complement the adenylate kinase (ADK) or gyrase subunit A (GyrA) genes of the bacteria.

Sequences for growth inhibition of Envinia amylovora in vitro and in vivo are presented below. Two Envinia genes were targeted: MurA and GyrA.

Erwinia MurA (in vitro) (SEQ ID NO: 134):
gaactggtgaaaaccatgcgcgcctcgatttgggcattgggcccgctggtggcacgttttgg
ccaggggcaagtatcactgcccggtggttgcgctatcggcgcacggccggttgatcttcata
tcaccggccttgagcagctcggcgccgagatcaaactggaagaaggttacgttaaagcctct
gtcgcgggtcgcctgaaaggggcgcatatcgttatggataaggtcagcgtgggtgcaaccgt
cactatcatgagtgcggcgacgctggcaacgggcaccaccgttatcgagaatgctgcgcgtg
agccggaaattgtcgacactgccaacttcctcaacacgcttggggcgaaaatcaccggtgcc
ggcagcgatcgtatcaccatcgaaggtgttgatcgccttggtggcggtgtttatcgcgtact
tcctgaccgcatcgaaaccggtactttcctggtggcgggagcgatttccggcggtaaggtta
cctgccgtgcggcgcagcccgatacgctggatgctgtactggctaagctacgcgaagccggt
gcggacatcgagatgggagaagactggataagcctggacatgcacggtaagcggcctaaagc
ggtcaatttacgcacagcgccgcatcccggtttcccaaccgatatgcaggcgcagttcagtt
tgttgaacctggtggctgaaggcacgggggtgattactgaaaccatcttcgaaaaccgctt
Erwinia MurA (in vivo):
Fragment 1 (SEQ ID NO: 134, residues 1-402):
gaactggtgaaaaccatgcgcgcctcgatttgggcattgggcccgctggtggcacgttttgg
ccaggggcaagtatcactgcccggtggttgcgctatcggcgcacggccggttgatcttcata
tcaccggccttgagcagctcggcgccgagatcaaactggaagaaggttacgttaaagcctct
gtcgcgggtcgcctgaaaggggcgcatatcgttatggataaggtcagcgtgggtgcaaccgt
cactatcatgagtgcggcgacgctggcaacgggcaccaccgttatcgagaatgctgcgcgtg
agccggaaattgtcgacactgccaacttcctcaacacgcttggggcgaaaatcaccggtgcc
ggcagcgatcgtatcaccatcgaaggtgtt
Fragment 2 (SEQ ID NO: 134, residues 343-743):
aacacgcttggggcgaaaatcaccggtgccggcagcgatcgtatcaccatcgaaggtgttga
tcgccttggtggcggtgtttatcgcgtacttcctgaccgcatcgaaaccggtactttcctgg
tggcgggagcgatttccggcggtaaggttacctgccgtgcggcgcagcccgatacgctggat
gctgtactggctaagctacgcgaagccggtgcggacatcgagatgggagaagactggataag
cctggacatgcacggtaagcggcctaaagcggtcaatttacgcacagcgccgcatcccggtt
tcccaaccgatatgcaggcgcagttcagtttgttgaacctggtggctgaaggcacgggggtg
attactgaaaccatcttcgaaaaccgctt
Erwinia GyrA (in vitro) (SEQ ID NO: 135):
atcgtcaacctgctgccgctggaagccaacgagcgtattactgcaattctgccggtgcgtga
atatgccgaaggctggaatatctttatggctaccgccagcggaacggtgaagaaaaccgcgc
tgactgacttcagccgaccgcgcagcgccggtattattgccgtcaatctgcgtgatgacgat
gaactgatcggcgtgtcgctgacgaacggtagtgatgaagcgatgctgttttctgccgccgg
taaagtggtgcgattcgcggagagcgcggtgcgtacgatgggccgtaccgcctccggcgtac
gcggcatcaagctggctgaaggcgaccgcgtggtgtcgctgatcgtgccgcgtgatgatggc
gctatcatgaccgtgacgcaaaacggctacggtaaacgaacggctaacgtcgaatatcccac
caagtcgcgtgcgactcagggggttatctcgatcaaggtaaccgagcgtaacgggccggtta
tcggtgcggtgcaggtggtcgatggcgatcagatcatgatgatcaccgatgccggcacgctg
gtacgtacccgcgtgtccgaggtcagcgtggtagggcgtaacacccagg
Erwinia GyrA (in vivo):
Fragment 1 (SEQ ID NO: 135, residues 2-416):
tcgtcaacctgctgccgctggaagccaacgagcgtattactgcaattctgccggtgcgtgaa
tatgccgaaggctggaatatctttatggctaccgccagcggaacggtgaagaaaaccgcgct
gactgacttcagccgaccgcgcagcgccggtattattgccgtcaatctgcgtgatgacgatg
aactgatcggcgtgtcgctgacgaacggtagtgatgaagcgatgctgttttctgccgccggt
aaagtggtgcgattcgcggagagcgcggtgcgtacgatgggccgtaccgcctccggcgtacg
cggcatcaagctggctgaaggcgaccgcgtggtgtcgctgatcgtgccgcgtgatgatggcg
ctatcatgaccgtgacgcaaaacggctacggtaaacgaacggc
Fragment 2 (SEQ ID NO: 135, residues 207-607):
gacgaacggtagtgatgaagcgatgctgttttctgccgccggtaaagtggtgcgattcgcgg
agagcgcggtgcgtacgatgggccgtaccgcctccggcgtacgcggcatcaagctggctgaa
ggcgaccgcgtggtgtcgctgatcgtgccgcgtgatgatggcgctatcatgaccgtgacgca
aaacggctacggtaaacgaacggctaacgtcgaatatcccaccaagtcgcgtgcgactcagg
gggttatctcgatcaaggtaaccgagcgtaacgggccggttatcggtgcggtgcaggtggtc
gatggcgatcagatcatgatgatcaccgatgccggcacgctggtacgtacccgcgtgtccga
ggtcagcgtggtagggcgtaacacccagg

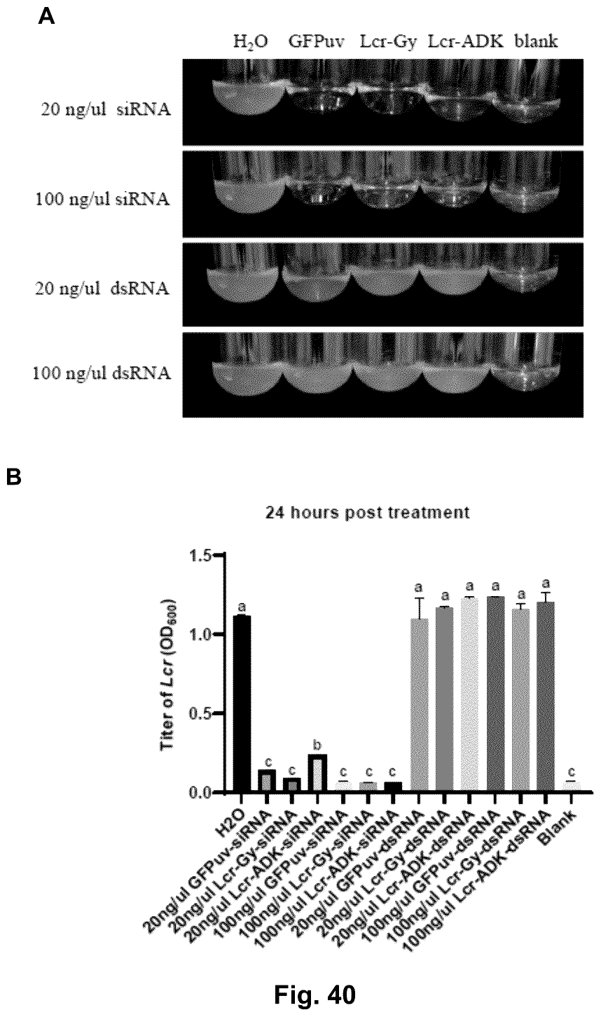

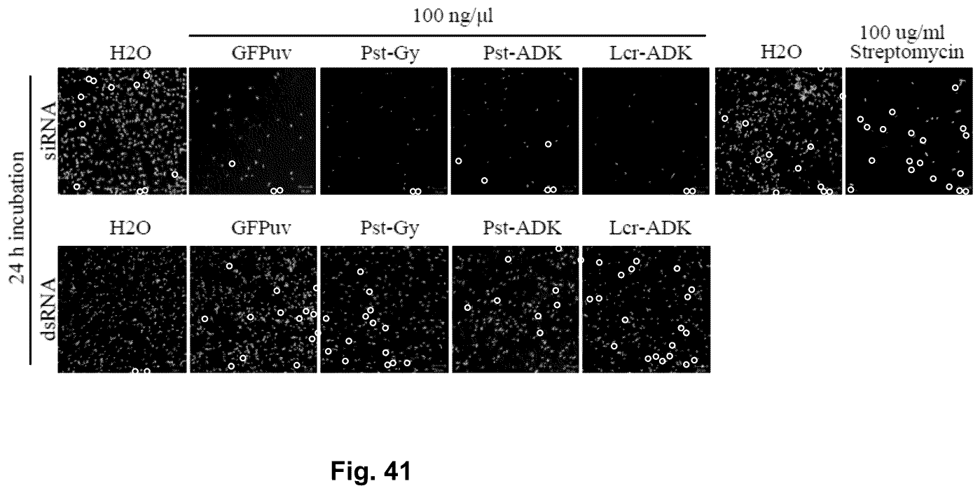

The inventors have discovered, however, that some bacteria take up, and can be controlled by, small RNA that are not a complement to any DNA or RNA associated with the bacteria (non-specific siRNA). For example, in relation to the three bacteria mentioned above, Envinia amylovora appears to be affected only by specific siRNA. Pseudomonas syringae, however, can be controlled by either specific siRNA or non-specific siRNA. The presence of non-specific siRNA does not kill the Pseudomonas syringae bacteria but causes them to be smaller and inhibits an increase in their population (see FIG. 40). Pseudomonas syringae are thereby rendered non-pathogenic by the non-specific siRNA. Liberibacter crescens can also be controlled by either specific siRNA or non-specific siRNA.

In some examples described herein, the small RNA used to control bacteria were in the range of 21-24 nt. This size is significant because the RNA silencing mechanism of a plant produces an abundance of 21-24 nt small RNA. Further, the transitive silencing mechanism of a plant causes the replication of small RNA into large double stranded RNA, which are then broken into numerous 21-24 nt small RNA. Thus, the effect of the 21-24 nt RNA used in the experiments suggests that small RNA produced by RNA silencing or transitive silencing by the plant itself may also control bacteria.

The polynucleotide sequence for the inserts of exemplary CYVaV constructs (CYm2250LD1pstGYR3-34sh and CYm2250LD1pstADK327-356sh) that target Gyrase A and Adenylate kinase, respectively, are presented below, wherein the lock and dock (LD1) sequence is shown in lowercase and underlined, and the siRNA small hairpin sequence is shown in uppercase.

LD1pstADK327-356sh (SEQ ID NO: 136):
gcgatatggattcagggactCGCCGTTGATGACGAAGAAATCGTCAAGCG
CGAACGCTTGACGATTTCTTCGTCATCAACGGCGagtccctgctcagggg
aaactttgtgtcctaagtcgc
LD1pstGYR3-34sh (SEQ ID NO: 137):
gcgatatggattcagggactGGGCGAACTGGCCAAAGAAATCCTCCCGGT
CGAAACCGGGAGGATTTCTTTGGCCAGTTCGCCCagtccctgctcagggg
aaactttgtgtcctaagtcgc

Bacterial gene sequences targeted by siRNA are presented below. Gene sequences of P. syringae tabaci_Gyrase subunit A (Pst_GY), P. syringae tabaci_Adenylate Kinase (Pst-ADK), and GFPuv were utilized for specific siRNA targets are presented below (wherein sequences underlined in solid line are forward primers for dsRNA synthesis, and sequences underlined in dashed line are reverse primers for dsRNA synthesis)

P. syringae tabaci_Gyrase subunit A (Pst_GY)
(SEQ ID NO: 138):
atgggcgaactggccaaagaaatcctcccggtcaatatcgaagacgagctgaagcagtccta
cctcgactacgcgatgagcgtaatcgtcggtcgagcactgcccgatgcgcgcgacggcttga
agcccgtgcaccggcgcgtgttgttcgcaatgagcgagctgggtaacgactggaacaagccg
tacaagaaatccgcccgtgtggttgqtgacgtgatcggtaagtatcacccgcacggcgatac
agccgtgtacgacaccatcgttcgtatggctcagccattctcgctacgctacctgctggtag
acggtcagggcaacttcggttcggtcgatggcgacaacgctgcggccatgcgatacaccgaa
gtgcgcatgaccaagctggcgcacgagctgctggccgacctgcacaaggaaaccgtggactg
ggtgccgaactacgacggcaccgaaatgatccccgcggtcatgccgacccgtattcccaacc
tgctggtcaacggttccagcggtatcgccgtgggcatggcgaccaacattccgccgcacaac
cttggtgaagtcatcgacggttgcctggcactgatcgacaaccccgagttgacgatcgatga
gctgatgcagtacatccccggcccggatttcccgacagcggcgatcatcaacggccgtgcgg

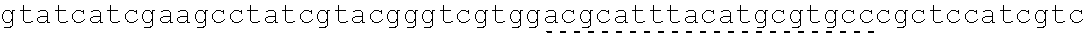

gaagacatcgacaaggtcggcggacgtcagcagatcgtcatcaccgaactgccgtaccagct
taacaaggcacgtctgatcgagaagatcgccgagctggtcaaggaaaagaaactcgaaggca
ttaccgagctgcgtgacgagtccgacaaggacggtatgcgcgtggtcatcgagctgcgtcgt
ggcgaagtgccagaggttgttctcaacaacctttatgcccagacccagctgcaaagcgtttt
cggtatcaacatcgttgccttgattgacggtcgtccacgcatcctgaacctcaaggacctgt
tggaagcctttgttcgtcaccgtcgcgaagtggttacccgccgtaccgtattcgagctgcgc
aaggcgcgtgagcgtggccatatccttgaaggtcaggctgttgcgttgtccaacatcgaccc
ggtcatcgccctcatcaaggcctctccgacacctgcagaagccaaggaggcgttgatcaaga
cgccttgggaatcaagcATGCGCGTGATTCTGCTAGGAgcagtggtcgaaatggtcgagcgt
gcaggtgccgattcgtgccgccctgagaacctggacccgcaatacggtctgcgtgaaggcaa
gtatttcctgtcaccggaacaggctcaggccattctggaactgcgtctgcatcgcctgaccg
gtctggaacacgagaagctgctgggcgagtaccaggaaatcctcaaccagatcggcgagctg
atccgcatcctcaacagcgcaacgcgcctgatggaagtgatccgcgaagagctggaagtgat
ccgctccgagtacggcgatgcccgtcgtaccgagattctcgatgcacgtctggacctgaccc
tgggtgacctgatcaccgaagaagagcgtgtggtcaccatctcccatggcggctatgccaag
acccagccattggcggtctatcaggctcagcgtcgtggcggcaagggcaagtcggccaccgg
catcaaggatgaggattacattgctcacctgctggtcgccaacagccacacgacactgctga
tgttctccagcaagggcaaggtgtactggctcaagacctatgagatcccggaagcgtcccgc
gctgcccgtggtcgtccgttggtcaacctgttgccgctgagcgacggcgaatacatcaccac
catgctgccggttgacctcgaagccatgcgcaagcgtgccgacgaagaaggcgaagccctcg
aaggcgagctggacgacgeggaaaacagcagcgagaccgaagaagagcgcaaggcccgtatc
aaggccgccgacaagaagaaggctccgttcatcttcatgtccaccgccaacggtaccgtcaa
gaagaccccgctggttgcattcagccgtcaacgcagttcgggcctcattgcccttgagctgg
acgagggcgacatcctgatctccgctgccattaccgatggcgaacaggaaatcatgctgttc
tccgatggcggcaaagtgacccgcttcaaggaatccgatgtgcgcgccatggggcgtaccgc
tcgcggcgtgcgtggcatgcgtctgccagaagggcaaaagctcatttcgatgctgatcccgg
aagaaggcagccagatcctcaccgcttccgagcgcggttacggcaagcgtacggccatttcc
gagttccccgagtacaagcgcggcggtcagggtgtcatcgccatggtcagcaacgagcgtaa
cggccgtctggttggcgcagttcaggtgcttgatggcgaagaaatcatgctgatttccgatc
agggcacgctggtgcgtacccggggggcgaagtgtccagtctgggccgtaacactcagggt
gtgaccctgatcaagctggccagcgacgagaaactggtcggtctggagcgtgttcaggagcc
gtcggaagtcgaaggcgaagagcttgaaggcgaagaagttatcgacggcgtgattgtcgatg
ccgctgaagctgaagtgggcgacgccggtgaagacctgcaagcggacgctgcgccagacgaa
gacgaacccagaactga
P. syringae tabaci_Adenylate Kinase (Pst-ADK)
(SEQ ID NO: 139):
atgcgcatgattctgctaggagctcccggggccggtaaaggtactcaggcaaaattcatcac
tgaaaatttcggcatcccgcaggtttcgacaggcgacatgctgcgcgctgcagtcaaggctg
aaaccgagcttggcctgaaggccaagagcgtcatggactcgggtggtctggtttccgatgac
ctgatcattggtctgatcaaggatcgtctggcccagccggattgtgcgaacggogttctgtt
cgacggcttcccgcgcaccattcctcaggccgaagccctgttgaaagcaggtctggaaatcg
accacgtgctggaaatcgccgttgatgacgaagaaatcgtcaagcgcatgtcgggccgccgg
gttcacgaaggctctggtcgcatctatcacaccattttcaacccgccgaaagtcgagggtat
cgatgatgtgactggtgaaccgctgttgcagcgcaaggacgacgtcgaagaaaccgtgcgtc
atcgcctgtcggtctaccatgcccagaccaagccgctggtcgagttctacagcaagctggaa
gcaaagaacggcaagcccaagtgcagccatattccaggtgttggctcggttgaagaaatcac

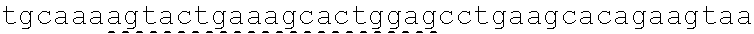

GFPuv
(SEQ ID NO: 140):
atgagtaaaggagaagaacttttcactggagttgtcccaattcttgttgaattagatggtga
tgttaatgggcacaaattttctgtcagtggagagggtgaaggtgatgcaacatacggaaaac
ttaccettaaatttatttgcactactggaaaactacctgttccatggccaacacttgtcact
actttctcttatggtgttcaatgcttttcccgttatccggatcatatgaaacggcatgactt
tttcaagagtgccatgcccgaaggttatgtacaggaacgcactatatctttcaaagatgacg
ggaactacaagacgcgtgctgaagtcaagtttgaaggtgatacccttgttaatcgtatcgag
ttaaaaggtattgattttaaagaagatggaaacattctcggacacaaactcgagtacaacta
taactcacacaatgtatacatcacggcagacaaacaaaagaatggaatcaaagctaacttca
aaattcgccacaacattgaagatggatcagttcaactagcagaccattatcaacaaaatact

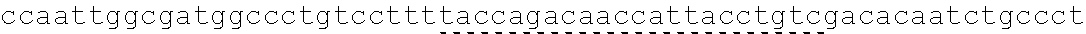

ttcgaaagatcccaacgaaaagcgtgaccacatggtccttcttgagtttgtaactgctgctg
ggattacacatggcatggatgagctctacaaataa

Since CYVaV and other iRNA do not have a silencing suppressor, the silencing mechanism and/or transitive silencing mechanism of a plant infected with CYVaV or another iRNA produces numerous non-specific siRNA. This suggests that infection of a plant by a virus, in particular by CYVaV or another iRNA, will cause the plant to produce abundant non-specific siRNA, which may control certain bacteria in the plant. In particular, bacterial canker in almond trees, or other disease caused by P. syringae, can be treated by infecting the plant with a virus tolerated by the plant such as CYVaV or another iRNA. In another example, citrus greening can be treated by infecting the plant with a virus tolerated by the plant such as CYVaV or another iRNA.

The wild type CYVaV or other iRNA alone may be sufficient to control the bacterial infection. Alternatively, CYVaV or other iRNA may be engineered to also include a specific siRNA to enhance. In another alternative, the CYVaV or other iRNA may be engineered to also include an siRNA or other insert that enhances the transitive silencing response of the plant. For example, CTV is widespread in citrus trees. CYVaV or other iRNA with an insert that complements a region of the CTV sequence, may be used to vaccinate or treat a citrus tree to inhibit or treat citrus greening. The CYVaV or other iRNA with an insert that complements a region of the CTV sequence would also be useful to inhibit or treat CTV infection in the same citrus trees.

Recently, highly targeted anti-bacterial enzymes have been developed for use in animals and humans as a replacement for current antibiotics. These enzymes are engineered from bacteriophage lysis proteins and are known as enzybiotics. As with the parental bacteriophage proteins, enzybiotics can lyse bacterial cell walls on contact, but are designed to be used external to both gram positive and gram negative bacteria. Enzybiotics are engineered to lyse only targeted bacterium, leaving other members of the microbiome unaffected. In some implementations, an iRNA vector is provided that includes a non-coding RNA insert that can be translated into an anti-bacterial protein like an enzybiotic.

In some implementations, an iRNA vector is provided that includes an RNA insert that interferes with the functionality of the insect vector at issue. Insects have an RNA silencing system similar to plants; small RNAs ingested by insects are taken up into cells and target critical mRNAs for degradation or blockage of translation within the insect. In some embodiments, a targeted insert is provided that is capable of silencing a critical reproductive function of the insect vector, resulting in sterilization of the insect. Of particular relevance are phloem-feeding insects that transmit phloem-limited pathogens, where a non-coding RNA insert into a phloem-limited vector is readily taken up by feeding insects.

In some implementations, an iRNA vector is provided that includes a non-coding RNA insert that targets a plant response to a pathogen. In some cases, bacteria deposited into a tree by an insect vector does not directly damage the tree. However, the host tree produces excessive callose in their phloem in order to isolate the bacteria, which can ultimately restrict the flow of photoassimilates and kill the tree. Thus, the RNA insert silences and/or depresses such callose production.

In some implementations, an iRNA vector is provided that includes a non-coding RNA insert that targets a virus, for example CTV. In some implementations, an iRNA vector is provided that includes a non-coding RNA insert that is taken up by a pathogenic bacteria or fungus making the non-coding RNA available to silence a critical function within the pathogen that can kill or reduce the virulence of that pathogen to its host.

In some implementations, an iRNA-based vector, e.g., an iRNA vector that includes a non-coding RNA insert, is grafted into rootstocks or seedlings in order to provide protection against a pathogen or in order to make that rootstock or seedling more robust. For example, planting citrus trees on sour orange root stock can be advantageous since trees grown on sour orange rootstock are, among other things, less affected by HLB than trees grown on many other rootstocks. The sour orange rootstock is also tolerant of a wide range of growing conditions. However, sour orange rootstock is also highly susceptible to CTV and many citrus growers abandoned sour orange rootstock after CTV outbreaks. Introducing an iRNA based vector adapted to target CTV into sour orange rootstock thereby produces rootstock that is tolerant to both CTV and HLB. The iRNA-based vector can be introduced into the sour orange rootstock, for example, by grafting a scion containing the iRNA based vector to the rootstock or by grafting a part of plant containing the iRNA-based vector to the rootstock or to a scion grafted to the rootstock. In some examples, seedlings are produce having sour orange rootstock, a scion of sour orange or another citrus species, and the iRNA-based vector containing a heterologous element that targets CTV. In some implementations, the heterologous element is a hairpin or single-stranded sequence, which includes a sequence complimentary to (though not necessarily exactly the same as) a sequence conserved within one or more strains of CTV.

Enhanced Stabilization: The ability to achieve a sufficiently stable insert in a viral vector has eluded prior researchers due in part to a lack of expertise in RNA structure. Some researchers attempted to use an empirical trial-by-error approach to choose insert locations, but without a sufficient understanding of the effect of the insert on the stability of the vector. In accordance with the present disclosure, the core principles behind what makes inserts stable were investigated and utilized to optimize the process of insert location and structure. First, an excellent correlation was discovered between vector instability and numerous structural alterations throughout the viral genome as assayed by SHAPE structure probing, which results in poor in vitro translation of the polymerase that requires a −1 ribosomal frameshift. The following hypothesis was then tested: what if RNA viruses can discern “self” from “non-self” ? In other words, what if RNA viruses have evolved their genomes such that every portion of the genome is maximally structured to support the most fit virus? This implies that every hairpin emanating from the genomic RNA nucleic acid “backbone” is maximally stable for its size and shape and properly configured for a successful virus.

It was determined that the insertion of haphazard hairpins whose design is based solely on siRNA generation, and which do not resemble in length, structure and/or stability endogenous, evolved hairpins, stresses and destabilizes the entire structure of a viral genome. Since RNA viral genomes are dynamic, with active and inactive structures precisely balanced to control key viral functions like frameshifting, destabilizing the genome can have adverse repercussions that are relieved if the inserts are deleted through recombination. If the genome contains foreign structures, the deletion or removal of inserts or portions thereof (e.g., replicase-mediated events) will eventually occur, thereby leading to the non-functionality of the commercial RNA vector product. Such deletion and/or removal of inserts or portions thereof increases the possibility of generating a more fit virus (though not necessarily functioning to target the desired pathogens/host gene expression), as the virus with a destabilizing insert is more poorly fit.

Following from the proposed hypothesis, designing inserts that “mimic” the structure (i.e., length and/or the size and location of looped or non-base-paired regions) and/or stability (i.e., the ΔG) of endogenous hairpins (which optionally include hairpin-like structures and structures with multiple hairpins or hairpin structures) should increase stability of the insert within the vector, particularly when inserted into suitable locations. Mimicked hairpins are designed by using the sequence of interest (i.e., an siRNA) as some or all of one side, for example the 5′ side, of the hairpin and an artificial sequence on the other side, for example the 3′ side, that provides the corresponding nucleotides for the base-pairs and loops, resulting in a structure that mimics an endogenous (natural) hairpin at some location on the wild-type vector or a related wild-type vector. A mimic insert may be inserted in the same location, i.e., as a replacement for the wild-type hairpin that was mimicked. Alternatively, a mimic insert may be inserted into a different location, with the wild-type hairpin either retained or deleted, if not required for viability of the vector.

In accordance with the present disclosure, hairpin inserts were engineered to have a structure that mimics the structure of a natural endogenous hairpin structure of the vector of interest or a relative thereof (e.g., a CYVaV vector having an insert that mimics a hairpin structure of CYVaV or another ulaRNA, for example a Class 2 ulaRNA such as OULV). As noted above, providing and maintaining stability of sequences inserted into the CYVaV vector, which are subsequently converted into siRNAs when the RNA virus vector is targeted by the RNA silencing defense system in infected plants, raises various challenges. While some insertions are randomly more stable than others (i.e., the inserts remain in the vector for a longer time), little was previously understood about parameters required for stabilization. Solving the secondary structure of the CYVaV vector revealed an extremely compact structure with few extensive single-stranded regions, which would generally be considered the most suitable locations for making insertions. Various locations (noted above) were identified for accepting particular hairpin inserts (e.g., that targeted nuclear-encoded GFP), although other hairpin structures had different stabilities in all of these particular locations. Thus, producing a vector that allows for little or no discernable insert loss (e.g., assayed first by PCR and then by sequencing the cloned population after four to six weeks) proved difficult. This indicated that these hairpins decreased the fitness of the vector and that deletion of almost all (but not precisely all as surrounding vector sequences could also be deleted) or part of the insert increased fitness.

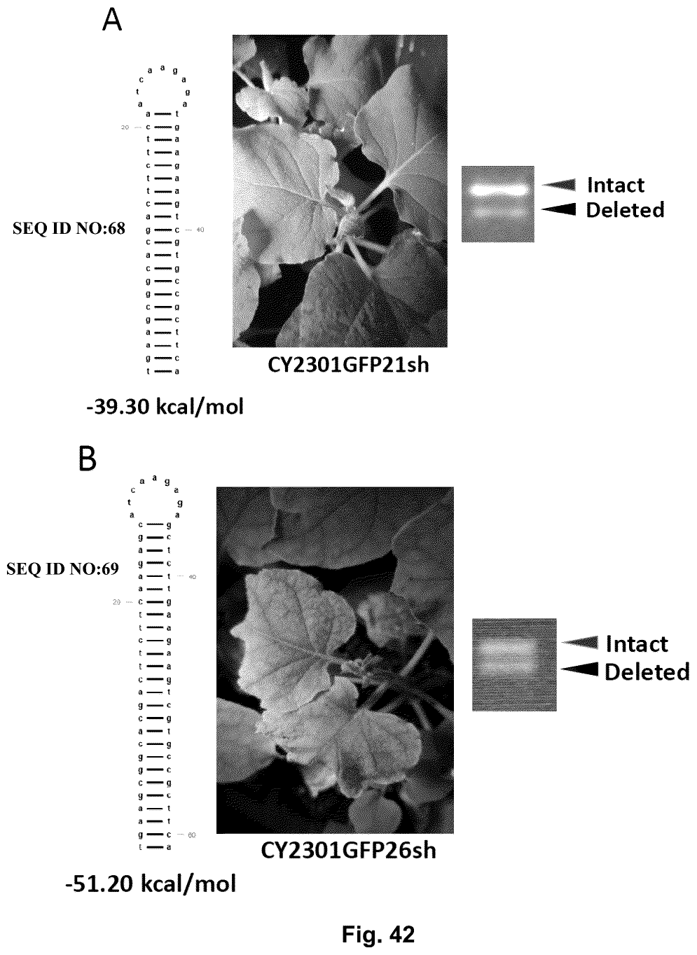

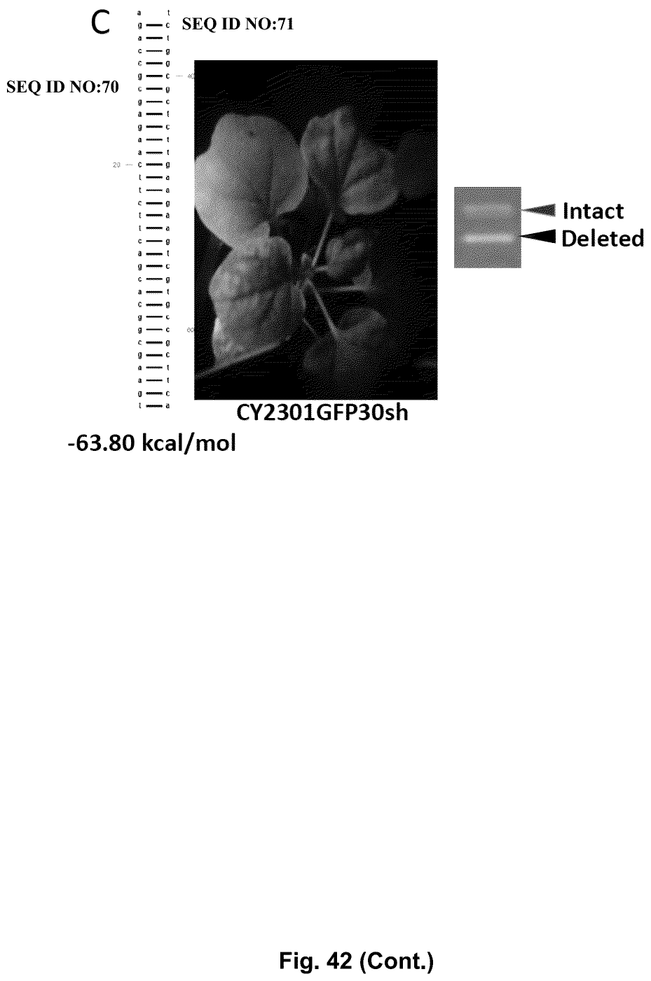

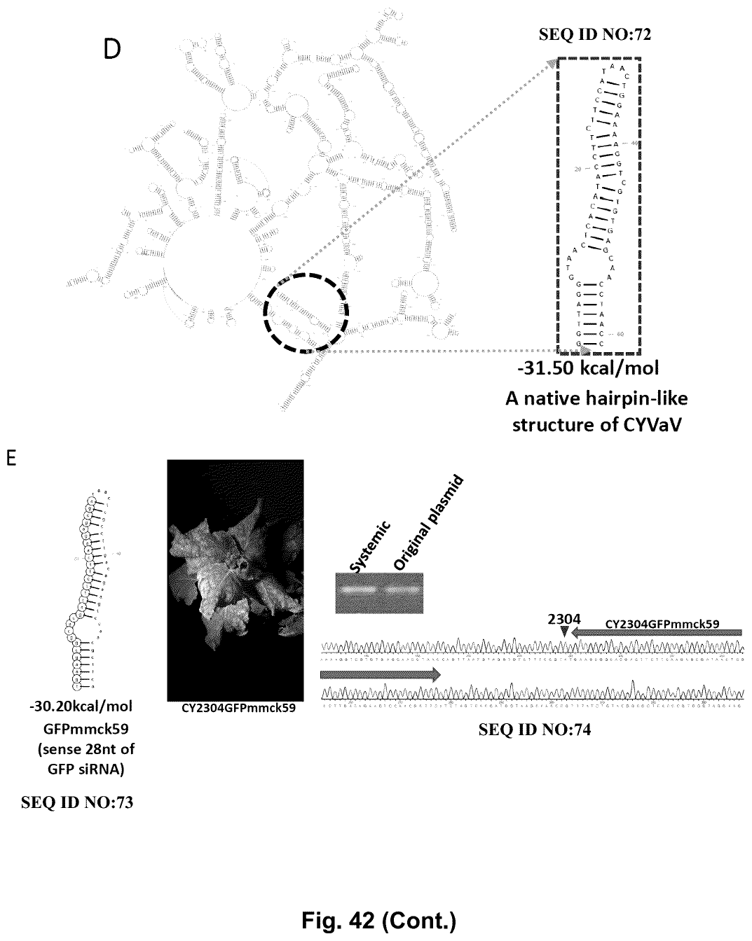

Intuitively, the more stable the structure of the hairpin insert (i.e., the higher or ‘more negative’ the ΔG), the less it should interfere with important viral structures (generally thought to be the reason for vector instability), providing that the polymerase is still able to melt the hairpin during replication. Surprisingly, it was discovered that the opposite is true (see FIG. 42, Panels A, B, and C). In particular, it was discovered that structurally stable hairpins were rapidly lost, as were inserted sequences with minimal structural stability. Also tested were non-structured inserts that were placed atop RNA scaffolds (e.g., lock and dock structures). While the lock and dock stabilized unstructured inserts and resulted in some very stable inserts, some inserts generated a small but discernable population of CYVaV that had lost various amounts of the insert and scaffold, particularly vectors with two such inserts.

In accordance with disclosed embodiments, hairpin inserts are engineered to mimic the structure (i.e., the overall length and the location of paired and unpaired “loop” regions) and/or sequence and/or ΔG of natural hairpin structures found in the vector molecule of interest. The utilization of such mimic hairpin(s) results in an extremely stable RNA hairpin insert(s) in an RNA virus vector, which remains stable for an extended period of time, preferably at least one year, or at least three years, or at least five years, or 5-10 years or more (e.g., for the life of the host plant). Thus, the disclosed methods and structures are suitable for producing commercial products in which the vector is capable of generating anti-pathogenic or other efficacious siRNAs for many years, if not for the productive lifespan of a host tree.

An RNA vector (e.g., CYVaV vector) including such mimic hairpins exhibited substantially increased stability as compared to a vector that included a hairpin structure that did not mimic the natural hairpin structure. The structure and sequence of a natural hairpin was determined; a mimic hairpin having a substantially similar structure and/or sequence and/or ΔG to the natural hairpin was then inserted into the vector molecule. Studies demonstrated that vectors possessing such mimic hairpin(s) inserted at the same location as the corresponding natural hairpin(s) (i.e., replacing the natural hairpin(s) at such location) were stable. Further studies demonstrated that vectors possessing such mimic hairpin(s) inserted at one or more different locations from the corresponding natural hairpin(s), but possessing substantially the same structure(s) and/or sequence(s) and/or ΔG as compared to the natural hairpin(s), were stable. It was found that hairpin structures having relatively conserved sequences across iRNAs (e.g., CYVaV and relatives thereof, other RNA molecules having least 50% or at least 70% identity with CYVaV) were particularly suitable as mimic hairpin structures. However, it was also found that a wild-type hairpin structure in a CYVaV relative, for example another Class 2 ulaRNA such as OULV, could be used to design a stable CYVaV insert even if the wild-type hairpin structure is not present in CYVaV. In some examples, an insert has a ΔG within 10 kcal/mol (plus or minus, or in a range of −5 to +15), or within 5 kcal/mol, or within 2 kcal/mol of the ΔG of a wild-type hairpin structure. Preferably, the insert structure is as close as possible (or slightly below) the hairpin being mimicked.

Referring to FIG. 42, Panel E, a mimicked hairpin (wherein the natural yet unnecessary hairpin at position 2219 was replaced with a mimic hairpin that resembles the natural hairpin) produced an extremely stable virus vector with no discernable loss of insert for the life of the plant. For example, random events that may lead to insert loss may generate a less fit virus, with such virus(es) lost from the population. The process of replacing a natural hairpin with an engineered mimic hairpin was repeatedly tested, with several mimics for each of three different natural hairpins, including one that is fully base-paired, and the mimic hairpins inserted into positions 2219 and 2204. In every case, the mimicked hairpin replacement at positions 2219 and 2204 resulted in an extremely stable virus vector. It was determined that the closer the ΔG of the mimicked hairpin is to the endogenous hairpin being mimicked, the more stable the insert. RNA structure probing clearly showed far fewer structural disruptions throughout the vector genome from the presence of a mimic hairpin as compared to random hairpins inserted at the same locations. This further demonstrated the importance of mimicry to achieve a vector that maintains a structure close to (though generally not identical to) the wild-type RNA genome structure.

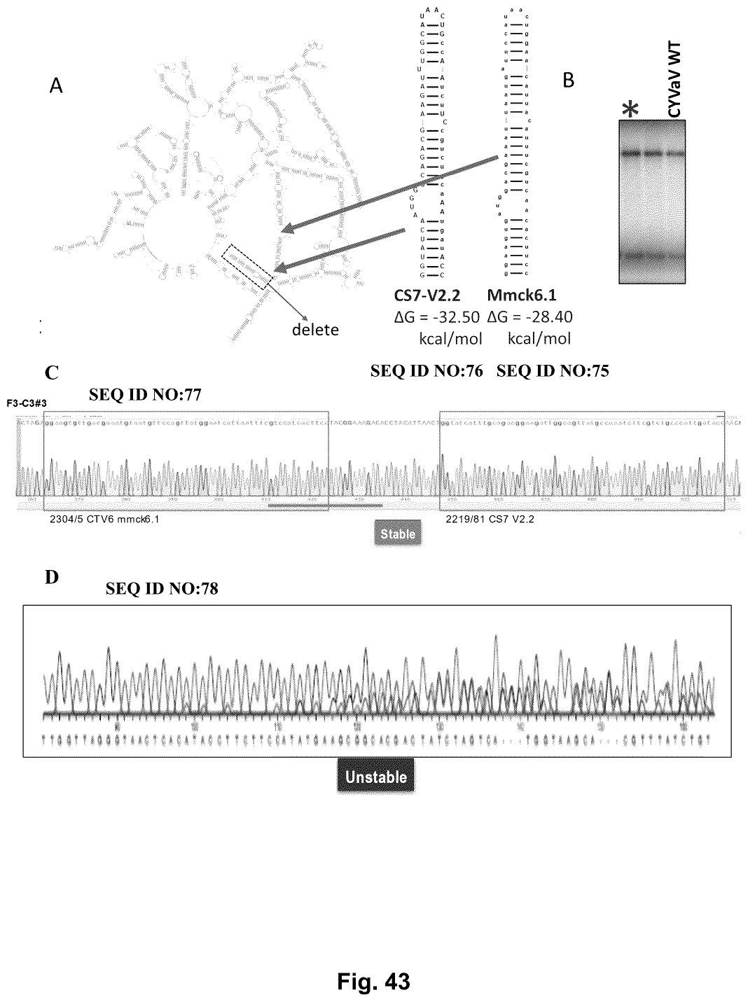

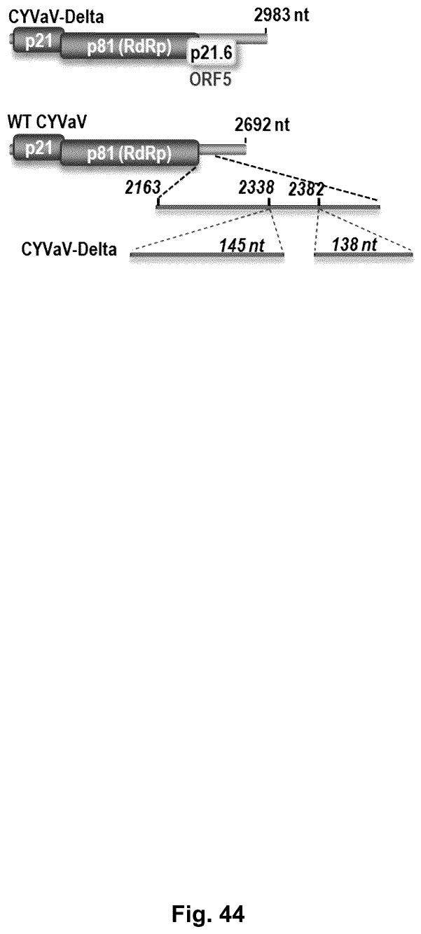

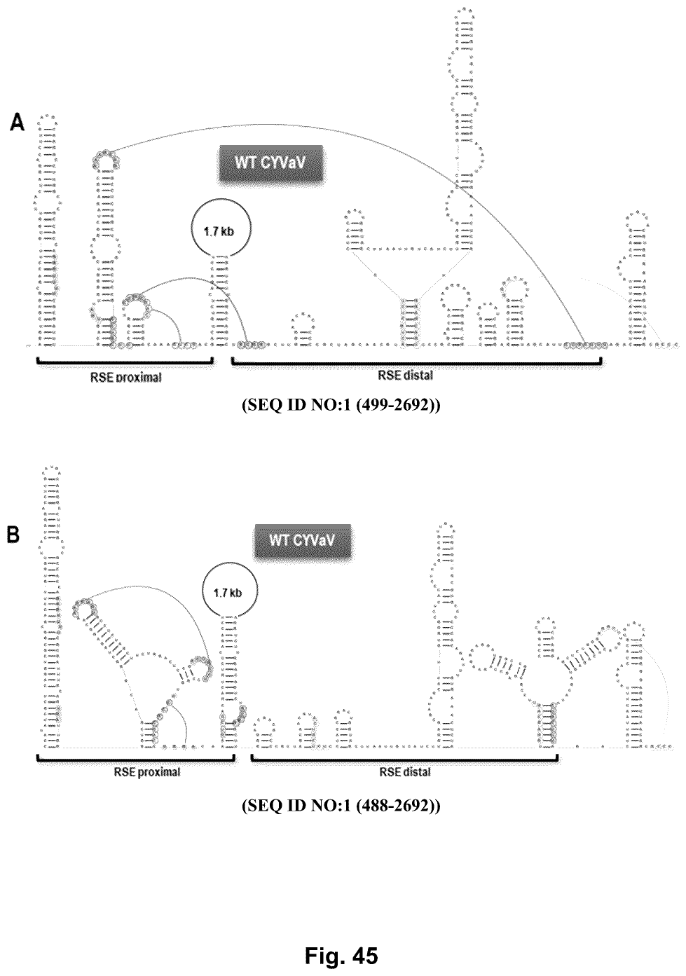

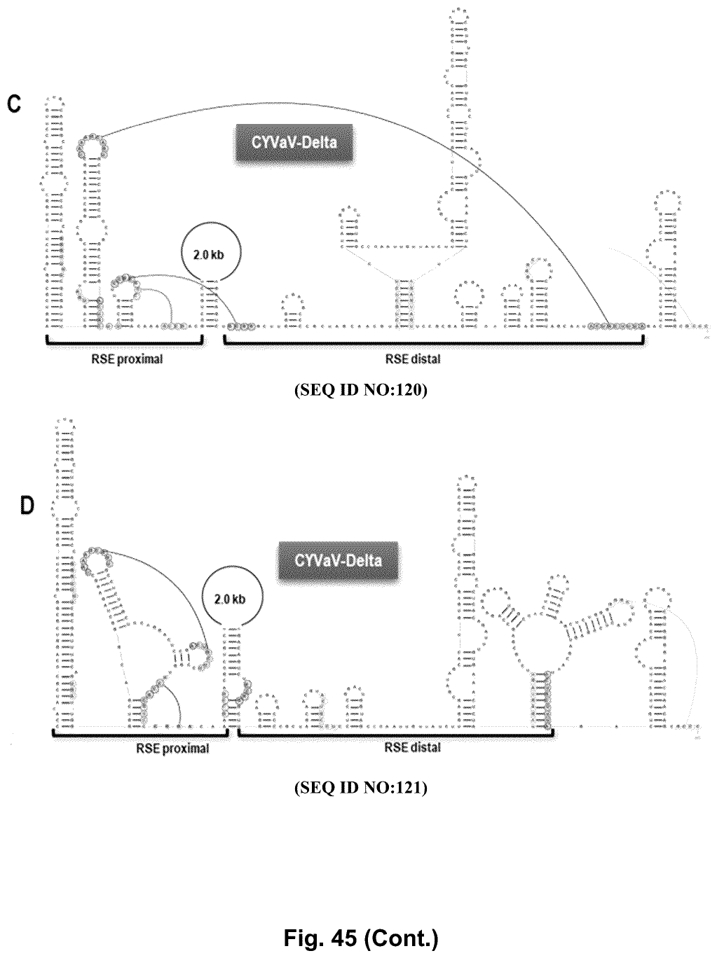

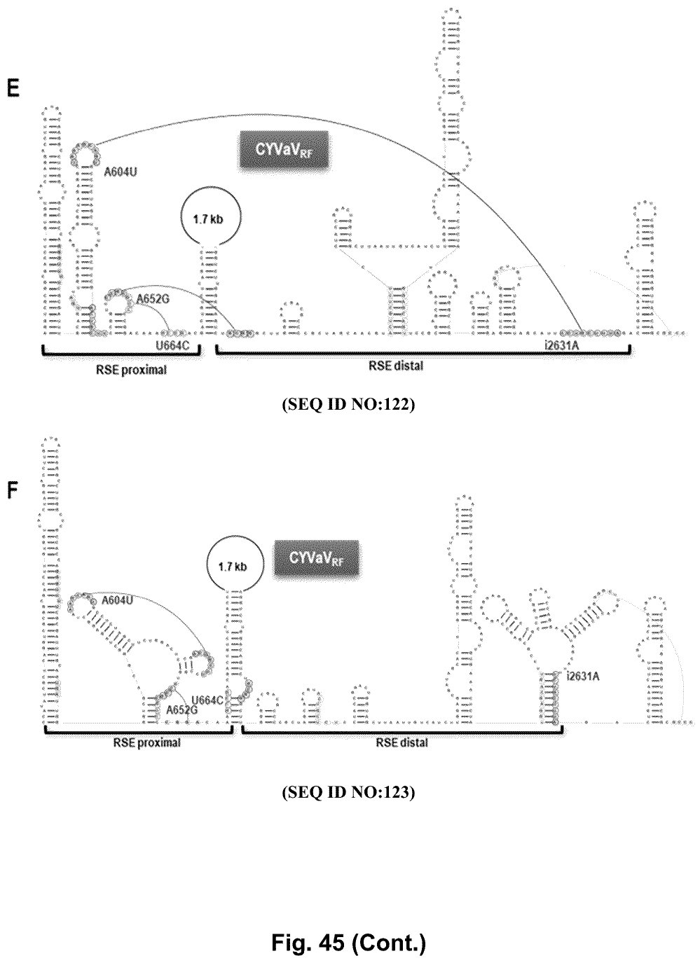

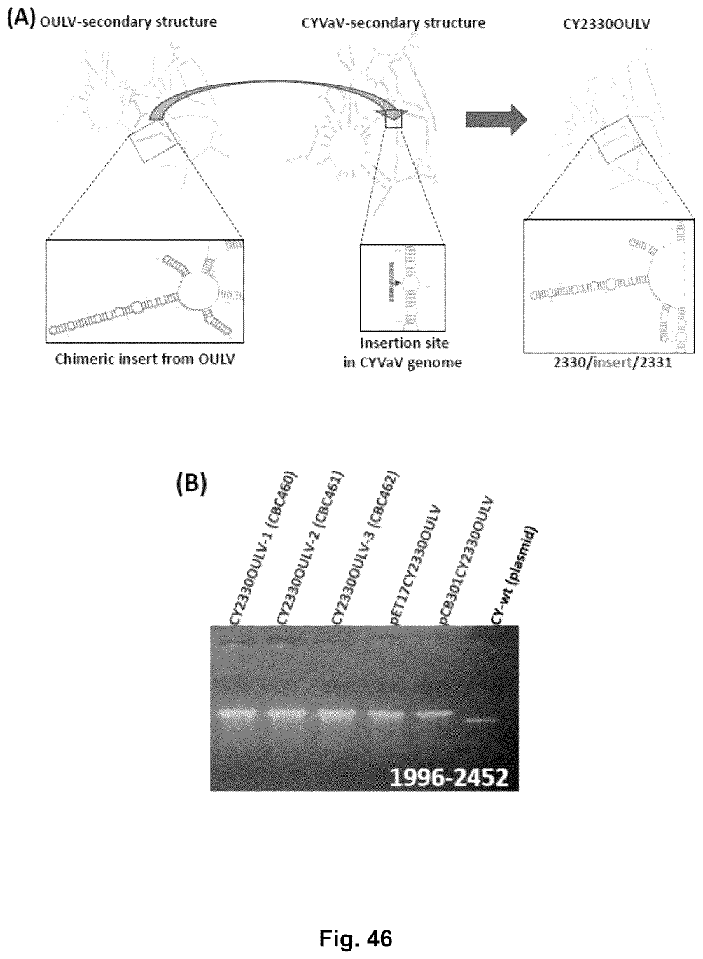

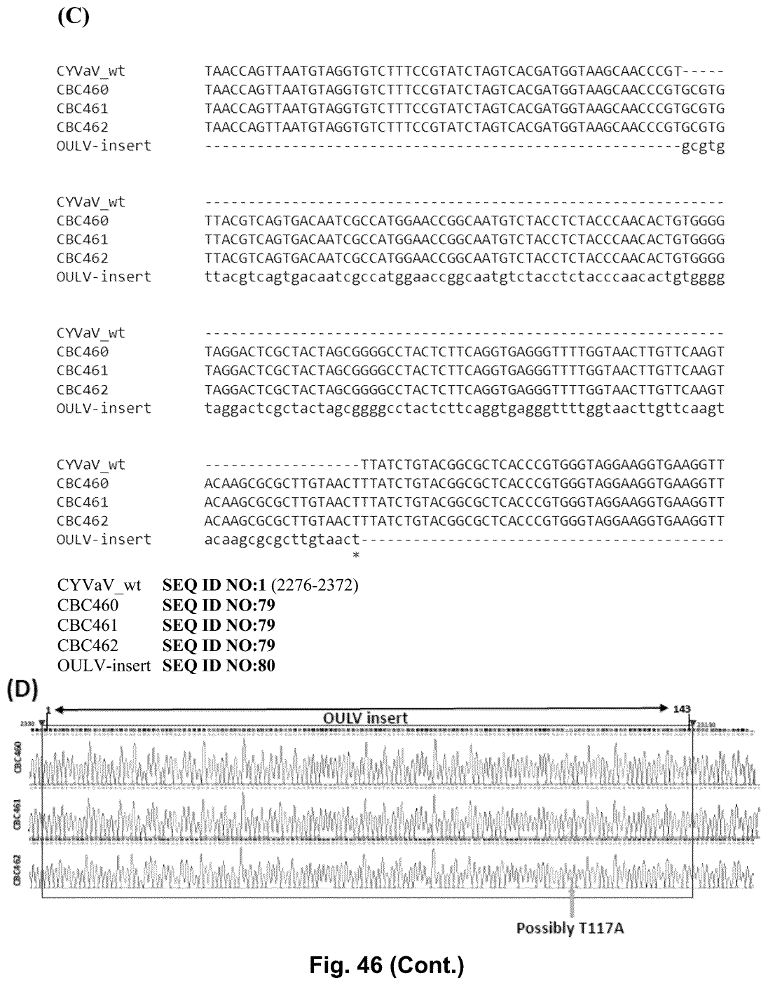

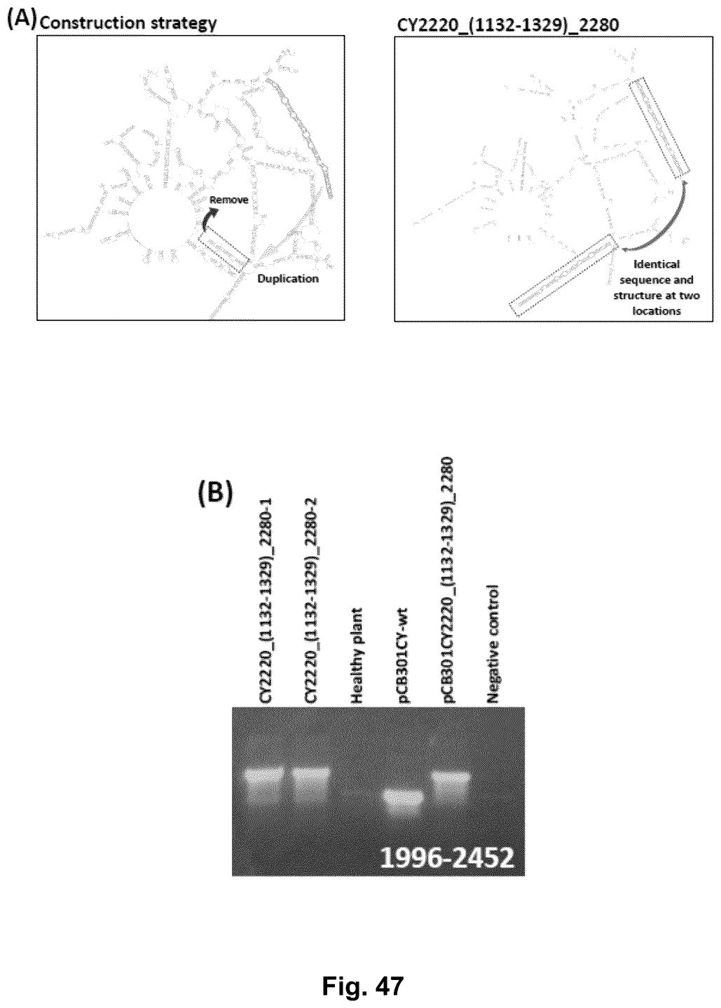

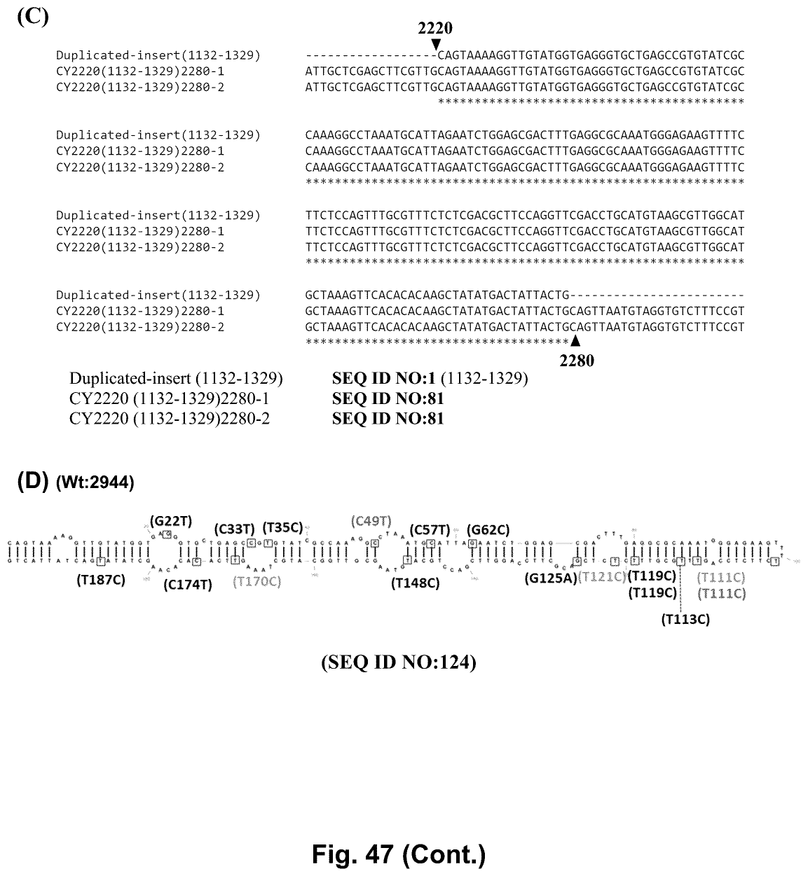

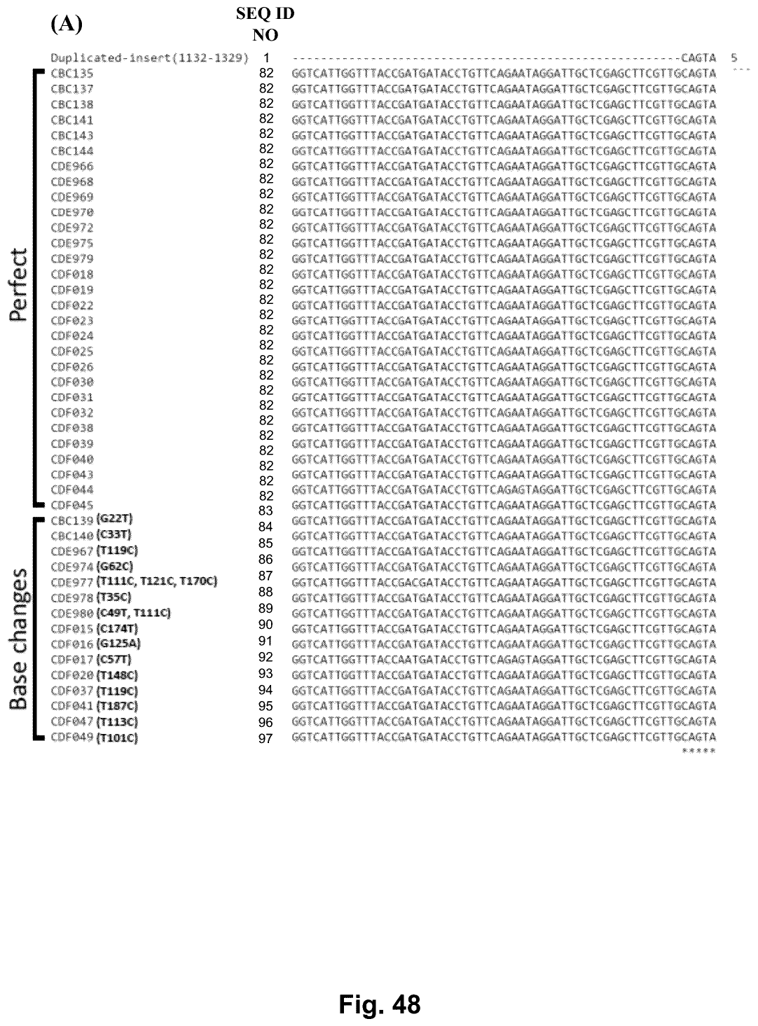

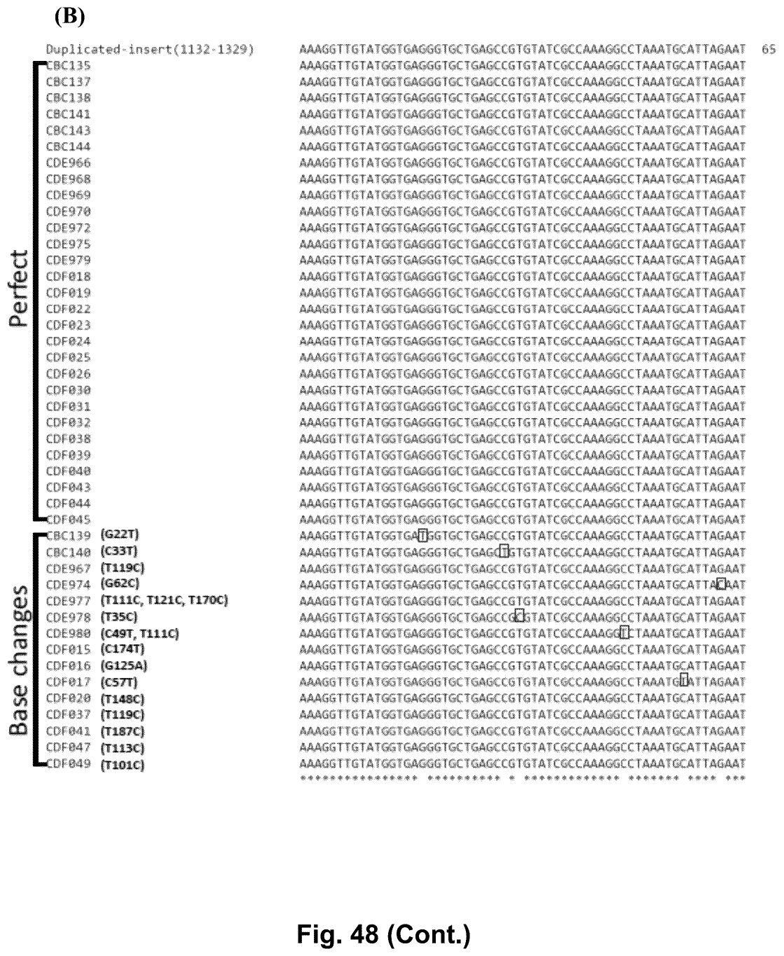

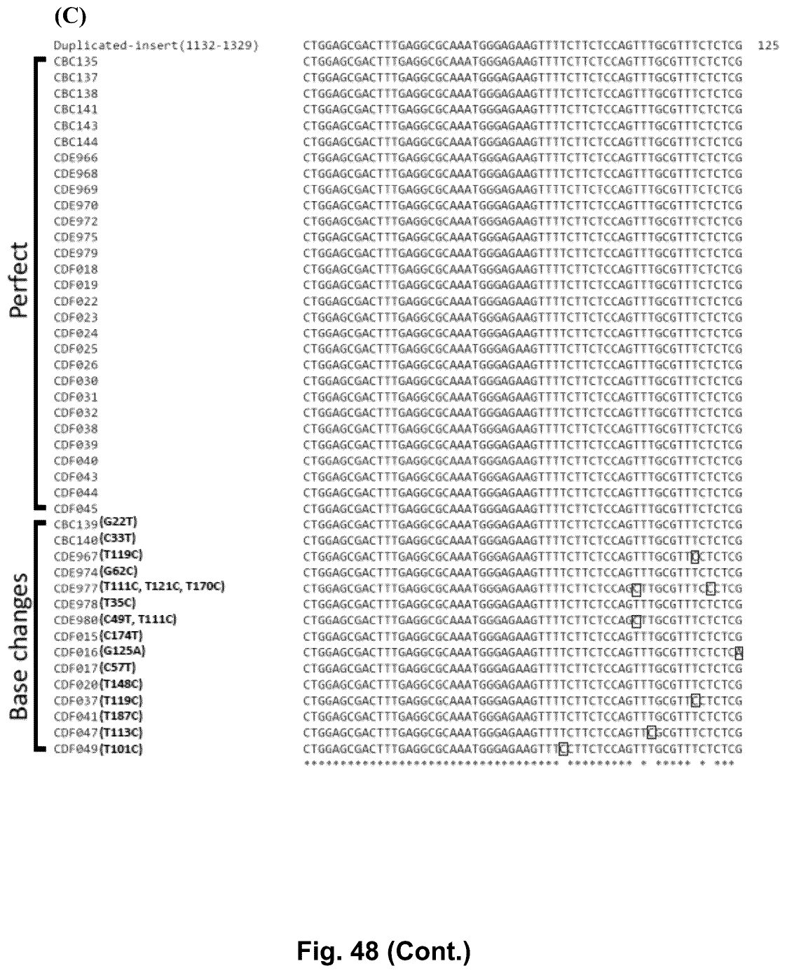

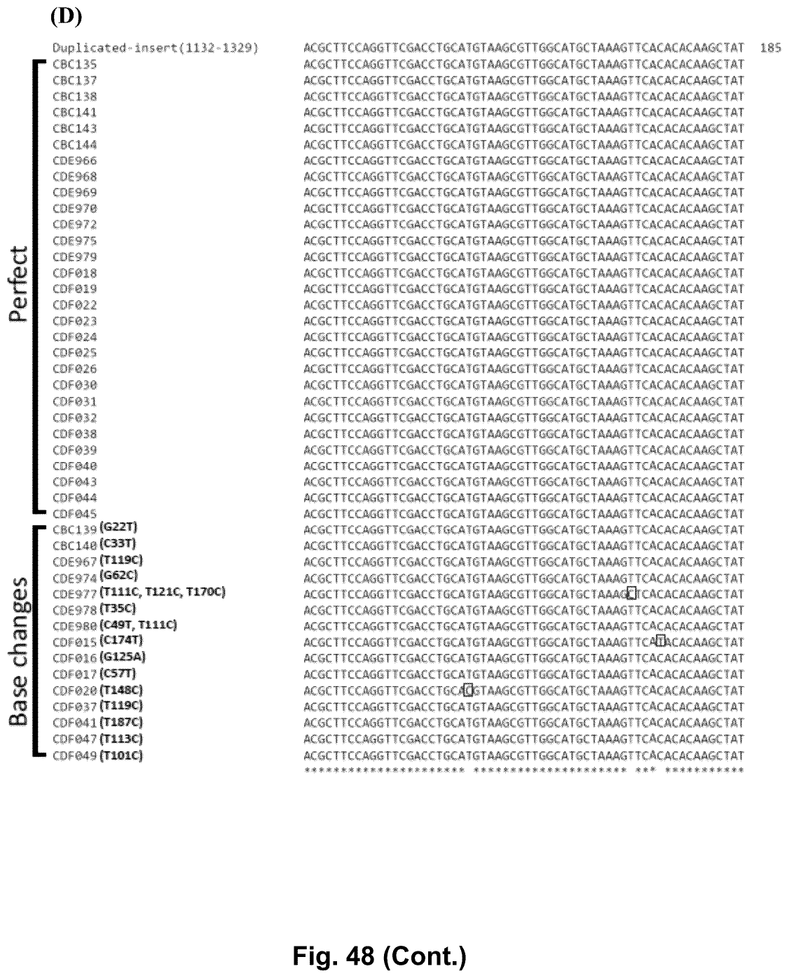

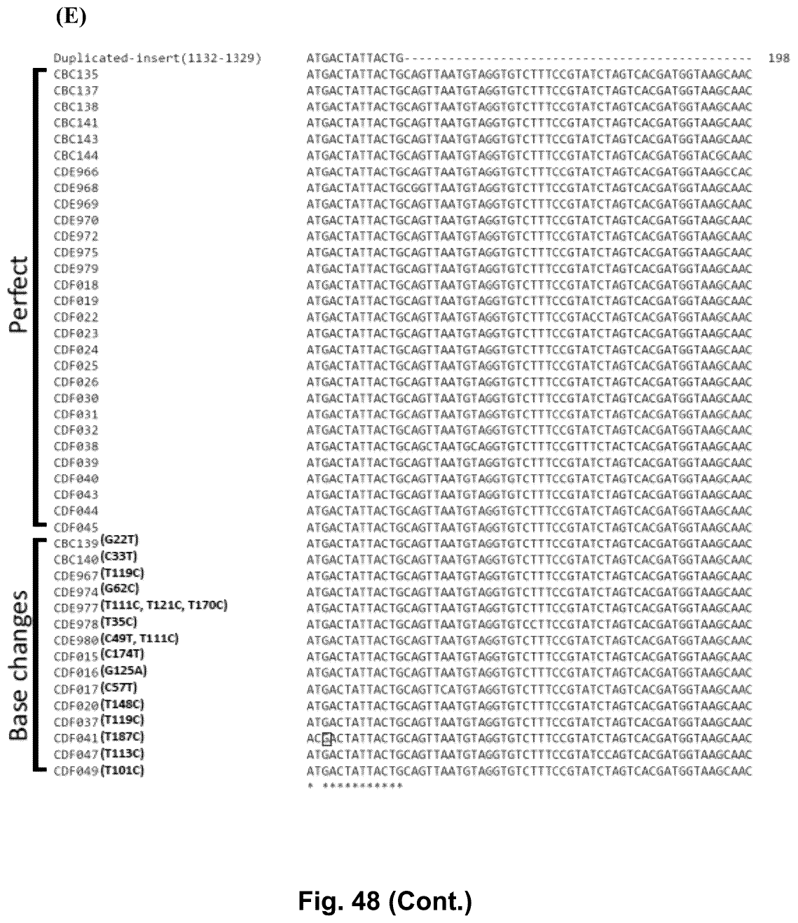

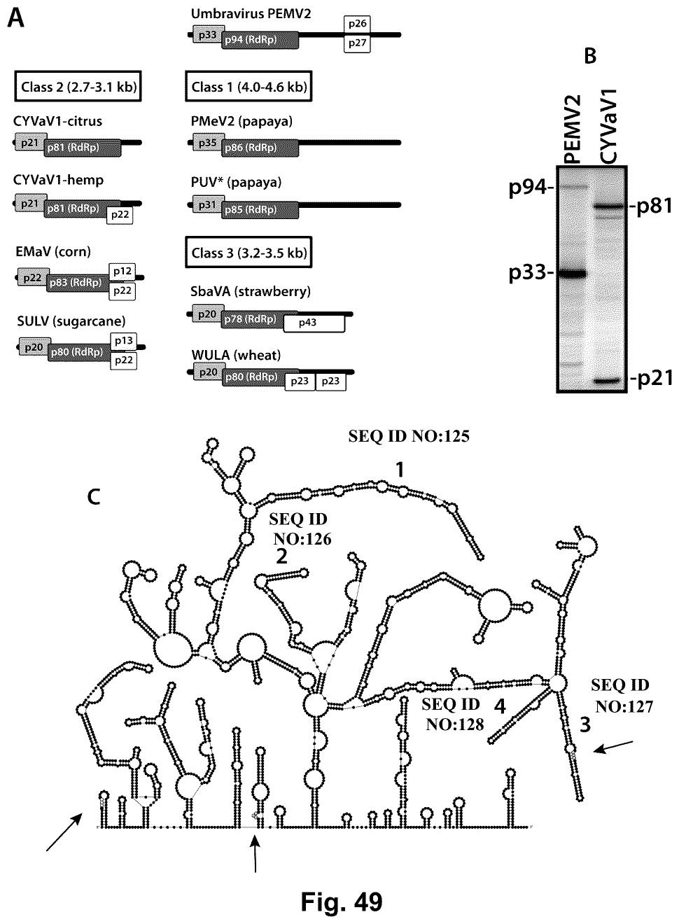

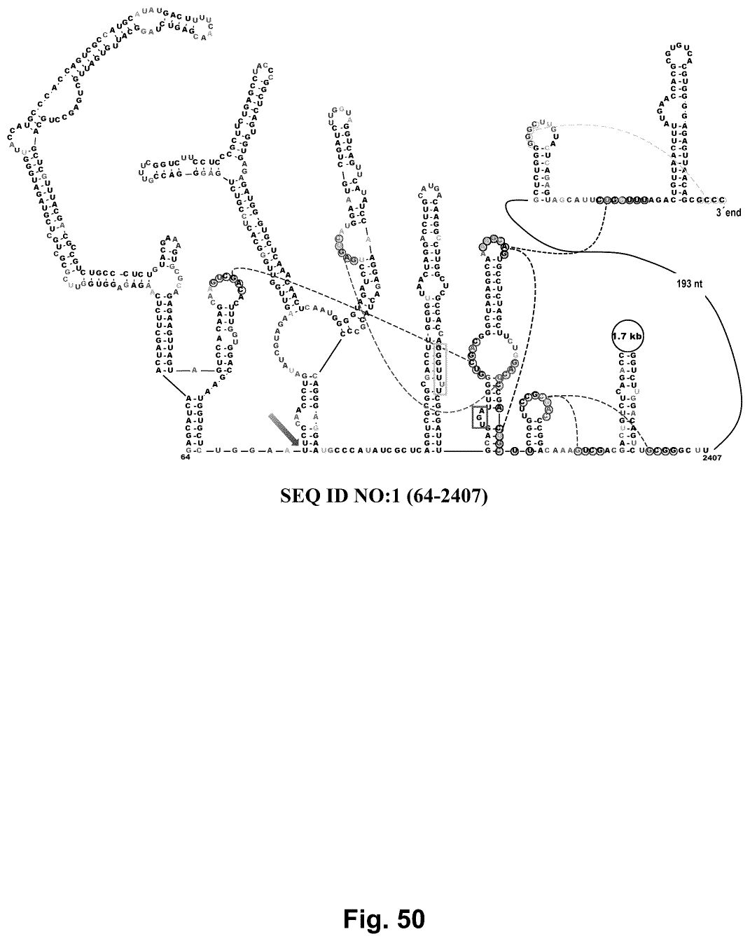

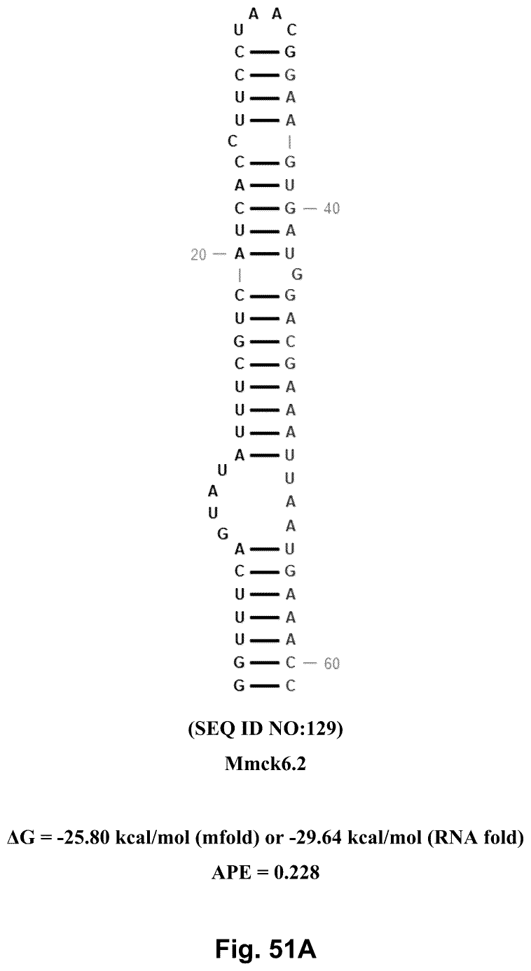

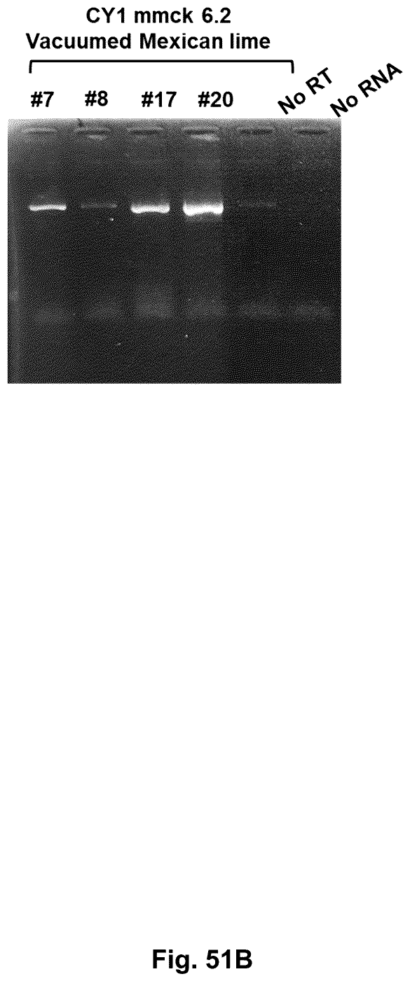

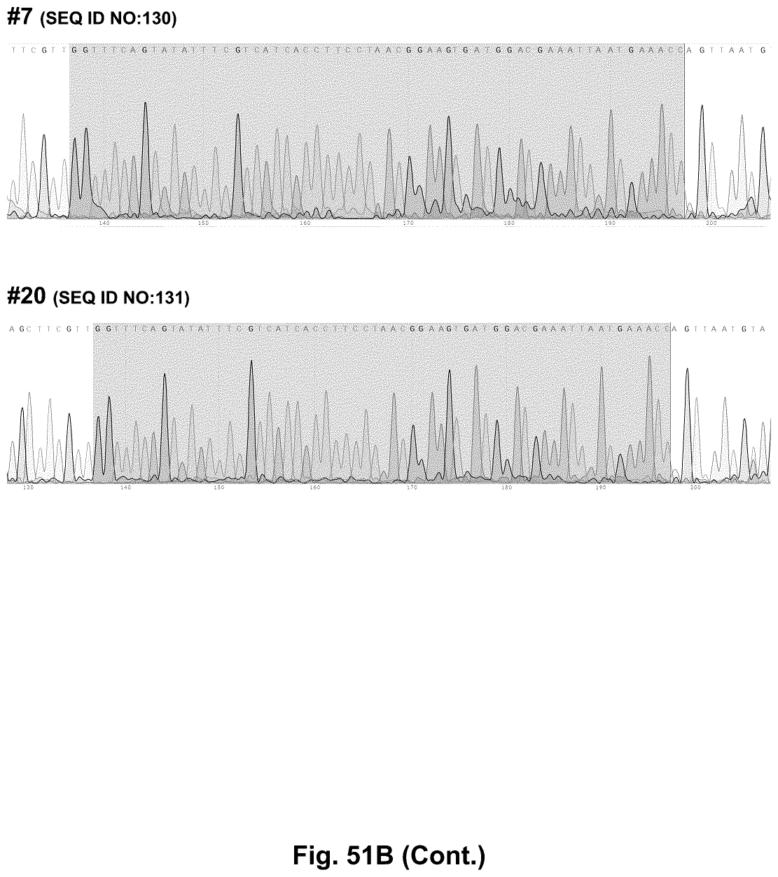

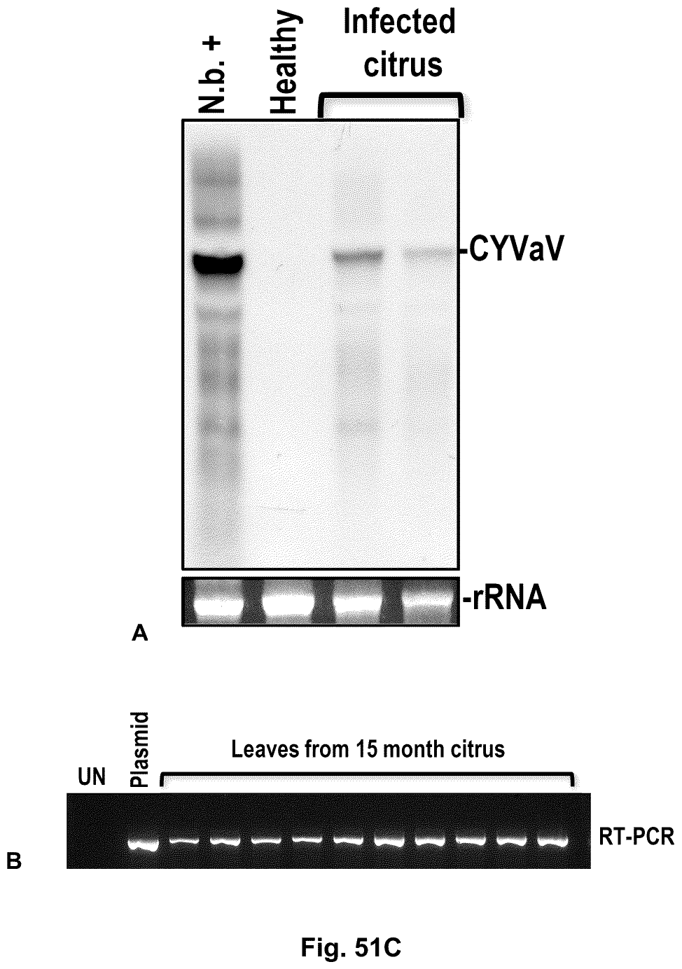

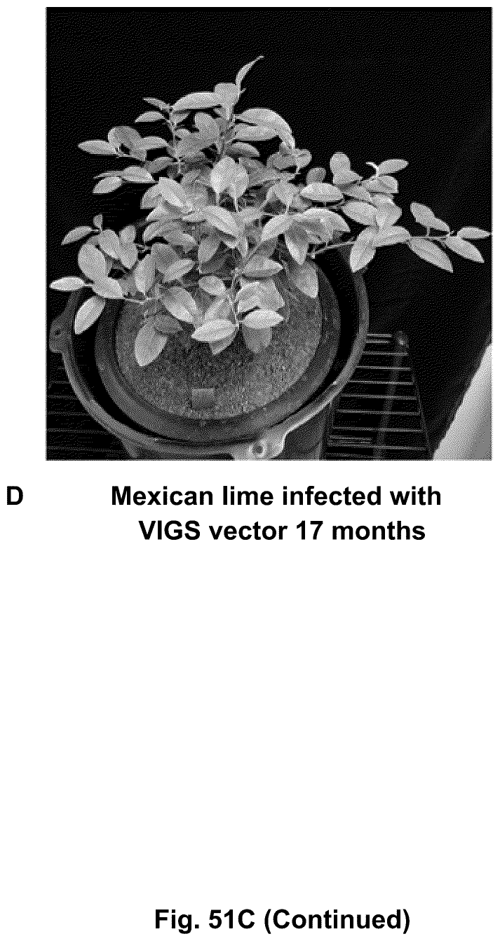

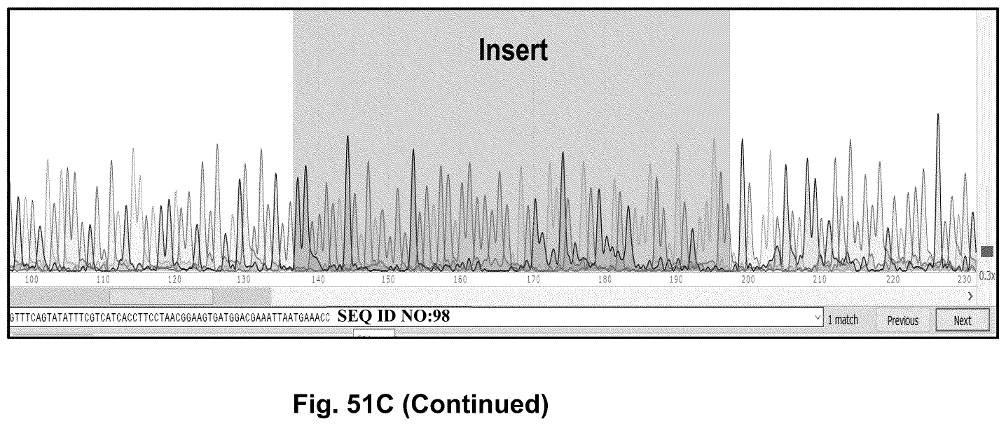

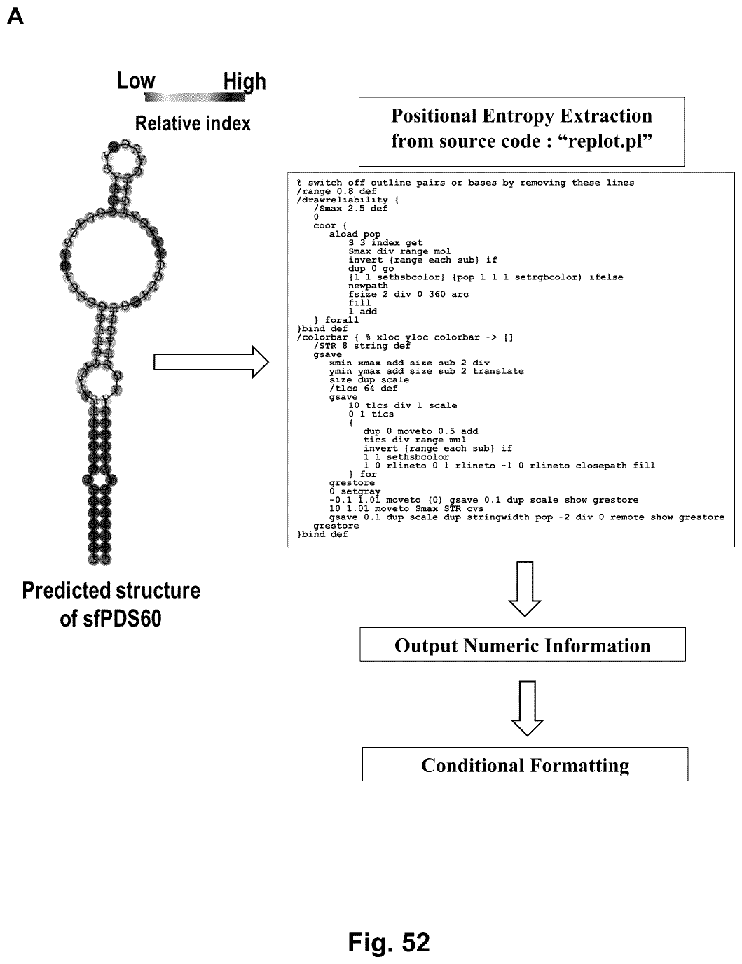

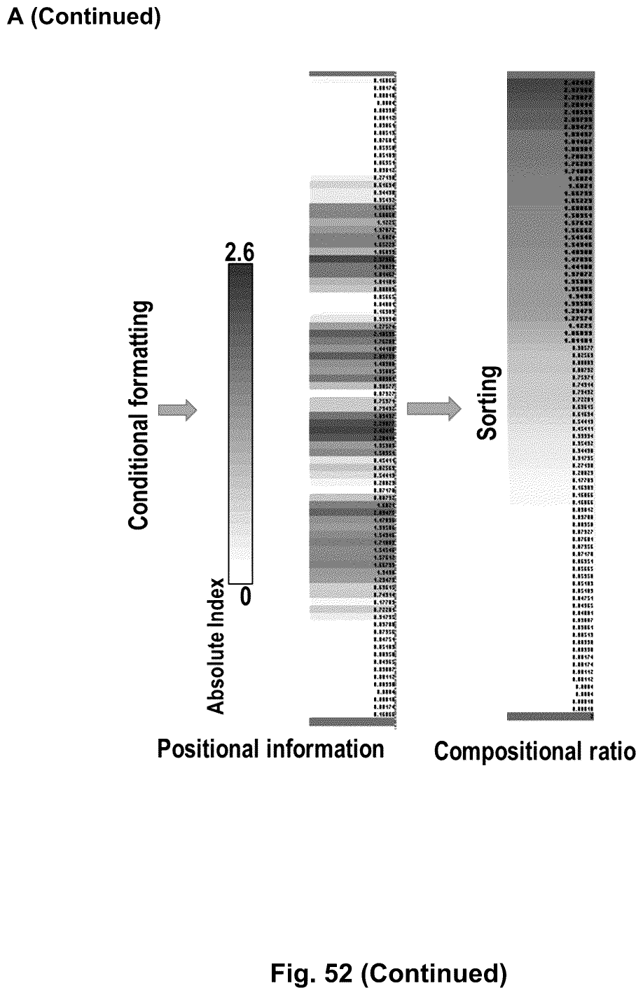

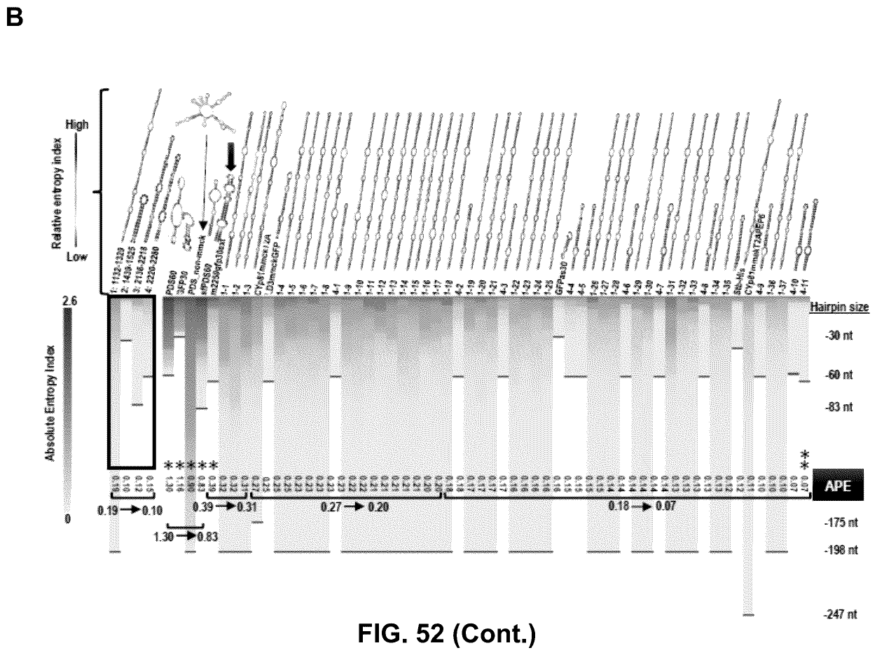

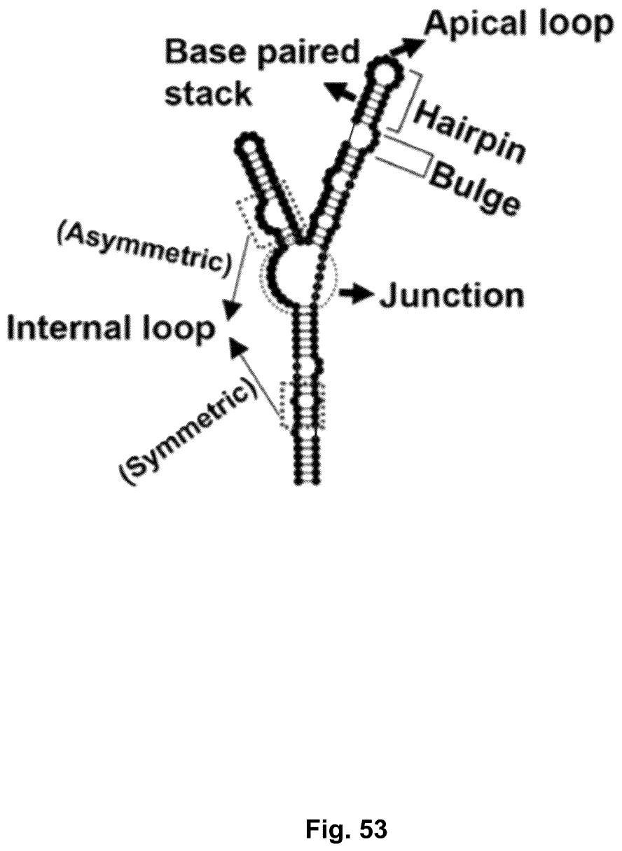

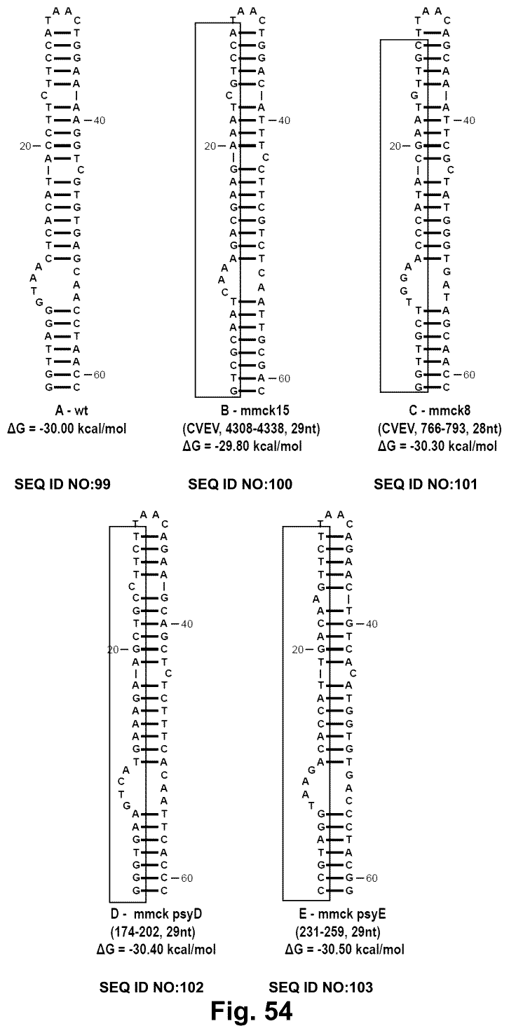

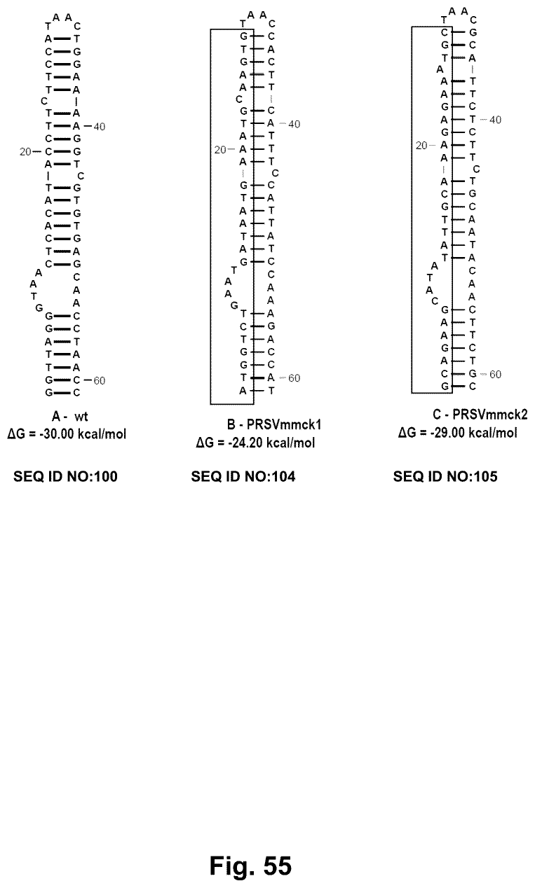

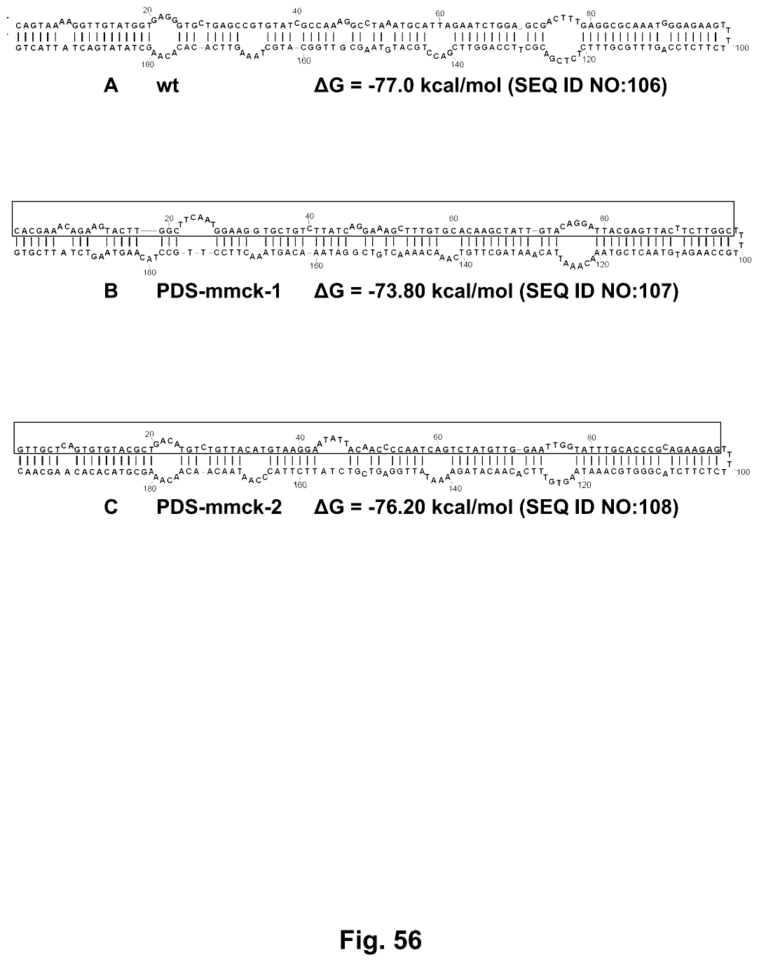

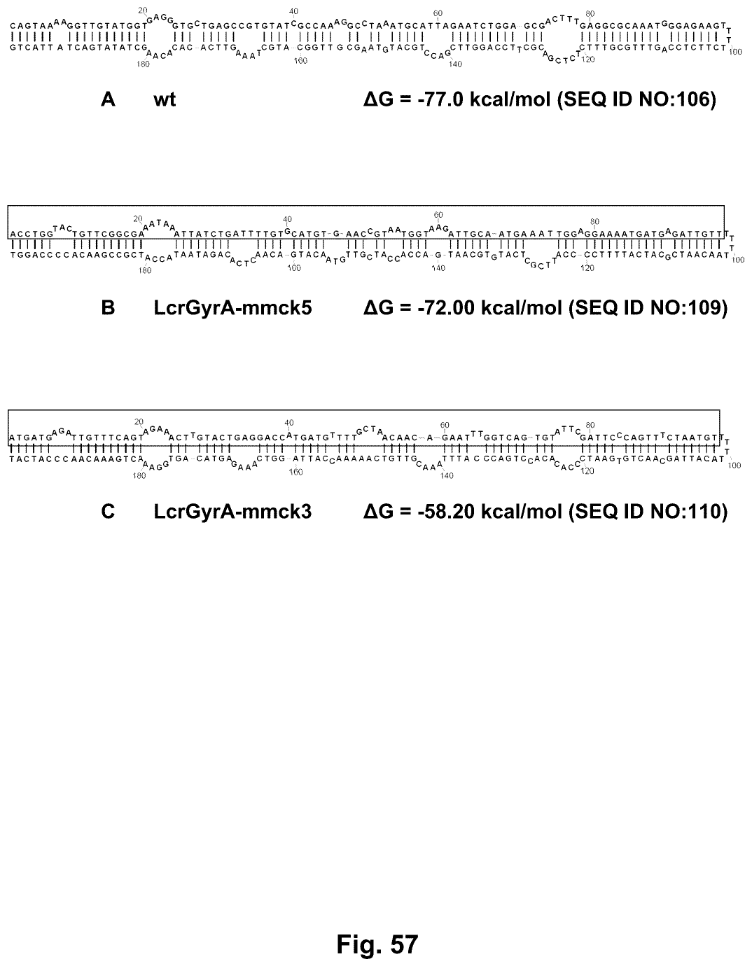

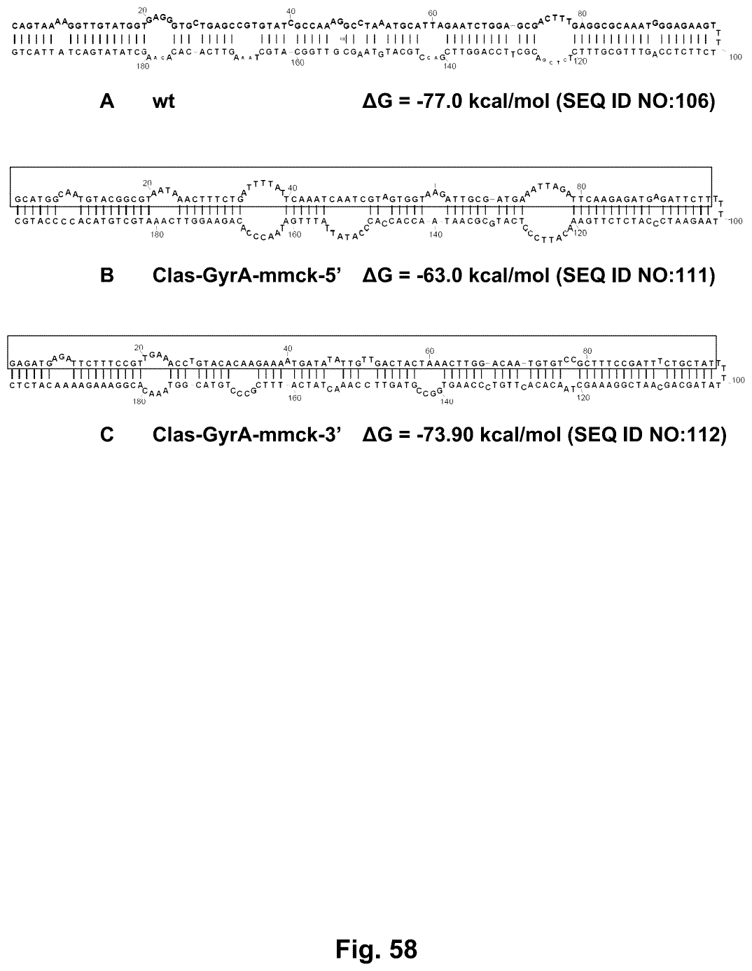

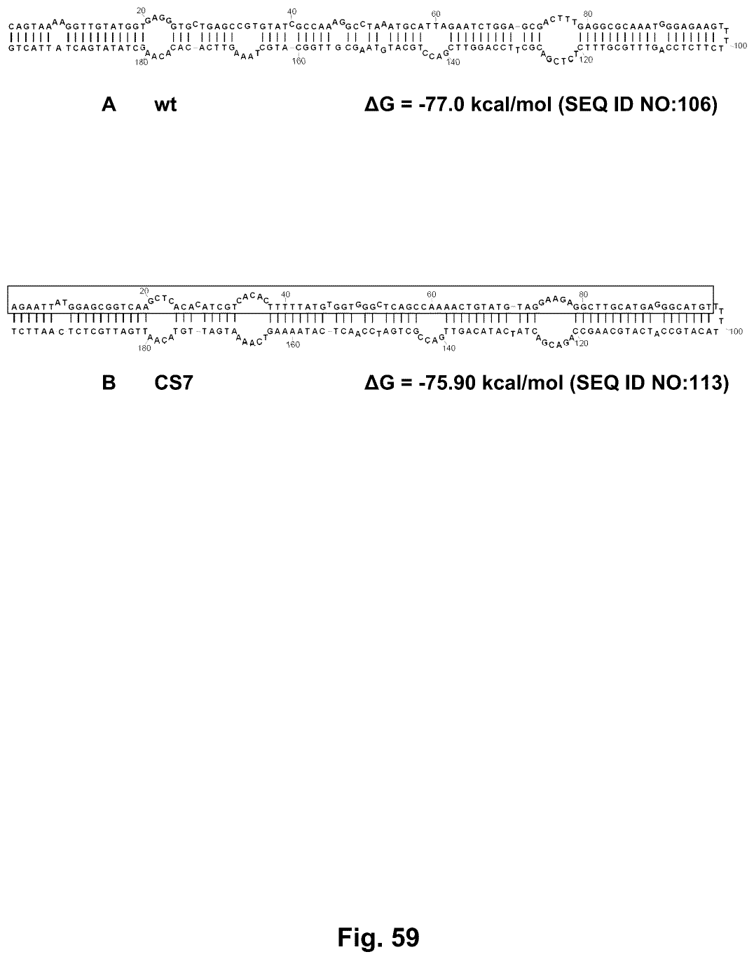

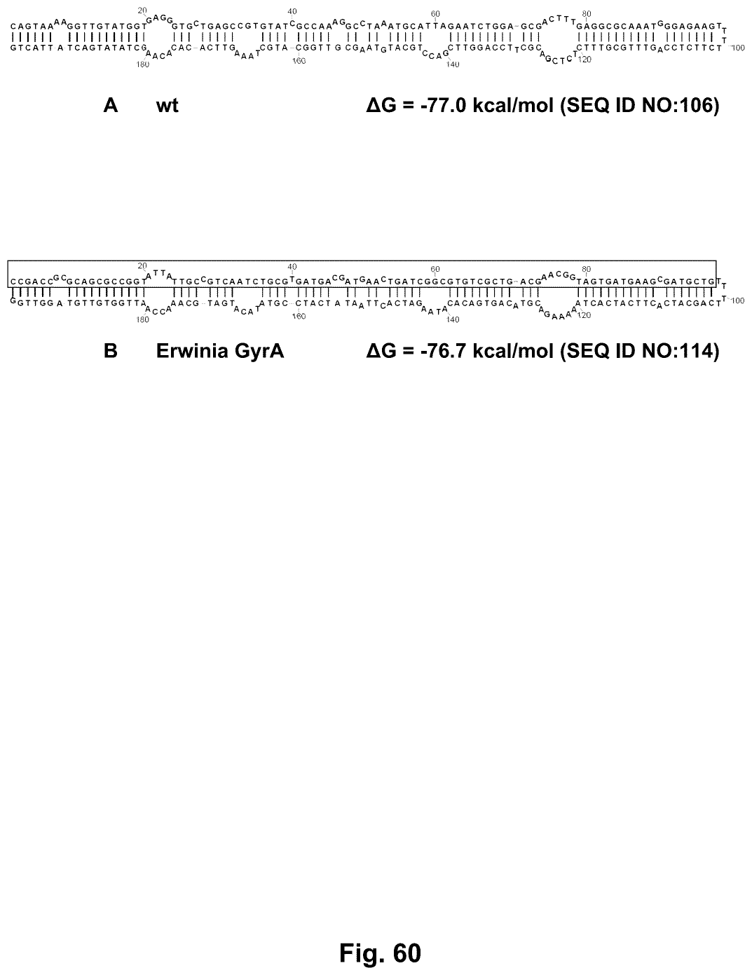

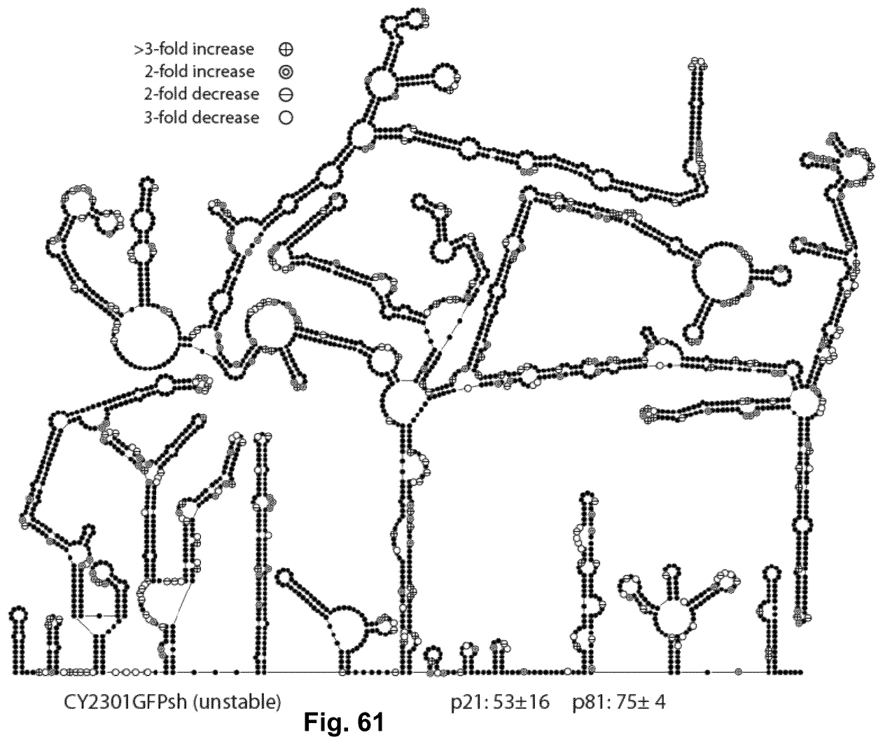

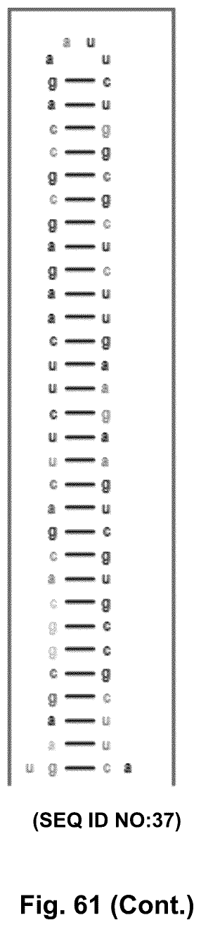

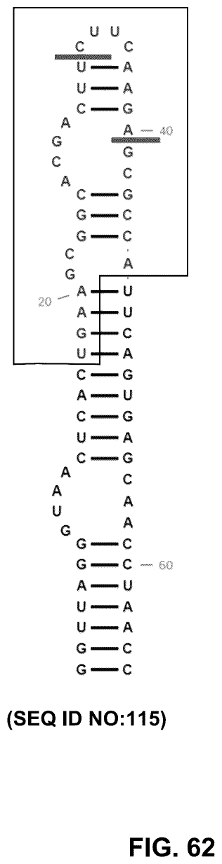

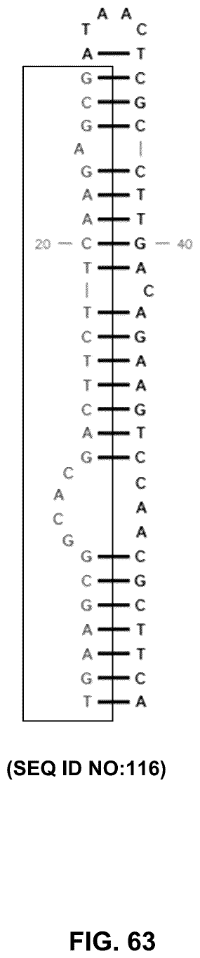

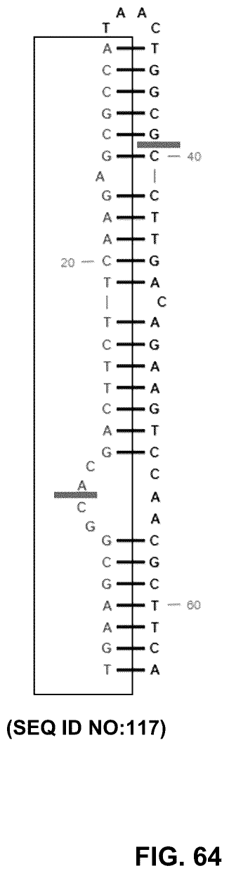

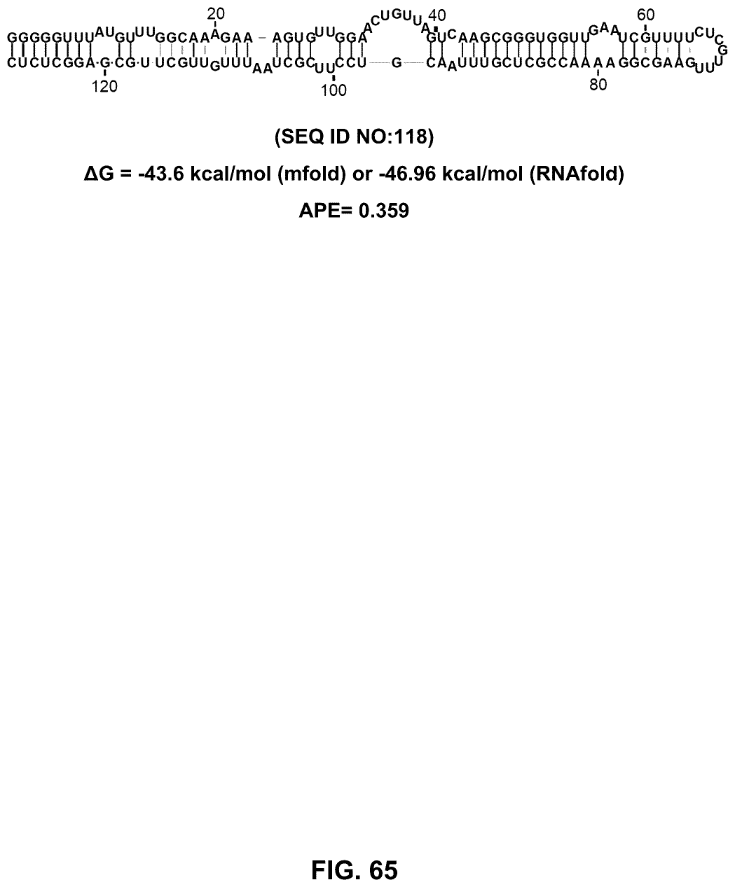

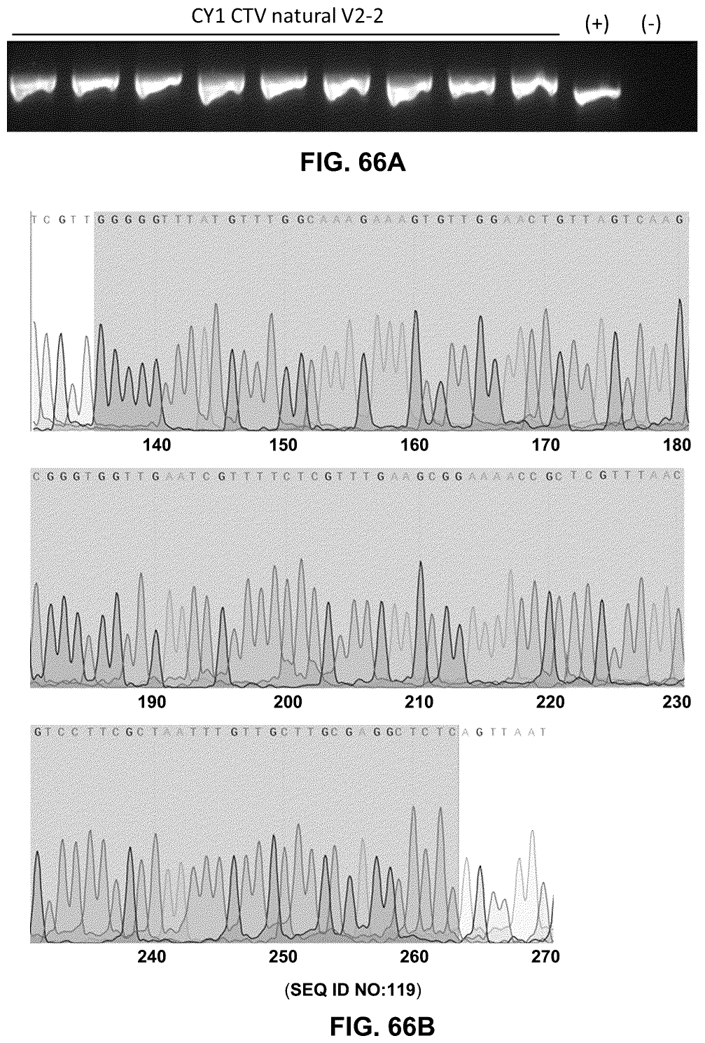

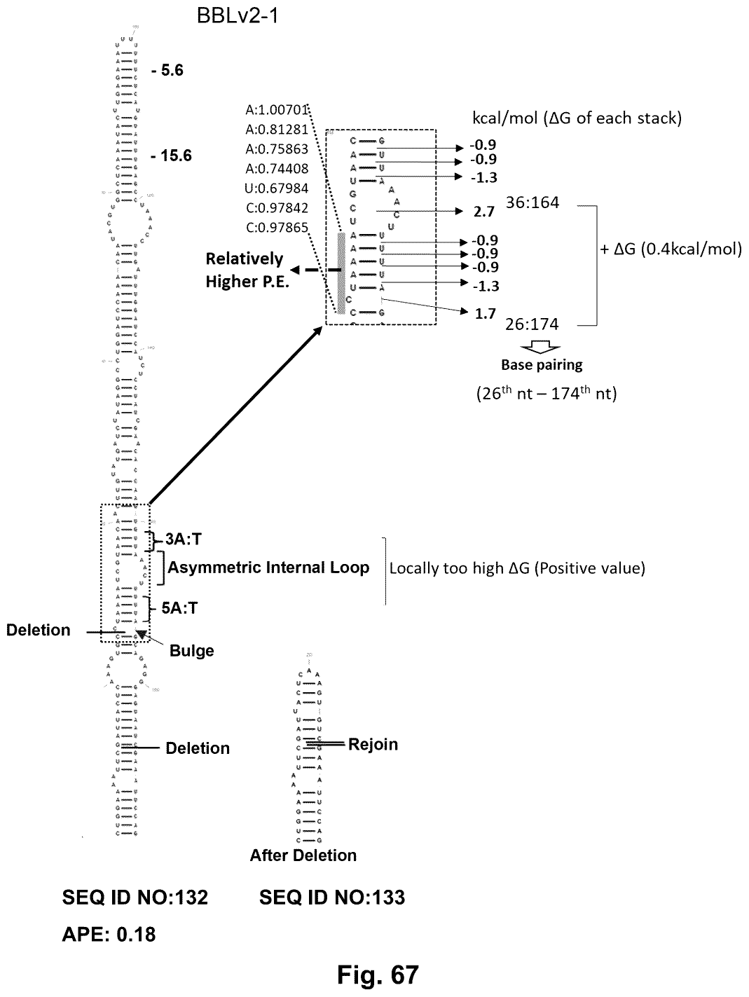

Mimicked hairpins were inserted in one of three different locations, either singly or with a second mimic hairpin. All tested structures were extremely stable, including constructs including two inserts (see FIG. 43). A wide variety of mimicked hairpins have been tested in a variety of locations, including constructs having mimic hairpins based on natural hairpin structures of CYVaV. In one implementation, relatively large hairpins of CYVaV were used (e.g., having a length of about 200 nt or more) in order to stack or incorporate multiple siRNAs into a single insert.

Referring to FIG. 43, the two inserts mimic Structure 4 of CYVaV (FIG. 49 Panel C). The ΔG of Structure 4 is −30.00 kcal/mol. The APE of Structure 4 is 0.15. The ΔG of insert CS7-V2.2 is −32.50 kcal/mol and the APE is 0.26. The ΔG of insert Mmck6.1 is −28.40 and the APE is 0.15. The sequence of Mmck6.1 is (SEQ ID NO:75):

ggaagugauggacgaaauuaaugauuccauaacuggaacauuacauuucg
ucaacacuucc

The first 24 nt of Mmck6.1, shown in bold above, are an siRNA sequence targeted at CTV. Insert CS7-V2.2 is a targeted against the callose synthase gene of the host plant. The sequence of CS7-V2.2 is (SEQ ID NO:76):

gguaucaaugggcagacgaagauuuggcauaacugccaaucuuccgucug
caaaugauacc

The first 27 nt of CS7-V2.2, shown in bold above, are an siRNA sequence targeted at the callose synthase gene of N. benthamiana.

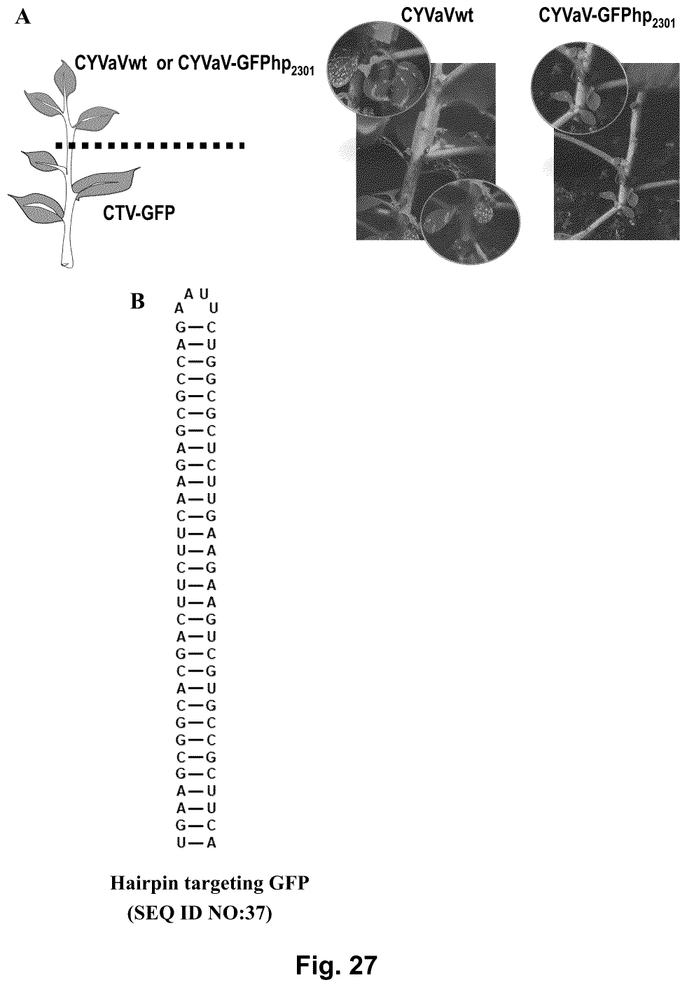

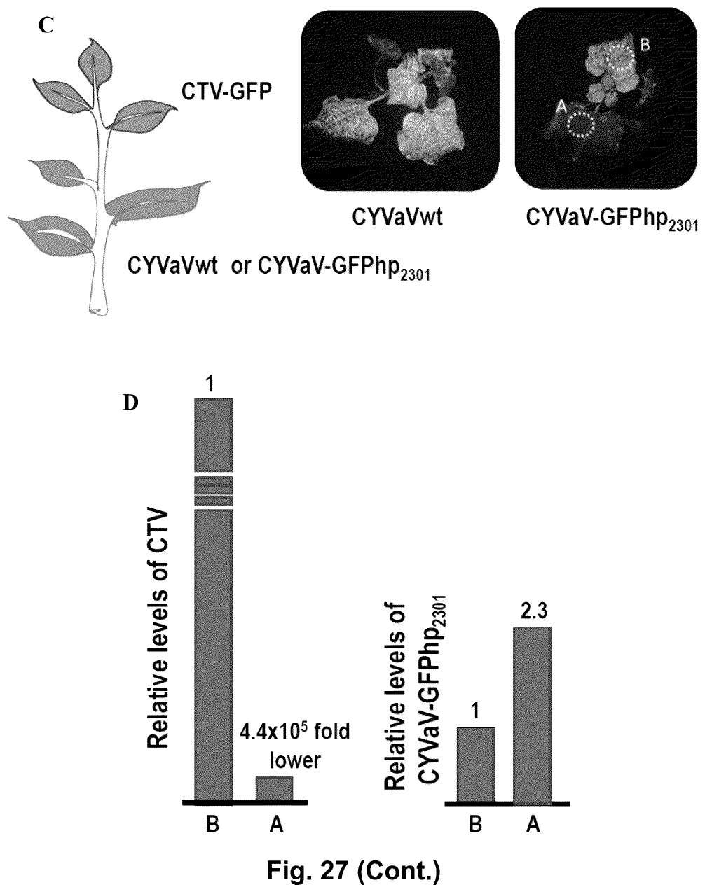

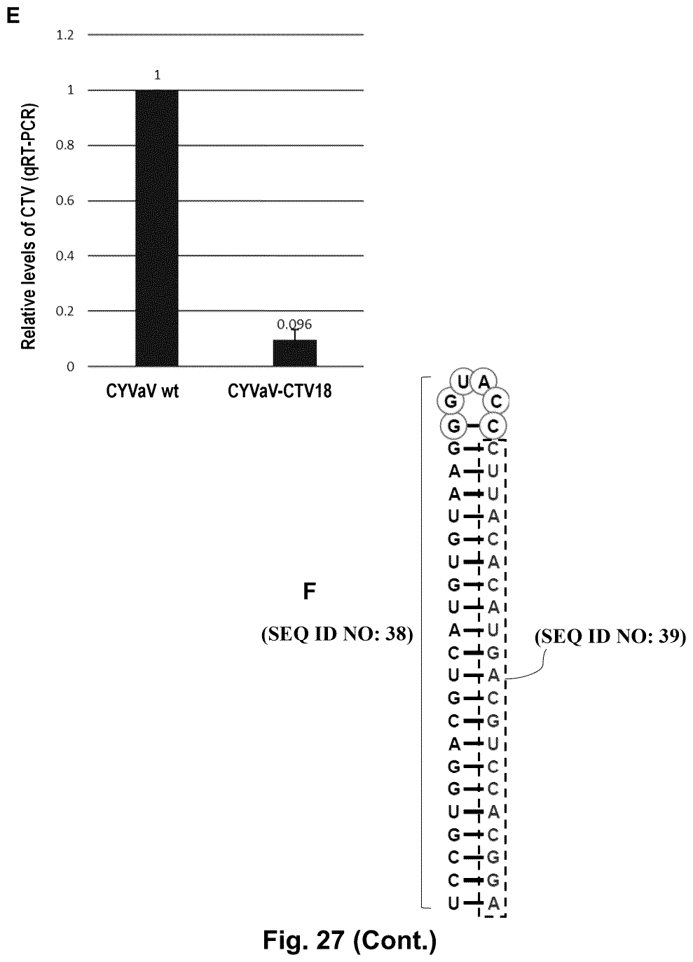

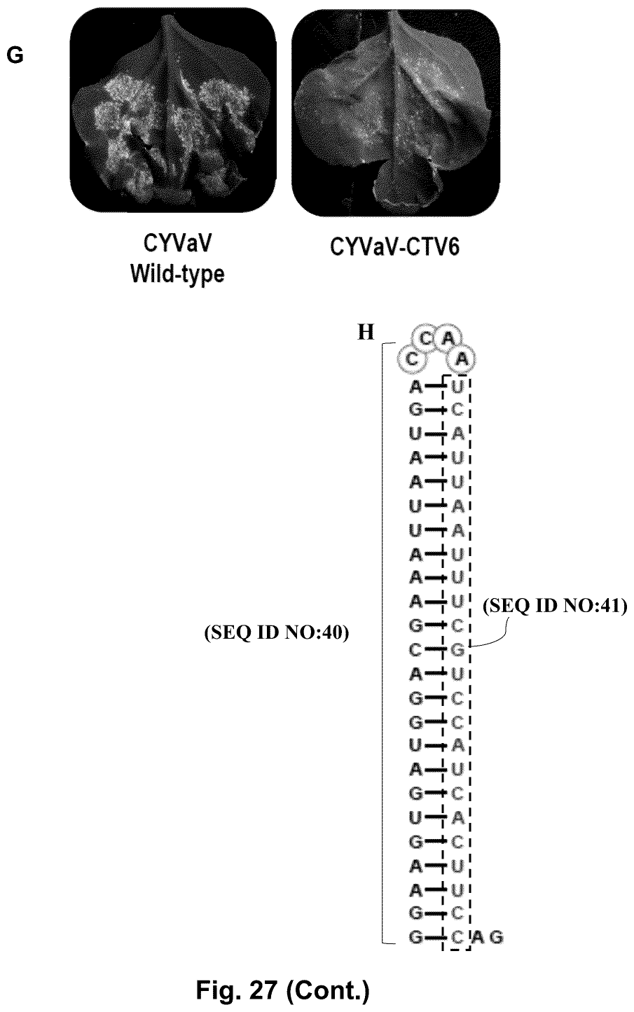

The (relatively) unstable insert shown in Panel D is CY2301GFP30sh, a conventional (i.e. fully base-paired) hairpin which is also shown in FIG. 42 Panel C and in FIG. 27 Panel B and further described by SEQ ID NO:37. Insertion of CY2301GFP30sh was shown to produce SHAPE changes and to effect stability of the RNA structure throughout the CYVaV genome (FIG. 61). CY2301GFP30sh was inserted at location 2301 of CYVaV. After 3-4 weeks of infection, a PCR assay identified a band corresponding to CYVaV without the CY2301GFP30sh insert. The batch sequencing of CY2301GFP30sh show in FIG. 43 Panel D shows heterogeneity, further indicating the presence of replicates without the insert. The APE, and positional entropy of individual bases, of CY2301GFP30sh is very low. However, whereas low positional entropy provides stability for inserts with hairpin-like structure, this factor does not predict stability of a conventional (i.e. fully base-paired) insert. The instability of conventional hairpins appears to be related to other considerations.

As demonstrated by the disclosed data, the mimic hairpin inserts are extremely stable within the vector (e.g., a CYVaV-based vector) and thus are particularly well suited for use as a vector, for example a VIGS vector, for targeting pathogens and endogenous gene expression to control diseases in long lived plants. As known in the art, VIGS is a post-transcriptional gene silencing (PTGS)-based technique that exploits the natural defense mechanisms employed by plants to protect against a viral pathogen. See, e.g., Pantaleo et al. (2007) Molecular Bases of Viral RNA Targeting by Viral Small Interfering RNA-Programmed RISC, J Virol 81(8):3797-3806; Ramegowda et al. (2014) Virus-induced gene silencing is a versatile tool for unraveling the functional relevance of multiple abiotic-stress-responsive genes in crop plants, Plant Genetics and Genomics, Vol. 5, Art. 323; Mei et al. (2016) A Foxtail mosaic virus Vector for Virus-Induced Silencing in Maize, Plant Physiol 171:760-772).

Conventional VIGS vectors have not been suitable for long-lived trees. Viruses have relatively limited host ranges, requiring the need to develop a different virus vector for each tree or vine (and sometimes requiring more than one virus vector for the same crop). As such, the cost for the development and approval of a virus that infects a single or limited crop type may be prohibitive. In addition, any virus utilized as a vector should be relatively mild or asymptomatic, thus eliminating numerous viruses as suitable vectors. In addition, sequences inserted into conventional viral vectors are generally unstable, with the insert typically remaining intact only for several days or weeks. However, stability is needed for many years for some hosts such as tree and vine crops.

CYVaV and CYVaV-like molecules are particularly well suited for use as a VIGS vector, or vector having an siRNA or other insert, particularly for use in treating tree and vine pathogens requiring long-term stability. CYVaV exhibits an exceptionally wide host range due to its lack of endogenous movement proteins and use of host movement proteins and little or no detrimental symptoms to the host, may be engineered to include extremely stable inserted sequences, and rarely (if ever) unintentionally spread from plant to plant. In addition, CYVaV does not encode any RNA silencing suppressor.

Reverse Fitness Engineering: In accordance with the present disclosure, a stable parental structure of an RNA vector (for example an RNA virus) is optionally modified in combination with adding a heterologous element. In some embodiments, the modification may include a structurally stabilizing modification and/or a structurally de-stabilizing modification (e.g., converting G:U pairs to G:C pairs in the parental structure). In some examples, the modification may include truncating a hairpin of the parental structure. In some examples, the modification may include inserting a scaffold into the parental structure. One or more of these examples may be combined. Without intending to be limited by theory, these modifications produce a structure that is more fit for one or more process in the infection cycle when a heterologous element is added then when the heterologous element is deleted. The RNA vector with intact heterologous element thereby replicates in greater numbers than any copies wherein the heterologous element is deleted. While described herein in relation to iRNA-based vectors used to treat plants, it is expected that these techniques may be applied to other RNA vector and used to treat plants or other organisms such as animals.

In some implementations, methods of engineering mimic hairpin structures in the vector are used in combination with methods of engineering “reverse fitness” into the vector in order to minimize or prevent the possibility that should the mimic hairpin(s) or portions thereof be lost, the remaining vector will thrive or dominate in the population. Reverse fitness engineering techniques provide an optional characteristic, given even inserts stabilized using the disclosed hairpin mimic process could still potentially be inadvertently lost in rare events and/or over an extended period of time. Such loss of insert(s) could generate a vector similar or substantially identical to the wild-type vector, which has evolved for millions of years to be a very fit molecule. Thus, in the absence of reverse fitness, the engineered structure, if it were to lose an insert, could otherwise dominate in the population over time. The engineering techniques disclosed herein substantially minimize or eliminate such adverse though rare possibility. Since the mimic inserts are extremely stable, adding a reverse fitness modification is not required to produce a useful vector but optionally may be added. Similar reverse fitness modifications may also optionally be used with other types of inserts or heterologous segments.

Given the low fidelity and high error rate of RNA virus replication, viruses evolve very rapidly. The most fit virus will eventually predominate within a population infecting a particular host under a specific set of environmental conditions. Over many years of research, a substantial number of alterations in different viruses and subviral RNAs have been generated. In all cases except one, the altered virus was less fit than the WT virus in direct competition assays. (In one exception involving a single base change, the mutant was equally fit as compared to the WT). The data indicates that, even though the disclosed mimic hairpin inserts in the vector constructs are extremely stable, chance recombination events may occur over time that could potentially eliminate the insert(s) and result in a more parental-looking vector with increased fitness. The structure resulting from such chance events could replace the therapeutic vector including the mimic inserts as the dominant species accumulating in the host (e.g., plants or trees). Given that some hosts such as trees may potentially live for hundreds of years with countless viral amplification events producing progeny, in some cases it may be useful to provide an exceptional level of stability for viral vectors to remain the dominant variant. In order to provide such stability, vectors including the mimic inserts were engineered to be more fit than the vector if the insert or a portion thereof is lost. In order to achieve such fitness level, hairpin mimics were inserted into a vector that contains additional alterations (i.e., in addition to the mimic hairpins) that increase fitness in the presence of the mimic inserts relative to fitness in the absence of the mimic inserts.

We discovered the specifics of how to engineer reverse fitness when we solved the active, intermediate, and inactive structures for the WT CYVaV −1PRF recoding site (the site where −1 programmed ribosomal frameshifting takes place leading to synthesis of the polymerase), and found that these structures differed slightly between CYVaV and all other Class 2 umbravirus-like (ula)RNAs. A primary difference between CYVaV and other Class 2 ulaRNAs is the presence of two natural inserts in other ulaRNA (FIG. 44 and FIG. 9C). In addition, a series of similar base alterations in specific regions of the genome that are associated with frameshifting accompanies the presence of the inserts (FIG. 45). These sequence differences affect the stability of the active and inactive frameshifting structures (see FIG. 45, showing the active and inactive structures for WT CYVaV and Class 2 ulaRNA CYVaV-Delta).

It was hypothesized that when WT CYVaV lost the inserts found in other Class 2 ulaRNAs, changes were needed to rebalance the stability of the active and inactive recoding structures so that the proper amount of polymerase is made, which is critical for virus fitness (FIG. 45). Achieving the proper level of polymerase is important—too little polymerase could make the virus less fit as it could replicate less, while too much polymerase is toxic to the host. Inserting hairpins into WT CYVaV requires a similar rebalancing of the active and inactive recoding structures to achieve suitable polymerase expression since inserted hairpins are destabilizing to the genome of CYVaV. However, if the inserts are mimics, rebalancing is not required but, if used, should be only a slight rebalancing is necessary. The key is that the alterations that properly rebalance CYVaV+mimic inserts are detrimental to CYVaV without the inserts. In other words, if the WT CYVaV is altered in a manner to modify its polymerase expression, this will result in a sub-optimized and less fit version. If a hairpin mimic is then inserted into the modified CYVaV in a manner that “corrects” for the suboptimization that resulted from the modification, then the CYVaV+mimic inserts version (e.g., the construct to be commercialized) will be optimally stable, or at least more fit than the modified vector without the insert. If the mimic inserts are ejected due to recombination events at some point in the future, the vector could revert back to the modified CYVaV. However, since such a modified CYVaV is designed to be less fit without the insert(s), it would be an evolutionary “dead-end” while the remaining modified CYVaV+mimic inserts in the host will continue to be the dominant version. Thus, in accordance with reverse fitness engineering techniques disclosed herein, the modified vector+mimic insert(s) is more stable than the modified vector without such insert(s).

Referring to Table 1 below, alterations were made in WT CYVaV (no inserts). i2631A and U664C stabilize the inactive structure. Stabilizing the active structure (to rebalance the two structures) then requires A652G to reform a required pseudoknot with U664C, and A604U. Other mutations that destabilize the inactive structure and compensate for i2631A or U664C may also be used.

TABLE 1
In vitro translation in wheat germ extracts of CYVaV harboring stabilizing and
destabilizing alterations

| | | | | | Infiltrated Plants | |
|---|---|---|---|---|---|---|
| | p21 | | p81 | | | Positive |
| Construct | Ave | Stnd | Ave | Stnd | Stability | Plants |
| CYVaV | 100% | 0% | 100% | 0% | Yes | 100% |
| CYVaV i2631A | 136% | 23% | 81% | 41% | Yes | 43% |
| CYVaV A604U | 152% | 3% | 75% | 41% | Yes | 50% |
| CYVaV U664C | 135% | 14% | 60% | 35% | | |
| CYVaV A652G | 82% | 18% | 26% | 19% | | |
| CYVaV i2631A + A604U | 153% | 24% | 78% | 42% | | |
| CYVaV A604U + U664C | 157% | 26% | 43% | 16% | | |
| CYVaV i2631A + U664C | 190% | 40% | 49% | 22% | N/A | 0% |
| CYVaV U664C + A652G | 105% | 27% | 80% | 60% | | |
| CYVaV i2631A + U664C + A652G | 152% | 89% | 50% | 27% | | |
| CYVaV i2631A + A604U + U664C | 180% | 5% | 34% | 25% | N/A | 0% |
| CYVaV i2631A + A604U + U664C + A652G | 130% | 19% | 99% | 45% | | |

Results for experiments comparing the effect of mutations on WT CYVaV and CYVaV containing a partially stable, non-mimic hairpin insert are shown in Table 2 below. Note that for all of these combinations, the vector+non-mimic insert generates more p81 polymerase. The bottom two combinations are very detrimental for translation of only WT CYVaV.

TABLE 2
In vitro translation in wheat germ extracts of CYVaV and CYVaV with a non-mimic
insert (partially stable) harboring stabilizing and destabilizing alterations.

| | | | | | CYVaV + Insert | | | |
|---|---|---|---|---|---|---|---|---|
| | | | | | (Non-Mimetic) | | | |
| | CYVaV WT | | | | Partially Stable | | | |
| | p21 | | p81 | | p21 | | p81 | |
| Mutations Added | Ave | Stnd | Ave | Stnd | Ave | Stnd | Ave | Stnd |
| CYVaV WT | 100% | 0% | 100% | 0% | 100% | 0% | 100% | 0% |
| Partially Stable (non-mimetic) | NA | NA | NA | NA | 91% | 29% | 74% | 41% |
| Insert | | | | | | | | |
| A604U | 152% | 3% | 75% | 41% | 111% | 44% | 92% | 23% |
| U664C + A652G + i2631A | 152% | 89% | 50% | 27% | | | | |
| U664C + A652G + i2631A + | 130% | 19% | 99% | 45% | | | | |
| A604U | | | | | | | | |
| U664C + i2631A + A604U | 180% | 5% | 34% | 25% | 142% | 68% | 89% | 13% |
| C2589A + i2631A | 69% | 37% | 70% | 18% | 197% | 26% | 88% | 1% |
| G2583A + C2589G + i2631A | 63% | 25% | 58% | 21% | 126% | 34% | 85% | 27% |

Based on these data, and our understanding of the effect of the mutations on the balance between inactive and active recoding structures and the likely contribution of the mimic hairpins to this balance (destabilizing the inactive structure), the disclosed combination of mutations result in more stable vector+mimic inserts as compared to the vector containing the mutations but lacking the mimic inserts or portions thereof. These alterations may also be added to the disclosed vector+two mimic hairpins.

Additional characteristics and features of the present disclosure will be further understood through reference to the following additional examples and discussion, which are provided by way of further illustration and are not intended to be limiting of the present disclosure.

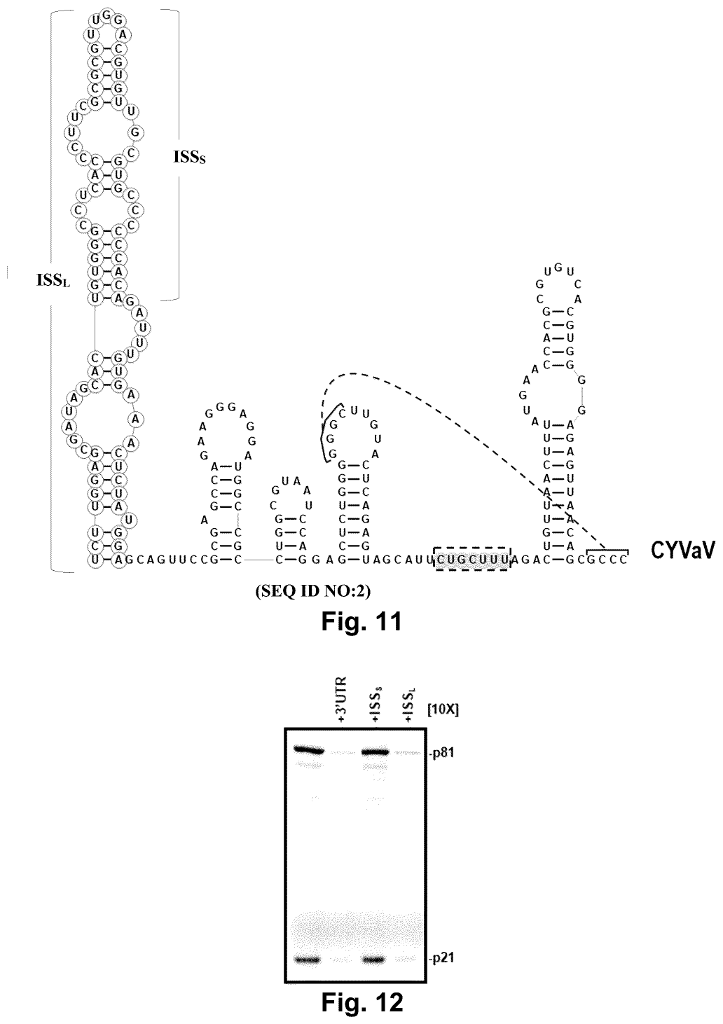

CYVaV Structure. Full length structure of CYVaV was determined by SHAPE structure probing and phylogenetic comparisons with the CYVaV relatives in Opuntia, Fig and Corn (FIG. 9A). The recoding site (see FIG. 10) and the ISS-like (I-shaped structure) 3′CITE (see FIG. 11) are identified, along with a region for accommodating an insert is, for example, shown by boxed double line region and discussed in further detail with regard to exemplary locations for inserts.

The genome organization of CYVaV exhibits some similarities to other RNA molecules, particular PEMV2 (FIG. 3, Panel A). However, umbravirus PEMV2 also possesses ORFs encoding for proteins p26 and p27 involved in movement. Levels of CYVaV plus (+) strands in infiltrated N. benthamiana leaves and systemic leaves are shown in FIG. 3, Panel B. Levels of the RNA-dependent RNA polymerase (RdRp) synthesized by frameshifting in vitro in wheat germ extracts of full-length CYVaV and PEMV2 are also shown (FIG. 3, Panel C). Note the significant difference in levels of p94 from PEMV2 as compared to p81 polymerase produced by CYVaV. The frameshifting site of CYVaV is one of the strongest known in virology and believed to be responsible for its exceptionally high accumulation.

CYVaV is encapsidated in virions of CVEV. CYVaV or CVEV or CYVaV+CVEV were agroinfiltrated into leaves of N. benthamiana. CYVaV was encapsidated in virions of CVEV, and virions were isolated one week later and the encapsidated RNAs subjected to PCR analysis (see FIGS. 5 and 6). Accumulation of CYVaV increased substantially in the presence of putative helper virus CVEV. rRNA loading controls are shown below. p14 silencing suppressor was co-infiltrated in all leaves. Yellowing symptoms were slightly more severe in citrus leaves with CYVaV+CVEV (FIG. 7, Panel B).

CYVaV is phloem-limited. Fluorescence in situ hybridization (FISH) imaging clearly detected plus strands of CYVaV, which was completely restricted to the sieve elements, companion cells and phloem parenchyma cells (FIG. 8).

CYVaV does not encode a silencing suppressor. N. benthamiana 16C plants were agroinfiltrated with a construct expressing GFP (which is silenced in these plants) and either constructs expressing CYVaV p21 or p81, or constructs expressing known silencing suppressors p19 (from TBSV) or p38 (from TCV) (FIG. 13, Panel A). Only p19 and p38 suppress the silencing of GFP, allowing the green fluorescence to be expressed (FIG. 13, Panel B). Northern blot probed with GFP oligonucleotide showed that GFP RNA is still silenced in the presence of p21 or p81 (FIG. 13, Panel C).

Replication of CYVaV in Arabidopsis protoplasts. An infectious clone of CYVaV was generated. Wild-type RNA transcripts (CYVaV) or transcripts containing a mutation in the recoding slippery site that eliminates the synthesis of the RdRp (CYVaV-fsm), and thus does not replicate, were inoculated onto Arabidopsis protoplasts. RNA was extracted and a Northern blot performed 30 hours later. Note that inoculated transcripts of CYVaV-fsm were still present in the protoplasts at 30 hours (whereas in a traditional virus they would be undetectable after 4 hours).

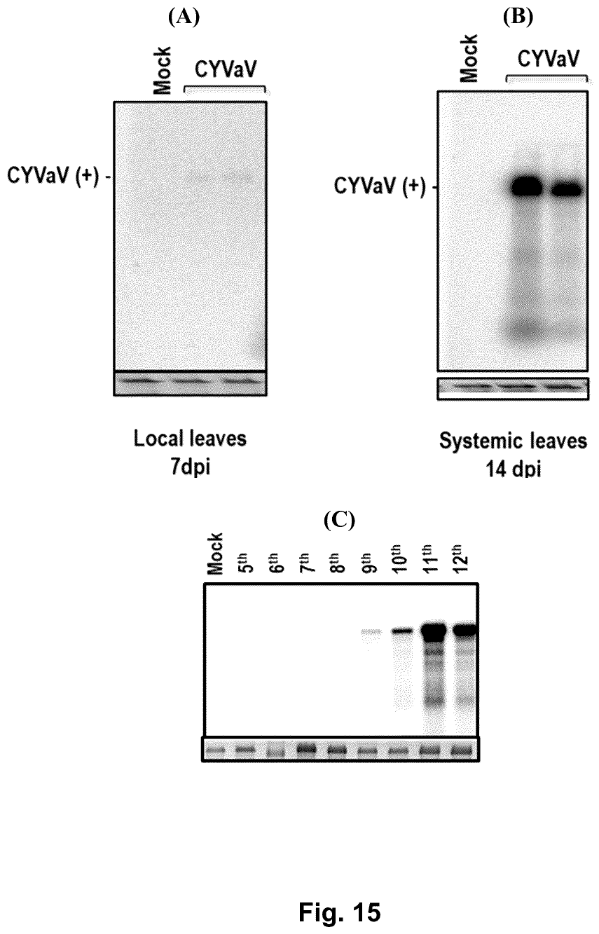

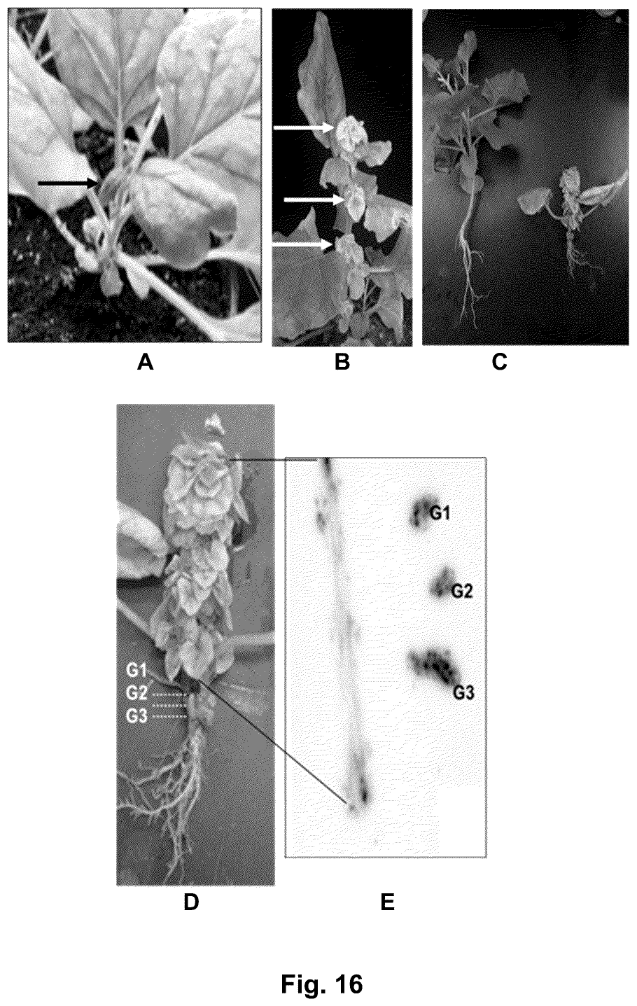

Replication of CYVaV in N. benthamiana. Level of CYVaV accumulating in the infiltrated leaves of N. benthamiana was determined by Northern blot (FIG. 15, Panel A). Plants infiltrated with CYVaV sporadically showed systemic symptoms (FIG. 15, Panel B; see also FIG. 16). These plants accumulated high levels of CYVaV. Level of CYVaV in individual leaves of a systemically infected plant was determined (FIG. 15, Panel C). Leaves 4 and 5 were agroinfiltrated with CYVaV. Note the substantial accumulation of CYVaV in the youngest leaves.

Symptoms of N. benthamiana systemically infected with CYVaV. Leaves 4 and 5 were agroinfiltrated with CYVaV. The first sign of a systemically infected plant is a “cupped” leaf (FIG. 16), which was nearly always leaf 9. In the following few weeks, leaf galls emerged at the apical meristem and each node of the plant. Systemically infected plants also had root galls containing a substantial amount of CYVaV as evidenced by Northern plant blot.

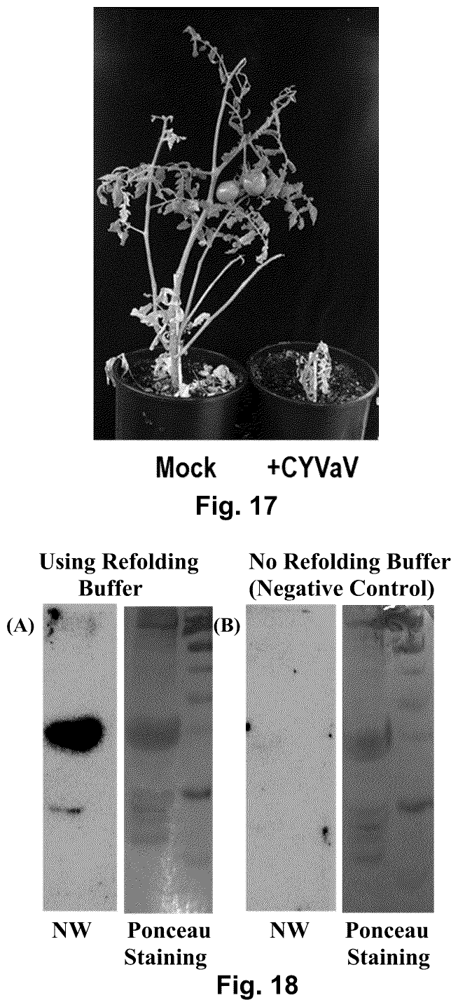

CYVaV demonstrates an exceptional host range. Sap from a systemically-infected N. benthamiana plant was injected into the petiole of tomato (FIG. 17). One of four plants showed very strong symptoms and was positive for CYVaV by PCR. Plant shown is at 53 days post-infection with a plant of the same age.

CYVaV binds to a highly abundant protein extracted from the phloem of cucumber. Labelled full-length CYVaV binds to a prominent protein as demonstrated in the Northwestern blot (FIG. 18). Proteins were renatured after SDS gel electrophoresis. This protein is believed to be a known, highly conserved RNA binding protein containing an RRM motif known to chaperone RNAs from companion cells into sieve elements in the phloem of cucumber. No binding was seen when the proteins remained denatured after electrophoresis.

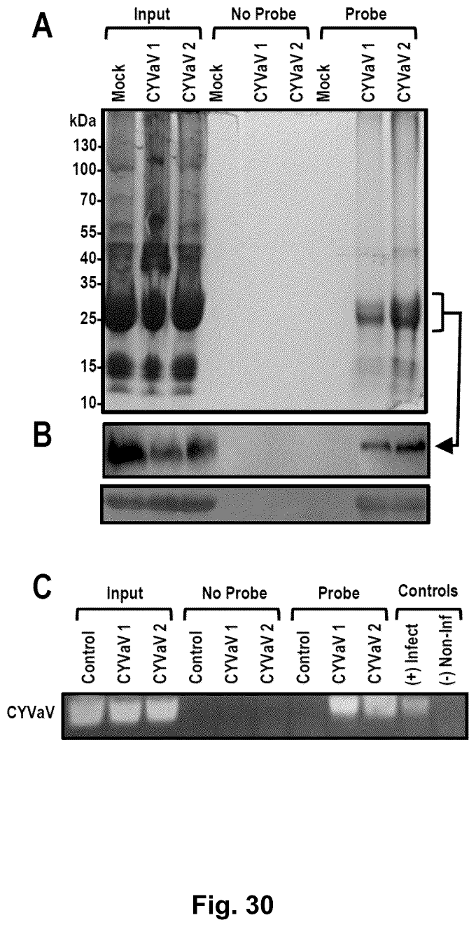

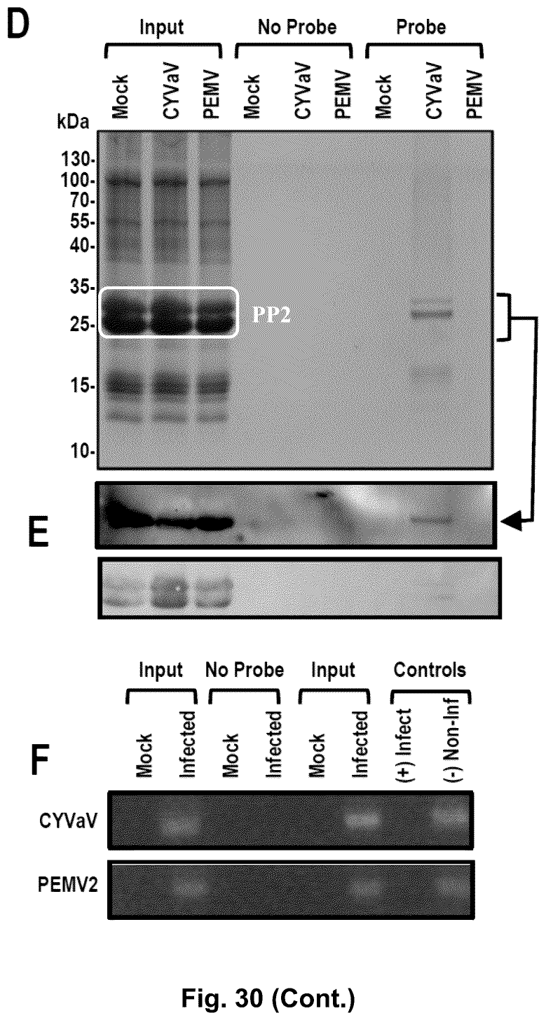

Referring to FIG. 30, CYVaV binds to phloem protein 2 (PP2). Panels A, B and C relate to experiments involving a mock (uninfected) cucumber plant and two cucumber plants infected with CYVaV. In Panel A, phloem exudates from the uninfected (mock) and two CYVaV-infected (CYVaV 1 and 2) plants were collected, crosslinked with formaldehyde (Input) and then used for pull down assays using streptavidin beads with and without attached 5′-biotinylated CYVaV probes (Probe and No Probe, respectively). SDS PAGE gel was stained with Coomassie Blue. As indicated in the three input lanes, an analysis of all proteins present in the sap includes significant amounts of protein with a molecular weight of about 25 kDa, which corresponds with the molecular weight of a common PP2. In the middle three lanes, essentially no proteins were found, indicating that PP2 does not bind to the streptavidin beads. In the right three lanes, a significant amounts of protein with a molecular weight of about 25 kDa was again found, indicating that PP2 was bound to CYVaV attached to the probe attached to the beads before being washed down from the beads. In Panel B, samples from the right three lanes of A were subjected to electrophoresis and then transferred to nitrocellulose membranes and analyzed by Western Blot using polyclonal antibody to cucumber PP2 (CsPP2) (upper panel). Panel B, lower panel, is the Ponceau S-stained membrane. In Panel C, total RNA recovered from the pull down assay before RNase treatment was subjected to RT-PCR to verify the presence of CYVaV. Additional controls were: (+), RNA from CYVaV-infected N. benthamiana; and (−), RNA from an uninfected cucumber plant. The assay indicates that CYVaV was bound to CsPP2 in the sap of the cucumber plant. FIG. 30, Panels D, E and F show a similar assay using N. benthamiana infected with CYVaV or PEMV2. For the PEMV2 pull down, PEMV2-specific probes were attached to beads. PEMV2 in this assay acts as a further control. The results indicate that CYVaV was bound to PP2 in the sap of the N. benthamiana plant by PEMV2 was not bound to PP2 in the sap of the N. benthamiana plant.

PP2 is believed to be involved with the movement or viroids but has not been reported to be involved in the coating or movement of any virus. Similarly, in the results described above, PP2 did not bind to PEMV2 in the sap of the plant. Without intending to be limited by theory, we believe that PP2 bound to CYVaV in the sap of a plant may also be responsible for the movement of CYVaV. While the early reports of CYVaV suggest that CYVaV does not move within a plant without a helper virus (CVEV) providing a movement protein, we have demonstrated that CYVaV moves systemically within a plant without a helper virus. However, a helper virus may still be required in nature for encapsidation to allow CYVaV to leave the phloem of a host plant and travel to another plant. In other experiments similar to the description above, CYVaV appears to bind to PP2 in the sap of tomato and melon plants. PP2 is found in essentially all plants and may allow iRNA-based vectors to move in, and systemically infect, a wide range of host plants.

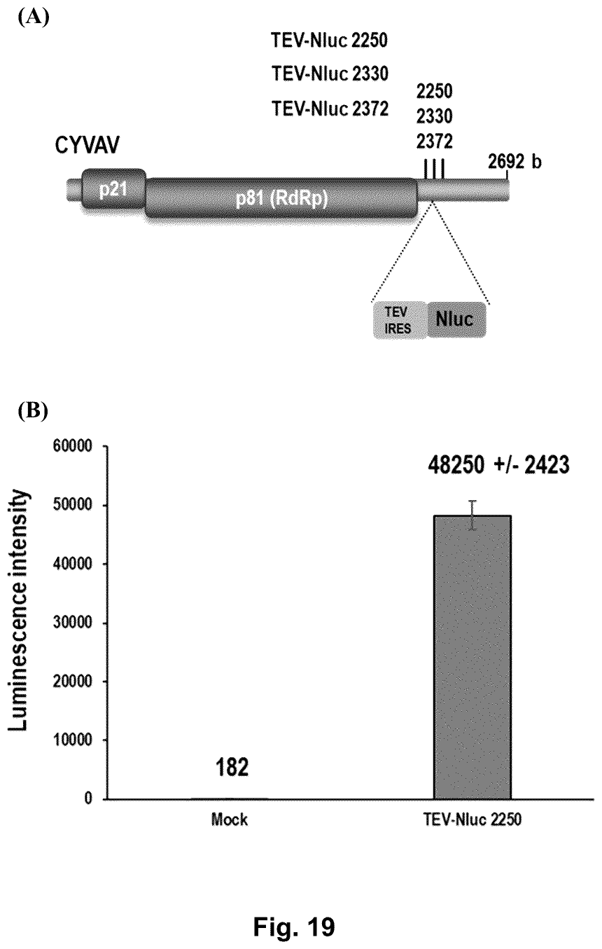

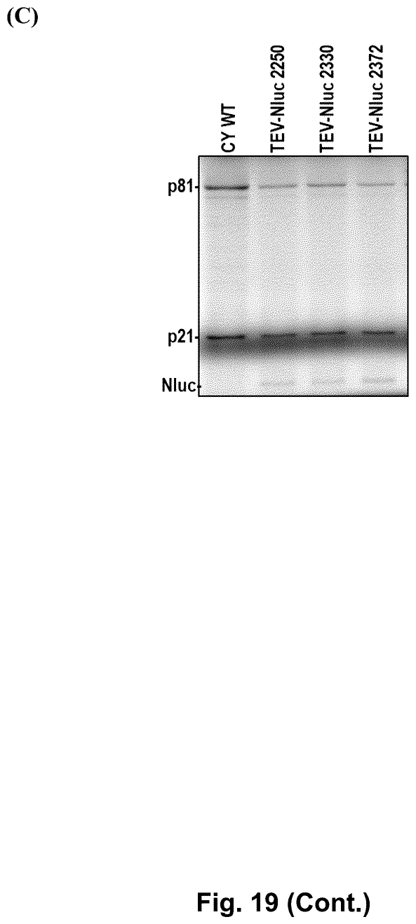

CYVaV can express an extra protein from its 3′UTR using a TEV IRES. Location of three separate inserts of nanoluciferase downstream of the Tobacco etch virus (TEV) internal ribosome entry site (IRES) were identified (FIG. 19). Invitro translation in wheat germ extracts of the three constructs was evaluated. Location of the nanoluciferase protein (Nluc) is near the bottom of the gel. Expression of nanoluciferase in protoplasts in vivo was investigated (FIG. 19, Panel C). Full-length RNA transcripts of the constructs shown in (A) were transformed into protoplasts. 18 hours later, total protein was extracted and nanoluciferase activity measured in a luminometer.

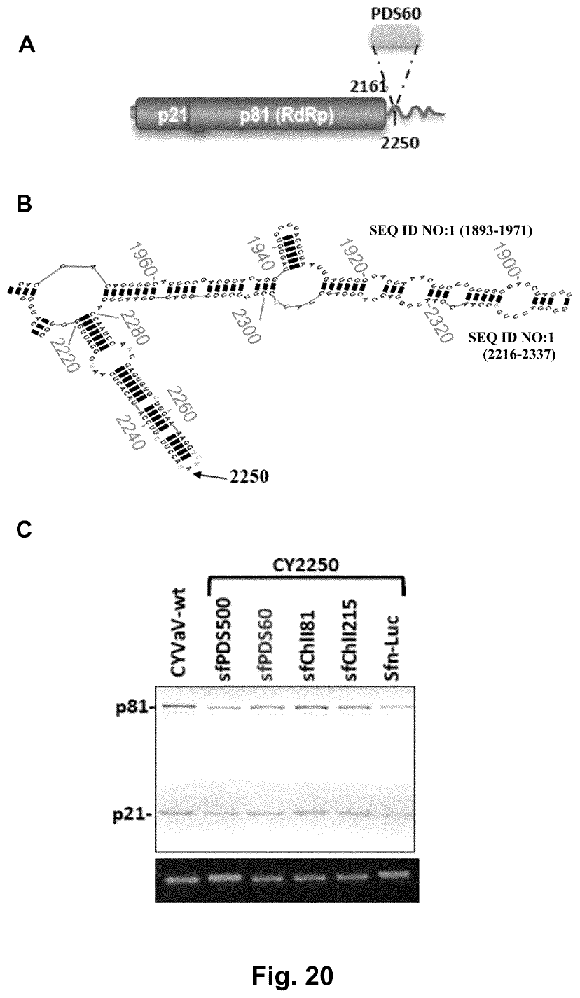

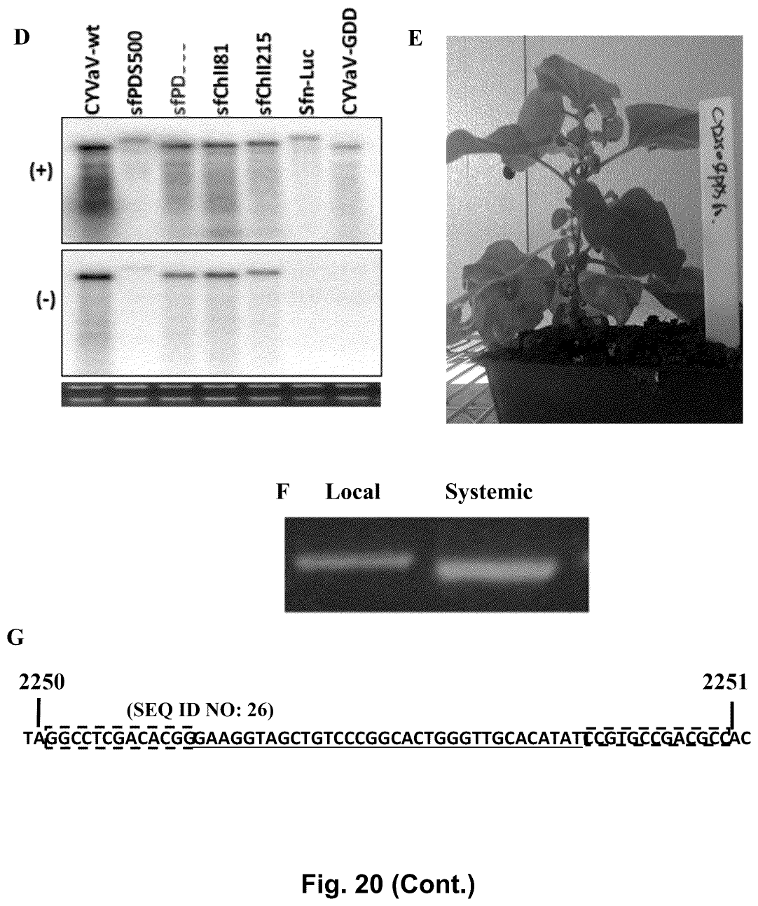

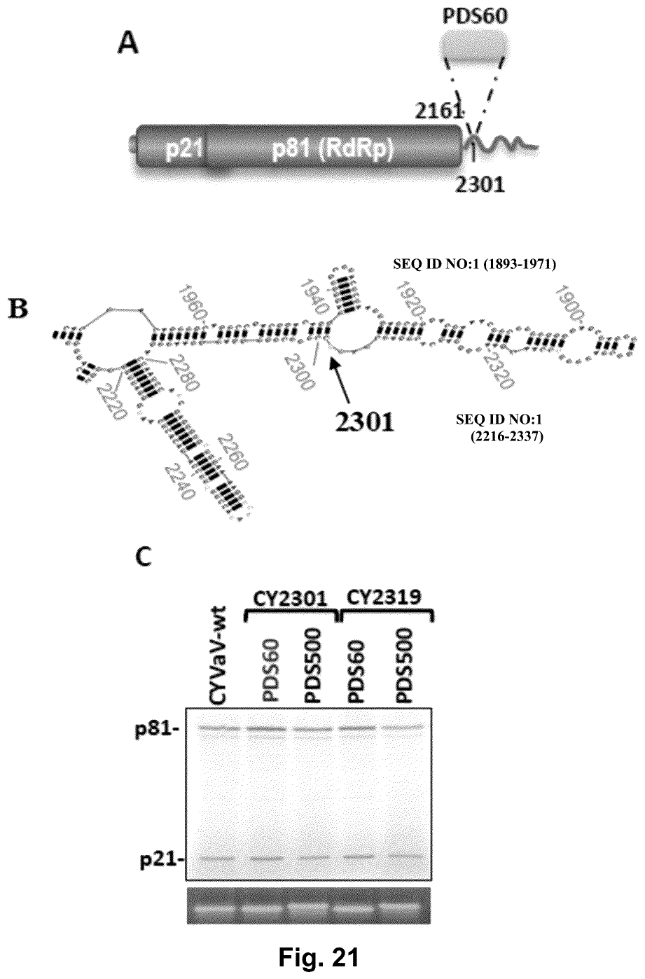

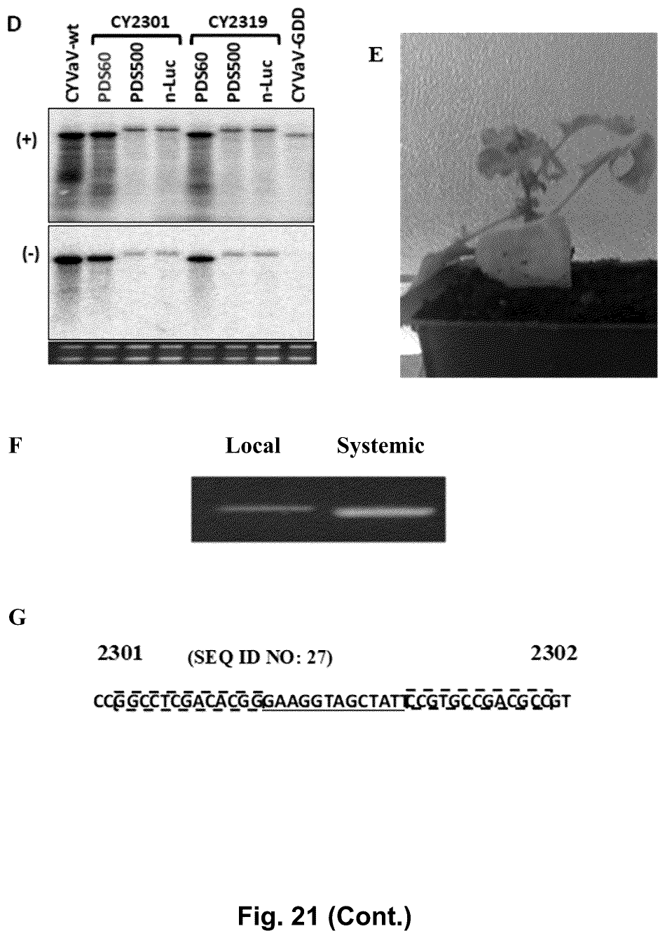

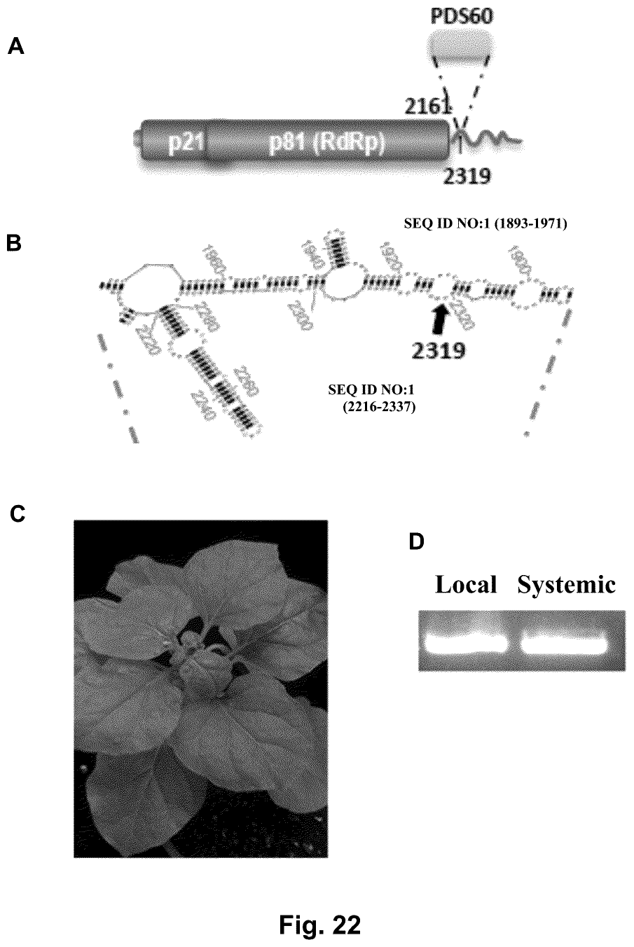

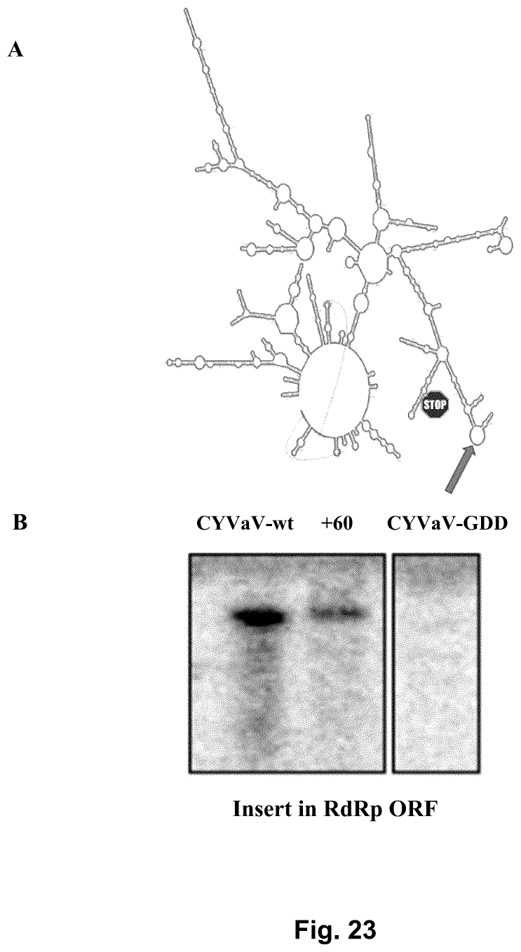

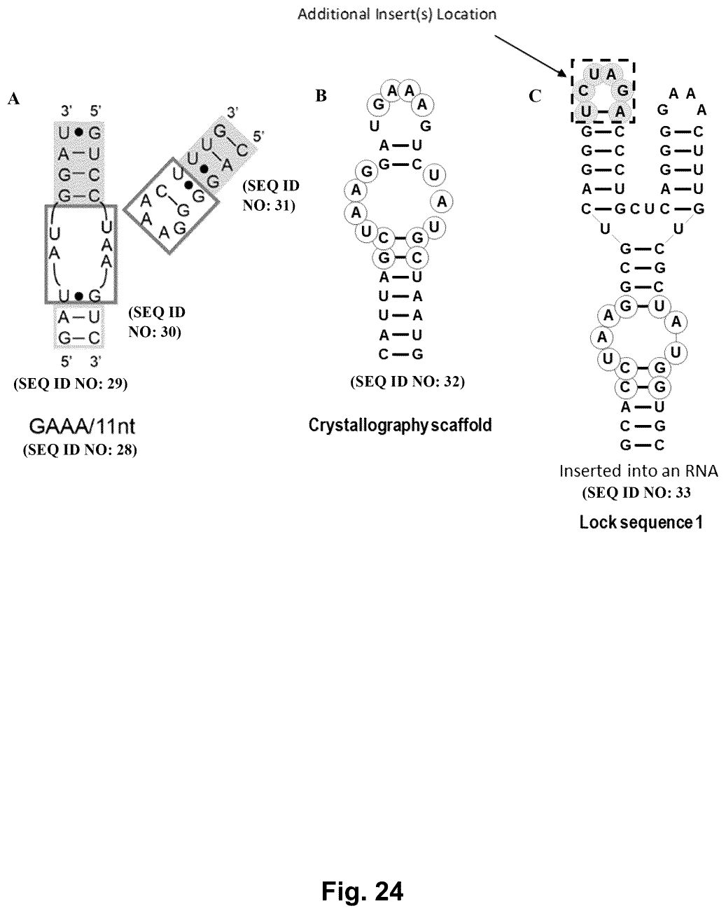

Exemplary locations for stable hairpin inserts at positions 2250, 2301 and 2319 were evaluated. The location for each of the inserts falls within an exemplary region noted above (see FIG. 9A). Wheat germ extract in-vitro translation assay of T7 transcripts from CYVaV-wt, and CYVaV VIGS vectors containing different amounts of sequence at position 2250 was conducted (FIG. 20). For example, construct sfPDS60 demonstrated excellent systemic movement in plants. Wheat germ extract in-vitro translation assay of T7 transcripts from CYVaV-wt, and CYVaV VIGS vectors containing different amounts of sequence at positions 2301 and 2319 was conducted (FIG. 21). Northern blot analysis of total RNA isolated from A. thaliana protoplasts infected by CYVaV wt and CYVaV VIGS vectors. CYVaV-GDD and negative control was conducted (FIG. 20, Panel D). Northern blot analysis of total RNA isolated from A. thaliana protoplasts infected by CYVaV wt and CYVaV VIGS vectors. CYVaV-GDD and negative control. was conducted (FIG. 21, Panel D). Constructs CY2250sfPDS60, CY2301PDS60, CY2301 sfPDS60, CY2319sfPDS60 (including inserts at positions 2250, 2301, 2319, respectively) all demonstrated excellent systemic movement with insertion. In addition, constructs CY2331PDS60 (including inserts at position 2331) also demonstrated the ability to move systemically throughout the host. A further construct, CY2083TAAPDS60, includes an insert at position 2083, which location is in the RdRp ORF (preceded by an inserted stop codon).

The sequences of the insertion regions (underlined below and as shown in FIG. 20, Panel G, and FIG. 21, Panel G) of the vector collected from systemic leaf is presented below:

(SEQ ID NO: 26)
taggcctcgacacgggaaggtagctgtcccggcactgggttgcacatat
tccgtgccgacgccac
(SEQ ID NO: 27)
ccggcctcgacacgggaaggtagctattccgtgccgacgccgt

A. iRNA-Based or ulaRNA-Based Vector Platform

In one embodiment, an iRNA-based or ulaRNA-based vector is provided for treating disease in the citrus industry caused by CLas bacteria (HLB). An isolate of CYVaV is utilized as a vector to target both the bacteria and the psyllid insects that deliver the bacteria into the trees. As discussed above, CYVaV is limited to the phloem where it replicates and accumulates to extremely high levels comparable to the best plant viruses. In addition, its relatively small size makes it exceptionally easy to genetically engineer. Thus, consideration of the structure and biology of CYVaV aided in the development of this novel infectious agent as a vector and model system for phloem transit.

The structure of the 3′UTR of CYVaV was determined based on SHAPE RNA structure mapping (FIG. 9A). In addition, a number of replication and translation elements were identified based on biochemical assays, as well as phylogenetic conservation (with umbraviruses) of their sequence and/or structure and position (FIG. 19, Panel A). An I-shaped element was also identified that serves as a cap-independent translation enhancer (3′CITE). A series of long-distance kissing-loop interactions (double arrows) were also identified, which are believed to be involved in stabilizing the RNA and accumulation in the absence of a silencing suppressor. Based on this structure, a number of areas were identified as suitable locations for sequence insertion, which should not disturb the surrounding structure.

Certain sites have been identified for potential inserts in the 3′ UTR and the RdRp ORF that can accommodate RNA hairpins, e.g., for generation of siRNAs that target feeding insects, sites that accommodate reporter ORFs and still allow for replication of an engineered CYVaV in agro-infiltrated N. benthamiana, and sites that trigger high level translation of reporter proteins in vitro. An engineered CYVaV incorporating the added ORF and siRNAs is introduced into a storage host tree, and then pieces thereof are usable for straight-forward introduction into field trees by grafting. Given the rarity of CYVaV (to date, it has only been identified in the four limequat trees by Weathers in the 1950s), there is little risk of superinfection exclusion.

Various insert locations were identified wherein replication or translation properties of the vector were not significantly reduced or eliminated. Insert locations adversely affecting such properties (likely due to disrupting the RNA structure or other important aspect of the CYVaV vector) were not pursued further. Four exemplary insert locations on the CYVaV-based vector were identified at positions 2250, 2301, 2319 and 2331. Alternatively or additionally, inserts may be located at positions 2330, 2336 and/or 2375. 50 nt hairpin inserts were successfully deployed in these locations with no disruption to translation in vitro or replication in protoplasts and CYVaV was able to move systemically in N. benthamiana.

Although CYVaV has no additional ORFs, both genomic (g)RNA and a subgenomic (sg)RNA of about 500 nt are detectable using probes to plus- and minus-strands. Investigation of the region that should contain an sgRNA promoter revealed an element with significant similarity to the highly conserved sgRNA promoter of umbraviruses and to a minimal but highly functional sgRNA promoter of carmovirus TCV. In addition, similar RNAs that also only express the RdRp and are related to Tombusviruses all generate a similar sized subgenomic RNA, and may simplify expression of peptides and proteins.

In order to determine where inserts are tolerated downstream of the sgRNA promoter in CYVaV, an evaluation of where critical elements exist in the 3′ UTR of CYVaV was conducted, so that such elements are avoided when inserting heterologous sequences. As described about, the 3′ CITE for CYVaV was identified, as well as several additional 3′ proximal hairpins that are highly conserved in umbraviruses and known to be critical for replication and translation. Using deletions/point mutations, the sequence downstream of the putative sgRNA promoter and upstream of the 3′ CITE (˜120 nt) was investigated for regions that do not impact either accumulation in protoplasts or systemic movement in N. benthamiana. A similar strategy was previously utilized by the present inventors to identify regions in the 3′ UTR of TCV that can accommodate hairpins targeted by RNase III-type enzymes (Aguado, L. C. et al. (2017). RNase III nucleases from diverse kingdoms serve as antiviral effectors. Nature 547:114-117).

After identifying suitable regions for accommodating deletions/mutations (e.g., regions not involved in critical functions), heterologous sequences of different lengths were inserted therein to evaluate CYVaV functionality with an extended 3′ UTR. Such investigation aids in determining maximal insert length to ensure that such insert will be tolerated by the CYVaV-based vector while still accumulating to robust levels and engaging in systemic movement. It is believed that the CYVaV-based vector may be able to accommodate an insert having a size of up to 2 kb. In this regard, the nearest related viruses (papaya umbra-like viruses, which like CYVaV, only encode a replicase-associated protein and the RdRp) are 1 to 2 kb larger, with all of the additional sequence length expanding their 3′ UTRs (Quito-Avila, D. F. et al. (2015), Detection and partial genome sequence of a new umbra-like virus of papaya discovered in Ecuador, Eur J Plant Pathol 143:199-204). Various size sequence fragments were evaluated, beginning at 50 nt (the size of an inserted hairpin for small RNA production), up to about 600 nt (the size of an enzybiotic ORF). Initial small RNA fragments include a reporter for knock down of phytoene desaturase, which turns tissue white. The longer size fragments include nano luciferase and GFP ORFs, which may also be used as reporters for examining expression level. Inserts are made in constructs containing the wild-type (WT) sgRNA promoter and the enhanced sgRNA promoter.

Lock and Dock Sequence for stabilizing the base of inserts. Referring to FIG. 24, Panel A, the basic structure of the lock and dock sequence is shown. Tetraloop GNRA sequence (e.g., GAAA) docking with its docking sequence generates an extremely stable structure. Sequences shown in FIG. 24, Panel A, are presented below:

(SEQ ID NO: 28)
gaaa
(SEQ ID NO: 29)
gauauggau
(SEQ ID NO: 30)
guccuaaguc
(SEQ ID NO: 31)
caggggaaacuuug

The use of a scaffold comprising a docked tetraloop as a crystallography scaffold is provided (FIG. 24, Panel B). The sequence shown in FIG. 24, Panel B, is presented below:

(SEQ ID NO: 32)
cauuagcuaaggaugaaagucuaugcuaaug

A lock and dock structure in accordance with disclosed embodiments is shown in FIG. 24, Panel C. Inserts (hairpins or non-hairpin sequences) may be added to the restriction site at the identified additional insert location. Circled bases are docking sequences for the tetraloop. The sequence shown in FIG. 24, Panel C, is presented below:

(SEQ ID NO: 33)
gcaccuaaggcgucagggucuagacccugcucaggggaaacuuugucgc
uauggugc

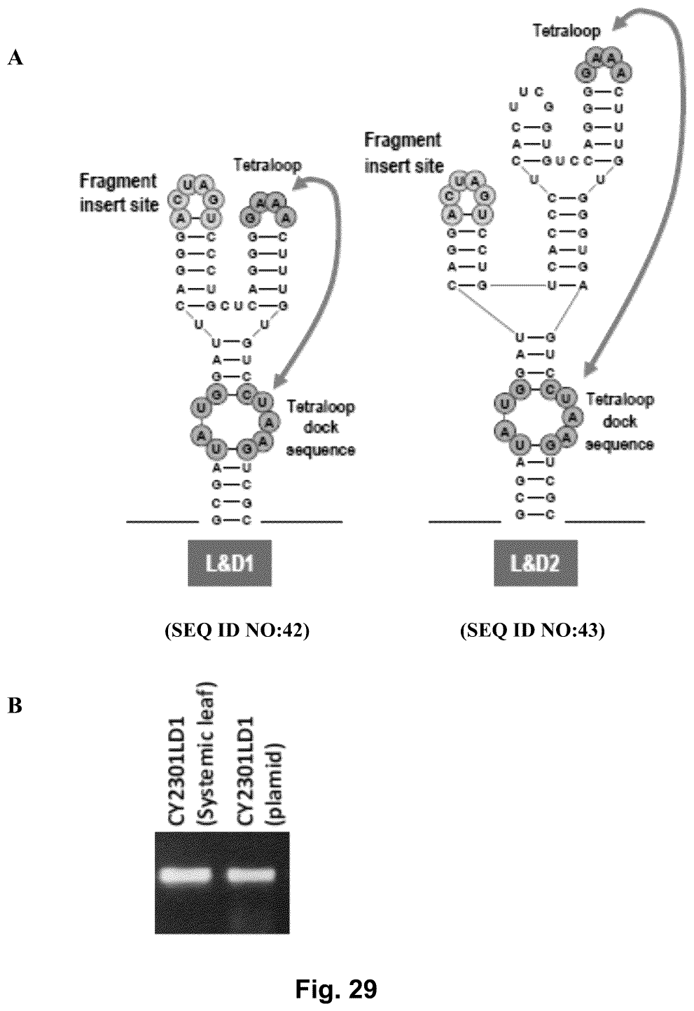

Lock and dock elements can be inserted into iRNA to stabilize the resulting vector despite the presence of hairpins or other inserts. FIG. 29 shows additional examples of lock and dock structures. Each of the two lock and dock structures shown, L&D1 (SEQ ID NO:42) and L&D2 (SEQ ID NO:43), were separately inserted into position 2301 in CYVaV to make two examples of CYVaV based vectors, one having L&D1 and the other having L&D2. The sequences shown in FIG. 29, Panel A, are presented below:

(SEQ ID NO: 42)
gcgauauggauucagggacuagucccugcucaggggaaacuuugugucc
uaagucgc
(SEQ ID NO: 43)
gcgauauggaucaggacuaguccugucacccucacuucgguguccaggg
gaaacuuugugggugaguccuaagucgc

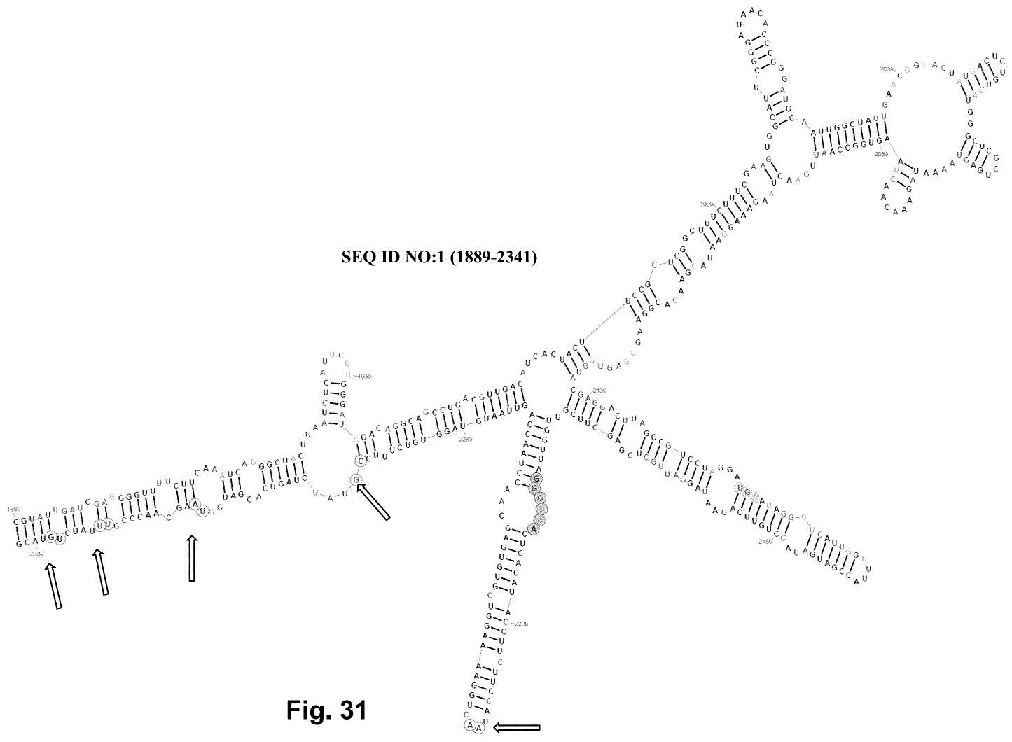

Replication, movement and stability of both of the CYVaV based vectors, each with a lock and dock structure, was demonstrated by systemically infecting N. benthamiana plants CYVaV-L&D1 and CYVaV-L&D2. In other examples, L&D1 or L&D2 may be inserted at position 2250, 2319, 2330, 2336 and 2375 (see FIG. 31).

The term “lock and dock” (L&D) is used to indicate that the structure has a highly stable locked or lockable portion and a docking portion suitable for the addition of one or more inserts. In the examples shown, the highly stable portion is provided by way of a tetraloop GNRA sequence (wherein N is A, C, G, or U; R is A or G), e.g., GAAA, and a tetraloop dock sequence (alternatively called a tetraloop lock sequence). In use, the structure folds with the tetraloop GNRA becoming associated (though not bonded in the sense of forming Watson-Crick pairs) with the tetraloop dock sequence to generate an extremely stable structure, called the “lock”. The “dock”, represented in the Figure by the fragment insert side or a portion of the lock and dock including the fragment insert site, is separated from the iRNA backbone by the lock. One or more inserts added to the dock are inhibited from interfering with folding of the iRNA backbone by the lock. Inserts (hairpins or non-hairpin sequences) may be added to the fragment insert site. In other examples, the two-way stem shown is replaced with a three-way stem to provide a lock and dock structure having a lock and two docks. The examples shown include a dividing (e.g. two-way or three-way) stem, the base and one arm of which are within a tetraloop or other locking structure, and another arm of the dividing stem having an insert site.

In addition to particular iRNA constructs, the disclosed scaffolds and lock and dock structures may be utilized for attaching a heterologous segment(s) to and/or stabilizing any RNA vector, including plant or animal vectors. An RNA-based vector may be modified via the addition of one or more lock and dock structures, such as a tetraloop GNRA docking structure. Optionally, a parental or wild-type RNA molecule suitable for use as a vector may be modified by truncating a sequence non-specific hairpin located at a particular position. Generally, the hairpin is truncated by removing an upper or distal portion of the hairpin; however, a lower portion of the hairpin (e.g., 3-5 base pairs proximate to the main structure of the RNA molecule) is retained in the truncated hairpin. The resulting truncated hairpin forms or defines an insertion site. In some embodiments an insert, which may include a scaffold such as a lock and dock structure (e.g., a tetraloop sequence), is then attached to the insertion site. The lock and dock structure may comprise a heterologous segment(s), which is thereby attached to the modified RNA molecule. In some embodiments and at particular positions, a heterologous segment(s) may be attached directly to the insertion site of the truncated hairpin and without a lock and dock or other scaffold structure intermediate the insertion site and the heterologous segment(s).

In one example, a 30 base non-hairpin sequence was inserted into L&D1, which was in turn inserted into position 2301 in CYVaV to make a CYVaV based vector. The CYVaV vector was agroinfiltrated into an N. benthamiana plant and achieved systemic movement in the plant.

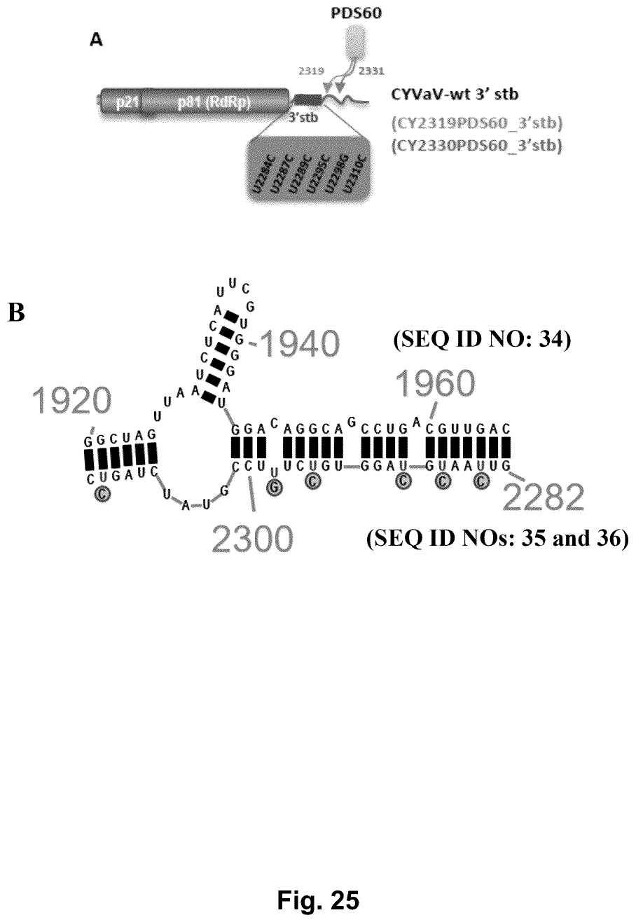

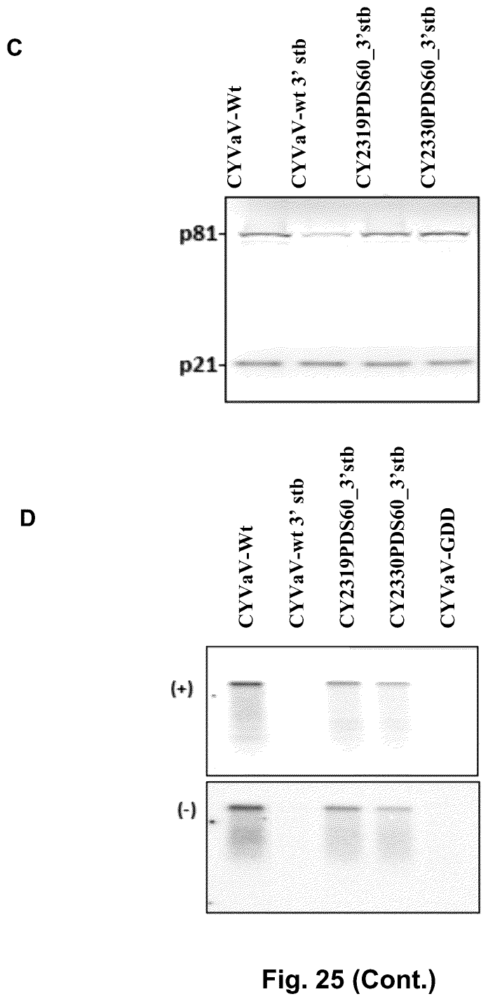

Stabilizing the local 3′UTR structure is detrimental; however insertion of a destabilizing insert nearby restores viability. Referring to FIG. 25, Panel A, a representation of CYVaV-wt is shown. CYVaV-wt 3′ stb is the parental stabilized construct containing 6 nt changes converting G:U pairs to G:C pairs. Two insertions of 60 nucleotides were added to the stabilized parental construct at positions 2319 and 2330 forming CY2319PDS60_3′ stb and CY2330PDS60_3′stb. Nucleotide changes made to stabilize the structure and generate CYVaV-wt 3′ stb are circled in Panel B. The sequences shown in FIG. 25, Panel B, is presented below:

(SEQ ID NO: 34)
ggcuaguuaaucucauucgugggauggacaggcagccugacguugac
(SEQ ID NO: 35)
guuaauguaggugucuuuccguaucuaguc (unmodified G:U
pairs)
(SEQ ID NO: 36)
gucaacgcaggugccuguccguaucuagcc (converted G:C
pairs)

B. Targets for Treatment and Management

An anti-biotic insert for delivery by the disclosed vector is provided, which comprises either an enzybiotic or small peptide engineered to destroy the CLas bacterium. Enzybiotics prefer sugar rich, room temperature environments such as found in the plant phloem. The enzybiotic is translated in companion cells during the engineered CYVaV infection cycle. Proteins produced in the cytoplasm of the phloem are naturally able to exit into the sieve element (the default pathway for translated proteins), where CLas and other plant pathogenic bacteria take up residence. In the sieve element, the enzyme molecules move with the photo-assimilate up and down the trunk and lyse any bacteria upon contact. Since enzybiotics are targeted towards a specific class of bacteria, they preferably do not disturb the microbiome of the host tree. Various agents that target CLas have been developed (e.g., Hailing Jin, University of California, Riverside, CA). Thus, numerous inserts that target CLas bacterium are known in the art and may be utilized with the CYVaV vectors of the present disclosure.

As a further embodiment, it can be beneficial to target multiple pathways for destroying the disease and the disease psyllid vector. As a result, in certain embodiments the disclosed vectors include the enzybiotic and/or peptides described above, as well as inserts that trigger the production of siRNAs that interfere with either gene expression of the tree or the disease-carrying psyllid. In the case of the ACP, the RNA could kill the vector or render it wingless and thus harmless.

C. iRNA-Based or ulaRNA-Based Vector Targeting Host Gene Expression

An iRNA-based or ulaRNA-based virus-induced gene-silencing (VIGS) vector (the acronym VIGS being used herein for convenience, although the iRNA is not necessarily a virus) is provided that effectively targets host gene expression. An CYVaV-based vector was constructed that included a hairpin that targets green fluorescent protein (GFP) mRNA expressed in N. benthamiana 16C plants. The hairpin sequence (SEQ ID NO:37; FIG. 27, Panel B) targeting GFP was inserted and tested separately in two positions: 2301 and 2250. In the N. benthamiana 16C plants, GFP is expressed in every cell from the strongest plant promoter available (cauliflower mosaic virus 35S (CaMV 35S) promoter with a double enhancer). This is far more mRNA that needs to be targeted than any natural host mRNA.

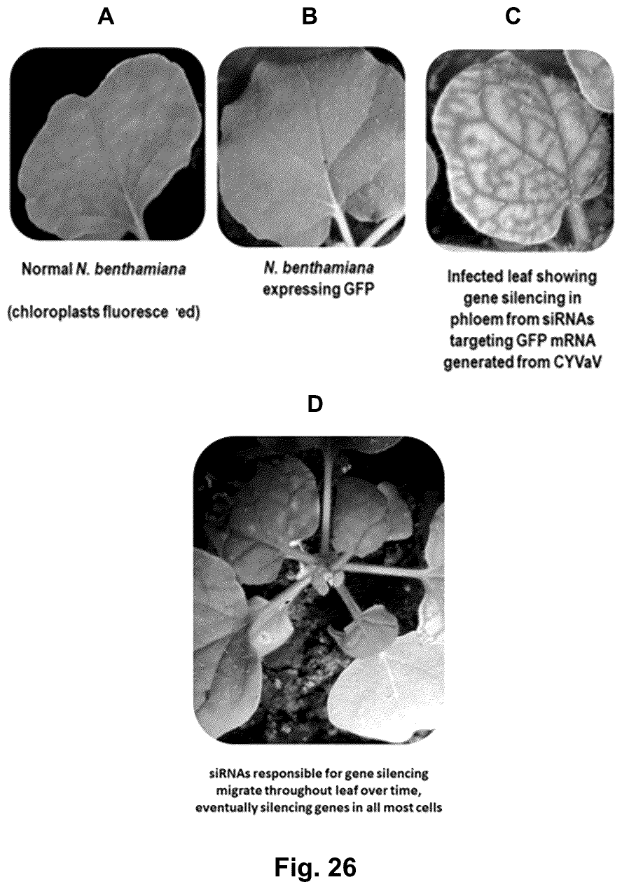

In a normal, non-infected leaf without an gene for GFP (FIG. 26, Panel A), chloroplasts fluoresce bright red when observed under ultraviolet light (shown as dark grey in Panel A). In comparison, a leaf expressing relatively high levels of GFP (FIG. 26, Panel B), appeared dull orange with green stems in coloration under UV light (shown as lighter grey in Panel B).

Leaves expressing GFP were infected with the constructed iRNA-based VIGS vector including the GFP-suppressing hairpin at position 2301 (CYVaV-GFPhp₂₃₀₁). The infected leaves demonstrated effective gene silencing (FIG. 26, Panel C). siRNAs targeted and silenced GFP mRNA first in the phloem, as readily apparent from leaf vasculature (FIG. 26, Panel C). As the VIGS construct migrated throughout the plant (FIG. 26, Panel D), siRNAs responsible for GFP gene silencing in turn were distributed throughout the leaves and plant over time, and continued to silence the target gene in all cells. GFP was significantly reduced, first in phloem (visible as bright red fluorescence in leaf veins under UV light; shown in Panel C as dark grey vein coloration). As the VIGS construct continued to migrate throughout the plant, gene suppression continued throughout the entire leaf and plant structures (visible as bright red fluorescence of entire leaves, as well as bright red coloration of younger leaves and all new leaves; shown in Panel D as dark grey coloration). Note that the same leaf in Panel C is also identified in Panel D (identified by white arrows in Panels C and D), and appeared almost completely red when observed under UV light.

Thus, gene silencing effectively spread throughout much of the entire host plant over time (see FIG. 26, Panel D, image taken 14 days after infection with CYVaV-GFPhp₂₃₀₁). Similar results were obtained by infecting leaves expressing GFP with the VIGS construct including the same GFP-suppressing hairpin (FIG. 27, Panel B) at position 2250 (CYVaV-GFPhp₂₂₅₀).

D. CYVaV-Based Vector Targeting Expression of Callose Synthase.

A vector comprising an RNA insert is provided that triggers the reduction of callose production and build-up in a host tree. A sufficiently large amount of the gene that produces callose in the phloem in response to bacteria is silenced via insertion of an siRNA sequence that is excised by the plant.

CYVaV-based vector may be utilized as a virus-induced gene-silencing (VIGS) vector to down-regulate expression of callose synthase in the phloem. VIGS has been widely used to down-regulate gene expression in mature plants to examine plant functional genomics (Senthil-Kumar et al. (2008). Virus-induced gene silencing and its application in characterizing genes involved in water-deficit-stress tolerance. J Plant Physiol 165(13):1404-1421). A complementary sequence is inserted into CYVaV at a suitable location as identified above (either anti-sense or a RNase III-cleavable hairpin). A citrus version of the gene is known (Enrique et al. (2011). Novel demonstration of RNAi in citrus reveals importance of citrus callose synthase in defense against Xanthomonas citri subsp. citri. Plant Biotech J 9:394-407).

Callose is a β 1,3-glucan that is synthesized in various tissues during development and biotic and abiotic stress (Chen, X. Y. and Kim, J. Y. (2009). Callose synthesis in higher plants. Plant Sig Behav 4(6):489-492). Deposition of callose in the sieve plates of sieve elements inhibits photoassimilate flow in the phloem, leading to over accumulation of starch in source (young) leaves, which contributes to the death of trees during bacterial infections such as HLB. All plants contain 12-14 callose synthase genes; one member of this gene family, CalS7 (Arabidopsis nomenclature), is mostly responsible for rapid callose deposition in sieve pores of the phloem in response to wounding and various pathogens (Xie et al. (2011). CalS7 encodes a callose synthase responsible for callose deposition in the phloem. Plant J 65(1):1-14). Complete inhibition of GSL7 impacted both normal phloem transport and inflorescence development in Arabidopsis (Barratt et al. (2011). Callose Synthase GSL7 Is Necessary for Normal Phloem Transport and Inflorescence Growth in Arabidopsis. Plant Physiol 155(1):328-341). A CYVaV-based vector is utilized to down-regulate the N. benthamiana and orange tree orthologues of CalS7 in mature plants in order to investigate the consequences of reduced (but not eliminated) sieve plate callose deposition. Alternatively, or in addition, the vector provides for an insert that expresses a callose-degrading enzyme.

E. iRNA-Based or ulaRNA-Based Vector Targeting CTV

An iRNA-based or ulaRNA-based VIGS vector was constructed that targets CTV. As demonstrated by the data, disclosed constructs may be utilized for immunization as well as reduction of virus levels in host plants with mature infections. N. benthamiana infected with CTV-GFP (CTV expressing GFP) was used as root stock grafted to wild-type CYVaV (CYVaVwt) and CYVaV-GFPhp₂₃₀₁ scions (FIG. 27, Panel A). The hairpin targeting GFP (FIG. 27, Panel B) was inserted at position 2301 in the construct (CYVaV-GFPhp₂₃₀₁). The sequence shown in FIG. 27, Panel B, is presented below:

(SEQ ID NO: 37)
ugaagcggcacgacuucuucaagagcgccagaauucuggcgcucuugaa
gaagucgugccgcuuca

The CYVaV-GFPhp₂₃₀₁ hairpin targeted the GFP ORF of CTV, thereby cleaving CTV. In contrast, the CYVaVwt scion had no effect on CTV-GFP infecting newly emerging rootstock leaves, as evidenced by green fluorescent flecks visible under UV light in the young leaves (FIG. 27, Panel A, center image). However, green flecks were absent in stipules when CYVaV-GFPhp₂₃₀₁ was present in the scion (FIG. 27, Panel A, right image), demonstrating that movement of CYVaV-GFPhp₂₃₀₁ down into the root stock inhibited progression of the CTV infection.

When WT CYVaV was present in the root stock, new leaves from the CTV-GFP scion still fluoresced green under UV light, thus showing that widespread CTV infection was continuing unabated (FIG. 27, Panel C, middle image). However, when CYVaV-GFPhp₂₃₀₁ was in the root stock, the upper leaves in all CTV-GFP-infected scions were either partially or nearly fully absent of GFP flecks (FIG. 27, Panel C, right image). RT-PCR of the red and green regions in the leave absent of GFP flecks (FIG. 27, Panel C, circled areas ‘A’ and ‘B’) showed that high levels of CYVaV-GFPhp₂₃₀₁ correlated with red fluorescence (region A), with this tissue having between 3,000-fold and 440,000-fold less CTV compared to green region (region B). In particular, relative levels of CTV in region A were 4.4×10⁵ fold lower as compared to CTV levels in region B. In addition, relative levels of CYVaV-GFPhp₂₃₀₁ in region A were 2.3 times greater than CYVaV-GFPhp₂₃₀₁ levels in region B (FIG. 27, Panel D).

As noted above, CTV is composed of two capsid proteins and with a genome of more than 19 kb. 76 CTV isolates have been characterized, which all contain regions of conserved nucleotides. Two sequence portions (18 and 6) of a CTV isolate are identified in Table 3 below, showing fully conserved polynucleotides (underlined below) as well as less-conserved nucleotides (in bold) with other nucleotides present in some isolates (listed as identified and bolded nucleotides in each sequence from left to right). For example, in the sequence portion for CTV18 shown in Table 3, the 3 non-conserved nucleotides include, from left to right: guanine (G) which position instead includes adenine (A) in 10 CTV isolates; cytosine (C) which position instead includes uracil (U) in about half of the CTV isolates; and G which position instead includes A in 6 CTV isolates. In the sequence portion for CTV6, the 6 non-conserved nucleotides include, from left to right: G which position instead includes A in 1 CTV isolate; G which position instead includes A in 3 CTV isolates; U which position instead includes C in 3 CTV isolates; A which position instead includes G in 9 CTV isolates; U which position instead includes C in 1 CTV isolate; and A which position instead includes G in 1 CTV isolate.

TABLE 3
Sequence Portions of CTV Isolates

| | CTV | Sequence (conserved nucleotides | Non-conserved nucleotides |
|---|---|---|---|
| # | Position | in known CTV isolates underlined): | (bolded in sequence): |
| 18 | 15173 | UCCGUGGACGUCAUGUGUAAG | G: A in 10 isolates |
| | | (SEQ ID NO: 66) | C: U in ~half isolates |
| | | | G: A in 6 isolates |
| 6 | 17856 | GGAAGUGAUGGACGAAAUUAAUGA | G: A in 1 isolate |
| | | (SEQ ID NO: 67) | G: A in 3 isolates |
| | | | U: C in 3 isolates |
| | | | A: G in 9 isolates |
| | | | U: C in 1 isolate |
| | | | A: G in 1 isolate |

Fully CTV-infected N. benthamiana were agroinfiltrated with CYVaV-based vector carrying a hairpin at position 2301 that targeted a conserved sequence in the CTV genome (SEQ ID NO:38; FIG. 27, Panel F). The CYVaV-CTV18 hairpin contained a polynucleotide sequence (SEQ ID NO:39; identified in the dashed line box, Panel F) complementary to a corresponding sequence of CTV18 in all of its variants. The sequences identified in FIG. 27, Panel F, are identified below:

(SEQ ID NO: 38)
uccguggacgucauguguaaggguacccuuacacaugacguccacgga
(SEQ ID NO: 39)
cuuacacaugacguccacgga

After four days, CTV levels in plants infected with the CYVaV-CTV18 vector were about 10-fold lower in the infiltrated tissue as compared with tissue infiltrated with CYVaV wild-type (FIG. 27, Panel E).

Leaves co-infiltrated with CTV-GFP and CYVaV wild-type or CYVaV-CTV6 containing another CTV genome-targeting hairpin (SEQ ID NO:40; FIG. 27, Panel H) also showed significant reductions in CTV-GFP at 6 days post-infiltration (FIG. 27, Panel G). The CYVaV-CTV6 hairpin contained a polynucleotide sequence (SEQ ID NO:41; identified in the dashed line box, Panel H) that is complementary to a corresponding sequence of CTV6 in all of its variants. The sequences identified in FIG. 27, Panel H, are identified below:

(SEQ ID NO: 40)
ggaagugauggacgaaauuaaugaccaaucauuaauuucguccaucacu
uccag
(SEQ ID NO: 41)
ucauuaauuucguccaucacuucc

CTV levels in plants infected with the CYVaV-CTV6 vector were visibly lower in infiltrated tissue as compared with tissue infiltrated with CYVaV wt.

F. Stability of Hairpin Targeting GFP without and with L&D

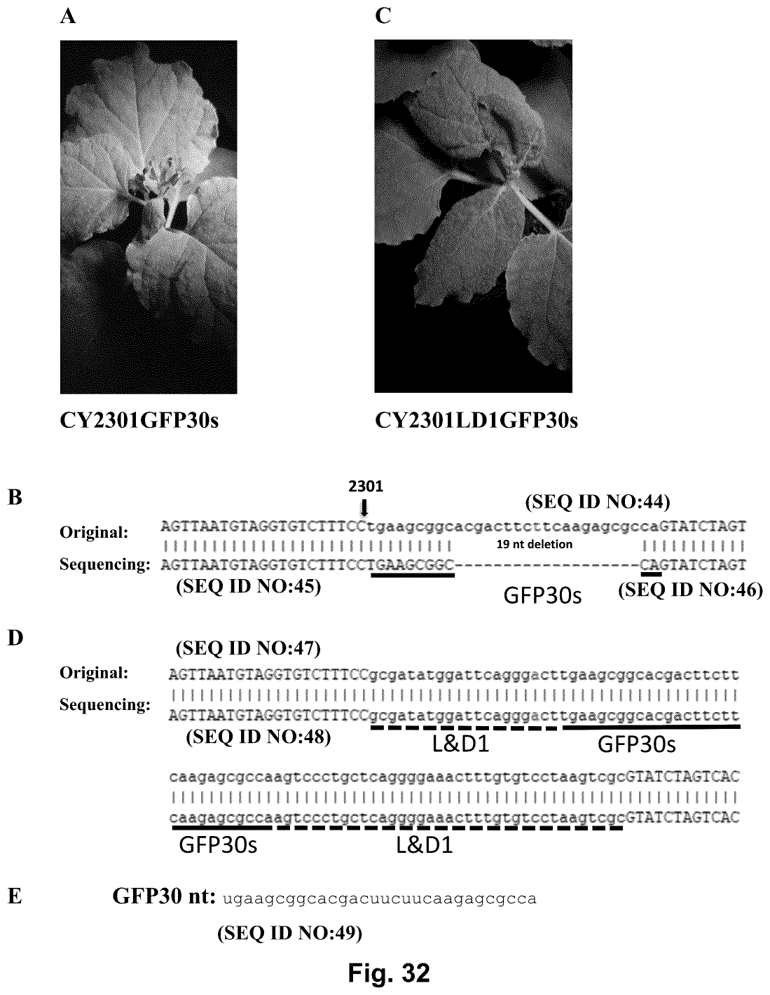

The stability of a 30 nt hairpin targeting GFP (SEQ ID NO:49; FIG. 32, Panel E) was evaluated when inserted at position 2301 without any lock and dock structure (CY2301GFP30) and with L&D1 (CY2301 LD1GPF30s).

N. benthamiana 16C plant infected with CYVaV with the 30 nt hairpin insert at position 2301 (CY2301GFP30s) is shown in FIG. 32, Panel A. Virus-induced gene silencing (VIGS) effect was not detected. Sequence alignment between input CYVaV (CY2301GFP30) and the CYVaV accumulating in systemic tissue is shown in FIG. 32, Panel B. The later CYVaV contains a 19 nt deletion acquired during infection showing the construct was not stable. The sequences identified in FIG. 32, Panel B, are shown below:

(SEQ ID NO: 4)
agttaatgtaggtgtctttcctgaagcggcacgacttcttcaagagcgc
cagtatctagt
(SEQ ID NO: 45)
agttaatgtaggtgtctttcctgaagcggc
(SEQ ID NO: 46)
cagtatctagt

N. benthamiana 16C plant infected with CYVaV with L&D1 and the 30 nt hairpin insert (SEQ ID NO:49) at position 2301 (CY2301 LD1GFP30s) is shown in FIG. 32, Panel C. Obvious GFP silencing (plant fluorescing red, shown as darker gray in Panel C) by the VIGS vector was observed. Sequence alignment between CY2301LD1GFP30s infected plant and the original construct is shown in FIG. 32, Panel D. As shown, L&D1 substantially enhanced stability of the 30 nt hairpin insert. The sequences shown in FIG. 32, Panels D and E, are shown below:

(SEQ ID NO: 47)
agttaatgtaggtgtctttccgcgatatggattcagggacttgaagcg
gcacgacttcttcaagagcgccaagtccctgctcaggggaaactttgt
gtcctaagtcgcgtatctagtcac
(SEQ ID NO: 48)
agttaatgtaggtgtctttccgcgatatggattcagggacttgaagcg
gcacgacttcttcaagagcgccaagtccctgctcaggggaaactttgt
gtcctaagtcgcgtatctagtcac
(SEQ ID NO: 49)
ugaagcggcacgacuucuucaagagcgcca

G. Stability of L&D1 and L&D1+Hairpin Targeting Callose Synthase

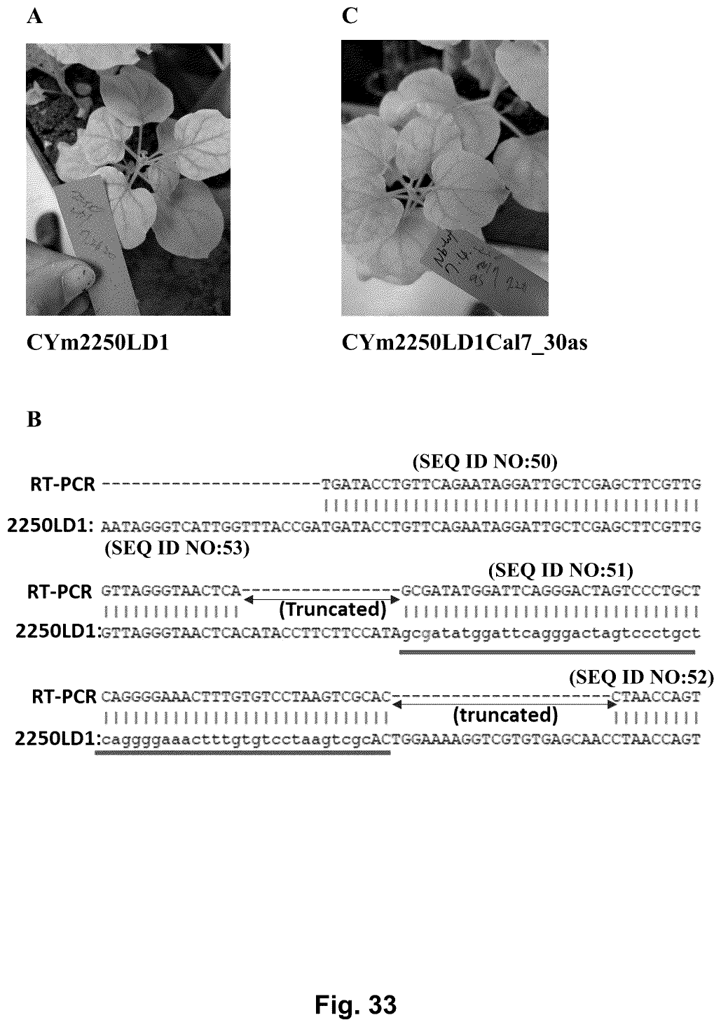

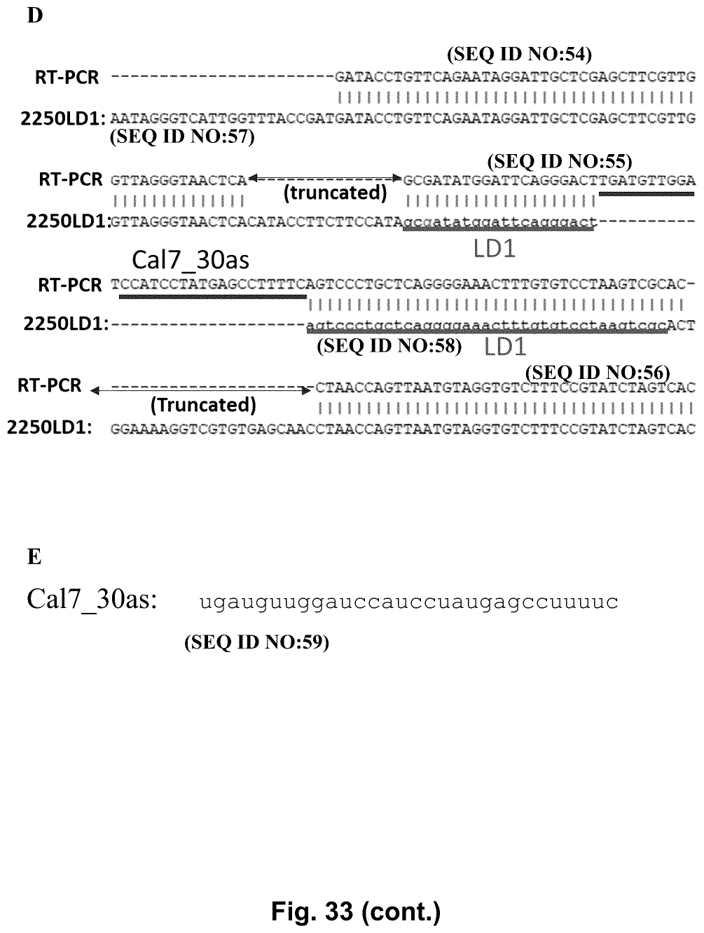

The stability of L&D1 inserted at position 2250 (CYm2250LD1), and of L&D1+a 30 nt hairpin (SEQ ID NO:59; FIG. 33, Panel E) targeting Callose Synthase (CYm2250LD1Ca1_30as), were evaluated.

N. benthamiana plant infected by CYm2250LD1 is shown in FIG. 33, Panel A, which contains L&D1 at the end of a truncated hairpin. The addition of these inserts at the end of the complete wild-type hairpin (at position 2250) were not found to be stable. Sequencing alignment (FIG. 33, Panel B) between CYm2250LD1 in infected tissue (RT-PCR) and the original construct shows complete stability. The sequences shown in FIG. 33, Panel B, are shown below:

(SEQ ID NO: 50)
tgatacctgttcagaataggattgctcgagcttcgttggttagggtaa
ctca
(SEQ ID NO: 51)
gcgatatggattcagggactagtccctgctcaggggaaactttgtgtc
ctaagtcgcac
(SEQ ID NO: 52)
ctaaccagt
(SEQ ID NO: 53)
aatagggtcattggtttaccgatgatacctgttcagaataggattgct
cgagcttcgttggttagggtaactcacataccttcttccatagcgata
tggattcagggactagtccctgctcaggggaaactttgtgtcctaagt
cgcactggaaaaggtcgtgtgagcaacctaaccagt

N. benthamiana 16C plant infected by CYm2250LD1asCa17_30as (CYVaV containing L&D1 with the 30 nt insert (SEQ ID NO:59) targeting Callose Synthase is shown in FIG. 33, Panel C. Sequence alignment (FIG. 33, Panel D) between CYm2250LD1Ca1730as accumulating in the infected plant (RT-PCR) and the original construct showing that the 30 nt insert was stable within L&D1. The 30 nt Callose synthase 7 siRNA sequence (antisense orientation) that targets the Callose Synthase that is active in phloem is shown in FIG. 33, Panel E. The sequences shown in FIG. 33, Panels D and E, are shown below:

(SEQ ID NO: 54)
gatacctgttcagaataggattgctcgagcttcgttggttagggtaac
tca
(SEQ ID NO: 55)
gcgatatggattcagggacttgatgttggatccatcctatgagccttt
tcagtccctgctcaggggaaactttgtgtcctaagtcgcac
(SEQ ID NO: 56)
ctaaccagttaatgtaggtgtctttccgtatctagtcac
(SEQ ID NO: 57)
aatagggtcattggtttaccgatgatacctgttcagaataggattgct
cgagcttcgttggttagggtaactcacataccttcttccatagcgata
tggattcagggact
(SEQ ID NO: 58)
agtccctgctcaggggaaactttgtgtcctaagtcgcactggaaaagg
tcgtgtgagcaacctaaccagttaatgtaggtgtctttccgtatctag
tcac
(SEQ ID NO: 59)
ugauguuggauccauccuaugagccuuuuc

In some examples, iRNA with a truncated hairpin (of the iRNA) and an insert have been stable over long test periods, for example over 40 days. Without intending to be limited by theory, truncating a hairpin of the iRNA (e.g., CYVaV), for example a structurally required hairpin, in combination with adding an insert to the hairpin of the iRNA results in the hairpin of the iRNA resembling its original size and/or retaining its structural integrity. It should be understood however, that the inserted hairpin or unstructured short RNA sequence need not be the same or similar size to truncated hairpin.

H. iRNA-Based or ulaRNA-Based Vector Containing Multiple Inserts

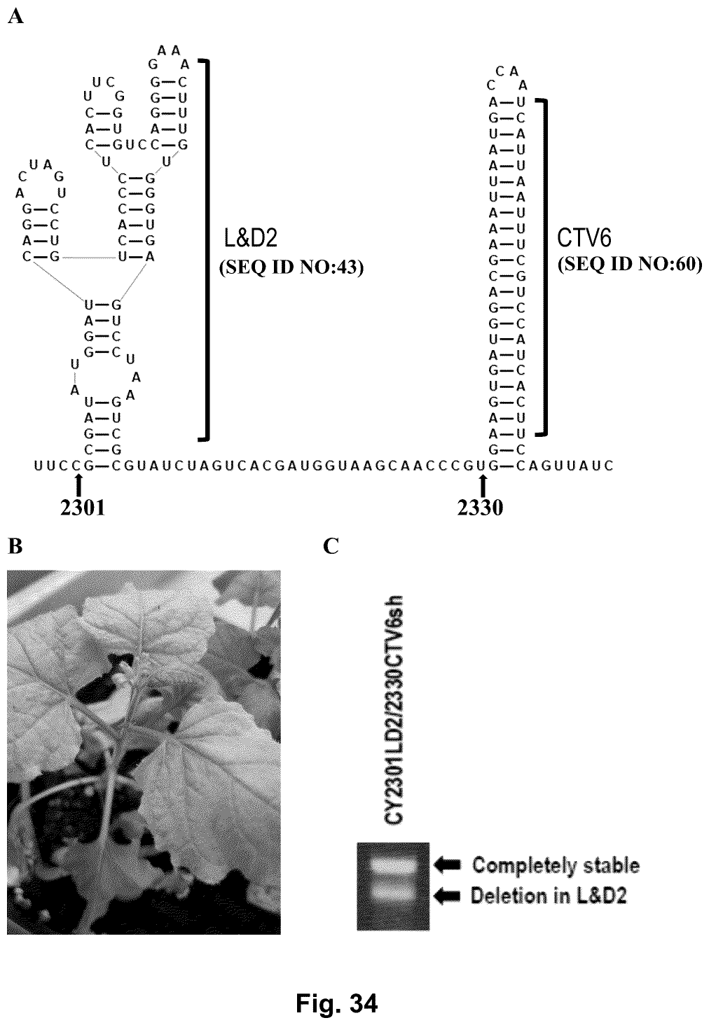

An iRNA-based or ulaRNA-based vector was constructed that includes an insert at position 2301 and another insert at position 2330 (CY2301LD2/2330CTV6sh). The insert at position 2330 is a hairpin targeting CTV6 (SEQ ID NO:60) and the other insert at position 2301 is an empty L&D2 structure (SEQ ID NO:43; FIG. 34, Panel A). The sequences shown in FIG. 34, Panel A, are shown below:

(SEQ ID NO: 43)
gcgauauggaucaggacuaguccugucacccucacuucgguguccagg
ggaaacuuugugggugaguccuaagucgc
(SEQ ID NO: 60)
ggaagugauggacgaaauuaaugaccaaucauuaauuucguccaucac
uucc

N. benthamiana infected with CY2301LD2/2330CTV6sh is shown in FIG. 34, Panel B. RT-PCR result from CY 2301LD2/2330CTV6sh-infected plant is shown in FIG. 34, Panel C. The top band had both inserts and was the same as the original infiltrated construct. The lower band has a deletion in L&D2. The data show that two inserts were tolerated and the construct was infectious.

I. Enhanced Stability Lock and Dock Structure

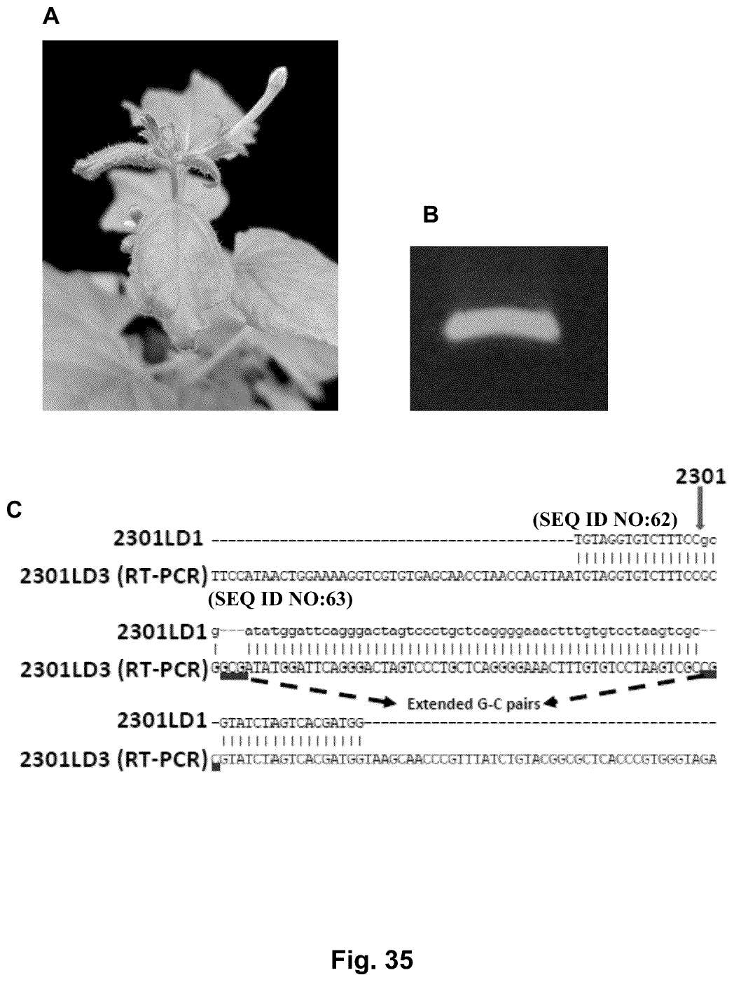

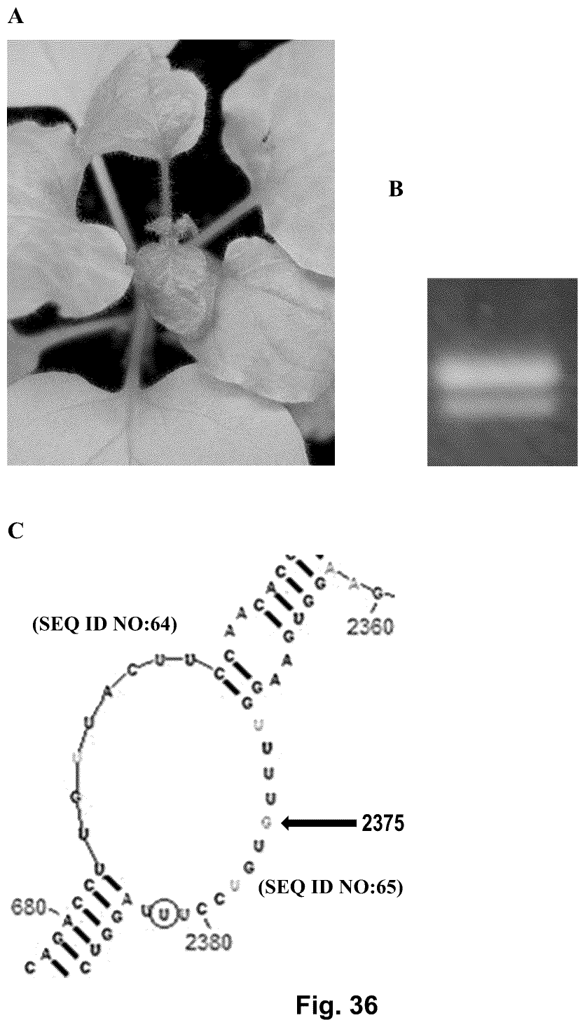

Extending base-pairing at the base of the disclosed lock and dock structures improved stability of larger unstructured inserts. Base-pairing was extended in L&D1 to include three additional base pairs (G-C, C-G, G-C) (FIG. 35, Panel C) thereby resulting in a third lock and dock structure (L&D3). The sequence of L&D3 is provided below:

(SEQ ID NO: 61)
gcggcgauauggauucagggacuagucccugcucaggggaaacuuugug
uccuaagucgccgc

N. benthamiana plant infected with L&D3 at position 2301 (CY2301LD3) is shown in FIG. 35, Panel A. RT-PCR from the symptomatic leaf of infected plant showing a single band (no obvious deletions) is shown in FIG. 35, Panel B. Sequence alignment (FIG. 35, Panel D) of CYVaV with L&D1 in position 2301 and with RT-PCR sequencing of CY2301LD3 from infected plant tissue is shown. No instability was detected. The sequences shown in FIG. 35, Panel C, are shown below:

(SEQ ID NO: 62)
tgtaggtgtctttccgcgatatggattcagggactagtccctgctcagg
ggaaactttgtgtcctaagtcgcgtatctagtcacgatgg
(SEQ ID NO: 63)
ttccataactggaaaaggtcgtgtgagcaacctaaccagttaatgtagg
tgtctttccgcggcgatatggattcagggactagtccctgctcagggga
aactttgtgtcctaagtcgccgcgtatctagtcacgatggtaagcaacc
cgtttatctgtacggcgctcacccgtgggtaga

J. iRNA-Based or ulaRNA-Based Vector Containing siRNAs

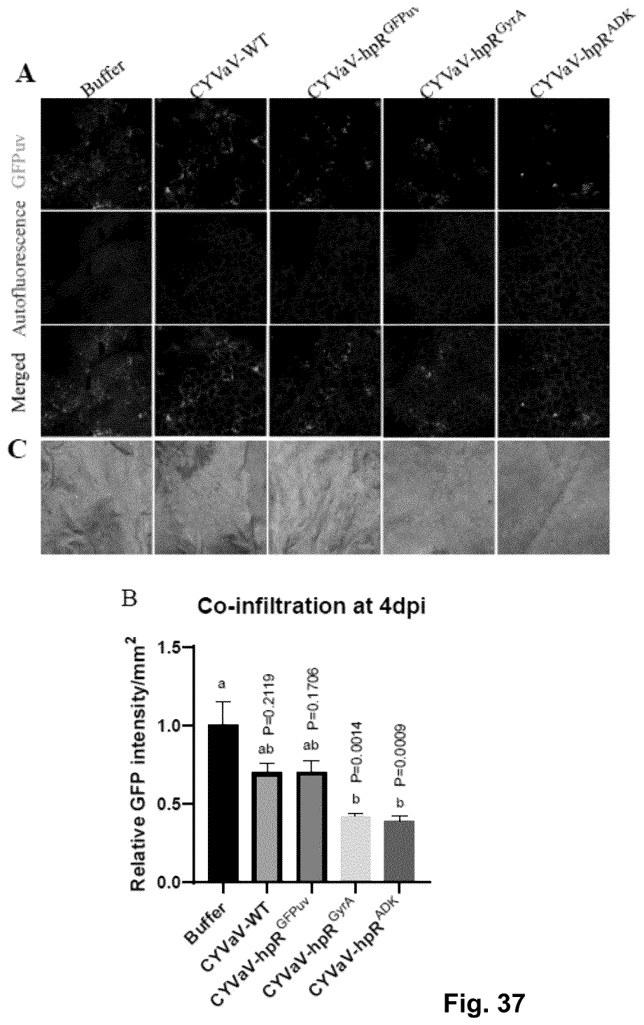

Referring to FIG. 37, CYVaV-vectored siRNAs demonstrated the capability of silencing gene expression of Pseudomonas syringae in co-infiltrated leaves of N. benthamiana. CYVaV-derived siRNAs silenced gene expression of Pseudomonas syringae expressing GFP (GFP-Pst). Buffer (10 mM MgCl₂) or Agrobacterium strain GV3101 (OD₆₀₀=0.65) with a T-DNA containing either CYVaV-WT or CYVaV with hairpins targeting either GFP or two critical P. syringae genes were coinfiltrated with GFP-Pst (OD₆₀₀=0.00005) into leaves of N. benthamiana plants. The genes targeted were adenylate kinase (ADK) and gyrase subunit A (GyrA), which are highly conserved and essential for all bacteria species. Growth of GFP-Pst was monitored by examining GFP fluorescence using confocal imaging at 4 days post-infiltration (FIG. 37, Panel A). Quantitative analysis of the intensity of GFP expressed by GFP-Pst was conducted (FIG. 37, Panel B). Mean±SE (n=8; 8 leaves from 4 plants). The different letters indicate significant differences as determined by ANOVA post hoc (P<0.05). Images show infiltrated area from representative leaves photographed 9 days after infection with GFP-Pst (FIG. 37, Panel C).

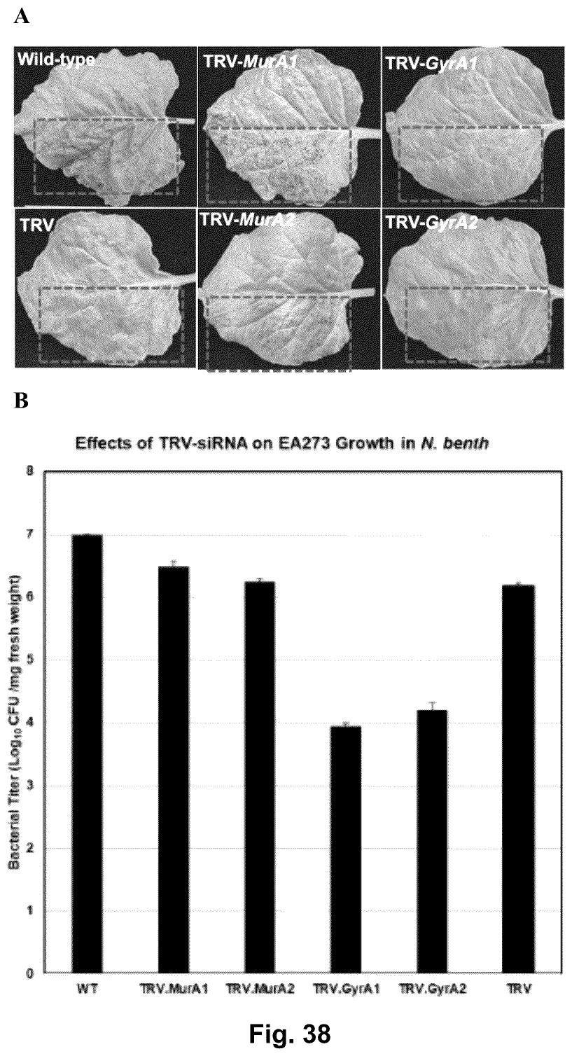

Referring to FIG. 38, TRV-derived siRNAs targeting GyrA gene substantially reduced Erwinia amylovora infection in systemically infected leaves of N. benthamiana. TRV-derived siRNAs demonstrated the ability to effectively silence gene expression of Erwinia amylovora. Agrobacterium strain GV3101 harboring either TRV1 or TRV2 (final OD₆₀₀=0.6, TRV1:TRV2=1:1) with siRNAs targeting two Erwinia amylovora critical genes (MurA and GyrA) were co-infiltrated into 2 week-old of N. benthamiana plants. The infiltrated plants were topped 2 weeks after infiltration to increase TRV in upper systemic leaves. As shown in FIG. 38, Panel A, half of the systemic leaves were challenged with Erwinia amyloyora strain 273 (EA273, diluted to OD₆₀₀=0.0005 in 10 mM MgCl₂) by infiltration 7 days later. Graphs were taken and the bacterial titers were quantified 3 days after infection (FIG. 38, Panel B). WT, EA273 on wild-type N. benthamiana; TRV, EA273 on TRV-infected N. benthamiana; TRV.MurA1, EA273 on TRV-MurA1 (MurA gene fragment 1) infected N. benthamiana; TRV.MurA2, EA273 on TRV-MurA2 (MurA gene fragment 2 and different from MurA1) infected N. benthamiana; TRV.GyrA1, EA273 on TRV-GyrA1 (EA GyrA gene fragment 1) infected N. benthamiana; TRV.GyrA2, EA273 on TRV-GyrA2 (GyrA gene fragment 2 and different from GyrA1) infected N. benthamiana. Note that siRNAs targeting GyrA reduce Erwinia levels 1000-fold.

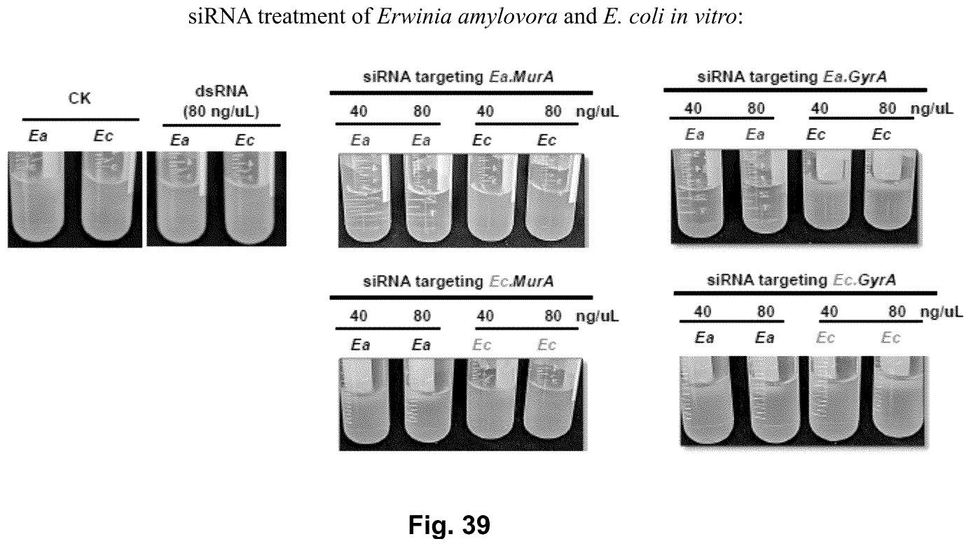

Referring to FIG. 39, siRNA treatment of Erwinia amylovora and E. coli in vitro demonstrated that specific siRNAs inhibited growth of Erwinia but not E. coli. Further, siRNAs targeting E. coli genes would not target the genes in Erwinia and vice versa. Erwinia was not affected by the non-specific siRNAs.

Referring to FIG. 40, non-specific siRNAs did significantly inhibit growth of Liberibacter crescens (Lcr) proliferation in vitro. siRNAs were derived from −500 bp long dsRNA via in vitro transcription of GFPuv, Lcr-Gy and Lcr-ADK genes. Water or siRNAs at ng/μl and 100 ng/μl were added to fresh medium (blank) or Lcr cultures with a density of OD₆₀₀=0.001. bacterial growth was photographed at 16 days after treatment. (FIG. 40, Panel A). Bacterial growth measured by a UV spectrophotometer (FIG. 40, Panel C). Non-specific relatively short siRNAs (e.g., 21-24 bp siRNAs) surprisingly inhibited growth of L. crescens in vitro. However, the relatively long 500 bp dsRNA did not inhibit growth. Significant inhibition of bacterial growth was also demonstrated via siRNA treatment of Pseudomonas syringae (FIG. 41). siRNA targeting GFPuv, Pst-Gy, Pst-ADK and Lcr-ADK do not kill the bacteria directly, but still showed significant growth inhibition.

K. Determination of Positional Entropy and Average Positional Entropy

Determining the positional entropy of the bases in an insert starts with determining the secondary structure, i.e. the arrangement of paired and unpaired bases, of the insert. Although RNA folding software may be inaccurate for large sequences, the output of RNA folding software is typically accurate for small sequences, for example inserts of less than 300 nt or less than 200 nt. Additional methods such as SHAPE reactivity may be used to determine the secondary structure of large or difficult inserts, or to confirm the accuracy of or modify a software-produced secondary structure drawing.

Using the RNAfold software as an example, the sequence of an insert is entered into the program. The available results include a minimum free energy (MFE) prediction, an optimal secondary structure of the insert sequence in the MFE state in dot-bracket notation, and a graphical drawing of the dot-bracket notation of the MFE structure. Selecting “EPS” in the download options for the MFE structure drawing encoding positional entropy produces a file with a list of the positional entropy of each base in the insert. The positional entropy of the individual bases are added together and divided by the number of bases to produce the average positional entropy (APE).

Unless stated otherwise, the values of DG, PE and APE described herein are produced using RNAfold version 2.4.18 available from, or accessed through, the University of Vienna. The program is further described in Mathews D H, Disney M D, Childs J L, Schroeder S J, Zuker M, Turner D H. (2004) Incorporating chemical modification constraints into a dynamic programming algorithm for prediction of RNA secondary structure. Proc Natl Acad Sci USA 101(19):7287-92; Gruber A R, Lorenz R, Bernhart S H, Neubock R, Hofacker I L. The Vienna RNA Websuite. Nucleic Acids Research, Volume 36, Issue suppl_2, 1 Jul. 2008, Pages W70-W74, DOI: 10.1093/nar/gkn188; and, Lorenz R, Bernhardt S. H., Honer zu Siederdissen C, Tafer H, Flamm C, Stadler P F and Hofacker I L, “ViennaRNA package 2.0”, Algorithms for Molecular Biology, 6:1 pages 26, 2011.

L. Stability Assay

Stability of the insert is determined after allowing the insert to replicate in plants for a period of time. The vector is then harvested from the plant and assayed through RT-PCR. A stable insert will show only one band indicative of the intact vector. The appearance of a second band indicates vector replicates that have deleted the insert, and the insert is not entirely stable. In an entirely stable vector, batch sequencing will show no heterogeneity.

M. Natural Hairpin-Like Structures in CYVaV1

FIG. 49 Panel C shows the secondary structure of full-length CYVaV1. Each “dot” represents a nucleotide. Numbers denote natural CYVaV1 hairpin-like structures that served as templates for the mimic hairpins. When duplicated and inserted into different locations in the CYVaV1 3′UTR, these hairpin-like structures were stably maintained at all time points assayed until senescence of the host plant.

Structures 1 to 4 in FIG. 49 Panel C are identified, based on the position numbers of their nucleotides, as CY1132-1329; CY1493-1525; CY2136-2218 and CY2220-2280 respectively. CY1132-1329 has 6 (3%) of nucleotides with positional entropy of over 1.0 and a standard deviation of positional entropies of 0.30. The average APE is 0.19 and the highest positional entropy (PE) is 1.49. The DG is −87.9 kcal/mol. CY1493-1525 has 1 nucleotide (3%) with positional entropy of over 1.0 and a standard deviation of positional entropies of 0.25. The average APE is 0.10 and the highest PE is 1.00. The DG is −13.1 kcal/mol. CY2136-2218 has 1 (1.2%) of nucleotides with positional entropy of over 1.0 and a standard deviation of positional entropies of 0.25. The average APE is 0.11 and the highest PE is 1.12. The DG is −41.9 kcal/mol. CY2220-2280 has no nucleotides (0%) with positional entropy of over 1.0 and a standard deviation of positional entropies of 0.23. The average APE is 0.15 and the highest PE is 0.80. The DG is −29.9 kcal/mol.

The sequence of CY1132-1329 (structure 1, 198 nucleotides) is (SEQ ID NO:125):

cagtaaaaggttgtatggtgagggtgctgagccgtgtatcgccaaaggc
ctaaatgcattagaatctggagcgactttga

The sequence of CY1493-1525 (structure 2, 33 nucleotides) is (SEQ ID NO:126):

tttatgcttagtgaacttggcattaagcatgaa

The sequence of CY2136-2218 (structure 3, 83 nucleotides) is (SEQ ID NO:127):

acgaggacttaggcgtcctaggatgaatagggtcattggtttaccgatg
atacctgttcagaataggattgctcgagctt

The sequence of CY2220-2280 (structure 4, 61 nucleotides) is (SEQ ID NO:128):

ggttagggtaactcacataccttcttccataactggaaaaggtcgtgtg
agcaacctaacc

In various examples: CY1132-1329 was inserted at 2219/2281 of CYVaV; CY1493-1525 was inserted at 2319/2320 and 2304/2305 of CYVaV (in two separate examples); CY2136-2218 was inserted at 2304/2305 of CYVaV; and, CY2220-2280 was inserted at 2304/2305 of CYVaV. In each case, the resulting vector was stable in planta at the end of a trial.

A line of best fit through these four hairpin-like structures has the formula DG in kcal/mol=−0.44× length of the structure in nt −1.89. When comparing the DG of a synthetic construct to the DG of a natural hairpin-like structure of similar length, this formula (or a different formula derived from another wild type vector) may be used when a specific natural structure of similar length does not exist. In some examples, stable hairpin-like structures of a selected length are stable when their DG is in a range of +/−10 kcal/mol, or −5 to +15 kcal/mol, of the DG determined by the formula for the selected length. A line of best fit through 58 stable synthetic inserts and the four hairpin-like like structures of CYVaV has the formula DG in kcal/mol=−0.43×length of the structure in nt+1.17. Optionally, hairpin-like structures can be made in a range of −10 to +10 kcal/mol, or −5 to +15 kcal/mol, of the DG determined by this formula for the selected length.

N. Example of a Stable Insert Targeted Against CVEV

Two inserts were prepared including siRNA sequences targeted against regions of the CVEV genome. Insert mmck15 (FIG. 54 Panel B) has an siRNA sequence of 29nt complementary to bases 4308-4338 of CVEV. Insert mmck8 (FIG. 54 Panel C) has an siRNA sequence of 28nt complementary to bases 766-793 of CVEV. Sequences are given below. SiRNA portions of the sequences are at the beginning of the sequences and shown in bold type and inside a box in the figure.

The sequence of the wt structure is (SEQ ID NO:99):

ggttagggtaactcacataccttcttccataactggaaaaggtcgtgtg
acaacctaacc

The sequence of mmck15 is (SEQ ID NO:100):

gtcgcaatcaaagacgaagaaatcgtccataactggacatttccttcgt
ctcaattgcgac

The sequence of mmck8 is (SEQ ID NO:101):

ggttgcttggaacccatacgaatgttgcttaacagcaaattcgctatgg
gtgatagcaacc

Wild type structure 4 (FIG. 54 Panel A) has a ΔG of −30.00 kcal/mol and an APE of Mmck15 has a ΔG of −29.80 kcal/mol, an APE of 0.18, a standard deviation of PE of 1.6% of nucleotides with a PE over 1, and a highest PE of any nucleotide of 1.0. Mmck8 has a ΔG of −30.30 kcal/mol, an APE of 0.16, a standard deviation of PE of 0.24, 1.6% of nucleotides with a PE over 1, and a highest PE of any nucleotide of 1.0. Both mimics were stable after 4 months (the entire duration of the experiment) in N. benthamiana. Secondary structure diagrams of wild type structure 4 (Panel A), mmck 15 (Panel B) and mmck (Panel C) are shown in FIG. 54.

N. benthamiana plants were agroinfiltrated with CYVaV_{CVEV}. After 21 days, the plants were agroinfiltrated with CVEV. After 10 days, the CVEV levels in the CYVaV_{CVEV} treated plants were 17.6 and 29.5 relative to an untreated control (100).

Although CYVaV has been found in nature only four times, it naturally infects limequat trees and potential other citrus trees. CYVaV has not coat protein and therefore likely requires a helper virus to travel from tree to tree. The helper virus is believed to be citrus vein enation virus (CVEV). Even though CVEV does not produce significant symptoms in plants, and CYVaV introduced into a citrus tree is unlikely to travel from tree to tree, the safety of a CYVaV derived RNA vector may be increased by adding an insert to the vector that targets CVEV. With a reduced population of CVEV, the likelihood of a CYVaV traveling to another tree is reduced. A second inset in the RNA vector may target a pathogen or gene expression in the plant. For example, in case of citrus greening, the plant is harmed by overproduction of callose in response to the infection. Excess callose, in combination with phloem protein 2 (PP2) clogs citrus sieve elements, thereby damaging the plant. CYVaV de-polymerizes the PP2 and reduces clogging of the citrus sieve elements. Thus wild type CYVaV may be used to treat citrus trees against citrus greening. Alternatively, an RNA vector may be derived from CYVaV and further contain inserts targeted against one or more of CVEV, the production of callose, or the L. asiaticus bacteria.

O. Further Examples of Inserts Mimicking Structure 4

Referring to FIG. 54, the wild type Structure 4 is shown as Panel A. Panel D is an insert called mmckpsvD with a 29nt siRNA targeting bases 174-202 of peanut stunt virus. Its ΔG is −30.40 kcal/mol and it has an APE of 0.17, a standard deviation of PE of 0.28, 3.3% of nucleotides with a PE over 1, and a highest PE of any nucleotide of 1.0. Panel E is an insert called mmckpsvE with a 29nt siRNA targeting bases 231-259 of peanut stunt virus. Its ΔG is −30.50 kcal/mol and has an APE of 0.12, a standard deviation of PE of 0.25, 1.6% of nucleotides with a PE over 1, and a highest PE of any nucleotide of 1.0. Both mimics were stable after 4 months in N. benthamiana (the entire duration of the experiment). SiRNA portions of the sequences are boxed in the figure.

The sequence of mmckpsvD (SEQ ID NO:102) is shown below. The first 29 nucleotides are the targeting sequence.

gggtgaagtcatgaaagaagctgccttcttaacagaagcagctctcttt
cacaattcaccc

The sequence of mmckpsvE (SEQ ID NO:103) is shown below. The first 29 nucleotides are the targeting sequence.

ccgtaggtaagacaccattgacaagttcttaacagaactgtcacatggt
gtgaccctacgg

Referring to FIG. 55, the wild type Structure 4 is again shown as Panel A. Panel B is an insert called PRSVmmck1 with an siRNA targeting papaya ring spot virus. Its ΔG is −24.20 kcal/mol and has an APE of 0.13, a standard deviation of PE of 0.27, 1.6% of nucleotides with a PE over 1, and a highest PE of any nucleotide of 1.0. Panel C is an insert called PRSVmmck2 with an siRNA targeting papaya ring spot virus. Its ΔG is −29.00 kcal/mol and it has APE of 0.14, a standard deviation of PE of 0.31, 3.3% of nucleotides with a PE over 1, and a highest PE of any nucleotide of 1.6. Both mimics were stable after 4 months in N. benthamiana (the entire duration of the experiment). SiRNA portions of the sequences are boxed in the figure.

The sequence of PRSVmmck1 (SEQ ID NO:104) is shown below. The first 29 nucleotides are the targeting sequence.

atggtctgaatgataatgaaatgcaagtgtaaccacttcatttccatta
tccaaagaccat

The sequence of PRSVmmck2 (SEQ ID NO:105) is shown below. The first 29 nucleotides are the targeting sequence.

gcagaagcatatattgcaaagagaaatgctaacgcattctcttctgcaa
tacaacttctgc

P. Examples of Inserts Mimicking Structure 1

FIGS. 56-60 each show the wild type Structure 1 found at 1132-1329 of CYVaV in Panel A. Its ΔG is −77.0 kcal/mol and its APE is 0.19.

FIG. 56 shows PDS-mmck-1 (Panel B) and PDS-mmck-2 (Panel C). These inserts target phytoene desaturase (PDS). PDS is a widely used reporter gene to demonstrate silencing an endogenous gene in plants. Silencing the PDS gene produces a photobleached phenotype. ΔG of PDS-mmck-1 is −73.80 kcal/mol and has an APE of 0.16, a standard deviation of PE of 0.27, 3.0% of nucleotides with a PE over 1, and a highest PE of any nucleotide of 1.6. ΔG of PDS-mmck-2 is −73.80 kcal/mol and it has an APE of 0.21, a standard deviation of PE of 0.32, 4.0% of nucleotides with a PE over 1, and a highest PE of any nucleotide of 1.5. SiRNA portions of the sequences are boxed in the figure.

The sequence of the wt structure is (SEQ ID NO:106):

cagtaaaaggttgtatggtgagggtgcttgagccgtgtatcgccaaagg
cctaaatgcattagaatctggagcgactttgaggcgcaaatgggagaag
ttttcttctccagtttgcgtttctctcgacgcttccaggttcgacctgc
atgtaagcgttggcatgctaaagttcacacacaagctatatgactatta
ctg

The sequence of the PDS-198mmck-1 insert (SEQ ID NO:107) is shown below (target sequence shown in bold):

cacgaaacagaagtacttggcttcaatggaaggtgctgtcttatcagga
aagctttgtgcacaagctattgtacaggattacgagttacttcttggct
tttgccaagatgtaactcgtaaacaaattacaaatagcttgtcaaacaa
aactgtcggataaacagtaaacttccttgcctacaagtaagtctattcg
tg

The sequence of the PDS-198mmck-2 insert (SEQ ID NO:108) is shown below (target sequence shown in bold):

gttgctcagtgtgtacgctgacatgtctgttacatgtaaggaatattac
aaccccaatcagtctatgttggaattggtatttgcacccgcagaagagt
tttctcttctacgggtgcaaataagtgtttcacaacatagaaaatattg
gagtcgtctattcttacccaataacaacaacaaagcgtacacacaagca
ac

FIG. 57 shows LcrGyrA-mmck5 (Panel B) and LcrGyr-mmck3 (Panel C). These inserts target the GyrA gene of Liberibacter crescens. ΔG of LcrGyrA-mmck5 is −72.00 kcal/mol and it has an APE of 0.32, a standard deviation of PE of 0.40, 6.1% of nucleotides with a PE over 1, and a highest PE of any nucleotide of 1.9. ΔG of LcrGyrA-mmck3 is −72.3 kcal/mol (by RNAfold) or −58.20 kcal/mol (by mfold) and it has an APE of 0.31, a standard deviation of PE of 0.47, 13.6% of nucleotides with a PE over 1, and a highest PE of any nucleotide of 1.9. LcrGyrA-mmck3 is unusual in that its MFE when calculated by two different programs (mfold and RNAfold) varies, whereas for most hairpin-like structures the MFE calculated by different programs is similar. Values described herein are from RNAfold unless stated otherwise. LcrGyrA-mmck3 also had a higher standard deviation of PE and more high PE bases than most other haipin-like structures tested. It is not known whether a difference in stability between, for example, LcrGyrA-mmck3 and LcrGyrA-mmck5 may have emerged after a longer experiment. SiRNA portions of the sequences are boxed in the figure.

The sequence of the LcrGyrA-mmck5 insert (SEQ ID NO:109) is shown below (target sequence shown in bold):

acctggtactgttcggcgaaataaattatctgattttgtgcatgtgaac
cgtaatggtaagattgcaatgaaattggaggaaaatgatgagattgttt
tttaacaatcgcatcattttccccattcgctcatgtgcaatgaccacca
tcgttgtaacatgacaactcacagataataccatcgccgaacaccccag
gt

The sequence of the LcrGyrA-mmck3 insert (SEQ ID NO:110) is shown below (target sequence shown in bold):

atgatgagattgtttcagtagaaacttgtactgaggaccatgatgtttt
gctaacaacagaatttggtcagtgtattcgattcccagtttctaatgtt
tttacattagcaactgtgaatcccacacacctgacccatttaaacgttg
tcaaaaaccattaggtcaaagagtacagtggaaactgaaacaacccatc
at

FIG. 58 shows Clas-GyrAmmck-5′ (Panel B) and Clas-GyrAmmck-3′ (Panel C). These inserts target the GyrA gene of Liberibacter asiaticus. ΔG of Clas-GyrAmmck-5′ is −66.30 kcal/mol and it has an APE of 0.23, a standard deviation of PE of 0.36, 5.1% of nucleotides with a PE over 1, and a highest PE of any nucleotide of 1.2. ΔG of Clas-GyrAmmck-3′ is −73.90 kcal/mol and it has an APE of 0.20, a standard deviation of PE of 0.35, 3.0% of nucleotides with a PE over 1, and a highest PE of any nucleotide of 1.6. SiRNA portions of the sequences are boxed in the figure.

The sequence of the Clas-GyrAmmck-5′ insert (SEQ ID NO:111) is shown below (target sequence shown in bold):

gcatggcaatgtacggcgtaataaactttctgattttattcaaatcaat
cgtagtggtaagattgcgatgaaattagattcaagagatgagattcttt
tttaagaatcccatctcttgaaacattccctcatgcgcaataaccacca
cccatattatttgataacccacagaaggttcaaatgctgtacaccccat
gc

The sequence of the Clas-GyrAmmck-3′ insert (SEQ ID NO:112) is shown below (target sequence shown in bold):

gagatgagattctttccgttgaaacctgtacacaagaaaatgatatatt
gttgactactaaacttggacaatgtgtccgctttccgatttctgctatt
tttatagcagcaatcggaaagctaacacacttgtcccaagtggccgtag
ttccaaactatcatttcgccctgtacggtaaacacggaaagaaaacatc
tc

FIG. 59 shows CS7 (Panel B). This insert targets the callose synthase 7 gene of Nicotiana benthamiana. ΔG of CS7 is −75.90 kcal/mol and it has an APE of 0.23, a standard deviation of PE of 0.36, 5.6% of nucleotides with a PE over 1, and a highest PE of any nucleotide of 1.5. The targeted sequence of the CS7 gene is similar to a sequence of the orthologue from citrus (CscalS7), which is responsible for producing callose in sieve elements in response to pathogens. SiRNA portions of the sequences are boxed in the figure.

The sequence of the CS7 insert (SEQ ID NO:113) is shown below:

agaattatggagcggtcaagctcacacatcgtcacactttttatgtggt
gggctcagccaaaactgtatgtaggaagaggcttgcatgagggcatgtt
tttacatgccatcatgcaagccagacgactatcatacagttgaccgctg
atccaactcataaaagtcaaaatgattgtacaattgattgctctcaatt
ct

FIG. 60 shows insert Erwinia GyrA (Panel B). This insert targets the GyrA gene of Erwinia. ΔG of Erwinia GyrA is −76.70 kcal/mol and it has an APE of 0.13, a standard deviation of PE of 0.29, 4.0% of nucleotides with a PE over 1, and a highest PE of any nucleotide of 1.5. SiRNA portions of the sequences are boxed in the figure.

The sequence of the Erwinia GyrA insert (SEQ ID NO:114) is shown below (target sequence shown in bold):

ccgaccgcgcagcgccggtattattgccgtcaatctgcgtgatgacgat
gaactgatcggcgtgtcgctgacgaacggtagtgatgaagcgatgctgt
tttcagcatcacttcatcactaaaaagacgtacagtgacacataagatc
acttaatatcatccgtatacatgatgcaaaccaattggtgttgtaggtt
gg

All of the inserts mimicking structure 1 described in this section were inserted between 2219 and 2281 of CYVaV after removal of 2220-2280 of CYVaV. The inserts were stable in N. benthamiana after four months (the entire duration of the experiment) except that the trial for Erwinia GyrA has not reached four months yet. Erwinia GyrA was stable after 1 month and the trial was ongoing.

Q. Example of a Stable Insert Targeted Against CTV

FIG. 51A shows the sequence and secondary structure of a hairpin-like structure called mmck6.2. This insert include a sequence targeting CTV, which is the 27 bases extending on the right hand side of the insert from the bottom of the insert to just below the apical loop as the insert is shown in FIG. 51A. The MFE (ΔG) of the insert is −29.64 kcal/mol as determined by the RNAfold software. The APE is 0.23.

The sequence of mmck6.2 is (SEQ ID NO:129):

gguuucaguauauuucgucaucaccuuccuaacggaagugauggacgaa
auuaaugaaacc

To investigate the stability of mmck6.2 in CYVaV1, we inserted mmck6.2 into the CYVaV1 genome by replacing the original viral sequence between 2220 to 2280 with mmck6.2. We then inoculated the resulting recombinant CYVaV1 into Mexican lime citrus plants by vacuum-infiltration. Three months post-inoculation, we performed RT-PCR analysis of the systemic leaves of the inoculated plants to identify recombinant CYVaV1 positive plants. We have monitored the viral fitness and the stability of the insert in the positive plants over a long period of time (17 months at the time of writing). The experiment is ongoing.

After one year post-inoculation, we performed RT-PCR analysis and Sanger sequencing of the systemic leaves of the positive plants. The results (FIG. 51B) showed that all 4 positive plants have a single RT-PCR product. The Sanger sequencing of the RT-PCR product confirmed that the insert is intact. This suggests that mmck 6.2 is stable in the CYVaV1 genome and can be maintained over an extended period of time in citrus plants. The polynucleotide sequences shown in FIG. 51B are SEQ ID NO:130 (Analysis of #7) and SEQ ID NO:131 (Analysis of #20). The sequence of mmck6.2 (SEQ ID NO:129) is underlined:

SEQ ID NO: 130:
ttcgttgguuucaguauauuucgucaucaccuuccuaacggaagugaug
gacgaaauuaaugaaaccagttaatg
SEQ ID NO: 131:
agcttcgttgguuucaguauauuucgucaucaccuuccuaacggaagug
auggacgaaauuaaugaaaccagttaatgta

FIG. 51C shows: Panel A: Northern blot of RNA extracted from two infected citrus plants after ˜one year of infection. Panel B: Reverse-transcriptase (RT)-PCR is used to amplify a segment of the infecting VIGS vector. For this plant, 10 leaves were assayed. Only a single product of the correct size for the vector with the insert was found. Panel C: Total RNA from young leaves of the infected plant is subjected to batch sequencing of the insert region. Data is from 15-month citrus showing no indication of single “double peaks” that would denote base changes or multiple double peaks that would indicate a portion of the population contain deletions. In addition, at least 10 clones are generated for individual sequencing and no changes from the initial VIGS vector must be found. SEQ ID NO: 99 shown in Panel E is missing the first G of the insert only because of the boundaries of the screenshot. The first peak in the insert band (not included in the sequence) is this missing G. Panel D: A photograph of a mexican lime plant infected with recombinant CYVaV1 including mmck6.2 showing a lack of symptoms of infection in the plant.

R. APE of Exemplary Inserts

FIG. 52 Panel A show a flow chart for how the data in Panel B was generated. The predicted structure is determined by entering the sequence for the insert into RNAfold. The RNAfold software produces a list of positional entropy values of the individual bases. The positional entropy values are given a grey scale and sorted form highest PE (darkest grey) or lowest PE (light grey)

APE values for various hairpin-like structures (and some additional non-hairpin-like structures) inserted into CYVaV are shown in Panel B. Sorted positional entropy is shown for each insert (dark grey to lightest grey). The first four hairpin-like structures (boxed) were duplicates of naturally occurring hairpin-like structures 1 through 4 of CYVaV (see FIG. 49, Panel C). Other hairpin-like structures and non-hairpin-like inserts are arranged in order of descending APE (from 1.30 to 0.07). Mimic hairpin-like structures begin with a number (1 or 4) that identifies the hairpin-like structure of CYVaV being mimicked. Thick lines beneath each lane denote the length of each insert (number of nucleotides shown at right). Single and double asterisks denote inserts that were not stable in the VIGS vector. The double asterisk denotes an unstable hairpin-like structure that had three consecutive G:C pairs at the base and five consecutive G:C pairs in the apical region contributing to the very low APE of 0.07. Eliminating two nucleotides on the 3′ side of the hairpin in the apical region generated a stable hairpin with no consecutive apical G:C pairs and the APE value changed to 0.13. The thick black arrow denotes unstable hairpin-like structure (M2250gfp30ext) with a relatively low APE value of 0.39 that contained a clustering of higher entropy nucleotides.

S. Example of a Stable Hairpin-Like Structure (GFPmmck59)

FIG. 63 shows the sequence and secondary structure of GFPmmck59. This insert has 59 nucleotides including a region targeted against green fluorescent protein (GFP) expression. Some plants or recombinant pathogens used in experimental trials have been modified to express GFP. Suppressing GFP expression can indicate replication, movement or effectiveness of a vector with a GFP suppressing insert.

This insert is stable. The minimum free energy (MFE) is −30.20 kcal/mol. GFPmmck59 has 0 (0%) bases with positional entropy (PE) greater than 1.0. The APE is 0.07 and the highest PE of any base is 0.995. The standard deviation of PE is 0.21.

The sequence of GFPmmck59 is shown below (SEQ ID NO:116). The first 27 bases of the sequence are the targeting sequence.

tgaagcggcacgacttcttcaagagcgataactcgccttgacagaagtc
caacgcttca

T. Example of an Unstable Hairpin-Like Structure with Low APE (GFPmmck63)

FIG. 64 shows the sequence and secondary structure of GFPmmck63. This insert has 63 nucleotides including a region targeted against green fluorescent protein (GFP) expression. Despite having a very low APE of 0.07, no bases with PE greater than 1, and a standard deviation of PE, this insert is relatively unstable. The MFE 15−36.60 kcal/mol. GFPmmck63 has 3 (1.6%) bases with positional entropy (PE) greater than 1.0. The APE is 0.25 and the highest PE of any base is 1.14. The standard deviation of PE is 0.37.

One region of this insert has 5 consecutive G-C pairs. A variant with fewer G-C pairs had a higher APE, but was stable. This example suggests that a large number (e.g. more than 4) consecutive G-C pairs leads to instability, even in a hairpin-like insert meeting other guidelines.

The sequence of GFPmmck63 is shown below (SEQ ID NO:117). The first 30 bases of the sequence are the targeting sequence.

tgaagcggcacgacttcttcaagagcgccataactggcgccttgacaga
agtccaacgcttca

U. Second Example of an Unstable Hairpin-Like Structure (M2250GFP30ext)

FIG. 62 shows the sequence and secondary structure of M2250GFP30ext. This insert is relatively unstable. The MFE is −28.57 kcal/mol. M2250GFP30ext has 9 (13.8%) bases with positional entropy (PE) greater than 1.0. The APE is 0.39 and the highest PE of any base is 1.97. The standard deviation of PE is 0.52.

The sequence of M2250GFP30ext is shown below (SEQ ID NO:115):

ggttagggtaactcactgaagcggcacgacttcttcaagagcgccattc
agtgagcaacctaacc

V. Third Example of an Unstable Hairpin-Like Structure with Low APE

FIG. 67 shows the sequence and secondary structure of BBLv2-1. BBLv2-1 has 198 nucleotides. 3 nucleotides (1.5%) have PE greater than 1. The APE is 0.18 and the standard deviation of PE is 0.3 but this insert is unstable.

The sequence of BBLv2-1 is shown below (SEQ ID NO:132):

cuggaaaauucgauuacucaaagugccuaaaaucguaacaacuuguaug
aucuauaggccuggauccaaacaauacguggcucaaauacuugagaaau
uuuuuucucauguauuugagccuaaaccuugauuuggauccaucuccua
ucgaacaccaaguguuaaacuuuuuagcagagggaguaaucgaaauucc
ag

The sequence of BBLv2-1 after deletion is shown below (SEQ ID NO:133):

cuggaaaauucgauuacucaaagugucgaaauuccag

An analysis of BBLv2-1 indicated that modification of the vector in an infected plant involved removal of a region having a bulge (no bases on one side, 1 base on the other side), 5 A-T base pairs (A-U base pairs are included as A-T base pairs), an asymmetric loop with 7 bases (counting both sides) and 3 more A-T base pairs. The DG of the asymmetric loop was positive (2.7 kcal/mol) and the DG of the region from the bulge to the asymmetric loop (including both the bulge and the asymmetric loop) had a positive DG of 0.4 kcal/mol. Without intending to be limited by theory, instability of this inert may have been caused by the locally positive DG of this region.

This example suggests a number of potential design parameters such as that the number of consecutive A-U base pairs should be less than 5 or that the DG of a region including two loops and the stack between them should not be positive.

W. Example of a Hairpin-Like Structure of CTV.

The stability of hairpin-like structures that mimic hairpin-like structures in the CYVaV1 and OULV genomes has been previously demonstrated by their ability to remain stable, for example when inserted to replace the sequence between 2220 to 2280 (i.e. inserted at 2219/2281) or inserted at 2304/2305, 2319/2320 or 2330/2331 of CYVaV1. To investigate whether this rule also applies to hairpins that originate from an unrelated virus, we subjected every 2 kb of the CTV genome to secondary RNA structure prediction using mfold software obtained from the “UNAFold Web Server” (http://www.unafold.org/mfold/applications/rna-folding-form-v2.php) in search of hairpins that resemble a naturally-occurring hairpin of CYVaV1 in terms of size and shape. As a result, we identified a 128 nt hairpin-like structure, CTV-insert-natural-V2.2, which forms a stack-loop structure with internal asymmetrical loops. The overall structure of this hairpin resembles a smaller version of a hairpin-like structure 1 of CYVaV1. CTV-insert-natural-V2.2 has a ΔG of −43.6 kcal/mol (mfold) or −46.96 (RNAfold) and APE of 0.359.

The sequence of CTV-insert-natural-V2.2 is (SEQ ID NO:118):

ggggguuuauguuuggcaaagaaaguguuggaacuguuagucaagcggg
ugguugaaucguuuucucguuugaagcggaaaaccgcucguuuaacguc
cuucgcuaauuuguugcuugcgaggcucuc

FIG. 65 shows the sequence and secondary structure of CTV-insert-natural-V2.2 as predicted by “mfold” and drawn by “RNA2Drawer.”

To test the stability of CTV-insert-natural-V2.2 in CYVaV1, we inserted this hairpin-like structure into the CYVaV1 genome by replacing the original viral sequence between 2220 to 2280 of CYVaV1 with CTV-insert-natural-V2.2. We then inoculated the resulting recombinant CYVaV1 into N. benthamiana plants by syringe infiltration and monitored viral fitness as well as the stability of the insert over a period of time. Three weeks post-inoculation, we performed RT-PCR analysis and Sanger sequencing of the systemic leaves of all symptomatic plants. The results (FIG. 66) show that all 9 symptomatic plants tested have a single RT-PCR product that migrates slower than CYVaV1 wt control, suggesting the presence of recombinant CYVaV1 with CTV-insert-natural-V2.2. The Sanger sequencing of the RT-PCR product confirmed that the insert is intact in all 9 symptomatic plants. This experiment is ongoing.

In some embodiments, an insert is provided that targets one or more viral and/or fungal and/or bacterial pathogens. In some embodiments, a hairpin or short RNA sequence (about 100 nt or less, e.g. between about 20 nt and about 80 nt, or between about 30 nt and about 60 nt, or about 30 nt) insert is provided that generates an siRNA that directly targets CVEV, since CVEV is known to slightly intensify the yellowing impacts of CYVaV and to enable transport of CYVaV between trees. In some embodiments, a hairpin insert is provided that targets CTV, since CTV is a highly destructive viral pathogen of citrus (second only to CLas). In other embodiments, an insert is provided that targets another citrus (or other) virus. In some embodiments, an insert is provided that targets a fungal pathogen(s), given that such pathogen(s) are able to take up siRNAs from the phloem. In some embodiments, an insert is provided that targets a bacterial pathogen, given that such pathogen(s) are able to take up siRNAs from the phloem.

In some embodiments, the CYVaV-based (or other iRNA) vector includes an insert(s) engineered to modify a phenotypic property of a plant that emanates from gene expression in companion cells. In one implantation, an insert is provided that triggers dwarfism, so that the fruit is easier to harvest and growth space requirements are reduced. Additional and/or other traits may also be targeted as desired. The iRNA vectors of the present disclosure comprising 1, 2, 3 or more inserts demonstrate stability and functionality.

In some embodiments, an RNA vector is the same as, essentially the same as, or substantially similar to, an RNA vector that is produced by a method described herein but made differently, for example, by a synthetic manufacturing method that might or might not pass through an equivalent of a wild type or parental form. For example, rather than actually truncating or stabilizing a wild type RNA vector, an RNA may be manufactured synthetically that has the same nucleic acid sequence as a truncated or stabilized wild type RNA vector. In this case, it may not be necessary to manufacture the full wild type vector and then truncate or stabilize it but rather the truncated or stabilized structure can be manufactured directly. Similarly, it is not necessary to produce an RNA backbone and then add a heterologous insert to the RNA backbone. Instead, an RNA vector may be manufactured directly with the insert present. Thus descriptions of actions or states based on verbs such as to insert, to truncate, or to stabilize, or referring to starting from parental or wild type structures, should be interpreted notionally so as to include a resulting nucleic acid sequence whether that action was actually performed or not and whether the specified starting material was actually used or not. For example, an optionally truncated or stabilized parental structure with an added heterologous element may instead be made by determining its nucleic acid sequence and synthetically manufacturing an equivalent or similar molecule was created by some other sequence of steps or method.

International Publication Number WO 2021/097086 A1, Plant Vectors, Compositions and Uses Relating Thereto, published on May 20, 2021, is incorporated herein by reference. As described therein, an RNA vector may be derived from citrus yellow vein associated virus (CYVaV) or one of its relatives. Relatives of CYVaV include other umbravirus-like associated RNAs (ulaRNA) as described in Liu J. et al. (2021), Structural Analysis and Whole Genome Mapping of a New Type of Plant Virus Subviral RNA: Umbravirus-Like Associated RNAs, Viruses 13:646, which is incorporated herein by reference.

All identified publications and references mentioned herein are hereby incorporated by reference to the same extent as if each such publication was specifically and individually indicated to be incorporated by reference in its entirety. While the disclosure has been described in connection with exemplary embodiments, it will be understood that it is capable of further modifications and this application covers any variations, uses, or adaptations following, in general, the principles of the disclosure and including such departures as come within known or customary practice within the art to which the disclosure pertains and as may be applied to the features hereinbefore set forth.

RNA sequences presented in this specification or the figures may use either “t” or “u” to denote uracil. Sequences may be presented in upper case or lower case letters. There is no significance attached to the case (upper case or lower case) of the sequence unless noted in the description of the sequence.

1. A ribonucleic acid (RNA) vector comprising a heterologous segment(s), wherein said heterologous segment(s) comprises a structure and/or sequence that substantially mimics one or more aspects of a corresponding structure and/or sequence of a wild-type RNA molecule.

2. The RNA vector of claim 1, wherein the exogenous segment mimics one or more aspects of the secondary structure of the corresponding structure and/or sequence.

3. The RNA vector of claim 1 wherein the wild-type RNA molecule is a wild-type of the RNA vector or a relative of the RNA vector.

4. The RNA vector of claim 1 wherein the corresponding structure and/or sequence comprises paired portions and optionally looped portions and/or is a hairpin portion (which may be a hairpin-like portion or a structure containing multiple hairpins or hairpin-like portions) of the wild type RNA vector.

5. The RNA vector of claim 1 wherein the heterologous segment has a minimum free energy similar to the corresponding structure and/or sequence of a wild-type RNA molecule.

6. The RNA vector of claim 1 wherein the heterologous segment has an active portion optionally wherein the active portion is provided on one side of the heterologous element.

7. The RNA vector of claim 6 wherein the active portion comprises one or more siRNAs.

8. The RNA vector of claim 6 wherein the active portion is effective against a plant pathogen or to suppress expression or a gene in the plant.

9. The RNA vector of claim 1 wherein the RNA vector is an iRNA or ulaRNA and the wild-type RNA molecule in an iRNA or ulaRNA.

10. The RNA vector of claim 9 wherein the RNA vector is derived from CYVaV and the wild-type RNA molecule is a Class 2 ulaRNA, for example CYVaV or OULV.

11. The RNA vector of claim 10 wherein the wild-type RNA molecule is CYVaV.

12. The RNA vector of claim 1 wherein the heterologous segment has one or more of a) a minimum free energy within 10 kcal/mol, or within 5 kcal/mol of a wild-type hairpin-like structure of similar length or a line of best fit between multiple wild-type hairpin-like structures, b) a length within 4 bases, or within 2 bases, or the same as a wild-type hairpin-like structure, and c) an arrangement of paired and looped regions that is substantially the same as, or the same as, a wild-type hairpin-like structure.

13. The RNA vector of claim 1 having a base alteration associated with frameshifting.

14. The RNA vector of claim 13 wherein the base alteration is one or more of i2631A, U664C, A652G and A604U.

15. The RNA vector of claim 1 wherein the heterologous segment has a sequence with 60 or more, 70 or more, 80 or more or 100 or more, bases or changes in bases relative to the wild-type hairpin-like structure.

16. The RNA vector of claim 1 wherein the heterologous segment contains two or more base-paired regions and one or more non-base-paired regions.

17. The RNA vector of claim 16 wherein the heterologous segment comprises one or more non-base-paired regions each located between two base-paired regions.

18. The RNA vector of claim 17 wherein the non-base-paired region is double sided and optionally forms a “loop” between two base-paired regions.

19. The RNA vector of claim 1 wherein the heterologous segment comprises at least one non-base-paired region with bases, or base changes relative to the wild-type RNA vector, optionally in the form of a “loop” between two base-paired regions.

20. The RNA vector of claim 1 wherein the heterologous segment is inserted a) in the location of, and as a replacement for, a hairpin-like structure that the heterologous element mimics, b) in the location of, and as a replacement for, a hairpin-like structure that the heterologous element does not mimic, or c) at a location that previously did not have any hairpin-like structure.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A ribonucleic acid (RNA) vector drived from an RNA virus whose nucleotide sequence has been modified to comprise a heterologous RNA segment, wherein said heterologous RNA segment:
(A) comprises a length of between about 40-300 nucleotides; and,
(B) is capable of forming a hairpin-like structure, wherein:
(1) the nucleotides of said hairpin-like structure exhibit an Average Positional Entropy (APE) similar to the APE of one or more naturally occurring hairpin-like structure of said virus, a relative of said virus, or another RNA virus; and/or
(2) said hairpin-like structure exhibits a minimum free energy (ΔG) relative to length that is within 10 kcal/mol, or within a range between −5 and +15 kcal/mol, of the minimum free energy relative to length exhibited by said one or more naturally occurring hairpin-like structures of said virus.

40. The RNA vector of claim 39 wherein the exogenous segment has a minimum free energy within 10 kcal/mol, or within a range between −5 and +15 kcal/mol, of a naturally occurring hairpin-like structure of sad virus that is within 10% of the size of the exogenous segment.

41. The RNA vector of claim 39 wherein the exogenous segment has a minimum free energy within 10 kcal/mol, or within a range between −5 and +15 kcal/mol of the minimum free energy predicted by the length of the exogenous segment and a line of best fit though a plot of minimum free energy vs. length for a plurality of naturally occurring hairpin-like structures of said virus.

42. The RNA vector of claim 39 wherein the virus is a plus-sense RNA plant virus.

43. The RNA vector of claim 39 wherein the virus is a ulaRNA, CTV or TRV.

44. (canceled)

45. (canceled)

46. (canceled)

47. (canceled)

48. (canceled)

49. (canceled)


SEQUENCE LISTING (140 sequences)

SEQ ID NO: 1 - RNA, 2692 nt, unidentified
  source 1..2692: mol_type = viral cRNA; note = Citrus yellow vein-associated virus [CYVaV]
gggtaaatatggatccttcatctttgccccgtgcctgttggcatcatgccagacaggtgtttcgagcatcaactagcttctcaagagaggtggttcgcgctgctcgtagatgggttaccatgcccaccagtcgccatgcatatgacttttcaacgagtctaggcattgtgattgctgagcctgcagctcgtttacgacgccgtctgccctctgtacgaaagtgcgcagagaagttagtagtccacaagcaagtcgacactttggtggacgaatggtgctctggaattcccaaccctgatatcgtagaagttggttgggcactccgtctgagggaccgtttcggtcttcctcccgcttctgagcctacccggctcagtggtgagagatgggtgctcaaacaactcaatggggtagatcctgagtcatggaatgctgatcttggtaggtcagttcatatccaaggagactacgccccagggaggaatgcccatatcgctcaggtcgcggcgaccttgtggttaactaggaccttgcatgacaaggccttggctcgccaccagggttttcgcgatttgcagtgattggggtcgacgggctagaggcaaaagcagtgcctctagcttctggactccgactgcttccggttccgcgacccggacaaagtcgacgactgtctcagaccttgttacttccaacacctcgtgctcaattcgtgaatcacgcgtgctcggctaacaaccttggacgtgtgatgaccacacgtgtgttgcagtacaagggccgagatccgatccttccctcttctgaagcccttcaccgacttaaccttcggatagctgagctatataggtctagaccttctaccgtctatccattaagttatgaagggtttctcaattgctatgaaggccgacagcgtactcgttacgcccaagccgtcgagcagttgatgcggtccactcttgagccgaaagatgcgcgagttgaaacgttcattaagaacgagaaatttgactgggcgttgaaaggggaggaggctgatcctcgagcaatccaaccaaggaagccgaaatatttggctgaggttggacggtggttcaaacctttggagcgaatcatctacaaggatctcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgtaagtctcccactctccagcgctatctcaaatggacactccgcaaccatggcgtcgcctcctgcaaagaattgtcatatgagtatgaggttgttggccggagaatgagtggtgacatggacactgcattgggcaactgcgtcattatgtcgatacttacatggtttatgcttagtgaacttggcattaagcatgaattattcgataatggtgacgattgtttgttcatttgcgagtctcacgacgtccccagccccgaggtaattacaaactggttttcggactttgggtttgtggttaggttggaaggcgtcacgtccgtgtttgagcgtattgagttttgccaaacttccccagtatggactgagaggggttggctgatgtgtaggaatattaagtcattgagtaaagaccttacgaatgttaattcgtgcacgggctccacgattgaatatacccactggttgaaagcagtgggaaagtgcgggtcaatactcaatgctggtgtacctatatttcagtcctttcacaacatgctggaaaggcttggcactaactctcgtattgatcgaggggttttcttcaaatcagggctagttaatctcattcgtgggatggacaggcagcctgacgttgacatcactacttccgctcggctttctttcgaagtggcattcgggataacacccgggatgcaattggctattgaacggtactatgactctgtcatgggctcgctgagtaaaatagaaacaactaagtggccaattgaactaagaaaggaatacgaacacggaagtgagtggtacgaggacttaggcgtcctaggatgaatagggtcattggtttaccgatgatacctgttcagaataggattgctcgagcttcgttggttagggtaactcacataccttcttccataactggaaaaggtcgtgtgagcaacctaaccagttaatgtaggtgtctttccgtatctagtcacgatggtaagcaacccgtttatctgtacggcgctcacccgtgggtaggaaggtgaaggttttgtgtcctttaggtcttggacagtctgcgggcttgggaacgacgccccgctagcaacgtactgctctcctaccggactggtagcttaattgtcatcttggagcgatagcactgtgggcctcacccttcgcgcgttggacgtgttgcgtgccccccacagatttgtgaaactctatggagcagttccgcgagccagaagggaggatggccgcctggcgtaatccaggagctctggggggcttgtactcagagtagcattctgctttagactgttaactttatgaaccacgcgtgtcacgtggggagagttaacagcgccc

SEQ ID NO: 2 - RNA, 225 nt, unidentified
  source 1..225: mol_type = viral cRNA; note = Citrus yellow vein-associated virus [CYVaV]
  misc_feature 1..225: note = Polynucleotide Sequence of the 3 prime end of CYVaV
tcttggagcgatagcactgtgggcctcacccttcgcgcgttggacgtgttgcgtgccccccacagatttgtgaaactctatggagcagttccgcgagccagaagggaggatggccgcctggcgtaatccaggagctctggggggcttgtactcagagtagcattctgctttagactgttaactttatgaaccacgcgtgtcacgtggggagagttaacagcgccc

SEQ ID NO: 3 - RNA, 84 nt, unidentified
  source 1..84: mol_type = viral cRNA; note = Citrus yellow vein-associated virus [CYVaV]
  misc_feature 1..84: note = Polynucleotide Sequence of the 3 Prime Cap Independent Translation Enhancer (3 CITE) of CYVaV (bases 2468 to 2551)
tcttggagcgatagcactgtgggcctcacccttcgcgcgttggacgtgttgcgtgccccccacagatttgtgaaactctatgga

SEQ ID NO: 4-5 - skipped (2 entries)

SEQ ID NO: 6 - RNA, 570 nt, unidentified
  source 1..570: mol_type = viral cRNA; note = Citrus yellow vein-associated virus [CYVaV]
  misc_feature 1..570: note = p21 Encoding Sequence of CYVaV
atggatccttcatctttgccccgtgcctgttggcatcatgccagacaggtgtttcgagcatcaactagcttctcaagagaggtggttcgcgctgctcgtagatgggttaccatgcccaccagtcgccatgcatatgacttttcaacgagtctaggcattgtgattgctgagcctgcagctcgtttacgacgccgtctgccctctgtacgaaagtgcgcagagaagttagtagtccacaagcaagtcgacactttggtggacgaatggtgctctggaattcccaaccctgatatcgtagaagttggttgggcactccgtctgagggaccgtttcggtcttcctcccgcttctgagcctacccggctcagtggtgagagatgggtgctcaaacaactcaatggggtagatcctgagtcatggaatgctgatcttggtaggtcagttcatatccaaggagactacgccccagggaggaatgcccatatcgctcaggtcgcggcgaccttgtggttaactaggaccttgcatgacaaggccttggctcgccaccagggttttcgcgatttgcag

SEQ ID NO: 7 - protein, 190 aa, unidentified
  source 1..190: note = Citrus yellow vein-associated virus [CYVaV]
  REGION 1..190: note = Amino Acid Sequence of CYVaV p21 Protein
MDPSSLPRACWHHARQVFRASTSFSREVVRAARRWVTMPTSRHAYDFSTSLGIVIAEPAARLRRRLPSVRKCAEKLVVHKQVDTLVDEWCSGIPNPDIVEVGWALRLRDRFGLPPASEPTRLSGERWVLKQLNGVDPESWNADLGRSVHIQGDYAPGRNAHIAQVAATLWLTRTLHDKALARHQGFRDLQ

SEQ ID NO: 8 - RNA, 1407 nt, unidentified
  source 1..1407: mol_type = viral cRNA; note = Citrus yellow vein-associated virus [CYVaV]
  misc_feature 1..1407: note = p81 Encoding Sequence of CYVaV
atgaccacacgtgtgttgcagtacaagggccgagatccgatccttccctcttctgaagcccttcaccgacttaaccttcggatagctgagctatataggtctagaccttctaccgtctatccattaagttatgaagggtttctcaattgctatgaaggccgacagcgtactcgttacgcccaagccgtcgagcagttgatgcggtccactcttgagccgaaagatgcgcgagttgaaacgttcattaagaacgagaaatttgactgggcgttgaaaggggaggaggctgatcctcgagcaatccaaccaaggaagccgaaatatttggctgaggttggacggtggttcaaacctttggagcgaatcatctacaaggatctcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgtaagtctcccactctccagcgctatctcaaatggacactccgcaaccatggcgtcgcctcctgcaaagaattgtcatatgagtatgaggttgttggccggagaatgagtggtgacatggacactgcattgggcaactgcgtcattatgtcgatacttacatggtttatgcttagtgaacttggcattaagcatgaattattcgataatggtgacgattgtttgttcatttgcgagtctcacgacgtccccagccccgaggtaattacaaactggttttcggactttgggtttgtggttaggttggaaggcgtcacgtccgtgtttgagcgtattgagttttgccaaacttccccagtatggactgagaggggttggctgatgtgtaggaatattaagtcattgagtaaagaccttacgaatgttaattcgtgcacgggctccacgattgaatatacccactggttgaaagcagtgggaaagtgcgggtcaatactcaatgctggtgtacctatatttcagtcctttcacaacatgctggaaaggcttggcactaactctcgtattgatcgaggggttttcttcaaatcagggctagttaatctcattcgtgggatggacaggcagcctgacgttgacatcactacttccgctcggctttctttcgaagtggcattcgggataacacccgggatgcaattggctattgaacggtactatgactctgtcatgggctcgctgagtaaaatagaaacaactaagtggccaattgaactaagaaaggaatacgaacacggaagtgagtggtacgaggacttaggcgtcctagga

SEQ ID NO: 9 - protein, 469 aa, unidentified
  source 1..469: note = Citrus yellow vein-associated virus [CYVaV]
  REGION 1..469: note = Amino Acid Sequence of CYVaV p81 Protein
MTTRVLQYKGRDPILPSSEALHRLNLRIAELYRSRPSTVYPLSYEGFLNCYEGRQRTRYAQAVEQLMRSTLEPKDARVETFIKNEKFDWALKGEEADPRAIQPRKPKYLAEVGRWFKPLERIIYKDLSKRLYGEGAEPCIAKGLNALESGATLRRKWEKFSSPVCVSLDASRFDLHVSVGMLKFTHKLYDYYCKSPTLQRYLKWTLRNHGVASCKELSYEYEVVGRRMSGDMDTALGNCVIMSILTWFMLSELGIKHELFDNGDDCLFICESHDVPSPEVITNWFSDFGFVVRLEGVTSVFERIEFCQTSPVWTERGWLMCRNIKSLSKDLTNVNSCTGSTIEYTHWLKAVGKCGSILNAGVPIFQSFHNMLERLGTNSRIDRGVFFKSGLVNLIRGMDRQPDVDITTSARLSFEVAFGITPGMQLAIERYYDSVMGSLSKIETTKWPIELRKEYEHGSEWYEDLGVLG

SEQ ID NO: 10-16 - skipped (7 entries)

SEQ ID NO: 17 - RNA, 166 nt, unidentified
  source 1..166: mol_type = viral cRNA; note = Citrus yellow vein-associated virus [CYVaV]
  misc_feature 1..166: note = Polynucleotide Sequences Of Recoding Frameshift Sites of CYVaV
tcgctcaggtcgcggcgaccttgtggttaactaggaccttgcatgacaaggccttggctcgccaccagggttttcgcgatttgcagtgattggggtcgacgggctagaggcaaaagcagtgcctctagcttctggactccgactgcttccggttccgcgacccgga

SEQ ID NO: 18 - RNA, 25 nt, unidentified
  source 1..25: mol_type = viral cRNA; note = Citrus yellow vein-associated virus [CYVaV]
  misc_feature 1..25: note = Polynucleotide Sequences Of Recoding Frameshift Sites of CYVaV
caaagtcgacgactgtctcagacct

SEQ ID NO: 19 - RNA, 34 nt, unidentified
  source 1..34: mol_type = viral cRNA; note = Citrus yellow vein-associated virus [CYVaV]
  misc_feature 1..34: note = Polynucleotide Sequences Of Recoding Frameshift Sites of CYVaV
aggtcttggacagtctgcgggcttgggaacgacg

SEQ ID NO: 20 - RNA, 3132 nt, unidentified
  source 1..3132: mol_type = viral cRNA
  misc_feature 1..3132: note = Polynucleotide Sequence of iRNA of Fig Tree
aaatatggattcgatatcaatgcccgtcgcctgctggtcaaaagccaggcaggtcttgcgtacaccagctaacttttccaaaggggtagtgaaggctgcgtaccggtgggtcaacatgcccagagccaaatatgtcagagatgtctccacgagtcttggcatagttgtcgctgagcctgttgctgccgtgcgccgttagatgccttcgataagcagccttgcggaggagttggtaacacgccagagcgtcgacactctggtggacgattggtgtctcggactttccaaccctgacaacaacgtggaggttggttgggcacttcgtctgagggaccgctttggtcttcctcccgcctctgagcccacaaggctcagtggtgagagatgggtgcttaaacaactcaatggggtagacccggagtcgtggaatgttgatctgcaaagcgttttcgaagacgctcaggatgacttccatcgggactacgccccaaggaggaatgcccaaatcgctcaaattgcggcaaccctatggcttacaaagaccttagtcgataaggctttagcacgccatcaggattttcgcagtttgcagtgattggggtcgacgggctagaggctaaagcagtgcctctggctgctggactccgactgcttccggttccgcggcccggacaaagccgacggctgtctcaaaccttgctactccctactccccgtgctcaatttgtcaatcacgctaactcaggtaataatttggggcgtgttttgaccacacgggtgatgcaatacaaaggccgagacccgatactaccctcccaggaagccctgcgcaaacttaaccttcggataggacagttgtataagtctagaccatccactgtctatcccctgagttatgatgggtttcttaattgttatgatggccgacagcgtactcgctacgctcatgccgtcgagcaattgatgggtgccgctctgaccccaaaagatgcgcgagttgagacgttcattaagaacgagaagtttgattggttgttgaagggagacgaggctgatcctcgtgcaatccaacctaggaagccgaaatatttggccgaggttggtcgatggttcaaaccgttggagcgaatcatctacaaggatctcagtttgcgtttgtacggtgataacgctgaaccttgcattgccaaaggcttaaatgcattggaatcaggggctacgttgagacgtaaatgggaaaagttcgctaatcctgtttgtgtttcattggatgcttctcgtttcgacctgcacgtaagtgttggcttgttaaagttcacgcataaattgtacaactattactgcaagtctcccactcttcaacgatatctcaaatggacactccgcaactccggtatcgcctcctgtaaggaaaaatcatatgcgtatgaggttgaaggccgtagaatgagtggcgacatggacaccgcattaggcaactgtatcatcatgagattattaacttggtttatgcttagcgaacttggcgtgcggcatgagcttttcgataatggtgatgactgtttgtttatttgtgaaaaagaagacgttcctagtgctgaggtaatcacgaactggtttacggattttgggtttgtggttaagctagaaggcgtcacgtccgtgtttgagcgcattgagttctgtcagacctcaccagtatggactgcgaggggatggctgatgtgtagaaacatcaagtcattgagtaaagatttaacgaatgttaattcgtgcactggttctgccgttgaatacactcattggttgaaggcggtgggcaagtgtggatctatactcaatgctggtgtgcccatatttcagtcctttcacaacatgttggtcaggttgggcacgaattcgcgtatagatcgcggggtattctttaggtgtggacttgttaatctcattctgggatggacagacaacctgaaagttgagatcactacttccgctcgtctttcttttgaagtggcattcgggatcactcccggcatgcaattggctattgagcaattttatgactcagtcgtgggccctctgggtaaaataaaatctgtaaaatggccaatagatctaagaaaggaatacgattacggaagcgcgtggttcgaagaccaaggcgtcctagggtgaacaaggaactcggattaccgatgacacctgttcaaactagaatggttcggtcaacgttgaccaaggagaccaacataccttctactgcaaatagcggtcgggaggctgtttgggcttgttggccaatcaactttagtgtctttccgcaactagcctcactcgtgaataaaccgttatactggcgtgtgtccagtgtgcaagttgcaatggagccggcgatgtctacttccacccaacattgtggagttggtctcagttcttctggggccttcactaacggtgatgggttcggtaacgtctttaagctcttgcgttcttgtaactatacgcggcgctctcccgtgggaggaaacgtgatggtcaaatggcccatctgcatgcccttcattcttaacgatgatgcgcacaagaacacaggattaaccgcctgtgtgatcattgcagtcaccaatactggtgtgctaactggtcaatcttggacggagattcttttgaatgtggagtatgtagtgggtgcatagacagtctgcgggcttgggaacgacgccccgctagcaacgtactgctctcctaccggactggtagccgtttagttatcttggagcgatagcactgtgagcctcactcaacgcgcgatggacgtggcgagtgcccctcagagatttgtgaaactctatagagctatttcgcgagccagaagggaggatggccacctggtgtaagccaggtatccccggggggcttgtactcggggtcgcattactgcttagaccacaaggtagggttcgcatcttggaactgaccctatgaccttgtgggtgccctaaccggactggtagccgtttaatatcttggagcgattagcacgtgtgagccctcactcaacggcgcgattggacgtggcgagtgcccctcagagtaatctgcagagctccggcagtcgtgggaggcaaggca

SEQ ID NO: 21 - RNA, 3275 nt, unidentified
  source 1..3275: mol_type = viral cRNA
  misc_feature 1..3275: note = Polynucleotide Sequence of iRNA of Fig Tree
ctcccacgactgccggagctctgcagaattccaccgggggtacctggcttacaccaggtggccatcctcccttctggctcgcggaatagctctatagagtttcacaaatctctgaggggcactcgccacgtccatcgcgcgttgagtgaggctcacagtgctatcgctcccagaattcgggataaatatggaagaaacttctttgcccaaagcctgctggatcaaaagccaggcaggtcttgcgtacaccagctaacttttccaaaggggtagtgaaggctgcgtaccggtgggtcaacatgcccagagccaaatatgtcagagatgtctccacgagtcttggcatagttgtcgctgagcctgttgctgccgtgcgccgtcagatgccttcgataagcagccttgcggaggagttggtaacacgccagagcgtcgacactctggtggacgattggtgtctcggactttccaaccctgacaacaacgtggaggttggttgggcacttcgtctgagggaccgctttggtctccctcccgcctctgagcccacaaggctcagtggtgagagatgggtgcttaaacaactcaatggagtagacccggaatcttggaatgacgactatgcgttcgaagacgctcaggaggattttcaacgggaatacgtcccgggaaggaatgcccatattgctgcaactgcggcaactctatggctgacaaagaccttgtatgacaaggctttagttcgccatcagggttttcgcagtttgcagtgattggggtcgacgggctggaggctaaagcagtgcctccagctgctggactccgactgcttccggttccgcggcccggacaaagccgacggctgtctcagaccttactacttcctactccccgtgctacttttgtcaatcatgcaaattcaggcaataatcttgagcgtgttttgaccacacgggtgatgcaatacaaaggccgagacccgatactaccctcccaggaagccctgcgcaaacttaaccttcggataggacagttgtataagtctagaccatccactgtctatcccctgagttatgatgggtttcttaattgttatgatggccgacagcgtactcgctacgctcatgccgtcgagcaattgatgggtgccgctctgaccccaaaagatgcgcgagttgagacgttcattaagaacgagaagtttgattggttgttgaagggagacgaggctgatcctcgtgcaatccaacctaggaagccgaaatatttggccgaggttggtcgatggttcaaaccgttggagcgaatcatctacaaggatctcagtttgcgtttgtacggtgataacgctgaaccttgcattgccaaaggcttaaatgcattggaatcaggggctacgttgagacgtaaatgggaaaagttcgctaatcctgtttgtgtttcattggatgcttctcgtttcgacctgcacgtaagtgttggcttgttaaagttcacgcataaattgtacgactattactgcaagtctcccactcttcaacgatatctcaaatggacactccgcaactccggtatcgcctcctgtaaggaaaaatcatatgcgtatgaggttgaaggccgtagaatgagtggcgacatggacaccgcattaggcaactgtatcatcatgacgatattaacttggtttatgcttagcgaacttggcgtgcggcatgagcttttcgataatggtgatgattgtttgttcatttgcgaagaaaaagacgtacctagccccgagacgatcatgaactggtttgcggattttgggtttgtggttaggttagaaggcgtcgtgtccgtgtttgagcgcattgagttctgccaaacatcgcctatatggactgatcgaggttggctgatgtgtagaaacatcaagtctttgagtaaggatcttacgaacgttaattcgtgcactggctccactgttgaatacacccattggttgaaagcagttggaaagtgtggatcggtgctcaatgcgggtgtgcctatatttcagtcatttcacaacatgttgatgcgattgggtacgaattcgcgtatagatcgcggggtattctttaggtgtggacttgttaatctcattcgtgggatggacagacaacctgaagttgagatcactacttccgctcgtctttcttttgaagtggcattcgggatcactcccggcatgcaattggctattgagcaattttatgactcagtcgtgggccctctgggtaaaataaaatctgtaaaatggccaatagatctaagaaaggaatacgattacggaagcgcgtggttcgaagaccaaggcgtcctagggtgaacaaggaactcggattaccgatgacacctgttcaaactagaatggttcggtcaacgttgaccaaggagaccaacataccttctactgcaaatagcggtcgggaggctgtttgggcttgttggccaatcaactttagtgtctttccgcaactagcctcactcgtgaataaaccgttatactggcgtgtgtccagtgtgcaagttgcaatggagcctgcaatgtcttcttccacccaacattgtggtgttggtctcagttcttctggggccttcacataacggtgatgggttcggtaacgtctttaagctcttgcgttcttgtaactatacgcggcgctctcccgtgggaggaaacgtgatggtcaaatggcctatctgcatgcccttcattcttaacgatgatgcgcacaagaacacaggattaaccgcctgtgtgatcattgcagtcaccaatactggtgtgctaactggtcaatcttggacggagattctgttgaatgtggagtatacgccccgctagcatcgtactgctctcctaccggactggtagccgtttagttatcttggagtgatagcactgtggggccacatttgacgcgcattggacgcagacaatgtccctccacagatttgtgaatctctatggagctgtaacctcggtctctctatagcttgtccgaacaggaaatggacataaaataattgctgttccaacacgttgtgttggtaaagaagttatagatgtggtgcgccagacaagtggatggcaacctggagtaatccaggcgctctggggggcttatactcggagtgcattactgctttagaccgttaatctcaagaaccatgtgtgtcgcatggggaggattaacggcgcccaattcccttgttagtttaggtacgccttggtcttcgaaccacgc

SEQ ID NO: 22 - RNA, 2985 nt, unidentified
  source 1..2985: mol_type = viral cRNA
  misc_feature 1..2985: note = Polynucleotide Sequence of iRNA of Maize
ggggtaaatatggagaaccagcacacccatgtttgcccacggtcgttcctgcgaacctgcagggcgatcctcgcggctccagccaactacggtcgtgatgtggtcaaaatcgcctacaaatgggcatcacgaaaccccgccaccgccccccgaagtgtccgagaatccatcggggtcgttgtcggaagcgctgtggacttcttgagcgctcctcgcaagcgtttagaagaccgcgcagagcagttggtgcaagacgaccgggtcgaccggatcgtccgcgagtgggagctaggaaccgctgactcccgaattccggaagttgagtgggcataccgtctgcgcgaccgcttcggcgtcgtgtccgccagcgagcctgctaggcaaactggtgagaggtgggtgctcaagcaactagagggattggaggggggggagttccgctgcatacccattgagccattctttggtgatgcaccggcccccgtccatagccctgggagcaacagcgtgattgctgctattgcggcgaccctttggatgacgcctacccgccttgaccgggcgttgagacgtcaccagggttttcgcaactagcggtgatcggagtcgacggagtgtctgctttagcggtgcaggcatcttctgaactccgaccgctacgggttgggcgaccccgtcaaagtcgacgtcgttcgtggtctctgactatgccagcacccaagtcctgtttcgtgaaccacgctaactctgaccacaatctcaaaacggtcatggaaaacagggtgctcaagtacaaaggccaagaacccgcaaagccccgggtagaagcctataagcagctctatgaaaggatacgaccgcgatatcgttctctacctgacacggtctatcctctatcatatgatggcttcctcaagtgctactccggacgtaggcgaacacgatacgaacaggccgtccaggagttgagaaacgcgccactcacacccgaagatgctgtcgtttccacgttcatcaagaacgagaaattcgattggctccaaaagaaagaacttgcggatcccagagctatccaacctcggaaaccgaaatacctggccgaagttgggaggtggttcaagcctctggagcacataatgtataaagacttggcaaaacggttgtacggtcaggatgcgttgccttgcatagcgaaagggctgaacgctagagaaacggctgaagtgctccgagccaaatgggacaagttcgcttctcccgtttgcgtctcgctggatgccagtcggttcgatctgcatgtaagtcctgacgcattgcggtttacgcaccgcctgtaccacaagtattgccaaagtcggcaactccgcaagtacctagaatggacgctgagaaacgctggcgtcgcctcatgtcctgaaagcgcttatcagtatgaggttgaggggagacgcatgagtggcgacatggacaccgcactcggcaactgcgtacttatgctctgcttgacatggaacttcctcgatcaacataacatcaagcatgagataatggacaacggagatgactgcttgttcatctgtgaagctgccgatgtgccaaccgacaagcaaatcatggactactacctcgactttgggttcgtggttcggttggaaggaaaggtgtctgtgttcgagcgaatagagttctgtcaaaccagtccggtgttgactgctaatggatggcgtatggttagaaatttgaagtccattgcgaaggacctctgcaatgtgaacatggcgactgggtcactcagtgaatacactgcgtggcttaaagccgtgggaatctgtggtagaatcctgaacgatggggttccaatcttctccgccttccacaacatgctggtgcgacatggaacgaactcacgaatagatagagcggtgttctgggaatgtggactgacaaacttgatcaaaggcatgagtttcgagcaactggaaatcactgtcgctgcgcgcgagtccttttatctggcatacggtatcacaccggcgagacaactcgcgattgaagagtattacgactcactccagggcccggtgggtaaaatacaacttcatgaatggccactacaactcaaagaggaatacgcgtgcggcgccgagtggttcgaaggagacggcgagcgggcttgaggcccgctggcttgcccttcgtgcccggcagctctcgcacggttcggactgcgctcgtcctcgagaaccacttgccgatgtcctcggcacagttgggtcaagaggccgttgcgtattctatcccgtgcaatgttcgaaacatgcctacgatcctgactctcgccaccactccgctctattggcgtatcaccgccatcactgtcgcgatggagcctgcaaagtccacatcgacccaaattgccggtgtggggaatgctgattcatttcagtctgccacctacaacggttttgggaacgtgtttaagaaaatgcgcgctttgaatttcgtgagacgctcggcgcccggaggcaatcttcaggtacgctggcctatcaatatggactggatctccgcatccgacacggacaaggatagcacaaaagtgccctcgctattctttgccgtgaccaacccaggtgtgatcgaaaccaaacaaggggacagtgaggcctggttggaatgggagttggagctggagtacatagttggaggctaggaacgactgcccgcttgagatcgactctcccgtggtgaggtaccacccactcagctgtgtcagccggttggagaaactctggtgcgatagcactgttggcccctgcctagcgtgtgctgtgggaaagccccaacagatttgtgaaacactggagttgtcgacccgcgagacgtgcggctcgagttgtcgcttccccgtgaggggggctgccggggggtagagaaatattcccggtatttatccgctaagacctacgcgcgacgaaactggcg

SEQ ID NO: 23 - RNA, 4252 nt, Pea enation mosaic virus 2
  source 1..4252: mol_type = viral cRNA
  misc_feature 1..4252: note = Polynucleotide Sequence of PEMV2
gggtatttatagagatcagtatgaactgtgtcgctaggatcaagcggtggttcacacctgacttcacccctggcgagggcgtgaagtctagagctcaactggaaagagagctggatcccacctgggcgcttctcgtgtgccaagaacgagcgcgtcgtgatgctgacagtattgctaatgagtggtacgagggcagcatggagtgcaacctccttatccctcggcccacaaccgaggatgtatttggcccctccatcgcccctgagcctgtggctctagtggaggaaactacccgttcccgcgcgccgtgcgtggatgtccctgccgaggagtcctgtaagtcagcggagattgatcctgttgatctcgccaagttcgactccctccatcgtcgcctgttggctgaagccaacccttgcagggaaatggttctgtgggtgcctcctggcctaccagcagagcgcgacgtcctgcccagggcacgtggggtgataatgatccccgaagtccctgcctctgcacataccttgtccgtgaaggttatggaggctgtgcggttggcacaggaagtcttggcatcccttgccaagagggccttagagaaaaggtctacaccaacccttaccgcccaggcccagccagaggctaccctgtcggggtgcgactacccgtatcaggagactggagcagcagccgcgtggataacgcctggctgcattgccatggagctcagagccaaatttggcgtctgcaaacgcacccccgcaaacttagagatggggagtcgcgtcgcccgcgagctcctgcgggataactgtgtcacttgcagggagaccacgtggtacaccagtgccattgctgtggacctgtggttgaccccgaccgtcgtcgacctggcctgtggccggcgagcggcggatttttggtaggggctgtgctgcctcggctgggggaagacaccagtgtgcggtttgacaacctgcaccccagcatcgaggtaatcaaggcggctaggccccgcccaacccagaggatgtcgttccaaatcgacgttgtgcgtcctcttggagattttggtgtgcacaacaactcccttgttaacctagccaggggaattaatgaaagggtgttctacacggacaatgctaggacagaacccctccagcctaaggttcccttcccctcatcacgggagctaaaaaccttcagagtcaccccttggaccatggatagggttgtggagagttacacagggtcccagcgcactcgctatgctaacgcgcgggacagcatattatccaaccctctgagtcccaaagatgcgcgggtcaagacgtttgtcaaagctgaaaagataaatttcacagccaaacctgaccccgcccctcgtgtgatacagcctagggatccacgattcaacattgtcctggctaaatacatcaagcctttggagccaatgttgtacaaagcactggggaaactttacaagtaccccgcagttgctaaggggtttaacgcggttgagacgggggagatcatcgccggcaagtggcggtgcttcaaagatcctgtcgtcgtgggattagacgcttcccgatttgatcagcatgtatctgtcgaggcgttgcagttcacccacgcggtgtacagagggttcatcaagtcacgggagtttaacaacctcctacagatgatgtacaccaaccgtggcctagggtccgctaaggacggattcgtccgttacaaggttaaaggtagacgcatgagcggtgacatggacacctccttgggcaactgtgtgctcatggtgttgctcaccaggaacctttgcaaggttctaggcatcccgcacgagctcttcaacaatggtgatgattgcatcgtctttttcgatcgttgccacttggagaagttcaacaatgctgtcaagacttattttgcggacctagggtttaagatgaaggtggaaccgccggttgacgtgttggagaaaatagagttctgccaaacgcagcctatctatgacggggagaagtggcgcaccgtgcgttgcatctcgagtatcggaaaagattgctcatccgttattagttgggaccaattggaggggtggtggaatgccatcgcccagagtggtctggctgtgtgtggcggaatgccgatatacacgtcgttctaccggtggctagcacgggccggtaagagtgggaccaagtgtcagtcacaccccttgtggaaaaacgaggggttgaattggtacaggatggggatggacctttctcatgaggttaatgttacccctcaggcgcgcctgtctttcttcgcgggttttggtatttcccccccgatgcaggtcgccattgaggcgctgtatgacaagctgcctccaccgtccccccaccatggtcctccggttaaggctgtaacacagcgagtgttcaccaattatttcacgccggaaagcgcctgtgttagcatgagcacgaatgaagacaacaaatctgactttgctgtttacggccctgtgcctacagtgatgtctctttgtgctcagtgttaggctcttaaattttagcgatggcgtgacacggttacaccctgaattgacagggtacagatcaagggaagccggggagtcaccaacccaccctgaatcgacagggcaaaaagggaagccgggcaccgcccacgtggaatcgaccacgtcaccttttcgcgtcgactatgccgtcaacaccctttcggcccgccagcctaggacaatggcggtagggaaatatatgacgataatcattaatgtcaataacgacgagcgcaagcaaccagaaggagctactggcagctctgtacggcgaggtgacaataaaagaactcgaggaaacaaacctcggagtcatcaccccggttcgcgcgaacgaaaaggttacaatcacccctctcctacccccaaaaactcaaagcagggtcagctccgtactgaagcggttcaggagcacccgaaacacggggggactgctttccgtagagaaagtggtggtagtgttcacccctcacatccccgacgacgtgctaggagaggtggagatatggctccacgacagcatcctcccccacctcgggagcgtcggaccaagactgaaactcaagctgagcgaagggcccaagctcttagcgttctacccaccctactcgattgcattgggggactcgatctcgggccagccgaggtccttctccattgtcaccgagctgttcgaaggcaacttcgcaccggggtgcagcccattcagcctgttcctcatgtggagtccacgcatcgaagcagtgacccacaactacttgagtcgtccaccacgtgctctgccaatttgcagaacgatggtgcgggacgcgttatcggaggtggcatcccaacagcaatacctgaagggagcgatgtcgaacaggtatgccatgcctctcactacgggtgatggccagcatagagccatgaagggggctcccagtgcccttccaccaacgggggtgtgtacccaggcttctaagtgaggcttcgcttcccgccggaagaccgcggcggttctgttcctcccacaggagtacggcaacaacccaccttgggaaagtggggaccccagcactaactcctttaactaggcgggcgtgttggttacagtaggaggggacagtgcgcatcgaaactgagccccaccacaactctcatccacggggtggttgggacgcaggtgtcggagggatcgccagccctcaggatagtgagctcccgcagagggataagctatctccctgcgacgtagtggtagaacacgtgggataggggatgaccttgtcgaccggttatcggtcccctgctccttcgagctggcaaggcgctcacaggttctacactgctactaaagttggtggtggatgtctcgcccaaaaagatcacaaacgcgcgggacaaggtcccttccaccttcgccgggtaaggctagagtcagcgctgcatgactataacttgcggccgatccagttgcacgactggtggtccccctcagtgtctcggttgtctgccgagtgggcggtggtcggattccaccacaccctgccacgaggtgcgtggagacttggccagtctaggctcgtcgtaattagttgcagcgacgttaatcaacccgtccgggcatataataggaccggttgtgcttcttcctcccttcttagccaggtggttacctccctggcgccc

SEQ ID NO: 24 - RNA, 313 nt, Pea enation mosaic virus 2
  source 1..313: mol_type = viral cRNA
  misc_feature 1..313: note = Polynucleotide Sequence of the Intergenic Plus Region of PEMV2
gttagcatgagcacgaatgaagacaacaaatctgactttgctgtttacggccctgtgcctacagtgatgtctctttgtgctcagtgttaggctcttaaattttagcgatggcgtgacacggttacaccctgaattgacagggtacagatcaagggaagccggggagtcaccaacccaccctgaatcgacagggcaaaaagggaagccgggcaccgcccacgtggaatcgaccacgtcaccttttcgcgtcgactatgccgtcaacaccctttcggcccgccagcctaggacaatggcggtagggaaatatatg

SEQ ID NO: 25 - RNA, 139 nt, Pea enation mosaic virus 2
  source 1..139: mol_type = viral cRNA
  misc_feature 1..139: note = Polynucleotide Sequences of Recoding Frameshift Sites of PEMV2
gaccgtcgtcgacctggcctgtggccggcgagcggcggatttttggtaggggctgtgctgcctcggctgggggaagacaccagtgtgcggtttgacaacctgcaccccagcatcgaggtaatcaaggcggctaggcccc

SEQ ID NO: 26 - RNA, 65 nt, unidentified
  source 1..65: mol_type = viral cRNA; note = Citrus yellow vein-associated virus [CYVaV]
  misc_feature 1..65: note = CYVaV Insertion Region of VIGS Vector and Flanking Sequences
taggcctcgacacgggaaggtagctgtcccggcactgggttgcacatattccgtgccgacgccac

SEQ ID NO: 27 - RNA, 43 nt, unidentified
  source 1..43: mol_type = viral cRNA; note = Citrus yellow vein-associated virus [CYVaV]
  misc_feature 1..43: note = CYVaV Insertion Region of VIGS Vector and Flanking Sequences
ccggcctcgacacgggaaggtagctattccgtgccgacgccgt

SEQ ID NO: 28-29 - skipped (2 entries)

SEQ ID NO: 30 - RNA, 10 nt, unidentified
  source 1..10: mol_type = viral cRNA; note = Citrus yellow vein-associated virus [CYVaV]
  misc_feature 1..10: note = Lock and Dock Sequence
gtcctaagtc

SEQ ID NO: 31 - RNA, 14 nt, unidentified
  source 1..14: mol_type = viral cRNA; note = Citrus yellow vein-associated virus [CYVaV]
  misc_feature 1..14: note = Lock and Dock Sequence
caggggaaactttg

SEQ ID NO: 32 - RNA, 31 nt, unidentified
  source 1..31: mol_type = viral cRNA; note = Citrus yellow vein-associated virus [CYVaV]
  misc_feature 1..31: note = Nucleotide Sequence of a Scaffold Comprising A Docked Tetraloop
cattagctaaggatgaaagtctatgctaatg

SEQ ID NO: 33 - RNA, 57 nt, unidentified
  source 1..57: mol_type = viral cRNA; note = Citrus yellow vein-associated virus [CYVaV]
  misc_feature 1..57: note = Nucleotide Sequence of a Lock and Dock Structure
gcacctaaggcgtcagggtctagaccctgctcaggggaaactttgtcgctatggtgc

SEQ ID NO: 34 - RNA, 47 nt, synthetic construct
  source 1..47: mol_type = other RNA
  misc_feature 1..47: note = CYVaV Insertion
ggctagttaatctcattcgtgggatggacaggcagcctgacgttgac

SEQ ID NO: 35 - RNA, 30 nt, synthetic construct
  source 1..30: mol_type = other RNA
  misc_feature 1..30: note = CYVaV Insertion
gttaatgtaggtgtctttccgtatctagtc

SEQ ID NO: 36 - RNA, 30 nt, synthetic construct
  source 1..30: mol_type = other RNA
  misc_feature 1..30: note = CYVaV Insertion
gtcaacgcaggtgcctgtccgtatctagcc

SEQ ID NO: 37 - RNA, 66 nt, synthetic construct
  source 1..66: mol_type = other RNA
  misc_feature 1..66: note = Hairpin Targeting GFP (CYVaV-GFPhp2301)
tgaagcggcacgacttcttcaagagcgccagaattctggcgctcttgaagaagtcgtgccgcttca

SEQ ID NO: 38 - RNA, 48 nt, synthetic construct
  source 1..48: mol_type = other RNA
  misc_feature 1..48: note = Sequence of Hairpin Targeting Conserved Sequence in the CTV Genome
tccgtggacgtcatgtgtaagggtacccttacacatgacgtccacgga

SEQ ID NO: 39 - RNA, 21 nt, synthetic construct
  source 1..21: mol_type = other RNA
  misc_feature 1..21: note = CYVaV-CTV18 Hairpin
cttacacatgacgtccacgga

SEQ ID NO: 40 - RNA, 54 nt, synthetic construct
  source 1..54: mol_type = other RNA
  misc_feature 1..54: note = CYVaV-CTV6 Hairpin
ggaagtgatggacgaaattaatgaccaatcattaatttcgtccatcacttccag

SEQ ID NO: 41 - RNA, 24 nt, synthetic construct
  source 1..24: mol_type = other RNA
  misc_feature 1..24: note = Sequence of CYVaV-CTV6 Hairpin
tcattaatttcgtccatcacttcc

SEQ ID NO: 42 - RNA, 57 nt, synthetic construct
  source 1..57: mol_type = other RNA
  misc_feature 1..57: note = Lock and Dock Structure
gcgatatggattcagggactagtccctgctcaggggaaactttgtgtcctaagtcgc

SEQ ID NO: 43 - RNA, 77 nt, synthetic construct
  source 1..77: mol_type = other RNA
  misc_feature 1..77: note = Lock and Dock Structure
gcgatatggatcaggactagtcctgtcaccctcacttcggtgtccaggggaaactttgtgggtgagtcctaagtcgc

SEQ ID NO: 44 - RNA, 60 nt, synthetic construct
  source 1..60: mol_type = other RNA
  misc_feature 1..60: note = CYVaV Hairpin (CY2301GFP30)
agttaatgtaggtgtctttcctgaagcggcacgacttcttcaagagcgccagtatctagt

SEQ ID NO: 45 - RNA, 30 nt, synthetic construct
  source 1..30: mol_type = other RNA
  misc_feature 1..30: note = CYVaV Hairpin (CY2301GFP30)
agttaatgtaggtgtctttcctgaagcggc

SEQ ID NO: 46 - RNA, 11 nt, synthetic construct
  source 1..11: mol_type = other RNA
  misc_feature 1..11: note = CYVaV Hairpin (CY2301GFP30)
cagtatctagt

SEQ ID NO: 47 - RNA, 120 nt, synthetic construct
  source 1..120: mol_type = other RNA
  misc_feature 1..120: note = CYVaV with L&D1 and 30 nt Hairpin Insert
agttaatgtaggtgtctttccgcgatatggattcagggacttgaagcggcacgacttcttcaagagcgccaagtccctgctcaggggaaactttgtgtcctaagtcgcgtatctagtcac

SEQ ID NO: 48 - RNA, 120 nt, synthetic construct; identical to SEQ ID NO: 47
  source 1..120: mol_type = other RNA
  misc_feature 1..120: note = CYVaV with L&D1 and 30 nt Hairpin Insert

SEQ ID NO: 49 - RNA, 30 nt, synthetic construct
  source 1..30: mol_type = other RNA
  misc_feature 1..30: note = CYVaV with L&D1 and 30 nt Hairpin Insert
tgaagcggcacgacttcttcaagagcgcca

SEQ ID NO: 50 - RNA, 52 nt, synthetic construct
  source 1..52: mol_type = other RNA
  misc_feature 1..52: note = L&D1 and L&D1 +Hairpin Targeting Callose Synthase
tgatacctgttcagaataggattgctcgagcttcgttggttagggtaactca

SEQ ID NO: 51 - RNA, 59 nt, synthetic construct
  source 1..59: mol_type = other RNA
  misc_feature 1..59: note = L&D1 and L&D1 +Hairpin Targeting Callose Synthase
gcgatatggattcagggactagtccctgctcaggggaaactttgtgtcctaagtcgcac

SEQ ID NO: 52 - skipped

SEQ ID NO: 53 - RNA, 180 nt, synthetic construct
  source 1..180: mol_type = other RNA
  misc_feature 1..180: note = L&D1 and L&D1 +Hairpin Targeting Callose Synthase
aatagggtcattggtttaccgatgatacctgttcagaataggattgctcgagcttcgttggttagggtaactcacataccttcttccatagcgatatggattcagggactagtccctgctcaggggaaactttgtgtcctaagtcgcactggaaaaggtcgtgtgagcaacctaaccagt

SEQ ID NO: 54 - RNA, 51 nt, synthetic construct
  source 1..51: mol_type = other RNA
  misc_feature 1..51: note = L&D1 and L&D1 +Hairpin Targeting Callose Synthase
gatacctgttcagaataggattgctcgagcttcgttggttagggtaactca

SEQ ID NO: 55 - RNA, 89 nt, synthetic construct
  source 1..89: mol_type = other RNA
  misc_feature 1..89: note = L&D1 and L&D1 +Hairpin Targeting Callose Synthase
gcgatatggattcagggacttgatgttggatccatcctatgagccttttcagtccctgctcaggggaaactttgtgtcctaagtcgcac

SEQ ID NO: 56 - RNA, 39 nt, synthetic construct
  source 1..39: mol_type = other RNA
  misc_feature 1..39: note = L&D1 and L&D1 +Hairpin Targeting Callose Synthase
ctaaccagttaatgtaggtgtctttccgtatctagtcac

SEQ ID NO: 57 - RNA, 110 nt, synthetic construct
  source 1..110: mol_type = other RNA
  misc_feature 1..110: note = L&D1 and L&D1 +Hairpin Targeting Callose Synthase
aatagggtcattggtttaccgatgatacctgttcagaataggattgctcgagcttcgttggttagggtaactcacataccttcttccatagcgatatggattcagggact

SEQ ID NO: 58 - RNA, 100 nt, synthetic construct
  source 1..100: mol_type = other RNA
  misc_feature 1..100: note = L&D1 and L&D1 +Hairpin Targeting Callose Synthase
agtccctgctcaggggaaactttgtgtcctaagtcgcactggaaaaggtcgtgtgagcaacctaaccagttaatgtaggtgtctttccgtatctagtcac

SEQ ID NO: 59 - RNA, 30 nt, synthetic construct
  source 1..30: mol_type = other RNA
  misc_feature 1..30: note = L&D1 and L&D1 +Hairpin Targeting Callose Synthase
tgatgttggatccatcctatgagccttttc

SEQ ID NO: 60 - RNA, 52 nt, synthetic construct
  source 1..52: mol_type = other RNA
  misc_feature 1..52: note = Lock and Dock Structure
ggaagtgatggacgaaattaatgaccaatcattaatttcgtccatcacttcc

SEQ ID NO: 61 - RNA, 63 nt, synthetic construct
  source 1..63: mol_type = other RNA
  misc_feature 1..63: note = Lock and Dock Structure 3 (L&D3)
gcggcgatatggattcagggactagtccctgctcaggggaaactttgtgtcctaagtcgccgc

SEQ ID NO: 62 - RNA, 89 nt, synthetic construct
  source 1..89: mol_type = other RNA
  misc_feature 1..89: note = L&D1 in Position 2301 of CYVaV
tgtaggtgtctttccgcgatatggattcagggactagtccctgctcaggggaaactttgtgtcctaagtcgcgtatctagtcacgatgg

SEQ ID NO: 63 - RNA, 180 nt, synthetic construct
  source 1..180: mol_type = other RNA
  misc_feature 1..180: note = L&D1 in Position 2301 of CYVaV
ttccataactggaaaaggtcgtgtgagcaacctaaccagttaatgtaggtgtctttccgcggcgatatggattcagggactagtccctgctcaggggaaactttgtgtcctaagtcgccgcgtatctagtcacgatggtaagcaacccgtttatctgtacggcgctcacccgtgggtaga

SEQ ID NO: 64 - RNA, 23 nt, synthetic construct
  source 1..23: mol_type = other RNA
  misc_feature 1..23: note = L&D1 in Position 2375 of CYVaV
cagacctttgttacttccaacac

SEQ ID NO: 65 - RNA, 29 nt, synthetic construct
  source 1..29: mol_type = other RNA
  misc_feature 1..29: note = L&D1 in Position 2375 of CYVaV
ctggatttcctgtgttttggaagtggaag

SEQ ID NO: 66 - RNA, 21 nt, Citrus tristeza virus
  source 1..21: mol_type = viral cRNA
  misc_feature 1..21: note = Conserved CTV Sequence (15173)
tccgtggacgtcatgtgtaag

SEQ ID NO: 67 - RNA, 24 nt, Citrus tristeza virus
  source 1..24: mol_type = viral cRNA
  misc_difference 1..24: note = Conserved CTV Sequence (17856)
ggaagtgatggacgaaattaatga

SEQ ID NO: 68 - RNA, 51 nt, synthetic construct
  source 1..51: mol_type = other RNA
  misc_feature 1..51: note = siRNA Targeting GFP in CYVaV Genome
tgaagcggcacgacttcttcaatcaagagatgaagaagtcgtgccgcttca

SEQ ID NO: 69 - RNA, 61 nt, synthetic construct
  source 1..61: mol_type = other RNA
  misc_feature 1..61: note = siRNA Targeting GFP in CYVaV Genome
tgaagcggcacgacttcttcaagagcatcaagagagctcttgaagaagtcgtgccgcttca

SEQ ID NO: 70 - RNA, 32 nt, synthetic construct
  source 1..32: mol_type = other RNA
  misc_feature 1..32: note = siRNA Targeting GFP in CYVaV Genome
tgaagcggcacgacttcttcaagagcgccaga

SEQ ID NO: 71 - RNA, 32 nt, synthetic construct
  source 1..32: mol_type = other RNA
  misc_feature 1..32: note = siRNA Targeting GFP in CYVaV Genome
tctggcgctcttgaagaagtcgtgccgcttca

SEQ ID NO: 72 - RNA, 61 nt, synthetic construct
  source 1..61: mol_type = other RNA
  misc_feature 1..61: note = siRNA Targeting GFP in CYVaV Genome
ggttagggtaactcacataccttcttccataactggaaaaggtcgtgtgagcaacctaacc

SEQ ID NO: 73 - RNA, 59 nt, synthetic construct
  source 1..59: mol_type = other RNA
  misc_feature 1..59: note = GFPmmck59
tgaagcggcacgacttcttcaagagcgataactcgccttgacagaagtccaacgcttca

SEQ ID NO: 74 - RNA, 167 nt, synthetic construct
  source 1..167: mol_type = other RNA
  misc_feature 1..167: note = GFPmmck59 Inserted at CYVaV Position 2304
aaaaggtcgtgtgagcaacctaaccagttaatgtaggtgtctttccgtatgaagcggcacgacttcttcaagagcgataactcgccttgacagaagtccaacgcttcatctagtcaggatggtaagcaacccgtttatctgtacggcgctcacccgtgggtaggaag

SEQ ID NO: 75 - RNA, 61 nt, synthetic construct
  source 1..61: mol_type = other RNA
  misc_feature 1..61: note = Mmck6.1
ggaagtgatggacgaaattaatgattccataactggaacattacatttcgtcaacacttcc

SEQ ID NO: 76 - RNA, 61 nt, synthetic construct
  source 1..61: mol_type = other RNA
  misc_feature 1..61: note = CS7-V2.2
ggtatcaatgggcagacgaagatttggcataactgccaatcttccgtctgcaaatgatacc

SEQ ID NO: 77 - RNA, 155 nt, synthetic construct
  source 1..155: mol_type = other RNA
  misc_feature 1..155: note = F3-C3#3
actagaggaagtgttgacgaaatgtaatgttccagttatggaatcattaatttcgtccatcacttcctacggaaagacacctacattaactggtatcatttgcagacggaagattggcagttatgccaaatcttcgtctgcccattgataccaac

SEQ ID NO: 78 - RNA, 83 nt, synthetic construct
  source 1..83: mol_type = other RNA
  misc_feature 1..83: note = Polynucleotide Insert into CYVaV
ttggttagggtaactcacataccttcttccatatgaagcggcacgactatctagtcannntggtaagcannncgtttatctgt

SEQ ID NO: 79 - RNA, 240 nt, synthetic construct
  source 1..240: mol_type = other RNA
  misc_feature 1..240: note = Polynucleotide Sequence of CBC460, CBC461 and CBC462
taaccagttaatgtaggtgtctttccgtatctagtcacgatggtaagcaacccgtgcgtgttacgtcagtgacaatcgccatggaaccggcaatgtctacctctacccaacactgtggggtaggactcgctactagcggggcctactcttcaggtgagggttttggtaacttgttcaagtacaagcgcgcttgtaactttatctgtacggcgctcacccgtgggtaggaaggtgaaggtt

SEQ ID NO: 80 - RNA, 144 nt, synthetic construct
  source 1..144: mol_type = other RNA
  misc_feature 1..144: note = Polynucleotide Sequence of OULV-insert
tgcgtgttacgtcagtgacaatcgccatggaaccggcaatgtctacctctacccaacactgtggggtaggactcgctactagcggggcctactcttcaggtgagggttttggtaacttgttcaagtacaagcgcgcttgtaact

SEQ ID NO: 81 - RNA, 240 nt, synthetic construct
  source 1..240: mol_type = other RNA
  misc_feature 1..240: note = Polynucleotide Sequence of CY2220 (1132-1329)2280-1 and CY2220 (1132-1329)2280-2
attgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgt

SEQ ID NO: 82 - RNA, 300 nt, synthetic construct
  source 1..300: mol_type = other RNA
  misc_feature 1..300: note = Polynucleotide Sequence CBC135
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 83 - RNA, 300 nt, synthetic construct
  source 1..300: mol_type = other RNA
  misc_feature 1..300: note = Polynucleotide Sequence CBC139
ggtcattggtttaccgatgatacctgttcagaataggattgctcgagcttcgttgcagtaaaaggttgtatggtgatggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 84 - RNA, 300 nt, synthetic construct
  source 1..300: mol_type = other RNA
  misc_feature 1..300: note = Polynucleotide Sequence CBC140
ggtcattggtttaccgatgatacctgttcagaataggattgctcgagcttcgttgcagtaaaaggttgtatggtgagggtgctgagctgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 85 - RNA, 300 nt, synthetic construct
  source 1..300: mol_type = other RNA
  misc_feature 1..300: note = Polynucleotide Sequence CDE967
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgttcctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 86 - RNA, 300 nt, synthetic construct
  source 1..300: mol_type = other RNA
  misc_feature 1..300: note = Polynucleotide Sequence CDE974
ggtcattggtttaccgatgatacctgttcagaataggattgctcgagcttcgttgcagtaaaaggttgtatggtgagggtgctgagccgtgtatcgccaaaggcctaaatgcattacaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 87 - RNA, 300 nt, synthetic construct
  source 1..300: mol_type = other RNA
  misc_feature 1..300: note = Polynucleotide Sequence CDE977
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagcttgcgtttccctcgacgcttccaggttcgacctgcatgtaagcgttggcatgctaaagctcacacacaagctatatgactattactgcagttaatgtaggtgtctttccgtatctagtcacgatggtaagcaac

SEQ ID NO: 88 - RNA, 300 nt, synthetic construct
  source 1..300: mol_type = other RNA
  misc_feature 1..300: note = Polynucleotide Sequence CDE978
ggtcattggtttaccgatgatacctgttcagaataggattgctcgagcttcgttgcagtaaaaggttgtatggtgagggtgctgagccgcgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 89 - RNA, 300 nt, synthetic construct
  source 1..300: mol_type = other RNA
  misc_feature 1..300: note = Polynucleotide Sequence CDE980
ggtcattggtttaccgatgatacctgttcagaataggattgctcgagcttcgttgcagtaaaaggttgtatggtgagggtgctgagccgtgtatcgccaaaggtctaaatgcattagaatctggagcgactttgaggcgcaaatgggagaagttttcttctccagcttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 90 - RNA, 300 nt, synthetic construct
  source 1..300: mol_type = other RNA
  misc_feature 1..300: note = Polynucleotide Sequence CDF015
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcatacacaagctatatgactattactgcagttaatgtaggtgtctttccgtatctagtcacgatggtaagcaac

SEQ ID NO: 91 - RNA, 300 nt, synthetic construct
  source 1..300: mol_type = other RNA
  misc_feature 1..300: note = Polynucleotide Sequence CDF016
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcaacgcttccaggttcgacctgcatgtaagcgttggcatgctaaagttcacacacaagctatatgactattactgcagttaatgtaggtgtctttccgtatctagtcacgatggtaagcaac

SEQ ID NO: 92 - RNA, 300 nt, synthetic construct
  source 1..300: mol_type = other RNA
  misc_feature 1..300: note = Polynucleotide Sequence CDF017
ggtcattggtttaccgatgatacctgttcagaataggattgctcgagcttcgttgcagtaaaaggttgtatggtgagggtgctgagccgtgtatcgccaaaggcctaaatgtattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 93 - RNA, 300 nt, synthetic construct
  source 1..300: mol_type = other RNA
  misc_feature 1..300: note = Polynucleotide Sequence CDF020
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcacgtaagcgttggcatgctaaagttcacacacaagctatatgactattactgcagttaatgtaggtgtctttccgtatctagtcacgatggtaagcaac

SEQ ID NO: 94 - RNA, 300 nt, synthetic construct; identical to SEQ ID NO: 85
  source 1..300: mol_type = other RNA
  misc_feature 1..300: note = Polynucleotide Sequence CDF037

SEQ ID NO: 95 - RNA, 300 nt, synthetic construct
  source 1..300: mol_type = other RNA
  misc_feature 1..300: note = Polynucleotide Sequence CDF041
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatacgactattactgcagttaatgtaggtgtctttccgtatctagtcacgatggtaagcaac

SEQ ID NO: 96 - RNA, 300 nt, synthetic construct
  source 1..300: mol_type = other RNA
  misc_feature 1..300: note = Polynucleotide Sequence CDF047
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagttcgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 97 - RNA, 300 nt, synthetic construct
  source 1..300: mol_type = other RNA
  misc_feature 1..300: note = Polynucleotide Sequence CDF049
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagtttccttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 98 - RNA, 58 nt, synthetic construct
  source 1..58: mol_type = other RNA
  misc_feature 1..58: note = Insertion of Mmck6.2 into CYVaV1
gtttcagtatttcgtcatcaccttcctaacggaagtgatggacgaaattaatgaaacc

SEQ ID NO: 99 - RNA, 60 nt, synthetic construct
  source 1..60: mol_type = other RNA
  misc_feature 1..60: note = WT Insert Structure
ggttagggtaactcacataccttcttccataactggaaaaggtcgtgtgacaacctaacc

SEQ ID NO: 100 - RNA, 61 nt, synthetic construct
  source 1..61: mol_type = other RNA
  misc_feature 1..61: note = Mmck8 Insert
  misc_feature 1..61: note = Mmck15 Insert
gtcgcaatcaaagacgaagaaatcgtccataactggacatttccttcgtctcaattgcgac

SEQ ID NO: 101 - RNA, 61 nt, synthetic construct
  source 1..61: mol_type = other RNA
ggttgcttggaacccatacgaatgttgcttaacagcaaattcgctatgggtgatagcaacc

SEQ ID NO: 102 - RNA, 61 nt, synthetic construct
  source 1..61: mol_type = other RNA
  misc_feature 1..61: note = MmckpsvD Insert
gggtgaagtcatgaaagaagctgccttcttaacagaagcagctctctttcacaattcaccc

SEQ ID NO: 103 - RNA, 61 nt, synthetic construct
  source 1..61: mol_type = other RNA
  misc_feature 1..61: note = MmckpsvE Insert
ccgtaggtaagacaccattgacaagttcttaacagaactgtcacatggtgtgaccctacgg

SEQ ID NO: 104 - RNA, 61 nt, synthetic construct
  source 1..61: mol_type = other RNA
  misc_feature 1..61: note = PRSVmmck1 Insert
atggtctgaatgataatgaaatgcaagtgtaaccacttcatttccattatccaaagaccat

SEQ ID NO: 105 - RNA, 61 nt, synthetic construct
  source 1..61: mol_type = other RNA
  misc_feature 1..61: note = PRSVmmck2 Insert
gcagaagcatatattgcaaagagaaatgctaacgcattctcttctgcaatacaacttctgc

SEQ ID NO: 106 - RNA, 199 nt, unidentified
  source 1..199: mol_type = viral cRNA; note = Citrus yellow vein-associated virus [CYVaV]
  misc_feature 1..199: note = Wild Type Structure 1 (1132-1329) of CYVaV
cagtaaaaggttgtatggtgagggtgcttgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactg

SEQ ID NO: 107 - RNA, 198 nt, synthetic construct
  source 1..198: mol_type = other RNA
  misc_feature 1..198: note = PDS-198mmck-1 Insert
cacgaaacagaagtacttggcttcaatggaaggtgctgtcttatcaggaaagctttgtgcacaagctattgtacaggattacgagttacttcttggcttttgccaagatgtaactcgtaaacaaattacaaatagcttgtcaaacaaaactgtcggataaacagtaaacttccttgcctacaagtaagtctattcgtg

SEQ ID NO: 108 - RNA, 198 nt, synthetic construct
  source 1..198: mol_type = other RNA
  misc_feature 1..198: note = PDS-198mmck-2 Insert
gttgctcagtgtgtacgctgacatgtctgttacatgtaaggaatattacaaccccaatcagtctatgttggaattggtatttgcacccgcagaagagttttctcttctacgggtgcaaataagtgtttcacaacatagaaaatattggagtcgtctattcttacccaataacaacaacaaagcgtacacacaagcaac

SEQ ID NO: 109 - RNA, 198 nt, synthetic construct
  source 1..198: mol_type = other RNA
  misc_feature 1..198: note = LcrGyrA-Mmck5 Insert
acctggtactgttcggcgaaataaattatctgattttgtgcatgtgaaccgtaatggtaagattgcaatgaaattggaggaaaatgatgagattgttttttaacaatcgcatcattttccccattcgctcatgtgcaatgaccaccatcgttgtaacatgacaactcacagataataccatcgccgaacaccccaggt

SEQ ID NO: 110 - RNA, 198 nt, synthetic construct
  source 1..198: mol_type = other RNA
  misc_feature 1..198: note = LcrGyrA-Mmck3 Insert
atgatgagattgtttcagtagaaacttgtactgaggaccatgatgttttgctaacaacagaatttggtcagtgtattcgattcccagtttctaatgtttttacattagcaactgtgaatcccacacacctgacccatttaaacgttgtcaaaaaccattaggtcaaagagtacagtggaaactgaaacaacccatcat

SEQ ID NO: 111 - RNA, 198 nt, synthetic construct
  source 1..198: mol_type = other RNA
  misc_feature 1..198: note = Clas-GyrAmmck-5 prime Insert
gcatggcaatgtacggcgtaataaactttctgattttattcaaatcaatcgtagtggtaagattgcgatgaaattagattcaagagatgagattcttttttaagaatcccatctcttgaaacattccctcatgcgcaataaccaccacccatattatttgataacccacagaaggttcaaatgctgtacaccccatgc

SEQ ID NO: 112 - RNA, 198 nt, synthetic construct
  source 1..198: mol_type = other RNA
  misc_feature 1..198: note = las-GyrAmmck-3 prime insert
gagatgagattctttccgttgaaacctgtacacaagaaaatgatatattgttgactactaaacttggacaatgtgtccgctttccgatttctgctatttttatagcagcaatcggaaagctaacacacttgtcccaagtggccgtagttccaaactatcatttcgccctgtacggtaaacacggaaagaaaacatctc

SEQ ID NO: 113 - RNA, 198 nt, synthetic construct
  source 1..198: mol_type = other RNA
  misc_feature 1..198: note = CS7 Insert
agaattatggagcggtcaagctcacacatcgtcacactttttatgtggtgggctcagccaaaactgtatgtaggaagaggcttgcatgagggcatgtttttacatgccatcatgcaagccagacgactatcatacagttgaccgctgatccaactcataaaagtcaaaatgattgtacaattgattgctctcaattct

SEQ ID NO: 114 - RNA, 198 nt, synthetic construct
  source 1..198: mol_type = other RNA
  misc_feature 1..198: note = Erwinia GyrA Insert
ccgaccgcgcagcgccggtattattgccgtcaatctgcgtgatgacgatgaactgatcggcgtgtcgctgacgaacggtagtgatgaagcgatgctgttttcagcatcacttcatcactaaaaagacgtacagtgacacataagatcacttaatatcatccgtatacatgatgcaaaccaattggtgttgtaggttgg

SEQ ID NO: 115 - RNA, 65 nt, synthetic construct
  source 1..65: mol_type = other RNA
  misc_feature 1..65: note = M2250GFP30ext
ggttagggtaactcactgaagcggcacgacttcttcaagagcgccattcagtgagcaacctaacc

SEQ ID NO: 116 - RNA, 59 nt, synthetic construct; identical to SEQ ID NO: 73
  source 1..59: mol_type = other RNA
  misc_feature 1..59: note = GFPmmck59

SEQ ID NO: 117 - RNA, 63 nt, synthetic construct
  source 1..63: mol_type = other RNA
  misc_feature 1..63: note = GFPmmck63
tgaagcggcacgacttcttcaagagcgccataatcggcgccttgacagaagtccaacgcttca

SEQ ID NO: 118 - RNA, 128 nt, Citrus tristeza virus
  source 1..128: mol_type = viral cRNA
  misc_feature 1..128: note = CTV-insert-natural-V2.2
gggggtttatgtttggcaaagaaagtgttggaactgttagtcaagcgggtggttgaatcgttttctcgtttgaagcggaaaaccgctcgtttaacgtccttcgctaatttgttgcttgcgaggctctc

SEQ ID NO: 119 - RNA, 138 nt, synthetic construct
  source 1..138: mol_type = other RNA
  misc_feature 1..138: note = CYVaV1 with Insert-Natural-V2.2
tcgttgggggtttatgtttgcaaagaaagtgttggaactgttagtcaagcgggtggttgaatcgtttctcgtttgaagcggaaaaccgctcgtttaacgtccttcgctaatttgttgcttgcgaggctctcagttaat

SEQ ID NO: 120 - RNA, 2485 nt, synthetic construct
  source 1..2485: mol_type = other RNA
  misc_feature 1..2485: note = Recoding Structure Element Region of CYVaV-delta
aggtcgcggcgaccttgtggctaactaagaccttgtctgacaaggccttggcccgccaccagggttttcgcgatttgcagtgattggggtcgacgggctagaggctaaagcagtgcctctagctgcaggactccgactgcttccggttccgcggcccggacaaagccgacggctgtctcagaccttgttactaccaacccctcgtgcacaatttgtgaatcacgcgaactcggctaataaccttgggcgcgtgatgaccacacgagtgatgcagtacaaaggccgagacccgatccaaccctctcaagaagccctaaccaaacttaaccttcggatagccgagctatataagtcgagaccatccaccgtctatccgttgagttatgaagggtttctcaactgctacgaaggccgacagcgtactcgttacgctcaagccgtcgagcagctgttgaggtccactctagaacctaaagatgcgagagttgaaacgttcattaagaacgagaagtttgattgggcgttgaaaggggaggaggctgatcctcgtgcaatccaaccaaggaagccgaaatatctggctgaggttggacggtggtttaaacctttggagcgaatcatctacaaggatcttagtaagcggttgtatggcgagggtggtgaaccttgtattgccaaaggcttaaatgcactagaatctggggcgactttgaggcgcaaatgggagaagttttcttctcctgtttgcgtctctcttgacgcttccaggttcgacctgcatgtaagtgttggcatgcttaagtttacacacaagctgtacgactactactgcaagtctcccaccctccaacgttatctcaaatggacactccgcaatcacggcactgcctcctgtaaagaattgtcatatggttatgaggtagaaggccgaagaatgagtggtgacatggacaccgcattgggcaactgcgtcatcatgtcgatactaacatggtttatgcttagtgagcttggcattaagcatgaattattcgacaatggtgacgactgcttgttcatttgcgagtcaaaagacgtccccagccccgaggtgatcacgaactggttttcggactttgggtttgtggtcaggttggaaggcgtcacgtccgtgtttgagcgtatagagttttgccaaacttccccagtatggactgagaggggttggctgatgtgtaggaacattaagtcgttgagtaaggaccttacgaatgtcaattcgtgcacgggctccacaattgaatatactcactggttaaaagccgtggggaagtgcgggtcaatactcaatgctggtgtgcctatatttcagtcctttcacaacatgctggaaaggcttggtactaactctcgtatagatcgcggggtgttctttaaatctgggctagtcaatctcattcgtgggatggacagacaacctgacgttaagatcaccacttccgctcgtctttctttcgaagtggcattcgggattacacctggaatgcaattggctattgaacggtactatgactcagtcatgggcccgctgggtaaaatagaaacaactaaatggccaatagaactaagaaaggaatacgagtacggaagcgagtggtacgaagacttaggcgtcctaggatgagcagggacatcggtttaccgatggcccccgttcagaataggatggctagggcttcgttggttagggaaacctatataccttcttccaccacaactggtaaggaggctgtgtgggcaacctggccaattaatgtaggtgtctttccgcaactagccacgatagtaagcaacccgttatactggcgtgtgaccagtgtgcaagtggcaatggagccagcaacgtcaacctctacccaactctgtggtgtaggtctcagtacatctggggcttacaactcaggggaagggttcgggaacacgtttaagctcttgcgctcttgtaactatacgagacgctcacccgtgggagggaacgtgatggtcaagtggcccatcagcatgccctacattcttaatgatgatgcgcacaagaccacaggtctaacagcttgtgtggtcattgctgtcaccaatccaggtgccatcactggccaatcttgggcggagattcaattgaatgtagagtacgtagtgggtacttagacagtctgcgggcttgggaacgacgccccgctagcaacgtactgctctcctaccggactggtagccgagctgttatcttggagcgatagcactgtgagcctcaccctgcgcgcgatggacgtgttgcgtgaccctcacagatttgtgaaactctatagagtagttccgcgagccagaagggaggatggcgacctggcgaaatccaggagctctggggggcttgtactcagagtagcaatactgctttagactgttagtttgatgaaccacgcgtgtcacgtggggagaactaacagcgccc

SEQ ID NO: 121 - RNA, 2496 nt, synthetic construct
  source 1..2496: mol_type = other RNA
  misc_feature 1..2496: note = Recoding Structure Element Region of CYVaV-delta
ccacatcgcgcaggtcgcggcgaccttgtggctaactaagaccttgtctgacaaggccttggcccgccaccagggttttcgcgatttgcagtgattggggtcgacgggctagaggctaaagcagtgcctctagctgcaggactccgactgcttccggttccgcggcccggacaaagccgacggctgtctcagaccttgttactaccaacccctcgtgcacaatttgtgaatcacgcgaactcggctaataaccttgggcgcgtgatgaccacacgagtgatgcagtacaaaggccgagacccgatccaaccctctcaagaagccctaaccaaacttaaccttcggatagccgagctatataagtcgagaccatccaccgtctatccgttgagttatgaagggtttctcaactgctacgaaggccgacagcgtactcgttacgctcaagccgtcgagcagctgttgaggtccactctagaacctaaagatgcgagagttgaaacgttcattaagaacgagaagtttgattgggcgttgaaaggggaggaggctgatcctcgtgcaatccaaccaaggaagccgaaatatctggctgaggttggacggtggtttaaacctttggagcgaatcatctacaaggatcttagtaagcggttgtatggcgagggtggtgaaccttgtattgccaaaggcttaaatgcactagaatctggggcgactttgaggcgcaaatgggagaagttttcttctcctgtttgcgtctctcttgacgcttccaggttcgacctgcatgtaagtgttggcatgcttaagtttacacacaagctgtacgactactactgcaagtctcccaccctccaacgttatctcaaatggacactccgcaatcacggcactgcctcctgtaaagaattgtcatatggttatgaggtagaaggccgaagaatgagtggtgacatggacaccgcattgggcaactgcgtcatcatgtcgatactaacatggtttatgcttagtgagcttggcattaagcatgaattattcgacaatggtgacgactgcttgttcatttgcgagtcaaaagacgtccccagccccgaggtgatcacgaactggttttcggactttgggtttgtggtcaggttggaaggcgtcacgtccgtgtttgagcgtatagagttttgccaaacttccccagtatggactgagaggggttggctgatgtgtaggaacattaagtcgttgagtaaggaccttacgaatgtcaattcgtgcacgggctccacaattgaatatactcactggttaaaagccgtggggaagtgcgggtcaatactcaatgctggtgtgcctatatttcagtcctttcacaacatgctggaaaggcttggtactaactctcgtatagatcgcggggtgttctttaaatctgggctagtcaatctcattcgtgggatggacagacaacctgacgttaagatcaccacttccgctcgtctttctttcgaagtggcattcgggattacacctggaatgcaattggctattgaacggtactatgactcagtcatgggcccgctgggtaaaatagaaacaactaaatggccaatagaactaagaaaggaatacgagtacggaagcgagtggtacgaagacttaggcgtcctaggatgagcagggacatcggtttaccgatggcccccgttcagaataggatggctagggcttcgttggttagggaaacctatataccttcttccaccacaactggtaaggaggctgtgtgggcaacctggccaattaatgtaggtgtctttccgcaactagccacgatagtaagcaacccgttatactggcgtgtgaccagtgtgcaagtggcaatggagccagcaacgtcaacctctacccaactctgtggtgtaggtctcagtacatctggggcttacaactcaggggaagggttcgggaacacgtttaagctcttgcgctcttgtaactatacgagacgctcacccgtgggagggaacgtgatggtcaagtggcccatcagcatgccctacattcttaatgatgatgcgcacaagaccacaggtctaacagcttgtgtggtcattgctgtcaccaatccaggtgccatcactggccaatcttgggcggagattcaattgaatgtagagtacgtagtgggtacttagacagtctgcgggcttgggaacgacgccccgctagcaacgtactgctctcctaccggactggtagccgagctgttatcttggagcgatagcactgtgagcctcaccctgcgcgcgatggacgtgttgcgtgaccctcacagatttgtgaaactctatagagtagttccgcgagccagaagggaggatggcgacctggcgaaatccaggagctctggggggcttgtactcagagtagcaatactgctttagactgttagtttgatgaaccacgcgtgtcacgtggggagaactaacagcgccc

SEQ ID NO: 122 - RNA, 2194 nt, synthetic construct
  source 1..2194: mol_type = other RNA
  misc_feature 1..2194: note = Recoding Structure Element Region of CYVaVRF
aggtcgcggcgaccttgtggttaactaggaccttgcatgacaaggccttggctcgccaccagggttttcgcgatttgcagtgattggggtcgacgggctagaggctaaagcagtgcctctagcttctggactccgactgcttccggttccgcggcccggacaaagccgacgactgtctcagaccttgttacttccaacacctcgtgctcaattcgtgaatcacgcgtgctcggctaacaaccttggacgtgtgatgaccacacgtgtgttgcagtacaagggccgagatccgatccttccctcttctgaagcccttcaccgacttaaccttcggatagctgagctatataggtctagaccttctaccgtctatccattaagttatgaagggtttctcaattgctatgaaggccgacagcgtactcgttacgcccaagccgtcgagcagttgatgcggtccactcttgagccgaaagatgcgcgagttgaaacgttcattaagaacgagaaatttgactgggcgttgaaaggggaggaggctgatcctcgagcaatccaaccaaggaagccgaaatatttggctgaggttggacggtggttcaaacctttggagcgaatcatctacaaggatctcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgtaagtctcccactctccagcgctatctcaaatggacactccgcaaccatggcgtcgcctcctgcaaagaattgtcatatgagtatgaggttgttggccggagaatgagtggtgacatggacactgcattgggcaactgcgtcattatgtcgatacttacatggtttatgcttagtgaacttggcattaagcatgaattattcgataatggtgacgattgtttgttcatttgcgagtctcacgacgtccccagccccgaggtaattacaaactggttttcggactttgggtttgtggttaggttggaaggcgtcacgtccgtgtttgagcgtattgagttttgccaaacttccccagtatggactgagaggggttggctgatgtgtaggaatattaagtcattgagtaaagaccttacgaatgttaattcgtgcacgggctccacgattgaatatacccactggttgaaagcagtgggaaagtgcgggtcaatactcaatgctggtgtacctatatttcagtcctttcacaacatgctggaaaggcttggcactaactctcgtattgatcgaggggttttcttcaaatcagggctagttaatctcattcgtgggatggacaggcagcctgacgttgacatcactacttccgctcggctttctttcgaagtggcattcgggataacacccgggatgcaattggctattgaacggtactatgactctgtcatgggctcgctgagtaaaatagaaacaactaagtggccaattgaactaagaaaggaatacgaacacggaagtgagtggtacgaggacttaggcgtcctaggatgaatagggtcattggtttaccgatgatacctgttcagaataggattgctcgagcttcgttggttagggtaactcacataccttcttccataactggaaaaggtcgtgtgagcaacctaaccagttaatgtaggtgtctttccgtatctagtcacgatggtaagcaacccgtttatctgtacggcgctcacccgtgggtaggaaggtgaaggttttgtgtcctttaggtcttggacagtctgcgggcttgggaacgacgccccgctagcaacgtactgctctcctaccggactggtagcttaattgtcatcttggagcgatagcactgtgggcctcacccttcgcgcgttggacgtgttgcgtgccccccacagatttgtgaaactctatggagcagttccgcgagccagaagggaggatggccgcctggcgtaatccaggagctctggggggcttgtactcagagtagcatactgctttagactgttaactttatgaaccacgcgtgtcacgtggggagagttaacagcgccc

SEQ ID NO: 123 - RNA, 2205 nt, synthetic construct
  source 1..2205: mol_type = other RNA
  misc_feature 1..2205: note = Recoding Structure Element Region of CYVaVRF
ccatatcgctcaggtcgcggcgaccttgtggttaactaggaccttgcatgacaaggccttggctcgccaccagggttttcgcgatttgcagtgattggggtcgacgggctagaggctaaagcagtgcctctagcttctggactccgactgcttccggttccgcggcccggacaaagccgacgactgtctcagaccttgttacttccaacacctcgtgctcaattcgtgaatcacgcgtgctcggctaacaaccttggacgtgtgatgaccacacgtgtgttgcagtacaagggccgagatccgatccttccctcttctgaagcccttcaccgacttaaccttcggatagctgagctatataggtctagaccttctaccgtctatccattaagttatgaagggtttctcaattgctatgaaggccgacagcgtactcgttacgcccaagccgtcgagcagttgatgcggtccactcttgagccgaaagatgcgcgagttgaaacgttcattaagaacgagaaatttgactgggcgttgaaaggggaggaggctgatcctcgagcaatccaaccaaggaagccgaaatatttggctgaggttggacggtggttcaaacctttggagcgaatcatctacaaggatctcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgtaagtctcccactctccagcgctatctcaaatggacactccgcaaccatggcgtcgcctcctgcaaagaattgtcatatgagtatgaggttgttggccggagaatgagtggtgacatggacactgcattgggcaactgcgtcattatgtcgatacttacatggtttatgcttagtgaacttggcattaagcatgaattattcgataatggtgacgattgtttgttcatttgcgagtctcacgacgtccccagccccgaggtaattacaaactggttttcggactttgggtttgtggttaggttggaaggcgtcacgtccgtgtttgagcgtattgagttttgccaaacttccccagtatggactgagaggggttggctgatgtgtaggaatattaagtcattgagtaaagaccttacgaatgttaattcgtgcacgggctccacgattgaatatacccactggttgaaagcagtgggaaagtgcgggtcaatactcaatgctggtgtacctatatttcagtcctttcacaacatgctggaaaggcttggcactaactctcgtattgatcgaggggttttcttcaaatcagggctagttaatctcattcgtgggatggacaggcagcctgacgttgacatcactacttccgctcggctttctttcgaagtggcattcgggataacacccgggatgcaattggctattgaacggtactatgactctgtcatgggctcgctgagtaaaatagaaacaactaagtggccaattgaactaagaaaggaatacgaacacggaagtgagtggtacgaggacttaggcgtcctaggatgaatagggtcattggtttaccgatgatacctgttcagaataggattgctcgagcttcgttggttagggtaactcacataccttcttccataactggaaaaggtcgtgtgagcaacctaaccagttaatgtaggtgtctttccgtatctagtcacgatggtaagcaacccgtttatctgtacggcgctcacccgtgggtaggaaggtgaaggttttgtgtcctttaggtcttggacagtctgcgggcttgggaacgacgccccgctagcaacgtactgctctcctaccggactggtagcttaattgtcatcttggagcgatagcactgtgggcctcacccttcgcgcgttggacgtgttgcgtgccccccacagatttgtgaaactctatggagcagttccgcgagccagaagggaggatggccgcctggcgtaatccaggagctctggggggcttgtactcagagtagcatactgctttagactgttaactttatgaaccacgcgtgtcacgtggggagagttaacagcgccc

SEQ ID NO: 124 - RNA, 198 nt, synthetic construct
  source 1..198: mol_type = other RNA
  misc_feature 1..198: note = Wt:29/44
cagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactg

SEQ ID NO: 125 - RNA, 80 nt, unidentified
  source 1..80: mol_type = viral cRNA; note = Citrus yellow vein-associated virus [CYVaV]
  misc_feature 1..80: note = CY1132-1329 (structure 1)
cagtaaaaggttgtatggtgagggtgctgagccgtgtatcgccaaaggcctaaatgcattagaatctggagcgactttga

SEQ ID NO: 126 - RNA, 33 nt, synthetic construct
  source 1..33: mol_type = other RNA
  misc_feature 1..33: note = CY1493-1525 (structure 2)
tttatgcttagtgaacttggcattaagcatgaa

SEQ ID NO: 127 - RNA, 80 nt, synthetic construct
  source 1..80: mol_type = other RNA
  misc_feature 1..80: note = CY2136-2218 (strucuture 3)
acgaggacttaggcgtcctaggatgaatagggtcattggtttaccgatgatacctgttcagaataggattgctcgagctt

SEQ ID NO: 128 - RNA, 61 nt, synthetic construct; identical to SEQ ID NO: 72
  source 1..61: mol_type = other RNA
  misc_feature 1..61: note = CY2220-2280 (structure 4)

SEQ ID NO: 129 - RNA, 61 nt, synthetic construct
  source 1..61: mol_type = other RNA
  misc_feature 1..61: note = Mmck6.2
ggtttcagtatatttcgtcatcaccttcctaacggaagtgatggacgaaattaatgaaacc

SEQ ID NO: 130 - RNA, 75 nt, synthetic construct
  source 1..75: mol_type = other RNA
  misc_feature 1..75: note = Mmck6.2
ttcgttggtttcagtatatttcgtcatcaccttcctaacggaagtgatggacgaaattaatgaaaccagttaatg

SEQ ID NO: 131 - RNA, 80 nt, synthetic construct
  source 1..80: mol_type = other RNA
  misc_feature 1..80: note = Mmck6.2
agcttcgttggtttcagtatatttcgtcatcaccttcctaacggaagtgatggacgaaattaatgaaaccagttaatgta

SEQ ID NO: 132 - RNA, 198 nt, synthetic construct
  source 1..198: mol_type = other RNA
  misc_feature 1..198: note = BBLv2-1
ctggaaaattcgattactcaaagtgcctaaaatcgtaacaacttgtatgatctataggcctggatccaaacaatacgtggctcaaatacttgagaaatttttttctcatgtatttgagcctaaaccttgatttggatccatctcctatcgaacaccaagtgttaaactttttagcagagggagtaatcgaaattccag

SEQ ID NO: 133 - RNA, 37 nt, synthetic construct
  source 1..37: mol_type = other RNA
  misc_feature 1..37: note = BBLv2-1
ctggaaaattcgattactcaaagtgtcgaaattccag

SEQ ID NO: 134 - RNA, 743 nt, Erwinia amylovora
  source 1..743: mol_type = transcribed RNA
  misc_feature 1..743: note = Polynucleotide Encoding Erwinia amylovora MurA
gaactggtgaaaaccatgcgcgcctcgatttgggcattgggcccgctggtggcacgttttggccaggggcaagtatcactgcccggtggttgcgctatcggcgcacggccggttgatcttcatatcaccggccttgagcagctcggcgccgagatcaaactggaagaaggttacgttaaagcctctgtcgcgggtcgcctgaaaggggcgcatatcgttatggataaggtcagcgtgggtgcaaccgtcactatcatgagtgcggcgacgctggcaacgggcaccaccgttatcgagaatgctgcgcgtgagccggaaattgtcgacactgccaacttcctcaacacgcttggggcgaaaatcaccggtgccggcagcgatcgtatcaccatcgaaggtgttgatcgccttggtggcggtgtttatcgcgtacttcctgaccgcatcgaaaccggtactttcctggtggcgggagcgatttccggcggtaaggttacctgccgtgcggcgcagcccgatacgctggatgctgtactggctaagctacgcgaagccggtgcggacatcgagatgggagaagactggataagcctggacatgcacggtaagcggcctaaagcggtcaatttacgcacagcgccgcatcccggtttcccaaccgatatgcaggcgcagttcagtttgttgaacctggtggctgaaggcacgggggtgattactgaaaccatcttcgaaaaccgctt

SEQ ID NO: 135 - RNA, 607 nt, Erwinia amylovora
  source 1..607: mol_type = transcribed RNA
  misc_feature 1..607: note = Polynucleotide Encoding Erwinia amylovora GyrA
atcgtcaacctgctgccgctggaagccaacgagcgtattactgcaattctgccggtgcgtgaatatgccgaaggctggaatatctttatggctaccgccagcggaacggtgaagaaaaccgcgctgactgacttcagccgaccgcgcagcgccggtattattgccgtcaatctgcgtgatgacgatgaactgatcggcgtgtcgctgacgaacggtagtgatgaagcgatgctgttttctgccgccggtaaagtggtgcgattcgcggagagcgcggtgcgtacgatgggccgtaccgcctccggcgtacgcggcatcaagctggctgaaggcgaccgcgtggtgtcgctgatcgtgccgcgtgatgatggcgctatcatgaccgtgacgcaaaacggctacggtaaacgaacggctaacgtcgaatatcccaccaagtcgcgtgcgactcagggggttatctcgatcaaggtaaccgagcgtaacgggccggttatcggtgcggtgcaggtggtcgatggcgatcagatcatgatgatcaccgatgccggcacgctggtacgtacccgcgtgtccgaggtcagcgtggtagggcgtaacacccagg

SEQ ID NO: 136 - RNA, 121 nt, synthetic construct
  source 1..121: mol_type = other RNA
  misc_feature 1..121: note = LD1pstADK327-356sh Insert Targeting GyrA
gcgatatggattcagggactcgccgttgatgacgaagaaatcgtcaagcgcgaacgcttgacgatttcttcgtcatcaacggcgagtccctgctcaggggaaactttgtgtcctaagtcgc

SEQ ID NO: 137 - RNA, 121 nt, synthetic construct
  source 1..121: mol_type = other RNA
  misc_feature 1..121: note = LD1pstGYR3-34sh Insert Targeting Adenylate Kinase
gcgatatggattcagggactgggcgaactggccaaagaaatcctcccggtcgaaaccgggaggatttctttggccagttcgcccagtccctgctcaggggaaactttgtgtcctaagtcgc

SEQ ID NO: 138 - RNA, 2808 nt, Pseudomonas syringae
  source 1..2808: mol_type = transcribed RNA
  misc_feature 1..2808: note = P. syringae tabaci_Gyrase subunit A (Pst_GY)
atgggcgaactggccaaagaaatcctcccggtcaatatcgaagacgagctgaagcagtcctacctcgactacgcgatgagcgtaatcgtcggtcgagcactgcccgatgcgcgcgacggcttgaagcccgtgcaccggcgcgtgttgttcgcaatgagcgagctgggtaacgactggaacaagccgtacaagaaatccgcccgtgtggttggtgacgtgatcggtaagtatcacccgcacggcgatacagccgtgtacgacaccatcgttcgtatggctcagccattctcgctacgctacctgctggtagacggtcagggcaacttcggttcggtcgatggcgacaacgctgcggccatgcgatacaccgaagtgcgcatgaccaagctggcgcacgagctgctggccgacctgcacaaggaaaccgtggactgggtgccgaactacgacggcaccgaaatgatccccgcggtcatgccgacccgtattcccaacctgctggtcaacggttccagcggtatcgccgtgggcatggcgaccaacattccgccgcacaaccttggtgaagtcatcgacggttgcctggcactgatcgacaaccccgagttgacgatcgatgagctgatgcagtacatccccggcccggatttcccgacagcggcgatcatcaacggccgtgcgggtatcatcgaagcctatcgtacgggtcgtggacgcatttacatgcgtgcccgctccatcgtcgaagacatcgacaaggtcggcggacgtcagcagatcgtcatcaccgaactgccgtaccagcttaacaaggcacgtctgatcgagaagatcgccgagctggtcaaggaaaagaaactcgaaggcattaccgagctgcgtgacgagtccgacaaggacggtatgcgcgtggtcatcgagctgcgtcgtggcgaagtgccagaggttgttctcaacaacctttatgcccagacccagctgcaaagcgttttcggtatcaacatcgttgccttgattgacggtcgtccacgcatcctgaacctcaaggacctgttggaagcctttgttcgtcaccgtcgcgaagtggttacccgccgtaccgtattcgagctgcgcaaggcgcgtgagcgtggccatatccttgaaggtcaggctgttgcgttgtccaacatcgacccggtcatcgccctcatcaaggcctctccgacacctgcagaagccaaggaggcgttgatcaagacgccttgggaatcaagcatgcgcgtgattctgctaggagcagtggtcgaaatggtcgagcgtgcaggtgccgattcgtgccgccctgagaacctggacccgcaatacggtctgcgtgaaggcaagtatttcctgtcaccggaacaggctcaggccattctggaactgcgtctgcatcgcctgaccggtctggaacacgagaagctgctgggcgagtaccaggaaatcctcaaccagatcggcgagctgatccgcatcctcaacagcgcaacgcgcctgatggaagtgatccgcgaagagctggaagtgatccgctccgagtacggcgatgcccgtcgtaccgagattctcgatgcacgtctggacctgaccctgggtgacctgatcaccgaagaagagcgtgtggtcaccatctcccatggcggctatgccaagacccagccattggcggtctatcaggctcagcgtcgtggcggcaagggcaagtcggccaccggcatcaaggatgaggattacattgctcacctgctggtcgccaacagccacacgacactgctgatgttctccagcaagggcaaggtgtactggctcaagacctatgagatcccggaagcgtcccgcgctgcccgtggtcgtccgttggtcaacctgttgccgctgagcgacggcgaatacatcaccaccatgctgccggttgacctcgaagccatgcgcaagcgtgccgacgaagaaggcgaagccctcgaaggcgagctggacgacgcggaaaacagcagcgagaccgaagaagagcgcaaggcccgtatcaaggccgccgacaagaagaaggctccgttcatcttcatgtccaccgccaacggtaccgtcaagaagaccccgctggttgcattcagccgtcaacgcagttcgggcctcattgcccttgagctggacgagggcgacatcctgatctccgctgccattaccgatggcgaacaggaaatcatgctgttctccgatggcggcaaagtgacccgcttcaaggaatccgatgtgcgcgccatggggcgtaccgctcgcggcgtgcgtggcatgcgtctgccagaagggcaaaagctcatttcgatgctgatcccggaagaaggcagccagatcctcaccgcttccgagcgcggttacggcaagcgtacggccatttccgagttccccgagtacaagcgcggcggtcagggtgtcatcgccatggtcagcaacgagcgtaacggccgtctggttggcgcagttcaggtgcttgatggcgaagaaatcatgctgatttccgatcagggcacgctggtgcgtacccgggtgggcgaagtgtccagtctgggccgtaacactcagggtgtgaccctgatcaagctggccagcgacgagaaactggtcggtctggagcgtgttcaggagccgtcggaagtcgaaggcgaagagcttgaaggcgaagaagttatcgacggcgtgattgtcgatgccgctgaagctgaagtgggcgacgccggtgaagacctgcaagcggacgctgcgccagacgaagacgaaccgcagaactga

SEQ ID NO: 139 - RNA, 663 nt, Pseudomonas syringae
  source 1..663: mol_type = transcribed RNA
  misc_feature 1..663: note = P. syringae tabaci_Adenylate Kinase (Pst-ADK)
atgcgcgtgattctgctaggagctcccggggccggtaaaggtactcaggcaaaattcatcactgaaaatttcggcatcccgcaggtttcgacaggcgacatgctgcgcgctgcagtcaaggctgaaaccgagcttggcctgaaggccaagagcgtcatggactcgggtggtctggtttccgatgacctgatcattggtctgatcaaggatcgtctggcccagccggattgtgcgaacggcgttctgttcgacggcttcccgcgcaccattcctcaggccgaagccctgttgaaagcaggtctggaaatcgaccacgtgctggaaatcgccgttgatgacgaagaaatcgtcaagcgcatgtcgggccgccgggttcacgaaggctctggtcgcatctatcacaccattttcaacccgccgaaagtcgagggtatcgatgatgtgactggtgaaccgctgttgcagcgcaaggacgacgtcgaagaaaccgtgcgtcatcgcctgtcggtctaccatgcccagaccaagccgctggtcgagttctacagcaagctggaagcaaagaacggcaagcccaagtgcagccatattccaggtgttggctcggttgaagaaatcactgcaaaagtactgaaagcactggagcctgaagcacagaagtaa

SEQ ID NO: 140 - RNA, 717 nt, unidentified
  source 1..717: mol_type = transcribed RNA
  misc_feature 1..717: note = GFPuv
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatcagttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataa